SEQUENCE LISTING (67 sequences)

SEQ ID NO: 1 - protein, 593 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 2 - DNA, 1779 bp, synthetic construct
  source 1..1779: mol_type = other DNA
agcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggcctacagagtggacaagagcccctacaatcagagcatgtactacgccggcctgcccgtggacgacttcgaggacagcgagagcaccgacaccgaggaggagttcggcaacgccatcggcggcagccacggaggcagcagctacaccgtgtacatcgacaagacaaga

SEQ ID NO: 3 - protein, 543 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAARVDK

SEQ ID NO: 4 - DNA, 1629 bp, synthetic construct
  source 1..1629: mol_type = other DNA
agcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaag

SEQ ID NO: 5 - DNA, 1629 bp, synthetic construct
  source 1..1629: mol_type = other DNA
agcgtgctgcggtacgacgacttccacatcgacgaggataagctggataccaactctgtgtacgagccctactaccattctgatcatgccgaaagcagctgggtcaatagaggcgagtctagcagaaaggcctatgaccacaacagcccttacatctggccacggaacgactacgacggctttctggaaaacgcccacgagcaccacggcgtttacaaccagggcagaggaatcgatagcggagaaagactgatgcagcctacacaaatgagcgcccaggaggacctcggcgacgataccggcatccacgtgattcctacactgaatggagatgatagacacaagatcgtgaatgtggatcagagacagtacggcgatgtgttcaagggcgacctgaaccctaagccccagggccagagactgattgaagtgtccgtcgaggaaaaccaccccttcaccctgagagcccccatccaaagaatctacggcgtgcggtatacagagacatggtccttcctgcctagcctgacctgtaccggcgacgccgctcctgccatccagcacatctgcctgaaacacaccacctgcttccaggacgtggtggtcgatgtggactgcgccgagaataccaaagaggaccagctggctgagatcagctacagattccaaggcaagaaagaggcggatcagccttggatcgtggtgaacaccagcacactgtttgatgaactggaactggacccccccgagatcgagcctggcgtgctgaaagtgctccgcaccgagaagcagtacctgggcgtgtatatctggaacatgcggggcagcgacggaaccagcacctacgccacattcctggtgacctggaagggcgacgagaagaccagaaaccccacccctgccgtgacaccccagcccagaggcgccgagttccacatgtggaattaccacagccacgtgttcagcgtgggcgacacatttagcctggccatgcacctgcagtacaagatccacgaggccccttttgacctgctgctggaatggctgtacgtgccaatcgaccccacctgtcagcctatgaggctgtacagcacctgtctgtaccaccctaacgctcctcagtgcctgtcccacatgaacagcggatgtaccttcaccagcccccacctggcccagcgggtggcctctaccgtgtaccagaactgcgagcacgccgataactacacagcctactgcctgggcatcagccacatggaacctagcttcggcctgatcctgcacgacggcggcacaacactgaagttcgtggacacccctgagtcactgtctggcctgtacgtgttcgtggtatacttcaacggccacgtggaagccgtggcctacaccgtcgtttccaccgtggaccatttcgtgaacgccatcgaggaaagaggatttcctcctacagccggccagcctccagctacaaccaagcctaaagaaatcacacctgtgaaccctggaacatccccactgattagatacgccgcttggaccggcgggctggccgccgtggtcctgctgtgcctggtgatcttccttatctgcaccgcaaagcggatgcgggttaaggccgccagagtggacaag

SEQ ID NO: 6 - protein, 556 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAARVDKSPYNQSMYGAGLP

SEQ ID NO: 7 - DNA, 1668 bp, synthetic construct
  source 1..1668: mol_type = other DNA
agcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaagagcccctacaatcagagcatgtacggcgccggcctgccc

SEQ ID NO: 8 - protein, 593 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAARVDKSPYNQSMYGAGLPVDDFEDSESTDTEEEFGNAIGGSHGGSSYTVYIDKTR

SEQ ID NO: 9 - DNA, 1779 bp, synthetic construct
  source 1..1779: mol_type = other DNA
agcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaagagcccctacaatcagagcatgtacggcgccggcctgcccgtggacgacttcgaggacagcgagagcaccgacaccgaggaggagttcggcaacgccatcggcggcagccacggaggcagcagctacaccgtgtacatcgacaagacaaga

SEQ ID NO: 10 - protein, 593 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAARVDKSPYNQSMYGAGLPVDDFEDAEAADAEEEFGNAIGGSHGGSSYTVYIDKTR

SEQ ID NO: 11 - DNA, 1779 bp, synthetic construct
  source 1..1779: mol_type = other DNA
agcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccacccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccacccaaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaagagcccctacaatcagagcatgtacggcgccggcctgcccgtggacgacttcgaggacgccgaggccgccgacgccgaggaggagttcggcaacgccatcggcggcagccacggaggcagcagctacaccgtgtacatcgacaagacaaga

SEQ ID NO: 12 - protein, 593 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYGAGLPVDDFEDAEAADAEEEFGNAIGGSHGGSSYTVYIDKTR

SEQ ID NO: 13 - DNA, 1779 bp, synthetic construct
  source 1..1779: mol_type = other DNA
agcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggcctacagagtggacaagagcccctacaatcagagcatgtacggcgccggcctgcccgtggacgacttcgaggacgccgaggccgccgacgccgaggaggagttcggcaacgccatcggcggcagccacggaggcagcagctacaccgtgtacatcgacaagacaaga

SEQ ID NO: 14 - protein, 8 aa, synthetic construct
GGGSGGGS

SEQ ID NO: 15 - DNA, 24 bp, synthetic construct
  source 1..24: mol_type = other DNA
ggaggaggaagtggaggaggaagt

SEQ ID NO: 16 - protein, 28 aa, synthetic construct
GYIPEAPRDGQAYVRKDGEWVLLSTFLG

SEQ ID NO: 17 - DNA, 84 bp, synthetic construct
  source 1..84: mol_type = other DNA
ggctatattccggaagcgccgcgcgatggccaggcgtatgtgcgcaaagatggcgaatgggtgctgctgagcacctttctgggc

SEQ ID NO: 18 - protein, 30 aa, synthetic construct
MGTVNKPVVGVLMGFGIITGTLRITNPVRA

SEQ ID NO: 19 - DNA, 90 bp, synthetic construct
  source 1..90: mol_type = other DNA
atgggcaccgtgaacaagcccgtggtgggcgtgctgatgggcttcggcatcatcaccggcaccctgagaatcaccaaccccgtgagagct

SEQ ID NO: 20 - DNA, 90 bp, synthetic construct
  source 1..90: mol_type = other DNA
atggggacagttaataaacctgtggtgggggtattgatggggttcggaattatcacgggaacgttgcgtataacgaatccggtcagagca

SEQ ID NO: 21 - DNA, 90 bp, synthetic construct
  source 1..90: mol_type = other DNA
atgggaaccgtgaacaagcctgtggttggagttctgatgggctttggcatcatcacaggaacactcagaatcaccaacccagtgcgggcc

SEQ ID NO: 22 - DNA, 90 bp, synthetic construct
  source 1..90: mol_type = other DNA
atgggcacggtgaacaagcccgtggtcggggtgctgatggggtttgggatcatcacagggactctgcgtatcacgaacccagtgcgtgcc

SEQ ID NO: 23 - protein, 18 aa, synthetic construct
MDWTWILFLVAAATRVHS

SEQ ID NO: 24 - DNA, 54 bp, synthetic construct
  source 1..54: mol_type = other DNA
atggactggacctggattctcttcttggtggcagcagccacgcgagtccactcc

SEQ ID NO: 25 - DNA, 54 bp, synthetic construct
  source 1..54: mol_type = other DNA
atggactggacctggattctgttcctggtggccgccgccacaagagtgcacagc

SEQ ID NO: 26 - DNA, 54 bp, synthetic construct
  source 1..54: mol_type = other DNA
atggactggacctggattctgttcctggtggctgccgctacccgggtgcacagc

SEQ ID NO: 27 - protein, 23 aa, synthetic construct
MDAMKRGLCCVLLLCGAVFVSNS

SEQ ID NO: 28 - DNA, 69 bp, synthetic construct
  source 1..69: mol_type = other DNA
atggacgccatgaagagaggcctgtgctgcgtgctgctgctgtgcggcgccgtgttcgtgagcaacagc

SEQ ID NO: 29 - DNA, 39 bp, synthetic construct
  source 1..39: mol_type = other DNA
gaaataagagagaaaagaagagtaagaagaaatataaga

SEQ ID NO: 30 - RNA, 39 nt, synthetic construct
  source 1..39: mol_type = other RNA
gaaataagagagaaaagaagagtaagaagaaatataaga

SEQ ID NO: 31 - DNA, 43 bp, synthetic construct
  source 1..43: mol_type = other DNA
cttgttctttttgcagaagctcagaataaacgctcaactttgg

SEQ ID NO: 32 - RNA, 43 nt, synthetic construct
  source 1..43: mol_type = other RNA
cttgttctttttgcagaagctcagaataaacgctcaactttgg

SEQ ID NO: 33 - DNA, 100 bp, synthetic construct
  source 1..100: mol_type = other DNA
gcaggagccagggctgggcataaaagtcagggcagagccatctattgcttacatttgcttctgacacaactgtgttcactagcaacctcaaacagacacc

SEQ ID NO: 34 - RNA, 100 nt, synthetic construct
  source 1..100: mol_type = other RNA
gcaggagccagggctgggcataaaagtcagggcagagccatctattgcttacatttgcttctgacacaactgtgttcactagcaacctcaaacagacacc

SEQ ID NO: 35 - DNA, 137 bp, synthetic construct
  source 1..137: mol_type = other DNA
agctctcaacacaacatatacaaaacaaacgaatctcaagcaatcaagcattctacttctattgcagcaatttaaatcatttcttttaaagcaaaagcaattttctgaaaattttcaccatttacgaacgatagccc

SEQ ID NO: 36 - RNA, 137 nt, synthetic construct
  source 1..137: mol_type = other RNA
agctctcaacacaacatatacaaaacaaacgaatctcaagcaatcaagcattctacttctattgcagcaatttaaatcatttcttttaaagcaaaagcaattttctgaaaattttcaccatttacgaacgatagccc

SEQ ID NO: 37 - DNA, 44 bp, synthetic construct
  source 1..44: mol_type = other DNA
cttgttctttttgcagaagctcagaataaacgctcaactttggc

SEQ ID NO: 38 - RNA, 44 nt, synthetic construct
  source 1..44: mol_type = other RNA
cttgttctttttgcagaagctcagaataaacgctcaactttggc

SEQ ID NO: 39 - DNA, 110 bp, synthetic construct
  source 1..110: mol_type = other DNA
gctggagcctcggtggccatgcttcttgccccttgggcctccccccagcccctcctccccttcctgcacccgtacccccgtggtctttgaataaagtctgagtgggcggc

SEQ ID NO: 40 - RNA, 110 nt, synthetic construct
  source 1..110: mol_type = other RNA
gctggagcctcggtggccatgcttcttgccccttgggcctccccccagcccctcctccccttcctgcacccgtacccccgtggtctttgaataaagtctgagtgggcggc

SEQ ID NO: 41 - DNA, 132 bp, synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 42 - RNA, 132 nt, synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 43 - DNA, 278 bp, synthetic construct
  source 1..278: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 44 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 45 - DNA, 110 bp, synthetic construct
  source 1..110: mol_type = other DNA
gctggagcctcggtggccatgcttcttgccccttgggcctcccccaagcccctcctccccttcctgcacccgtacccccgtggtctttgaataaagtctgagtgggcggc

SEQ ID NO: 46 - RNA, 110 nt, synthetic construct
  source 1..110: mol_type = other RNA
gctggagcctcggtggccatgcttcttgccccttgggcctcccccaagcccctcctccccttcctgcacccgtacccccgtggtctttgaataaagtctgagtgggcggc

SEQ ID NO: 47 - DNA, 24 bp, synthetic construct
  source 1..24: mol_type = other DNA
caaaggctcttttcagagccacca

SEQ ID NO: 48 - RNA, 24 nt, synthetic construct
  source 1..24: mol_type = other RNA
caaaggctcttttcagagccacca

SEQ ID NO: 49 - DNA, 2005 bp, synthetic construct
  source 1..2005: mol_type = other DNA
ggatccaagcttagcttgcttgttctttttgcagaagctcagaataaacgctcaactttggcgccgccaccatggactggacctggattctgttcctggtggccgccgccacaagagtgcacagcagcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaagtgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 50 - DNA, 2005 bp, synthetic construct
  source 1..2005: mol_type = other DNA
ggatccaagcttagcttgcttgttctttttgcagaagctcagaataaacgctcaactttggcgccgccaccatggactggacctggattctgttcctggtggctgccgctacccgggtgcacagcagcgtgctgcggtacgacgacttccacatcgacgaggataagctggataccaactctgtgtacgagccctactaccattctgatcatgccgaaagcagctgggtcaatagaggcgagtctagcagaaaggcctatgaccacaacagcccttacatctggccacggaacgactacgacggctttctggaaaacgcccacgagcaccacggcgtttacaaccagggcagaggaatcgatagcggagaaagactgatgcagcctacacaaatgagcgcccaggaggacctcggcgacgataccggcatccacgtgattcctacactgaatggagatgatagacacaagatcgtgaatgtggatcagagacagtacggcgatgtgttcaagggcgacctgaaccctaagccccagggccagagactgattgaagtgtccgtcgaggaaaaccaccccttcaccctgagagcccccatccaaagaatctacggcgtgcggtatacagagacatggtccttcctgcctagcctgacctgtaccggcgacgccgctcctgccatccagcacatctgcctgaaacacaccacctgcttccaggacgtggtggtcgatgtggactgcgccgagaataccaaagaggaccagctggctgagatcagctacagattccaaggcaagaaagaggcggatcagccttggatcgtggtgaacaccagcacactgtttgatgaactggaactggacccccccgagatcgagcctggcgtgctgaaagtgctccgcaccgagaagcagtacctgggcgtgtatatctggaacatgcggggcagcgacggaaccagcacctacgccacattcctggtgacctggaagggcgacgagaagaccagaaaccccacccctgccgtgacaccccagcccagaggcgccgagttccacatgtggaattaccacagccacgtgttcagcgtgggcgacacatttagcctggccatgcacctgcagtacaagatccacgaggccccttttgacctgctgctggaatggctgtacgtgccaatcgaccccacctgtcagcctatgaggctgtacagcacctgtctgtaccaccctaacgctcctcagtgcctgtcccacatgaacagcggatgtaccttcaccagcccccacctggcccagcgggtggcctctaccgtgtaccagaactgcgagcacgccgataactacacagcctactgcctgggcatcagccacatggaacctagcttcggcctgatcctgcacgacggcggcacaacactgaagttcgtggacacccctgagtcactgtctggcctgtacgtgttcgtggtatacttcaacggccacgtggaagccgtggcctacaccgtcgtttccaccgtggaccatttcgtgaacgccatcgaggaaagaggatttcctcctacagccggccagcctccagctacaaccaagcctaaagaaatcacacctgtgaaccctggaacatccccactgattagatacgccgcttggaccggcgggctggccgccgtggtcctgctgtgcctggtgatcttccttatctgcaccgcaaagcggatgcgggttaaggccgccagagtggacaagtgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - DNA, 2044 bp, synthetic construct
  source 1..2044: mol_type = other DNA
ggatccaagcttagcttgcttgttctttttgcagaagctcagaataaacgctcaactttggcgccgccaccatggactggacctggattctgttcctggtggccgccgccacaagagtgcacagcagcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaagagcccctacaatcagagcatgtacggcgccggcctgccctgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - DNA, 2155 bp, synthetic construct
  source 1..2155: mol_type = other DNA
ggatccaagcttagcttgcttgttctttttgcagaagctcagaataaacgctcaactttggcgccgccaccatggactggacctggattctgttcctggtggccgccgccacaagagtgcacagcagcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaagagcccctacaatcagagcatgtacggcgccggcctgcccgtggacgacttcgaggacagcgagagcaccgacaccgaggaggagttcggcaacgccatcggcggcagccacggaggcagcagctacaccgtgtacatcgacaagacaagatgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 53 - DNA, 2155 bp, synthetic construct
  source 1..2155: mol_type = other DNA
ggatccaagcttagcttgcttgttctttttgcagaagctcagaataaacgctcaactttggcgccgccaccatggactggacctggattctgttcctggtggccgccgccacaagagtgcacagcagcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccacccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccacccaaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggccgccagagtggacaagagcccctacaatcagagcatgtacggcgccggcctgcccgtggacgacttcgaggacgccgaggccgccgacgccgaggaggagttcggcaacgccatcggcggcagccacggaggcagcagctacaccgtgtacatcgacaagacaagatgataagaattctaggctggagcctcggtggccatgcttcttgccccttgggcctcccccaagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - DNA, 2155 bp, synthetic construct
  source 1..2155: mol_type = other DNA
ggatccaagcttagcttgcttgttctttttgcagaagctcagaataaacgctcaactttggcgccgccaccatggactggacctggattctgttcctggtggccgccgccacaagagtgcacagcagcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggcctacagagtggacaagagcccctacaatcagagcatgtacggcgccggcctgcccgtggacgacttcgaggacgccgaggccgccgacgccgaggaggagttcggcaacgccatcggcggcagccacggaggcagcagctacaccgtgtacatcgacaagacaagatgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 55 - protein, 623 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 56 - DNA, 1869 bp, synthetic construct
  source 1..1869: mol_type = other DNA
atgggcaccgtgaacaagcccgtggtgggcgtgctgatgggcttcggcatcatcaccggcaccctgagaatcaccaaccccgtgagagctagcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggcctacagagtggacaagagcccctacaatcagagcatgtactacgccggcctgcccgtggacgacttcgaggacagcgagagcaccgacaccgaggaggagttcggcaacgccatcggcggcagccacggaggcagcagctacaccgtgtacatcgacaagacaaga

SEQ ID NO: 57 - protein, 611 aa, synthetic construct
MDWTWILFLVAAATRVHSSVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 58 - DNA, 2156 bp, synthetic construct
  source 1..2156: mol_type = other DNA
gggatccaagcttagcttgcttgttctttttgcagaagctcagaataaacgctcaactttggcgccgccaccatggactggacctggattctgttcctggtggccgccgccacaagagtgcacagcagcgtgctgagatacgacgacttccatatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgaaacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcagtgacgccccaacctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgcccctcagtgccttagtcacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcatgacggcggcacgaccttaaagttcgtggacacccccgagagcctgagcggcctgtacgtgttcgtggtgtacttcaacggccacgtggaggccgtggcctacaccgtggtgagcaccgtggaccacttcgtgaacgcaatcgaggagcgcggctttccgccgactgccggtcaaccccccgccaccaccaagcccaaggagatcacccccgtgaatccgggcacaagccctcttatccggtatgccgcctggaccggcggcctggccgccgtggtgctgctgtgcctggtgatcttcctgatctgcaccgccaagagaatgagagtgaaggcctacagagtggacaagagcccctacaatcagagcatgtactacgccggcctgcccgtggacgacttcgaggacagcgagagcaccgacaccgaggaggagttcggcaacgccatcggcggcagccacggaggcagcagctacaccgtgtacatcgacaagacaagatgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - protein, 574 aa, synthetic construct
MDWTWILFLVAAATRVHSSVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAARVDKSPYNQSMYGAGLP

SEQ ID NO: 60 - DNA, 2047 bp, synthetic construct
  source 1..2047: mol_type = other DNA
gggggatccaagcttagcttgcttgttctttttgcagaagctcagaataaacgctcaactttggcgccgccaccatggactggacctggattctgttcctggtggcggccgccacaagggtgcacagcagcgtcctgcgctatgatgactttcacatagacgaggacaagctggataccaactctgtgtacgagccctactaccacagcgaccacgcggagagtagctgggtgaacaggggggagagcagcaggaaggcctacgaccacaattccccctacatctggccccggaacgactacgacggcttcctggagaacgcccacgagcaccacggggtgtacaaccagggccgcgggatcgattccggggagaggctgatgcagcctacccagatgagcgctcaggaggacctgggggatgacactggcatccatgtcatccccaccctgaacggggacgatcggcacaagatcgtgaatgtggatcagaggcagtatggggacgtgttcaagggcgacctgaatcccaagccccaggggcagcgcctgatcgaggtgagcgtggaggagaaccatcccttcacgctgagggccccgatccagcgcatctacggggtgcggtacacggagacttggagcttcctgccaagtctcacgtgtaccggcgacgccgcccccgccatccagcacatctgcctgaagcacaccacgtgttttcaggacgtggtggtggacgtggactgcgctgagaataccaaggaggatcagctggccgagatctcctataggtttcagggtaagaaggaggctgaccagccgtggatcgtggtgaacacctctaccctgttcgatgagctggagctcgaccctccggagatcgagcccggcgtgctgaaggtgctccgcacggagaagcagtacctgggagtctacatatggaacatgcgggggtcggacgggacttctacgtacgccaccttcctggtgacctggaagggcgacgagaagacccggaatccgacccccgccgtgacgccccagcctcggggcgccgagttccatatgtggaactaccacagtcacgtcttctccgtgggagacaccttcagcctggccatgcacctccagtacaagatccacgaggccccctttgatctgctgctggagtggctgtatgtgcccatcgaccctacctgtcagcctatgaggctgtacagcacgtgcctgtaccacccgaacgccccgcagtgtctgtcccacatgaacagcgggtgcacgttcaccagcccgcatctggcccagagggtggcctccaccgtgtaccagaactgtgagcatgctgacaattacactgcttactgtctggggatcagccacatggagcccagcttcgggctgatcctgcacgacggggggaccaccctgaagttcgtggacactcctgagagcctgagcgggctgtacgtgttcgtggtgtacttcaacgggcacgtggaggcggtggcctacaccgtggtgtccacggtggatcacttcgtgaatgccatcgaggagagagggttcccacccacggctggtcagcctcctgctaccacgaagcctaaggagatcacgccagtgaatcctggcacctcccctctgatcaggtacgctgcttggacaggggggctggccgccgtggtgctcctgtgcctggtgatctttctgatctgcaccgccaagcggatgcgggtcaaggcagcccgcgtggacaagagcccgtacaatcagagcatgtacggcgccgggctgccctgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 61 - DNA, 2047 bp, synthetic construct
  source 1..2047: mol_type = other DNA
gggggatccaagcttagcttgcttgttctttttgcagaagctcagaataaacgctcaactttggcgccgccaccatggactggacctggattctgttcctggtggcggccgccacaagggtgcacagcagcgtcctgcggtatgacgactttcatatcgacgaggacaagctggataccaactctgtgtacgagccctactaccacagcgaccacgcggagagtagctgggtgaacaggggggagtcttccaggaaggcctacgaccacaattccccctacatctggccccggaacgactacgacggcttcctggagaacgcccacgagcaccacggggtgtacaaccagggccgcgggatcgattccggggagaggctgatgcagcctacccagatgagcgctcaggaggacctgggggatgacactggcatccatgtcatccccaccctgaacggggacgatcggcacaagatcgtgaatgtggatcagaggcagtacggggacgtgttcaagggggatttgaacccaaagccccaggggcagaggctgattgaggtgagcgtggaggagaaccaccctttcactctgagggcccccatccagaggatctacggggtgcggtacactgaaacctggagcttcctgcccagcctgacctgtaccggcgacgccgctcccgccatccagcacatctgcctgaagcacaccacgtgttttcaggacgtggtggtggacgtggactgcgccgagaataccaaggaggatcagctggcagagatcagctaccggtttcaggggaagaaggaggccgaccagccctggatcgtggtgaacaccagcaccctcttcgatgagctggagctcgatcctcccgagatcgagcccggcgtgctgaaggtgctccgcacggagaagcagtacctgggagtctacatatggaacatgcgggggagcgacgggacttctacgtacgccaccttcctggtgacctggaagggcgacgagaagacccggaatcctacccccgccgtgacgccccagcctcggggcgccgagttccatatgtggaactaccacagtcacgtcttctccgtgggagacaccttcagcctggccatgcacctccagtacaagatccacgaggccccctttgatctgctgctggagtggctgtatgtgcccatcgatcctacctgtcagcctatgaggctgtacagcacctgcctgtaccaccccaacgccccacagtgcctgtcacacatgaacagcgggtgcaccttcaccagcccgcatctggcccagagggtggcctccaccgtgtaccagaactgtgagcatgctgacaattacactgcttactgtctggggatcagccacatggagcccagcttcgggctgatcctgcacgacggggggaccaccctgaagttcgtggacactcctgagagcctgagcgggctgtacgtgttcgtggtgtactttaacgggcacgtggaggccgtggcctacaccgtggtgagcaccgtggatcactttgtgaatgccatcgaggagagggggttccctcccaccgccgggcagcctccagccacgaccaagcccaaggagatcactcctgtgaaccccggcaccagccccctgatccggtacgctgcctggacaggcgggctggctgccgtggtgttgctgtgtctggtgatcttcctgatctgcaccgccaagcggatgcgggtgaaggctgcccgcgtggacaagtctccctacaatcagtccatgtatggcgccgggctcccctgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - DNA, 2053 bp, synthetic construct
  source 1..2053: mol_type = other DNA
gggggatccaagcttagcttgcttgttctttttgcagaagctcagaataaacgctcaactttggcgccgccaccatggactggacctggattctgttcctggtggccgctgccaccagggtgcacagctccgtgctgcggtacgatgattttcacatcgatgaggacaagctcgacaccaacagcgtgtacgagccttactaccactctgaccacgccgagtcctcctgggtgaataggggcgagtcttccaggaaggcctatgaccacaatagcccctacatctggccccggaacgactacgacggcttcctggagaacgcccacgagcaccacggggtgtacaaccagggccgcgggatcgattccggggagaggctgatgcagcctacccagatgtccgcccaggaggacctgggcgacgacactggcatccacgtgatccccaccctgaatggggacgatcgccacaagatcgtgaacgtggatcagaggcagtacggagacgtgtttaagggcgacctgaaccctaagccccaggggcagcggctgatcgaggtgagcgtggaggagaaccatcctttcaccctgcgggcccccattcagaggatctacggggtgcggtacaccgaaacctggagcttcctgcccagcctgacctgtaccggcgacgccgctcccgccatccagcacatctgcctgaagcataccacctgcttccaggacgtggtggtggacgtggactgcgccgagaataccaaggaggatcagctggcagagatcagctaccggtttcaggggaagaaggaggctgaccagccttggatcgtggtgaacaccagcacactgttcgatgagctggagctggacccacccgagatcgagccaggcgtgctgaaggtgctgcgcactgagaagcagtatctgggggtgtacatctggaacatgcgggggagcgatggcaccagcacatacgccaccttcctggtgacctggaagggggacgagaagactcggaaccccacccctgctgtgaccccacagcccaggggcgctgagttccacatgtggaactaccatagccacgtgttcagcgtgggggacaccttctccctggctatgcacctgcagtacaagatccacgaggccccctttgatctgctgctggagtggctgtacgtgcccatcgaccccacctgtcagcctatgaggctgtacagcacctgcctgtaccaccccaatgctcctcagtgcctgagccacatgaacagtggctgcaccttcaccagcccccatctggcccagagggtggcctccaccgtgtaccagaattgcgagcacgccgacaactacaccgcttactgtctggggatcagccacatggagcccagcttcgggctgatcctgcacgacgggggcaccaccctgaagttcgtggacactcccgagtccctgagcggcctgtacgtgttcgtggtgtacttcaacggacacgtggaggccgtggcctacaccgtggtgagcaccgtggatcacttcgtgaatgccatcgaggagagggggtttccccctaccgctggccagcctccagccacaaccaagcccaaggagatcacccccgtgaaccccggcaccagccccctgatccggtacgctgcctggacaggcgggctggctgccgtggtgctgctgtgcctggtgatcttcctgatctgcaccgccaagcggatgagggtgaaggccgccagggtggacaagagcccctacaatcagagcatgtacggggccgggctcccctgataagaattcgaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - DNA, 2047 bp, synthetic construct
  source 1..2047: mol_type = other DNA
gggggatccctgaactgggttaggtgccgctgttgctgctcgtgttgaatctagaaccgtagccagacgccaccatggactggacctggattctgttcctggtggccgccgccaccagggtgcacagctctgtcctgaggtacgacgacttccacatcgatgaggacaagctggacaccaacagcgtgtacgagccctactaccacagcgaccacgccgagtcctcctgggtgaataggggcgagtcttccaggaaggcctatgaccacaatagcccctacatctggccccggaacgactacgacggcttcctggagaacgcccacgagcaccacggggtgtacaaccagggccgcgggatcgattccggggagaggctgatgcagcctacccagatgtccgcccaggaggacctgggcgacgataccggcattcacgtgatccccaccctgaatggggacgatcggcacaagatcgtgaatgtggatcagcgccagtacggcgatgtgttcaagggcgacctgaatcccaagccccagggccagcggctgatcgaggtgagcgtggaggagaatcacccttttaccctgcgggcccccattcagaggatctacggggtgcggtacaccgaaacctggagcttcctgcccagcctgacctgtaccggcgacgccgcccccgccatccagcacatctgcctgaagcataccacctgcttccaggacgtggtggtggacgtggactgcgccgagaataccaaggaggatcagctggcagagatcagctaccggtttcaggggaagaaggaggccgaccagccttggatcgtggtgaacaccagcaccctgttcgacgagctggagctggaccctcccgagatcgagcccggcgtgctgaaggtgctgaggaccgagaagcagtacctgggggtgtacatctggaacatgcgggggagcgatggcaccagcacatacgccaccttcctggtgacctggaagggggacgagaagactcggaaccccacccctgctgtgaccccacagcccaggggcgctgagttccacatgtggaactaccatagccacgtgttcagcgtgggggacaccttctccctggctatgcacctgcagtacaagatccacgaggccccctttgatctgctgctggagtggctgtacgtgcccatcgaccccacctgtcagcctatgaggctgtacagcacctgcctgtaccaccccaacgccccacagtgcctgtcccacatgaacagcgggtgcaccttcaccagcccccacctggcccagagggtggcctccaccgtgtaccagaattgcgagcacgccgacaattacaccgcctactgtctggggatcagccacatggagcccagcttcgggctgatcctgcacgacggggggaccaccctgaagttcgtggacacccctgagagcctgagcgggctgtacgtgttcgtggtgtactttaacgggcacgtggaggccgtggcctacaccgtggtgtccaccgtggaccacttcgtgaacgccatcgaggagaggggcttccctcctaccgccgggcagccccctgccaccaccaagcccaaggagatcacccccgtgaaccccggcaccagccccctgatccggtacgccgcctggacaggcgggctggctgccgtggtgctgctgtgcctggtgatcttcctgatctgcaccgccaagcggatgcgggtgaaggccgccagggtggacaagagcccctacaatcagagcatgtacggggctgggctgccctgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 64 - DNA, 2018 bp, synthetic construct
  source 1..2018: mol_type = other DNA
gggggatccgctggttttggtgatttttgtctgattgcagccaccatggactggacctggattctgttcctggtggccgccgccaccagggtgcacagctctgtgctgcggtacgacgacttccacatcgacgaggacaagctggacaccaactcagtgtacgagccctattaccacagcgaccacgcggagagtagctgggtgaacaggggggagagcagcaggaaggcctacgaccacaattccccctacatctggccccggaacgactacgacggcttcctggagaacgcccacgagcaccacggggtgtacaaccagggccgcgggatcgattccggggagaggctgatgcagcctacccagatgagcgctcaggaggacctgggggatgacactggcatccatgtcatccccaccctgaacggggacgatcggcacaagatcgtgaatgtggatcagaggcagtatggggacgtgttcaagggggatctgaatccaaagccccaggggcagcggctgatcgaggtgtccgtggaggagaaccaccctttcactctgagggcccccatccagaggatctacggggtgcggtacactgaaacctggagcttcctgcccagcctgacctgtaccggcgacgccgctcccgccatccagcacatctgcctgaagcacaccacgtgttttcaggacgtggtggtggacgtggactgcgccgagaataccaaggaggatcagctggcagagatcagctaccggtttcaggggaagaaggaggccgaccagccctggatcgtggtgaacaccagcaccctcttcgatgagctggagctcgatcctcccgagatcgagcccggcgtgctgaaggtgctccgcacagagaagcagtacctgggagtctacatctggaacatgcgggggagcgacgggacttctacatacgccaccttcctggtgacctggaagggcgatgagaagacccggaatcctacccccgccgtgacccctcagcctcggggggccgagttccacatgtggaactaccacagtcacgtcttctctgtgggagacaccttcagcctggccatgcacctccagtacaagatccacgaggccccctttgatctgctgctggagtggctgtatgtgcccatcgatcctacctgtcagcctatgaggctgtacagcacctgcctgtaccaccccaacgccccacagtgcctgtcacacatgaacagcgggtgcaccttcaccagcccgcatctggcccagagggtggcctccaccgtgtaccagaactgtgagcatgctgacaattacactgcttactgtctggggatcagccacatggagcccagcttcgggctgatcctgcacgacggggggaccaccctgaagttcgtggacactcctgagagcctgagcgggctgtacgtgttcgtggtgtactttaacgggcacgtggaggccgtggcctacaccgtggtgagcaccgtggatcactttgtgaatgccatcgaggagagggggttccctcccacagccgggcagccccctgccaccaccaagcccaaggagatcacccccgtgaaccccggcaccagccccctgatccggtacgctgcctggacaggcgggctggctgccgtggtgctgctgtgcctggtgatcttcctgatctgcaccgccaagcggatgcgggtcaaggccgccagggtggacaagagcccctacaatcagagcatgtacggggctgggctgccctgataagaattctaggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - protein, 573 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAARVDK

SEQ ID NO: 66 - DNA, 2014 bp, synthetic construct
  source 1..2014: mol_type = other DNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgatgataataggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - DNA, 2011 bp, synthetic construct
  source 1..2011: mol_type = other DNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcactagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0295035 A1
(43) Pub. Date: Oct. 1, 2026

(54) MUTATED FRAGMENT OF VZV GLYCOPROTEIN E

(71) Applicant: SUZHOU ABOGEN BIOSCIENCES CO., LTD., Suzhou, Jiangsu (CN)

(72) Inventors: Haifeng SONG, Suzhou, Jiangsu (CN); Kai JI, Suzhou, Jiangsu (CN); Xishan LU, Suzhou, Jiangsu (CN)

(21) Appl. No.: 19/474,684

(22) PCT Filed: May 30, 2024
(86) PCT No.: PCT/CN2024/096215

§ 371 Date: Oct. 10, 2025

(30) Foreign Application Priority Data
May 31, 2023 (WO) PCT/CN2023/097333

(51) Int. Cl. A61K 39/25; A61K 9/1271; A61K 9/51; A61K 39/00; A61P 31/22; C07K 14/005

(52) U.S. Cl. CPC A61K 39/25; A61K 9/1271; A61K 9/5123; A61P 31/22; C07K 14/005; A61K 2039/55; A61K 2039/55555; A61K 2039/572; A61K 2039/575; C07K 2319/02; C12N 2710/16722; C12N 2710/16734; C12N 2800/22; C12N 2830/50

(57) ABSTRACT

Provided herein are fragments of the gE protein of varicella-zoster virus and therapeutic nucleic acid molecules for managing, preventing and/or treating a disease or disorder caused by varicella-zoster virus or by infection therewith. Also provided herein are therapeutic compositions, including vaccines and lipid nanoparticles, comprising the therapeutic nucleic acids and related therapeutic methods and uses.

### 1. CROSS REFERENCE TO RELATED APPLICATIONS

This application is a U.S. national stage filing under 35 U.S.C. § 371 of International Patent Application No. PCT/CN2024/096215, filed May 30, 2024, which claims the benefit of International Patent Application No. PCT/CN2023/097333, filed May 31, 2023, the contents of each of which are incorporated by reference herein in their entireties.

### 2. SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14639-045-999_SL.xml”, was created on Oct. 9, 2025, and is 110,019 bytes in size.

### 3. FIELD

The present disclosure generally relates to fragments of the glycoprotein E (gE) protein of varicella-zoster virus (VZV) and nucleic acid molecules that can be used for the management, prevention, and treatment of a disease or disorder caused by VZV or by infection therewith. The present disclosure also relates to lipid-containing compositions, including vaccines, of the nucleic acid molecules.

### 4. BACKGROUND

Varicella zoster virus (VZV), also known as human herpesvirus type 3, is a double stranded DNA virus, belonging to alpha herpes virus. VZV has only one serotype. VZV has a genome comprising 71 genes and encoding 67 different proteins, including 6 glycoproteins, which are now named gE, gB, gH, gI, gC, and gL. Glycoproteins gE, gB and gH are very abundant in infected cells, and also present in the envelop of virions. Antibodies induced by the three main glycoproteins can neutralize the virus. Specific humoral and cellular immunity and cytokines such as interferon play a major role in limiting the spread of VZV and in recovery, wherein specific cellular immunity is especially important.

Zostavax (MSD) is an attenuated virus vaccine. It can reduce the burden by 61.1% (65.5% in people aged 60-69 and 55.4% in people aged 70 or more). It can also reduce the duration of pain and discomfort caused by the virus. Shinrix (GSK) is a subunit vaccine. It comprises gE and AS01B, an adjuvant system for enhancing cellular immune response. It can reach an efficacy of more than 90%.

### 5. SUMMARY

In one aspect, provided herein are fragments of the gE protein of VZV, optionally comprising mutations, e.g., substitutions.

In some embodiments, the fragment comprises a truncation of at least 1 amino acid residue and at most 49 amino acid residues from the C-terminal as compared with the mature gE protein. In some embodiments, the fragment comprises a truncation of 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acid residue(s) from the C-terminal as compared with the mature gE protein. In some embodiments, the fragment comprises a truncation of 11-18 (e.g., 11, 12, 13, 14, 15, 16, 17, or 18) or 34-44 (e.g., 34, 35, 36, 37, 38, 39, 40, 41, 42, 43, or 44) amino acid residues from the C-terminal as compared with the mature gE protein. In some embodiments, the fragment comprises a truncation of 12-16 (e.g., 12, 13, 14, 15, or 16) or 35-39 (e.g., 35, 36, 37, 38, or 39) amino acid residues from the C-terminal as compared with the mature gE protein. In some embodiments, the fragment comprises a truncation of 14 or 37 amino acid residues from the C-terminal as compared with the mature gE protein.

In some embodiments, the fragment comprises the substitution Y569A. In some embodiments, the fragment comprises the substitution Y582G. In some embodiments, the fragment comprises the substitutions Y569A and Y582G. In some embodiments, the fragment comprises the substitution S593A. In some embodiments, the fragment comprises the substitution S595A. In some embodiments, the fragment comprises the substitution T596A. In some embodiments, the fragment comprises the substitution T598A. In some embodiments, the fragment comprises the substitutions S593A, S595A, T596A, and T598A. In some embodiments, the fragment comprises the substitutions Y569A, Y582G, S593A, S595A, T596A, and T598A. In such embodiments, the amino acid positions are numbered on the basis of the full length gE protein.

In some embodiments, the fragment comprises a truncation of 49, 48, 47, 46, 45, 44, 43, or 42 amino acid residues from the C-terminal as compared with the mature gE protein. In such embodiments, the fragment optionally comprises the substitution Y569A.

In some embodiments, the fragment comprises a truncation of 41, 40, 39, 38, 37, 36, 35, 34, 33, 32, or 31 amino acid residues from the C-terminal as compared with the mature gE protein. In such embodiments, the fragment optionally comprises the substitution Y582G, with or without the substitution Y569A. In such embodiments, the fragment comprises the substitutions Y569A and Y582G.

In some embodiments, the fragment comprises a truncation of 30 or 29 amino acid residues from the C-terminal as compared with the mature gE protein. In such embodiments, the fragment optionally comprises the substitution S593A, with or without the substitutions Y569A and/or Y582G. In such embodiments, the fragment comprises the substitutions Y569A, Y582G and S593A. In some embodiments, the fragment comprises a truncation of 28 amino acid residues from the C-terminal as compared with the mature gE protein. In such embodiments, the fragment optionally comprises the substitution S595A, with or without the substitutions Y569A and/or Y582G and/or S593A. In such embodiments, the fragment comprises the substitutions Y569A, Y582G, S593A, and S595A. In some embodiments, the fragment comprises a truncation of 27 or 26 amino acid residues from the C-terminal as compared with the mature gE protein. In such embodiments, the fragment optionally comprises the substitution T596A, with or without the substitutions Y569A and/or Y582G and/or S593A and/or S595A. In such embodiments, the fragment comprises the substitutions Y569A, Y582G, S593A, S595A, and T596A. In some embodiments, the fragment comprises a truncation of 25, 24, 23, 22, 21, 20, 19, 18, 17, 16, 15, 14, 13, 12, 11, 10, 9, 8, 7, 6, 5, 4, 3, or 2 amino acid residues or 1 amino acid residue from the C-terminal as compared with the mature gE protein. In such embodiments, the fragment optionally comprises the substitution T598A, with or without the substitutions Y569A and/or Y582G and/or S593A and/or S595A and/or T596A. In such embodiments, the fragment comprises the substitutions Y569A, Y582G, S593A, S595A, T596A, and T598A.

In some embodiments, the mature gE protein comprises the amino acid sequence set forth in SEQ ID NO: 1. In some embodiments, the full length gE protein comprises the amino acid sequence set forth in SEQ ID NO: 55.

In some embodiments, the fragment comprises the amino acid sequence set forth in SEQ ID NO: 3, 6, 8, 10, or 12, or an amino acid sequence having at least 80%, 85%, 90%, 91%, 92%, 93%, 94%, 95%, 96%, 97%, 98%, or 99% identity with the amino acid sequence set forth in SEQ ID NO: 3, 6, 8, 10, or 12.

In some embodiments, the N-terminal of the fragment is fused to the C-terminal of a signal peptide. In some embodiments, the N-terminal of the fragment is fused to the native signal peptide of the gE protein of VZV. In some embodiments, the native signal peptide comprise the amino acid sequence set forth in SEQ ID NO: 18. In some embodiments, the N-terminal of the fragment is fused to the C-terminal of a human tPA signal peptide. In some embodiments, the human tPA signal peptide comprise the amino acid sequence set forth in SEQ ID NO: 27. In some embodiments, the N-terminal of the fragment is fused to the C-terminal of a human IgE signal peptide. In some embodiments, the human IgE signal peptide comprise the amino acid sequence set forth in SEQ ID NO: 23.

In one aspect, provided herein are nucleic acids encoding the fragment as described herein.

In some embodiments, the fragment is encoded by the nucleotide sequence set forth in SEQ ID NO: 4, 5, 7, 9, 11, or 13, or a nucleotide sequence having at least 80%, 85%, 90%, 91%, 92%, 93%, 94%, 95%, 96%, 97%, 98%, or 99% identity with the nucleotide sequence set forth in SEQ ID NO: 4, 5, 7, 9, 11, or 13.

In some embodiments, the native signal peptide of the gE protein of VZV is encoded by the nucleotide sequence set forth in SEQ ID NO: 19, 20, 21, or 22, or a nucleotide sequence having at least 80%, 85%, 90%, 91%, 92%, 93%, 94%, 95%, 96%, 97%, 98%, or 99% identity with the nucleotide sequence set forth in SEQ ID NO: 19, 20, 21, or 22. In some embodiments, the human tPA signal peptide is encoded by the nucleotide sequence set forth in SEQ ID NO: 28, or a nucleotide sequence having at least 80%, 85%, 90%, 91%, 92%, 93%, 94%, 95%, 96%, 97%, 98%, or 99% identity with the nucleotide sequence set forth in SEQ ID NO: 28. In some embodiments, the human IgE signal peptide is encoded by the nucleotide sequence set forth in SEQ ID NO: 24, 25, or 26, or a nucleotide sequence having at least 80%, 85%, 90%, 91%, 92%, 93%, 94%, 95%, 96%, 97%, 98%, or 99% identity with the nucleotide sequence set forth in SEQ ID NO: 24, 25, or 26.

In some embodiments, provided herein is a protein comprising a mutant of a mature glycoprotein E (gE) of varicella-zoster virus (VZV), wherein the mutant comprises: (a) (i) a truncation of 37 amino acid residues from the C-terminus of the mature gE, and (ii) amino acid residue substitutions Y569A and Y582G, wherein amino acid residue positions 569 and 582 are the amino acid residue position numbers of full length VSV gE; (b) amino acid residue substitutions Y569A and Y582G, wherein amino acid residue positions 569 and 582 are the amino acid residue position numbers of full length VSV gE; (c) amino acid residue substitutions Y569A, Y582G, S593A, S595A, T596A, and T598A, wherein amino acid residue positions 569, 582, 593, 595, 596, and 598 are the amino acid residue position numbers of full length VSV gE; (d) amino acid residue substitutions Y582G, S593A, S595A, T596A, and T598A, wherein amino acid residue positions 582, 593, 595, 596, and 598 are the amino acid residue position numbers of full length VSV gE; or (e)(i) a truncation of 50 amino acid residues from the C-terminus of the mature gE protein, and (ii) amino acid residue substitution Y569A, wherein amino acid residue position 569 is the amino acid residue position number of full length VSV gE. In some embodiments, the mutant comprises the amino acid sequence of SEQ ID NO: 6, 8, 10, 12, or 3. In some embodiments, the amino acid sequence of the mutant consists of the amino acid sequence of SEQ ID NO: 6, 8, 10, 12, or 3. In some embodiments, the mutant comprises the amino acid sequence of SEQ ID NO: 6. In some embodiments, the amino acid sequence of the mutant consists of the amino acid sequence of SEQ ID NO: 6. In some embodiments, the mutant comprises an amino acid sequence that is at least 90%, at least 91%, at least 92%, at least 93%, at least 94%, or at least 95% identical to SEQ ID NO: 6, 8, 10, or 12. In some embodiments, the mutant comprises an amino acid sequence that is at least 96%, at least 97%, at least 98%, or at least 99% identical to SEQ ID NO: 6, 8, 10, or 12. In some embodiments, the mutant comprises an amino acid sequence that is at least 90%, at least 91%, at least 92%, at least 93%, at least 94%, or at least 95% identical to SEQ ID NO: 6. In some embodiments, the mutant comprises an amino acid sequence that is at least 96%, at least 97%, at least 98%, or at least 99% identical to SEQ ID NO: 6. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, the protein further comprises a VZV gE signal peptide. In some embodiments, the signal peptide of the VZV gE comprises the amino acid sequence set forth in SEQ ID NO: 18. In some embodiments, the signal peptide of the VZV gE comprises an amino acid sequence that is at least 95%, at least 96%, at least 97%, at least 98%, or at least 99% identical to the amino acid sequence set forth in SEQ ID NO: 18. In some embodiments, the protein further comprises a heterologous signal peptide, wherein the N-terminal of the mutant is fused to the C-terminal of the heterologous signal peptide. In some embodiments, the heterologous signal peptide is human tPA signal peptide. In some embodiments, the human tPA signal peptide comprises the amino acid sequence set forth in SEQ ID NO: 27. In some embodiments, the human tPA signal peptide comprises an amino acid sequence that is at least 95%, at least 96%, at least 97%, at least 98%, or at least 99% identical to the amino acid sequence set forth in SEQ ID NO: 27. In some embodiments, the amino acid sequence of human tPA signal peptide consists the amino acid sequence set forth in SEQ ID NO: 27. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23. In some embodiments, the amino acid sequence of the human IgE signal peptide consists of the amino acid sequence set forth in SEQ ID NO: 23. In some embodiments, the protein comprises the amino acid sequence set forth in SEQ ID NO: 59.

In some embodiments, provided herein is a protein comprising a mutant of a mature gE of VZV and the human IgE signal peptide, wherein the mutant comprises the amino acid sequence set forth in SEQ ID NO: 6, and wherein the amino acid sequence of the human IgE signal peptide comprises the amino acid sequence set forth in SEQ ID NO: 23. In some embodiments, provided herein is a protein comprising a mutant of a mature gE of VZV and the human IgE signal peptide, wherein the amino acid sequence of the mutant consists of the amino acid sequence set forth in SEQ ID NO: 6, and the amino acid sequence of the human IgE signal peptide consists of the amino acid sequence set forth in SEQ ID NO: 23. In some embodiments, provided herein is a protein consisting of a mutant of a mature gE of VZV and the human IgE signal peptide, wherein the amino acid sequence of the mutant consists of the amino acid sequence set forth in SEQ ID NO: 6, and the amino acid sequence of the human IgE signal peptide consists of the amino acid sequence set forth in SEQ ID NO: 23.

In some embodiments, provided herein is a protein comprising the amino acid sequence set forth in SEQ ID NO: 59. In some embodiments, provided herein is a protein comprising an amino acid sequence that is at least 85%, at least 86%, at least 87%, at least 88%, or at least 89% identical to the amino acid sequence set forth in SEQ ID NO: 59. In some embodiments, provided herein is a protein comprising an amino acid sequence that is at least 90%, at least 91%, at least 92%, at least 93%, or at least 94% identical to the amino acid sequence set forth in SEQ ID NO: 59. In some embodiments, provided herein is a protein comprising an amino acid sequence that is at least 95%, at least 96%, at least 97%, at least 98%, or at least 99% identical to the amino acid sequence set forth in SEQ ID NO: 59. In some embodiments, provided herein is a protein, wherein the amino acid sequence of the protein consists of the amino acid sequence set forth in SEQ ID NO: 59.

In some embodiments, provided herein is a fragment of a mature gE of VZV, wherein the fragment comprises a truncation of at least one and at most 50, 49, 48, 47, 46, 45, 44, 43, or 42 amino acid residues from the C-terminal of the mature gE. In some embodiments, the truncation is a truncation of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues from the C-terminal of the mature gE. In some embodiments, the truncation is a truncation of 11, 12, 13, 14, 15, 16, 17, 18, 34, 35, 36, 37, 38, 39, 40, 41, 42, 43, or 44 amino acid residues from the C-terminal of the mature gE. In some embodiments, the truncation is a truncation of 14 or 37 amino acid residues from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment further comprises the amino acid residue substitution Y569A, and wherein amino acid residue position 569 is the amino acid residue position number according to the full length VZV gE. In some embodiments, the fragment comprises an amino acid sequence having at least 80%, at least 85%, at least 90%, at least 91%, at least 92%, at least 93%, at least 94%, at least 95%, at least 96%, at least 97%, at least 98%, or at least 99% identity with the amino acid sequence set forth in SEQ ID NO: 6, 3, 8, 10, or 12. In some embodiments, the fragment comprises an amino acid sequence at least 80%, at least 81%, at least 82%, at least 83%, at least 84%, or at least 85% identical to the amino acid sequence set forth in SEQ ID NO: 6, 3, 8, 10, or 12. In some embodiments, the fragment comprises an amino acid sequence at least 90%, at least 91%, at least 92%, at least 93%, or at least 94% identical to the amino acid sequence set forth in SEQ ID NO: 6, 3, 8, 10, or 12. In some embodiments, the fragment comprises an amino acid sequence at least 95%, at least 96%, at least 97%, at least 98%, or at least 99% identical to the amino acid sequence set forth in SEQ ID NO: 6, 3, 8, 10, or 12. In some embodiments, the fragment comprises the amino acid sequence set forth in SEQ ID NO: 6, 3, 8, 10, or 12. In some embodiments, the amino acid sequence of the fragment consists of the amino acid sequence set forth in SEQ ID NO: 6, 3, 8, 10, or 12. In some embodiments, the fragment comprises the amino acid sequence set forth in SEQ ID NO: 6. In some embodiments, the amino acid sequence of the fragment consists of the amino acid sequence set forth in SEQ ID NO: 6.

In some embodiments, provided herein is a fragment of a mature gE of VZV, wherein the fragment comprises a truncation of at most 41, 40, 39, 38, 37, 36, 35, 34, 33, 32, or 31 amino acid residues from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment further comprises the amino acid residue substitution Y582G, and wherein amino acid residue position 582 is the amino acid residue position number according to the full length VZV gE protein. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fragment of a mature gE of VZV, wherein the fragment comprises a truncation of at most 30 or 29 amino acid residues from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment further comprises the amino acid residue substitution S593A, and wherein amino acid residue position 593 is the amino acid residue position number according to the full length VZV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fragment of a mature gE of VZV, wherein the fragment comprises a truncation of at most 28 amino acid residues from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment comprises the amino acid residue substitution S595A, and wherein amino acid residue position 595 is the amino acid residue position number according to the full length VSV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fragment of a mature gE of VZV, wherein the fragment comprises a truncation of at most 27 or 26 amino acid residues from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment further comprises the amino acid residue substitution T596A, and wherein amino acid residue position 596 is the amino acid residue position number according to the full length VZV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fragment of a mature gE of VZV, wherein the fragment comprises a truncation of at most 25, 24, 23, 22, 21, 20, 19, 18, 17, 16, 15, 14, 13, 12, 11, 10, 9, 8, 7, 6, 5, 4, 3, or 2 amino acid residues or at most one amino acid residue from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment further comprises the amino acid residue substitution T598A, and wherein amino acid residue position 598 is the amino acid residue position number according to the full length VZV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fragment of a mature gE of VZV, wherein the fragment comprises a truncation of 37 amino acid residues from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment comprises: (1) a truncation of 37 amino acid residues from the C-terminal of the mature gE, and (2) the amino acid residue substitutions Y569A and Y582G, and wherein amino acid residue positions 569 and 582 are amino acid residue position numbers according to the full length VZV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fusion protein comprising a fragment of a mature gE of VZV, and a heterologous signal peptide, wherein the N-terminal of the fragment is fused to the C-terminal of the heterologous signal peptide. In some embodiments, the heterologous signal peptide is a human tPA signal peptide. In some embodiments, the human tPA signal peptide comprises an amino acid sequence that is at least 95%, at least 96%, at least 97%, at least 98%, or at least 99% identical to the amino acid sequence set forth in SEQ ID NO: 28. In some embodiments, the human tPA signal peptide comprises the amino acid sequence set forth in SEQ ID NO: 27. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23. In some embodiments, the fragment comprises a truncation of at least one and at most 50, 49, 48, 47, 46, 45, 44, 43, or 42 amino acid residues from the C-terminal of the mature gE. In some embodiments, the truncation is a truncation of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues from the C-terminal of the mature gE. In some embodiments, the truncation is a truncation of 11, 12, 13, 14, 15, 16, 17, 18, 34, 35, 36, 37, 38, 39, 40, 41, 42, 43, or 44 amino acid residues from the C-terminal of the mature gE. In some embodiments, the truncation is a truncation of 14 or 37 amino acid residues from the C-terminal of the mature gE. In some embodiments, the fragment of the mature gE further comprises one or more amino acid substitutions. In some embodiments, the fragment further comprises one, two, three, four, five or all of the following amino acid substitutions: Y569A, Y582G, S593A, S595A, T596A, and T598A, wherein the amino acid position number is according to the full length VZV gE. In some embodiments, the fragment is one described herein. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fusion protein comprising a fragment of a mature gE of VZV and a human IgE signal peptide, wherein the fragment comprises a truncation of 37 amino acid residues from the C-terminal of the mature gE, and the N-terminal of the fragment is fused to the C-terminal of the human IgE signal peptide. In some embodiments, the human IgE signal peptide comprises an amino acid sequence that is at least 95%, at least 96%, at least 97%, at least 98%, or at least 99% identical to the amino acid sequence set forth in SEQ ID NO: 23. In some embodiments, the human IgE signal peptide comprises the amino acid sequence set forth in SEQ ID NO: 23. In some embodiments, the amino acid sequence of the human IgE signal peptide consists of the amino acid sequence set forth in SEQ ID NO: 23. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mature gE comprises the amino acid sequence set forth in SEQ ID NO: 1 and the full length VZV gE comprises the amino acid sequence set forth in SEQ ID NO: 55.

In some embodiments, provided herein is a fusion protein comprising a fragment of a mature gE of VZV and a human IgE signal peptide, wherein the fragment comprises the amino acid sequence set forth in SEQ ID NO: 6, wherein the human IgE signal peptide comprises the amino acid sequence set forth in SEQ ID NO: 23, and wherein the N-terminal of the fragment is fused to the C-terminal of the human IgE signal peptide. In some embodiments, the amino acid sequence of the fragment consists of the amino acid sequence set forth in SEQ ID NO: 6, and the amino acid sequence of the human IgE signal peptide consists of the amino acid sequence set forth in SEQ ID NO: 23. In some embodiments, provided herein is a fusion protein consisting of a fragment of a mature gE of VZV and a human IgE signal peptide, wherein the amino acid sequence of the fragment consists of the amino acid sequence set forth in SEQ ID NO: 6, wherein the amino acid sequence of the human IgE signal peptide consists of the amino acid sequence set forth in SEQ ID NO: 23, and wherein the N-terminal of the fragment is fused to the C-terminal of the human IgE signal peptide.

In some embodiments, provided herein is a nucleic acid encoding a protein described herein. In some embodiments, provided herein is a nucleic acid encoding a fragment of described herein. In some embodiments, provided herein is a nucleic acid encoding a fusion protein described herein. In some embodiments, provided herein is a nucleic acid comprising a protein described herein, a fragment described herein, or a fusion protein described herein, wherein the nucleic acid comprises the nucleotide sequence set forth in SEQ ID NO: 7, 9, 11, 13, 4, or 5. In some embodiments, provided herein is a nucleic acid comprising a protein described herein, a fragment described herein, or a fusion protein described herein, wherein the nucleic acid comprises a nucleotide sequence having at least 80%, at least 85%, at least 90%, at least 91%, at least 92%, at least 93%, at least 94%, at least 95%, at least 96%, at least 97%, at least 98%, or at least 99% identity with the nucleotide sequence set forth in SEQ ID NO: 7, 9, 11, 13, 4 or 5. In some embodiments, provided herein is a nucleic acid comprising a protein described herein, a fragment described herein, or a fusion protein described herein, wherein the nucleic acid comprises a nucleotide sequence that is at least 80%, at least 81%, at least 82%, at least 830%, or at least 84% identical to the nucleotide sequence set forth in SEQ ID NO: 7, 9, 11, 13, 4 or 5. In some embodiments, provided herein is a nucleic acid comprising a protein described herein, a fragment described herein, or a fusion protein described herein, wherein the nucleic acid comprises a nucleotide sequence that is at least 85%, at least 86%, at least 87%, at least 88%, or at least 89% identical to the nucleotide sequence set forth in SEQ ID NO: 7, 9, 11, 13, 4 or 5. In some embodiments, provided herein is a nucleic acid comprising a protein described herein, a fragment described herein, or a fusion protein described herein, wherein the nucleic acid comprises a nucleotide sequence that is at least 90%, at least 910%, at least 92%, at least 930%, or at least 94% identical to the nucleotide sequence set forth in SEQ ID NO: 7, 9, 11, 13, 4 or 5. In some embodiments, provided herein is a nucleic acid comprising a protein described herein, a fragment described herein, or a fusion protein described herein, wherein the nucleic acid comprises a nucleotide sequence that is at least 95%, at least 96%, at least 97%, at least 98%, or at least 99% identical to the nucleotide sequence set forth in SEQ ID NO: 7, 9, 11, 13, 4 or 5. In some embodiments, provided herein is a nucleic acid comprising a protein described herein, a fragment described herein, or a fusion protein described herein, wherein the nucleic acid comprises the nucleotide sequence of SEQ ID NO: 7.

In some embodiments, provided herein is a nucleic acid encoding a protein described herein, wherein the protein comprises a mutant of a mature gE of VZV and a human IgE signal peptide, and wherein the nucleotide sequence encoding the IgE signal peptide comprises the nucleotide sequence set forth in SEQ ID NO: 24, 25, or 26. In some embodiments, provided herein is a nucleic acid encoding a protein described herein, wherein the protein comprises a mutant of a mature gE of VZV and a human IgE signal peptide, and wherein the nucleotide sequence encoding the IgE signal peptide comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 24, 25, or 26.

In some embodiments, provided herein is a nucleic acid encoding a fusion protein described herein, wherein the fusion protein comprises a fragment of a mature gE of VZV and a human IgE signal peptide, and wherein the nucleotide sequence encoding the IgE signal peptide comprises the nucleotide sequence set forth in SEQ ID NO: 24, 25, or 26. In some embodiments, provided herein is a nucleic acid encoding a fusion protein described herein, wherein the fusion protein comprises a fragment of a mature gE of VZV and a human IgE signal peptide, and wherein the nucleotide sequence encoding the IgE signal peptide comprises a nucleotide sequence that is at least 80%, at least 85%, at least 90%, at least 910%, at least 92%, at least 93%, at least 94%, at least 95%, at least 96%, at least 97%, at least 98%, or at least 99% identical to the nucleotide sequence set forth in SEQ ID NO: 24, 25, or 26.

In some embodiments, provided herein is a nucleic acid encoding a protein described herein, wherein the protein comprises a mutant of a mature gE of VZV and a VZV gE signal peptide, wherein the nucleotide sequence of the signal peptide comprises the nucleotide sequence set forth in SEQ ID NO: 19, 20, 21, or 22. In some embodiments, provided herein is a nucleic acid encoding a protein described herein, wherein the protein comprises a mutant of a mature gE of VZV and a VZV gE signal peptide, wherein the nucleotide sequence of the signal peptide comprises a nucleotide sequence that is at least 80%, at least 85%, at least 90%, at least 910%, at least 92%, at least 93%, at least 94%, at least 95%, at least 96%, at least 97%, at least 98%, or at least 99% identical to the nucleotide sequence set forth in SEQ ID NO: 19, 20, 21, or 22.

In some embodiments, provided herein is a nucleic acid encoding a protein described herein, wherein the protein comprises a mutant of a mature gE of VZV and a human tPA signal peptide, wherein the nucleotide sequence encoding a human tPA signal peptide comprises the nucleotide sequence set forth in SEQ ID NO: 28. In some embodiments, provided herein is a nucleic acid encoding a protein described herein, wherein the protein comprises a fragment of a mature gE of VZV and human tPA signal peptide, wherein the nucleotide sequence encoding a human tPA signal peptide comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 28.

In some embodiments, provided herein is a nucleic acid encoding a fusion protein described herein, wherein the fusion protein comprises a fragment of a mature gE of VZV and a human tPA signal peptide, wherein the nucleotide sequence encoding a human tPA signal peptide comprises the nucleotide sequence set forth in SEQ ID NO: 28. In some embodiments, provided herein is a nucleic acid encoding a fusion protein described herein, wherein the fusion protein comprises a fragment of a mature gE of VZV and human tPA signal peptide, wherein the nucleotide sequence encoding a human tPA signal peptide comprises a nucleotide sequence that is at least 80%, at least 85%, at least 90%, at least 910%, at least 92%, at least 93%, at least 94%, at least 95%, at least 96%, at least 97%, at least 98%, or at least 99% identical to the nucleotide sequence set forth in SEQ ID NO: 28.

In some embodiments, disclosed herein are vectors or cells comprising the nucleic acids as described herein. In some embodiments, the vectors are preferably IVT plasmids. In some embodiments, disclosed herein are compositions comprising the fragment as described herein or the nucleic acid as described herein. In some embodiments, the composition is a pharmaceutical composition. In some embodiments, the composition is a vaccine.

In one aspect, provided herein are non-naturally occurring nucleic acid molecules that can be used for the prevention, management and treatment of a disease or disorder cause by VZV or by infection with VZV.

In some embodiments, the non-naturally occurring nucleic acids comprises a coding nucleotide sequence encoding the fragment as described herein. In some embodiments, the fragment consists of, essentially consists of or comprises an amino acid sequence having at least about 90%, 91%, 92%, 93%, 94%, 95%, 96%, 97%, 98%, or 99% sequence identity to the amino acid sequence set forth in SEQ ID NO:1 or a fragment thereof. In some embodiments, the fragment consists of, essentially consists of or comprises an amino acid sequence having at least about 90%, 91%, 92%, 93%, 94%, 95%, 96%, 97%, 98%, 99%, or 100% sequence identity to the amino acid sequence set forth in SEQ ID NO:3, 6, 8, 10 or 12. In some embodiments, the fragment consists of, essentially consists of or comprises the amino acid sequence set forth in SEQ ID NO:3, 6, 8, 10, or 12. In some embodiments, the coding nucleotide sequence consists of, essentially consists of or comprises a nucleotide sequence having at least about 50%, 55%, 60%, 65%, 70%, 75%, 80%, 85%, 90%, 91%, 92%, 93%, 94%, 95%, 96%, 97%, 98%, or 99% sequence identity to the nucleotide sequence set forth in SEQ ID NO:2 or a fragment thereof. In some embodiments, the coding nucleotide sequence has been codon optimized for expression in cells of a subject. In some embodiments, the subject is a non-human mammal. In some embodiments, the subject is a human. In some embodiments, the coding nucleotide sequence consists of, essentially consists of or comprises a nucleotide sequence having at least about 50%, 55%, 60%, 65%, 70%, 75%, 80%, 85%, 90%, 91%, 92%, 93%, 94%, 95%, 96%, 97%, 98%, 99%, or 100% sequence identity to the nucleotide sequence set forth in SEQ ID NO:4, 5, 7, 9, 11, or 13. In some embodiments, the coding nucleotide sequence consists of, essentially consists of or comprises the nucleotide sequence set forth in SEQ ID NO:4, 5, 7, 9, 11, or 13. In some embodiments, the fragment is fused to the gE native signal peptide. In some embodiments, the signal peptide consists of, essentially consists of or comprises an amino acid sequence having at least about 90%, 91%, 92%, 93%, 94%, 95%, 96%, 97%, 98%, 99%, or 100% sequence identity to the amino acid sequence set forth in SEQ ID NO:18. In some embodiments, the signal peptide is encoded by a coding nucleotide sequence consisting of, essentially consisting of or comprising a nucleotide sequence having at least about 50%, 55%, 60%, 65%, 70%, 75%, 80%, 85%, 90%, 91%, 92%, 93%, 94%, 95%, 96%, 97%, 98%, 99%, or 100% sequence identity to the nucleotide sequence set forth in SEQ ID NO:19, 20, 21, or 22. In some embodiments, the fragment is fused to a heterologous polypeptide. In some embodiments, the heterologous polypeptide is selected from a Fc region of human immunoglobulin, a signal peptide, and a peptide facilitating multimerization of the fusion protein. In some embodiments, the signal peptide is a signal peptide from IgE or tPA. In some embodiments, the signal peptide consists of, essentially consists of or comprises an amino acid sequence having at least about 90%, 91%, 92%, 93%, 94%, 95%, 96%, 97%, 98%, 99%, or 100% sequence identity to the amino acid sequence set forth in SEQ ID NO:23. In some embodiments, the signal peptide is encoded by a coding nucleotide sequence consisting of, essentially consisting of or comprising a nucleotide sequence having at least about 50%, 55%, 60%, 65%, 70%, 75%, 80%, 85%, 90%, 91%, 92%, 93%, 94%, 95%, 96%, 97%, 98%, 99%, or 100% sequence identity to the nucleotide sequence set forth in SEQ ID NO:24, 25 or 26. In some embodiments, the signal peptide consists of, essentially consists of or comprises an amino acid sequence having at least about 90%, 91%, 92%, 93%, 94%, 95%, 96%, 97%, 98%, 99%, or 100% sequence identity to the amino acid sequence set forth in SEQ ID NO:27. In some embodiments, the signal peptide is encoded by a coding nucleotide sequence consisting of, essentially consisting of or comprising a nucleotide sequence having at least about 50%, 55%, 60%, 65%, 70%, 75%, 80%, 85%, 90%, 91%, 92%, 93%, 94%, 95%, 96%, 97%, 98%, 99%, or 100% sequence identity to the nucleotide sequence set forth in SEQ ID NO:28. In some embodiments, the multimerization is dimerization or trimerization. In some embodiments, the non-naturally occurring nucleic acid further comprises a 5′ untranslated region (5′-UTR), wherein the 5′-UTR comprises the sequence set forth in any one of SEQ ID NOS: 29-38. In some embodiments, the non-naturally occurring nucleic acid further comprises a 3′ untranslated region (3′-UTR), wherein the 3′-UTR comprises the sequence set forth in any one of SEQ ID NOS: 39-46. In some embodiments, the 3′-UTR further comprises a poly-A tail or a polyadenylation signal. In some embodiments, the nucleic acid consists of, essentially consists of or comprises the nucleotide sequence set forth in SEQ ID NO: 49, 50, 51, 52, 53, 54, 60, 61, 62, 63 or 64 or comprises a nucleotide sequence having at least 80%, 85%, 90%, 91%, 92%, 93%, 94%, 95%, 96%, 97%, 98%, or 99% identity with the nucleotide sequence set forth in SEQ ID NO: 49, 50, 51, 52, 53, 54, 60, 61, 62, 63 or 64. In some embodiments, the non-naturally occurring nucleic acid comprises one or more functional nucleotide analogs. In some embodiments, the non-naturally occurring nucleic acid comprises one or more functional nucleotide analogs that are selected from pseudouridine (psd), 1-methyl-pseudouridine (m1) and 5-methylcytosine. In some embodiments, the nucleic acid is DNA or mRNA. In some embodiments, the nucleic acid consists of, essentially consists of or comprises the nucleotide sequence set forth in SEQ ID NO: 63 or comprises a nucleotide sequence having at least 80%, 85%, 90%, 91%, 92%, 93%, 94%, 95%, 96%, 97%, 98%, or 99% identity with the nucleotide sequence set forth in SEQ ID NO: 63, wherein all thymines (T) are substituted for uracil (U) or N1-methylpseudouridine, and/or the first nucleotide G is substituted for m⁷ GpppAmpU.

In some embodiments, disclosed herein are vectors or cells comprising the non-naturally occurring nucleic acid molecule as described herein. In some embodiments, the vectors are preferably IVT plasmids. In some embodiments, disclosed herein are compositions comprising the non-naturally occurring nucleic acid molecule as described herein.

In some embodiments, provided herein is a non-naturally occurring nucleic acid comprising a coding nucleotide sequence encoding a protein described herein. In some embodiments, provided herein is a non-naturally occurring nucleic acid comprising a coding nucleotide sequence encoding a fragment described herein. In some embodiments, provided herein is a non-naturally occurring nucleic acid comprising a coding nucleotide sequence encoding a fusion protein described herein. In some embodiments, the coding nucleotide sequence has been codon optimized for expression in cells of a subject. In some embodiments, the coding nucleotide sequence has been codon optimized for expression in cells of a non-human mammal. In specific embodiments, the coding nucleotide sequence has been codon optimized for expression in cells of a human. In some embodiments, the non-naturally occurring nucleic acid comprises the nucleotide sequence set forth in SEQ ID NO: 7, 9, 11, 13, 4 or 5. the non-naturally occurring nucleic acid comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 7, 9, 11, 13, 4 or 5. In some embodiments, the non-naturally occurring nucleic acid comprises the nucleotide sequence set forth in SEQ ID NO: 7.

In some embodiments, provided herein is a non-naturally occurring nucleic acid encoding a protein described herein, wherein the protein comprises a mutant of a mature gE of VZV and a human IgE signal peptide, and wherein the nucleotide sequence encoding the IgE signal peptide comprises the nucleotide sequence set forth in SEQ ID NO: 24, 25, or 26. In some embodiments, provided herein is a non-naturally occurring nucleic acid encoding a protein described herein, wherein the protein comprises a mutant of a mature gE of VZV and a human IgE signal peptide, and wherein the nucleotide sequence encoding the IgE signal peptide comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 24, 25, or 26.

In some embodiments, provided herein is a non-naturally occurring nucleic acid encoding a fusion protein described herein, wherein the fusion protein comprises a fragment of a mature gE of VZV and a human IgE signal peptide, and wherein the nucleotide sequence encoding the IgE signal peptide comprises the nucleotide sequence set forth in SEQ ID NO: 24, 25, or 26. In some embodiments, provided herein is a non-naturally occurring nucleic acid encoding a fusion protein described herein, wherein the fusion protein comprises a fragment of a mature gE of VZV and a human IgE signal peptide, and wherein the nucleotide sequence encoding the IgE signal peptide comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 24, 25, or 26.

In some embodiments, provided herein is a non-naturally occurring nucleic acid encoding a protein described herein, wherein the protein comprises a mutant of a mature gE of VZV and a VZV gE signal peptide, wherein the nucleotide sequence of the signal peptide comprises the nucleotide sequence set forth in SEQ ID NO: 19, 20, 21, or 22. In some embodiments, provided herein is a non-naturally occurring nucleic acid encoding a protein described herein, wherein the protein comprises a mutant of a mature gE of VZV and a VZV gE signal peptide, wherein the nucleotide sequence of the signal peptide comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 19, 20, 21, or 22.

In some embodiments, provided herein is a non-naturally occurring nucleic acid encoding a protein described herein, wherein the protein comprises a mutant of a mature gE of VZV and a human tPA signal peptide, wherein the nucleotide sequence encoding a human tPA signal peptide comprises the nucleotide sequence set forth in SEQ ID NO: 28. In some embodiments, provided herein is a non-naturally occurring nucleic acid encoding a protein described herein, wherein the protein comprises a fragment of a mature gE of VZV and human tPA signal peptide, wherein the nucleotide sequence encoding a human tPA signal peptide comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 28.

In some embodiments, provided herein is a non-naturally occurring nucleic acid encoding a fusion protein described herein, wherein the fusion protein comprises a fragment of a mature gE of VZV and a human tPA signal peptide, wherein the nucleotide sequence encoding a human tPA signal peptide comprises the nucleotide sequence set forth in SEQ ID NO: 28. In some embodiments, provided herein is a non-naturally occurring nucleic acid encoding a fusion protein described herein, wherein the fusion protein comprises a fragment of a mature gE of VZV and human tPA signal peptide, wherein the nucleotide sequence encoding a human tPA signal peptide comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 28.

In some embodiments, provided herein is a non-naturally occurring nucleic acid comprising the nucleotide sequence of SEQ ID NO: 63, 7, 51, 60, 62, 62, 64, 9, 11, 13, 52, 53, 54, 58, 49, 50, 4, or 5. In some embodiments, the non-naturally occurring nucleic acid consists of the nucleotide sequence of SEQ ID NO: 63, 7, 51, 60, 62, 62, 64, 9, 11, 13, 52, 53, 54, 58, 49, 50, 4, or 5. In some embodiments, the non-naturally occurring nucleic acid consists of, consists essentially of, or comprises the nucleotide sequence set forth in SEQ ID NO: 63, 7, 51, 60, 62, 62, 64, 9, 11, 13, 52, 53, 54, 58, 49, 50, 4, or 5. In some embodiments, the non-naturally occurring nucleic acid comprises a nucleotide sequence having at least 80%, at least 85%, at least 90%, at least 91%, at least 92%, at least 93%, at least 94%, at least 95%, at least 96%, at least 97%, at least 98%, or at least 99% identity with the nucleotide sequence set forth in SEQ ID NO: 63, 7, 51, 60, 62, 62, 64, 9, 11, 13, 52, 53, 54, 58, 49, 50, 4, or 5. In some embodiments, the non-naturally occurring nucleic acid consists of or consists essentially of a nucleotide sequence having at least 80%, at least 85%, at least 90%, at least 91%, at least 92%, at least 93%, at least 94%, at least 95%, at least 96%, at least 97%, at least 98%, or at least 99% identity with the nucleotide sequence set forth in SEQ ID NO: 63, 7, 51, 60, 62, 62, 64, 9, 11, 13, 52, 53, 54, 58, 49, 50, 4, or 5. In some embodiments, the non-naturally occurring nucleic acid consists of, consists essentially of, or comprises the nucleotide sequence set forth in SEQ ID NO: 63, 51, 60, 62, 62, or 64. In some embodiments, the non-naturally occurring nucleic acid comprises the nucleotide sequence set forth in SEQ ID NO: 63. In some embodiments, the non-naturally occurring nucleic acid consists of the nucleotide sequence set forth in SEQ ID NO: 63.

In some embodiments, a non-naturally occurring nucleic acid described herein further comprises a 5′ untranslated region (5′-UTR) and/or 3′ untranslated region (3′-UTR). In some embodiments, a non-naturally occurring nucleic acid described herein further comprises a 5′-UTR, wherein the 5′-UTR comprises the nucleotide sequence set forth in any one of SEQ ID NOS: 29-38. In some embodiments, a non-naturally occurring nucleic acid described herein further comprises a 3′-UTR, wherein the 3′-UTR comprises the nucleotide sequence set forth in any one of SEQ ID NOS: 39-46. In some embodiments, a non-naturally occurring nucleic acid described herein further comprises a 5′-UTR and a 3′-UTR, wherein the 5′-UTR comprises the nucleotide sequence set forth in any one of SEQ ID NOS: 29-38, and wherein the 3′-UTR comprises the nucleotide sequence set forth in any one of SEQ ID NOS: 39-46. In some embodiments, the 3′-UTR further comprises a poly-A tailor a poly adenylation signal.

In some embodiments, a non-naturally occurring nucleic acid described herein comprises DNA. In some embodiments, a non-naturally occurring nucleic acid described herein is DNA. In some embodiments, a non-naturally occurring nucleic acid described herein comprises one or more functional nucleotide analogs. In some embodiments, a non-naturally occurring nucleic acid described herein comprises mRNA, and wherein thymines are substituted for uracils or a functional analog thereof in the nucleic acid. In some embodiments, a non-naturally occurring nucleic acid described herein is mRNA, and wherein thymines are substituted for uracils or a functional analog thereof in the nucleic acid. In some embodiments, the nucleic acid comprises one or more functional nucleotide analogs that are selected from pseudouridine, 1-methyl-pseudouridine and 5-methylcytosine. In some embodiments, the nucleic acid consists of, consists essentially of, or comprises the nucleotide sequence set forth in SEQ ID NO: 63, except that all thymines (T) are substituted for uracil (U) or N1-methylpseudouridine. In some embodiments, the nucleic acid consists of, consists essentially of, or comprises the nucleotide sequence set forth in SEQ ID NO: 63, except that all thymines (T) are substituted for uracil (U) or N1-methylpseudouridine and the first nucleotide G is substituted for m⁷ GpppAmpU. In some embodiments, the nucleic acid consists of, consists essentially of, or comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 63; except that all thymines (T) are substituted for uracil (U) or N1-methylpseudouridine. In some embodiments, the nucleic acid consists of, consists essentially of, or comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 63; and the first nucleotide G is substituted for m⁷ GpppAmpU. In some embodiments, the nucleic acid consists of, consists essentially of, or comprises the nucleic acid consists of, consists essentially of, or comprises a nucleotide sequence that is at least 80%, at least 85%, at least 90%, at least 91%, at least 92%, at least 93%, at least 94%, at least 95%, at least 96%, at least 97%, at least 98%, or at least 99% identical to the nucleotide sequence set forth in SEQ ID NO: 63; except that all thymines (T) are substituted for uracil (U) or N1-methylpseudouridine, and the first nucleotide G is substituted for m⁷ GpppAmpU. In some embodiments, the nucleic acid consists of, essentially consists of or comprises the nucleotide sequence set forth in SEQ ID NO: 63, except that all thymines (T) are substituted for N1-methylpseudouridine, and the first nucleotide G is substituted for m⁷ GpppAmpU.

In some embodiments, provided herein is a vector comprising a nucleic acid described herein. In some embodiments, provide herein is a vector comprising a non-naturally occurring nucleic acid described herein. In some embodiments, the vector is an IVT (In Vitro Transcription) plasmid.

In some embodiments, provided herein is a host cell comprising the nucleic acid described herein. In some embodiments, provided herein is host cell comprising a non-naturally occurring nucleic acid described herein. In some embodiments, provided herein is a host cell comprising a vector described herein. In some embodiments, the host cell expresses a protein described herein, a fragment described herein, or a fusion protein described herein. In some embodiments, the host cell is in vitro, ex vivo, or isolated.

In some embodiments of the composition described herein, the composition further comprises at least one lipid described herein. In some embodiments of the composition described herein, the composition further comprises at least a first lipid (e.g., a cationic lipid) described herein and optionally a second lipid (e.g., a polymer conjugated lipid) described herein.

In some embodiments, the first lipid is a compound according to Series 01, 02, 03, and 04, e.g., a compound according to Formula (01-I), (01-II), (02-I), (03-I), or (04-I). In some embodiments, the first lipid is a compound listed in Table 01-1, 02-1, 03-1, or 04-1. In some embodiments, the second lipid is a compound according to Series 05, e.g., a compound according to Formula (05-I).

In some embodiments, the composition is formulated as lipid nanoparticles encapsulating the nucleic acid in a lipid shell. In some embodiments, the composition is a pharmaceutical composition. In some embodiments, the composition is a vaccine.

In some embodiments, provided herein is a composition (e.g., pharmaceutical composition) comprising a protein described herein. In some embodiments, provided herein is a composition (e.g., pharmaceutical composition) comprising a fragment described herein. In some embodiments, provided herein is a composition (e.g., pharmaceutical composition) comprising a fusion protein described herein. In some embodiments, the composition is a pharmaceutical composition. In some embodiments, the pharmaceutical composition is a vaccine. In some embodiments, a protein described herein, a fragment, or a fusion protein described herein is used to immunize a subject against VZV. In some embodiments, a nucleic acid described herein, a protein described herein, a fragment, or a fusion protein described herein is used to induce an immune response (e.g., an immune response, such as, e.g., described in Section 6) in a subject. In some embodiments, the immune response comprises a humoral immune response against VZV. In some embodiments, the immune response comprises a cellular response against VZV. In some embodiments, the immune response comprises antibody (e.g., neutralizing antibody) specific to VZV gE.

In some embodiments, provided herein is a composition (e.g., pharmaceutical composition) comprising a nucleic acid described herein. In some embodiments, provided herein is a composition (e.g., pharmaceutical composition) comprising a non-naturally occurring nucleic acid described herein. In some embodiments, provided herein is a composition (e.g., pharmaceutical composition) comprising a vector described herein. In some embodiments, the composition is a pharmaceutical composition. In some embodiments, the pharmaceutical composition is a vaccine. In some embodiments, a nucleic acid described herein, anon-naturally occurring nucleic acid, or a vector described herein is used to immunize a subject against VZV. In some embodiments, a nucleic acid described herein, a non-naturally occurring nucleic acid described herein, or a vector described herein is used to induce an immune response (e.g., an immune response, such as, e.g., described in Section 6) in a subject. In some embodiments, the immune response comprises a humoral immune response against VZV. In some embodiments, the immune response comprises a cellular response against VZV. In some embodiments, the immune response comprises antibody (e.g., neutralizing antibody) specific to VZV gE.

In some embodiments, provided herein is a pharmaceutical composition comprising anon-naturally occurring nucleic acid described herein, and at least a first lipid. In some embodiments, the first lipid is a lipid described herein. In some embodiments, the first lipid is a compound according to Formula 01-I or Formula 01-II; or a compound listed in Table 01-1; or a compound according to Formula 02-I; or a compound listed in Table 02-1; or a compound according to Formula 03-I; or a compound listed in Table 03-1; or a compound according to Formula 04-I; or a compound listed in Table 04-1. In some embodiments, the pharmaceutical composition of claim 72, further comprising a second lipid. In some embodiments, the second lipid is a compound according to Formula 05-I. In some embodiments, the pharmaceutical composition is formulated as lipid nanoparticles encapsulating the nucleic acid in a lipid shell. In some embodiments, the pharmaceutical composition is a vaccine.

In one aspect, provided herein are methods for managing, preventing or treating a disease or disorder caused by VZV or by infection with VZV in a subject, comprising administering to the subject a therapeutically effective amount of the fragment described herein, a therapeutically effective amount of the nucleic acid described herein, a therapeutically effective amount of the non-naturally occurring nucleic acid described herein, or a therapeutically effective amount of the pharmaceutical composition as described herein.

In some embodiments of the method described herein, the subject is a human or a non-human mammal. In some embodiments, the subject is a human adult, a human child or a human toddler. In some embodiments, the subject has the disease or disorder. In some embodiments, the subject is at risk of, or is susceptible to, infection by VZV. In some embodiments, the subject is an elderly human. In some embodiments, subject has been diagnosed positive for infection by VZV. In some embodiments, the subject is asymptomatic.

In some embodiments of the method described herein, the method comprises administering lipid nanoparticles encapsulating the nucleic acid to the subject, and wherein the lipid nanoparticles are endocytosed by the cells in the subject. In some embodiments, the nucleic acid is expressed by the cells in the subject.

In some embodiments of the method described herein, an immune response against VZV is elicited in the subject. In some embodiments, the immune response comprises production of cytokinein lymphocytes. In some embodiments, the immune response comprises increased proportion of cytokine-expressing lymphocytes. In some embodiments, the lymphocytes are CD4⁺ T cells and/or CD8⁺ T cells. In some embodiments, the cytokine is one or more of IFN-γ, IL-2, and TNF-α. In some embodiments, the production of cytokines in lymphocytes is increased. In some embodiments, the immune response comprises production of an antibody specifically binds to the viral gE protein. In some embodiments, the antibody is a neutralizing antibody against VZV or cells infected by VZV. In some embodiments, the serum titer of the antibody is increased in the subject.

In some embodiments of the method described herein, antibody binds to a viral particle or an infected cell and mark the viral particle of infected cell for destruction by the immune system of the subject. In some embodiments, endocytosis of viral particles bound by the antibody is induced or enhanced. In some embodiments, antibody-dependent cell-mediated cytotoxicity (ADCC) against infected cells in the subject is induced or enhanced. In some embodiments, antibody-dependent cellular phagocytosis (ADCP) against infected cells in the subject is induced or enhanced. In some embodiments, complement dependent cytotoxicity (CDC) against infected cells in the subject is induced or enhanced.

In some embodiments of the method described herein, the disease or disorder caused by VZV is varicella and/or zoster. In some embodiments of the method described herein, the disease or disorder caused by VZV is postherpetic neuralgia (PHN). In some embodiments of the method described herein, the disease or disorder caused by VZV is one or more of meningoencephalitis, myelitis, cranial nerve palsy, angiopathy, keratitis, retinopathy, ulcers, hepatitis and pancreatitis.

In some embodiments, provided herein is a method for managing, preventing or treating a disease or disorder caused by VZV orby infection with VZV in a subject, comprising administering to the subject a therapeutically effective amount of a protein described herein. In some embodiments, provided herein is a method for managing, preventing or treating a disease or disorder caused by VZV or by infection with VZV in a subject, comprising administering to the subject a therapeutically effective amount of a fragment described herein. In some embodiments, provided herein is a method for managing, preventing or treating a disease or disorder caused by VZV or by infection with VZV in a subject, comprising administering to the subject a therapeutically effective amount of a fusion protein described herein. In some embodiments, provided herein is a method for managing, preventing or treating a disease or disorder caused by VZV orby infection with VZV in a subject, comprising administering to the subject a therapeutically effective amount of a nucleic acid described herein. In some embodiments, provided herein is a method for managing, preventing or treating a disease or disorder caused by VZV orby infection with VZV in a subject, comprising administering to the subject a therapeutically effective amount of a non-naturally occurring nucleic acid described herein. In some embodiments, provided herein is a method for managing, preventing or treating a disease or disorder caused by VZV or by infection with VZV in a subject, comprising administering to the subject a therapeutically effective amount of a vector described herein. In some embodiments, provided herein is a method for managing preventing or treating a disease or disorder caused by VZV or by infection with VZV in a subject, comprising administering to the subject a therapeutically effective amount of a pharmaceutical composition described herein. In some embodiments, the method is for preventing a disease or disorder caused by VZV orby infection with VZV in the subject. In some embodiments, an immune response against the VZV is elicited in the subject. In some embodiments, the immune response comprises production of cytokine in lymphocytes. In some embodiments, the immune response comprises increased proportion of cytokine-expressing lymphocytes. In some embodiments, the lymphocytes are CD4⁺ T cells and/or CD8⁺ T cells, and/or, wherein the cytokine is one or more of IFN-γ, IL-2, and TNF-α. In some embodiments, the production of cytokine in lymphocytes is increased. In some embodiments, the immune response comprises production of an antibody (e.g., neutralizing antibody) that specifically binds to VZV gE. In some embodiments, the disease or disorder caused by VZV is (a) varicella or zoster; (b) postherpetic neuralgia (PHN); or (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella and zoster; (b) postherpetic neuralgia (PHN); or (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella or zoster; (b) postherpetic neuralgia (PHN); and (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella and zoster; (b) postherpetic neuralgia (PHN); and (c) one or more of meningoencephalitis, myelitis, cranial nerve palsy, angiopathy, keratitis, retinopathy, ulcers, hepatitis and pancreatitis. In some embodiments, the subject is a human. In some embodiments, the human is a human adult. In some embodiments, the adult is at least 40 years old. In some embodiments, the adult is at least 45 years old. In some embodiments, the adult is at least 50 years old. In some embodiments, the adult is at least 55 years old. In some embodiments, the adult is at least 60 years old. In some embodiments, the human is an elderly human.

In some embodiments, provided herein is a protein described herein, a fragment described herein, or a fusion protein described herein, for use in a method for managing preventing or treating a disease or disorder caused by VZV or by infection with VZV in a subject. In some embodiments, provided herein is a nucleic acid described herein, a non-naturally occurring described herein, or a vector described herein, for use in a method for managing, preventing or treating a disease or disorder caused by VZV or by infection with VZV in a subject. In some embodiments, provided herein is a pharmaceutical composition described herein, for use in a method for managing, preventing or treating a disease or disorder caused by VZV or by infection with VZV in a subject. In some embodiments, the disease or disorder caused by VZV is (a) varicella or zoster; (b) postherpetic neuralgia (PHN); or (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella and zoster; (b) postherpetic neuralgia (PHN); or (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella or zoster; (b) postherpetic neuralgia (PHN); and (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella and zoster; (b) postherpetic neuralgia (PHN); and (c) one or more of meningoencephalitis, myelitis, cranial nerve palsy, angiopathy, keratitis, retinopathy, ulcers, hepatitis and pancreatitis. In some embodiments, the subject is a human. In some embodiments, the human is a human adult. In some embodiments, the adult is at least 40 years old. In some embodiments, the adult is at least 45 years old. In some embodiments, the adult is at least 50 years old. In some embodiments, the adult is at least 55 years old. In some embodiments, the adult is at least 60 years old. In some embodiments, the human is an elderly human.

In some embodiments, provided herein is a use of a protein described herein, a fragment described herein, a fusion protein described herein, for the manufacture of a medicament for managing, preventing or treating a disease or disorder caused by VZV or by infection with VZV in a subject. In some embodiments, provided herein is a use of a nucleic acid described herein, a non-naturally occurring nucleic acid described herein, or a vector described herein, for the manufacture of a medicament for managing, preventing or treating a disease or disorder caused by VZV or by infection with VZV in a subject. In some embodiments, provided herein is a use of a pharmaceutical composition described herein, for the manufacture of a medicament for managing, preventing or treating a disease or disorder caused by VZV or by infection with VZV in a subject. In some embodiments, the disease or disorder caused by VZV is (a) varicella or zoster; (b) postherpetic neuralgia (PHN); or (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella and zoster; (b) postherpetic neuralgia (PHN); or (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella or zoster; (b) postherpetic neuralgia (PHN); and (c) one or more of meningoencephalitis, myelitis, cranial nerve palsy, angiopathy, keratitis, retinopathy, ulcers, hepatitis and pancreatitis. In some embodiments, the disease or disorder caused by VZV is (a) varicella and zoster; (b) postherpetic neuralgia (PHN); and (c) one or more of meningoencephalitis, myelitis, cranial nerve palsy, angiopathy, keratitis, retinopathy, ulcers, hepatitis and pancreatitis. In some embodiments, the subject is a human. In some embodiments, the human is a human adult. In some embodiments, the adult is at least 40 years old. In some embodiments, the adult is at least 45 years old. In some embodiments, the adult is at least 50 years old. In some embodiments, the adult is at least 55 years old. In some embodiments, the adult is at least 60 years old. In some embodiments, the human is an elderly human.

### 6. BRIEF DESCRIPTION OF THE FIGURES

FIG. 1 shows the expression of the candidates by HEK293 T transfected in vitro. The suffix psd represents pseudo-U modification, and the suffix m1 represents 1-N-pseudo-U modification.

FIG. 2 shows the gE specific IgG titer induced by the candidates. The suffix psd represents pseudo-U modification, and the suffix m1 represents 1-N-pseudo-U modification.

FIG. 3 shows the gE peptide pool specific T cell response induced by the candidates. The suffix psd represents pseudo-U modification, and the suffix m1 represents 1-N-pseudo-U modification.

FIG. 4 shows the predicted secondary structure of full-length VZV gE. A, wild type; B, mutant comprising substitutions Y569A, Y582G, S593A, S595A, T596A, and T598A.

FIG. 5 shows the median fluorescence intensity of transfected cells in vitro by the candidates.

FIG. 6 shows the expression rate of transfected cells in vitro by the candidates.

FIG. 7 shows the gE specific IgG titer induced by candidates.

FIG. 8 shows the gE peptide pool specific T cell response induced by candidates.

FIG. 9 shows the location of the target protein in the cell. Green represents the gE protein, red denotes the Golgi apparatus, and blue indicates the cell nucleus.

FIG. 10 showstheanalysisofinflammatoryresponseinhibitionbyRIG-Iactivation and IFN-β release using mRNA in vitro.

FIG. 11 shows the expression rate of transfected cells in vitro by the candidates.

FIG. 12 shows the median fluorescence intensity of transfected cells in vitro by the candidates.

FIG. 13A shows the gE specific IgG titer after the first immunization in Naïve mice.

FIG. 13B shows the gE specific IgG titer after the boost immunization in Naïve mice.

FIG. 13C shows the gE specific IgG titer after the first immunization in LAV-experienced mice.

FIG. 13D shows the gE specific IgG titer after the boost immunization in LAV-experienced mice.

FIG. 14A shows the CD4+ T cells response induced by vaccines in Naïve mice.

FIG. 14B shows the CD4+ T cells response induced by vaccines in LAV-experienced mice.

### 7. DETAILED DESCRIPTION

Provided herein are therapeutic nucleic acid molecules useful for the prevention, management and treatment of a disease or disorder caused by VZV or by infection with VZV. Also provided herein are pharmaceutical composition comprising the therapeutic nucleic acid molecules, including pharmaceutical composition formulated as lipid nanoparticles and related therapeutic methods and uses for preventing, managing and treating of a disease or disorder cause by VZV or by infection with VZV. Additional features of the present disclosure will become apparent to those skilled in the art upon consideration of the following detailed description of particular embodiments.

### 7.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003).

### 7.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein and unless otherwise specified, the term “lipid” refers to a group of organic compounds that include, but are not limited to, esters of fatty acids and are generally characterized by being poorly soluble in water, but soluble in many nonpolar organic solvents. While lipids generally have poor solubility in water, there are certain categories of lipids (e.g., lipids modified by polar groups, e.g., DMG-PEG2000) that have limited aqueous solubility and can dissolve in water under certain conditions. Known types of lipids include biological molecules such as fatty acids, waxes, sterols, fat-soluble vitamins, monoglycerides, diglycerides, triglycerides, and phospholipids. Lipids can be divided into at least three classes: (1) “simple lipids,” which include fats and oils as well as waxes; (2) “compound lipids,” which include phospholipids and glycolipids (e.g., DMPE-PEG2000); and (3) “derived lipids” such as steroids. Further, as used herein, lipids also encompass lipidoid compounds. The term “lipidoid compound,” also simply “lipidoid”, refers to a lipid-like compound (e.g. an amphiphilic compound with lipid-like physical properties).

The term “lipid nanoparticle” or “LNP” refers to a particle having at least one dimension on the order of nanometers (nm) (e.g., 1 to 1,000 nm), which contains one or more types of lipid molecules. The LNP provided herein can further contain at least one non-lipid payload molecule (e.g., one or more nucleic acid molecules). In some embodiments, the LNP comprises a non-lipid payload molecule either partially or completely encapsulated inside a lipid shell. Particularly, in some embodiments, wherein the payload is a negatively charged molecule (e.g., mRNA encoding a viral protein), and the lipid components of the LNP comprise at least one cationic lipid. Without being bound by the theory, it is contemplated that the cationic lipids can interact with the negatively charged payload molecules and facilitates incorporation and/or encapsulation of the payload into the LNP during LNP formation. Other lipids that can form part of a LNP as provided herein include but are not limited to neutral lipids and charged lipids, such as steroids, polymer conjugated lipids, and various zwitterionic lipids. In certain embodiments, a LNP according to the present disclosure comprises one or more lipids of Series 01, 02, 03, and 04, e.g., one or more lipids of Formula (01-I), (01-II), (02-I), (03-I), and (04-I) (and sub-formulas thereof) as described herein.

The term “cationic lipid” refers to a lipid that is either positively charged at any pH value or hydrogen ion activity of its environment, or capable of being positively charged in response to the pH value or hydrogen ion activity of its environment (e.g., the environment of its intended use). Thus, the term “cationic” encompasses both “permanently cationic” and “cationisable.” In certain embodiments, the positive charge in a cationic lipid results from the presence of a quaternary nitrogen atom. In certain embodiments, the cationic lipid comprises a zwitterionic lipid that assumes a positive charge in the environment of its intended use (e.g., at physiological pH). In certain embodiments, the cationic lipid is one or more lipids of Series 01, 02, 03, and 04, e.g., one or more lipids of Formula (01-I), (01-II), (02-I), (03-I), and (04-1) (and sub-formulas thereof) as described herein. The term “anionic lipid” refers to a lipid that is either negatively charged at any pH value or hydrogen ion activity of its environment or capable of being negatively charged in response to the pH value or hydrogen ion activity of its environment (e.g., the environment of its intended use). Exemplary anionic lipids include one or more phosphate group(s) which bear a negative charge, for example at physiological pHs.

The term “polymer conjugated lipid” refers to a molecule comprising both a lipid portion and a polymer portion. An example of a polymer conjugated lipid is a pegylated lipid (PEG-lipid), in which the polymer portion comprises a polyethylene glycol.

The term “neutral lipid” encompasses any lipid molecules existing in uncharged forms or neutral zwitterionic forms at a selected pH value or within a selected pH range. In some embodiments, the selected useful pH value or range corresponds to the pH condition in an environment of the intended uses of the lipids, such as the physiological pH. As non-limiting examples, neutral lipids that can be used in connection with the present disclosure include, but are not limited to, phosphotidylcholines such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), 2-((2,3-bis(oleoyloxy)propyl)dimethylammonio)ethyl hydrogen phosphate (DOCP), sphingomyelins (SM), ceramides, steroids such as sterols and their derivatives. Neutral lipids as provided herein may be synthetic or derived (isolated or modified) from a natural source or compound.

The term “charged lipid” encompasses any lipid molecules that exist in either positively charged or negatively charged forms at a selected pH or within a selected pH range. In some embodiments, the selected pH value or range corresponds to the pH condition in an environment of the intended uses of the lipids, such as the physiological pH. As non-limiting examples, neutral lipids that can be used in connection with the present disclosure include, but are not limited to, phosphatidylserines, phosphatidic acids, phosphatidylglycerols, phosphatidylinositols, sterol hemisuccinates, dialkyl trimethylarnmonium-propanes, (e.g., DOTAP, DOTMA), dialkyl dimethylaminopropanes, ethyl phosphocholines, dimethylaminoethane carbamoyl sterols (e.g., DC-Chol), 1,2-dioleoyl-sn-glycero-3-phospho-L-serine sodium salt (DOPS-Na), 1,2-dioleoyl-sn-glycero-3-phospho-(1′-rac-glycerol) sodium salt (DOPG-Na), and 1,2-dioleoyl-sn-glycero-3-phosphate sodium salt (DOPA-Na). Charged lipids as provided herein may be synthetic or derived (isolated or modified) from a natural source or compound.

As used herein, and unless otherwise specified, the term “alkyl” refers to a straight or branched hydrocarbon chain radical consisting solely of carbon and hydrogen atoms, which is saturated. In one embodiment, the alkyl group has, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond. Examples of alkyl groups include, but are not limited to, methyl, ethyl, n-propyl, 1-methylethyl (isopropyl), n-butyl, n-pentyl, 1,1-dimethylethyl (t-butyl), 3-methylhexyl, 2-methylhexyl, and the like. Unless otherwise specified, an alkyl group is optionally substituted.

As used herein, and unless otherwise specified, the term “alkenyl” refers to a straight or branched hydrocarbon chain radical consisting solely of carbon and hydrogen atoms, which contains one or more carbon-carbon double bonds. The term “alkenyl” also embraces radicals having “cis” and “trans” configurations, or alternatively, “E” and “Z” configurations, as appreciated by those of ordinary skill in the art. In one embodiment, the alkenyl group has, for example, from two to twenty-four carbon atoms (C₂-C₂₄ alkenyl), four to twenty carbon atoms (C₄-C₂₀ alkenyl), six to sixteen carbon atoms (C₆-C₁₆ alkenyl), six to nine carbon atoms (C₆-C₉ alkenyl), two to fifteen carbon atoms (C₂-C₁₅ alkenyl), two to twelve carbon atoms (C₂-C₁₂ alkenyl), two to eight carbon atoms (C₂-C₈ alkenyl) or two to six carbon atoms (C₂-C₆ alkenyl) and which is attached to the rest of the molecule by a single bond. Examples of alkenyl groups include, but are not limited to, ethenyl, prop-1-enyl, but-1-enyl, pent-1-enyl, penta-1,4-dienyl, and the like. Unless otherwise specified, an alkenyl group is optionally substituted.

As used herein, and unless otherwise specified, the term “alkynyl” refers to a straight or branched hydrocarbon chain radical consisting solely of carbon and hydrogen atoms, which contains one or more carbon-carbon triple bonds. In one embodiment, the alkynyl group has, for example, from two to twenty-four carbon atoms (C₂-C₂₄ alkynyl), four to twenty carbon atoms (C₄-C₂₀ alkynyl), six to sixteen carbon atoms (C₆-C₁₆ alkynyl), six to nine carbon atoms (C₆-C₉ alkynyl), two to fifteen carbon atoms (C₂-C₁₅ alkynyl), two to twelve carbon atoms (C₂-C₁₂ alkynyl), two to eight carbon atoms (C₂-C₈ alkynyl) or two to six carbon atoms (C₂-C₆ alkynyl) and which is attached to the rest of the molecule by a single bond. Examples of alkynyl groups include, but are not limited to, ethynyl, propynyl, butynyl, pentynyl, and the like. Unless otherwise specified, an alkynyl group is optionally substituted.

As used herein, and unless otherwise specified, the term “alkylene” or “alkylene chain” refers to a straight or branched divalent hydrocarbon chain linking the rest of the molecule to a radical group, consisting solely of carbon and hydrogen, which is saturated. In one embodiment, the alkylene has, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene). Examples of alkylene groups include, but are not limited to, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless otherwise specified, an alkylene chain is optionally substituted.

As used herein, and unless otherwise specified, the term “alkenylene” refers to a straight or branched divalent hydrocarbon chain linking the rest of the molecule to a radical group, consisting solely of carbon and hydrogen, which contains one or more carbon-carbon double bonds. In one embodiment, the alkenylene has, for example, from two to twenty-four carbon atoms (C₂-C₂₄ alkenylene), two to fifteen carbon atoms (C₂-C₁₅ alkenylene), two to twelve carbon atoms (C₂-C₁₂ alkenylene), two to eight carbon atoms (C₂-C₈ alkenylene), two to six carbon atoms (C₂-C₆ alkenylene) or two to four carbon atoms (C₂-C₄ alkenylene). Examples of alkenylene include, but are not limited to, ethenylene, propenylene, n-butenylene, and the like. The alkenylene is attached to the rest of the molecule through a single or double bond and to the radical group through a single or double bond. The points of attachment of the alkenylene to the rest of the molecule and to the radical group can be through one carbon or any two carbons within the chain. Unless otherwise specified, an alkenylene is optionally substituted.

As used herein, and unless otherwise specified, the term “cycloalkyl” refers to a non-aromatic monocyclic or polycyclic hydrocarbon radical consisting solely of carbon and hydrogen atoms, and which is saturated. Cycloalkyl group may include fused or bridged ring systems. In one embodiment, the cycloalkyl has, for example, from 3 to 15 ring carbon atoms (C₃-C₁₅ cycloalkyl), from 3 to 10 ring carbon atoms (C₃-C₁₀ cycloalkyl), or from 3 to 8 ring carbon atoms (C₃-C₈ cycloalkyl). The cycloalkyl is attached to the rest of the molecule by a single bond. Examples of monocyclic cycloalkyl radicals include, but are not limited to, cyclopropyl, cyclobutyl, cyclopentyl, cyclohexyl, cycloheptyl, and cyclooctyl. Examples of polycyclic cycloalkyl radicals include, but are not limited to, adamantyl, norbornyl, decalinyl, 7,7-dimethyl-bicyclo[2.2.1]heptanyl, and the like. Unless otherwise specified, a cycloalkyl group is optionally substituted.

As used herein, and unless otherwise specified, the term “cycloalkylene” is a divalent cycloalkyl group. Unless otherwise specified, a cycloalkylene group is optionally substituted.

As used herein, and unless otherwise specified, the term “cycloalkenyl” refers to a non-aromatic monocyclic or polycyclic hydrocarbon radical consisting solely of carbon and hydrogen atoms, and which includes one or more carbon-carbon double bonds. Cycloalkenyl may include fused or bridged ring systems. In one embodiment, the cycloalkenyl has, for example, from 3 to 15 ring carbon atoms (C₃-C₁₅ cycloalkenyl), from 3 to 10 ring carbon atoms (C₃-C₁₀ cycloalkenyl), or from 3 to 8 ring carbon atoms (C₃-C₈ cycloalkenyl). The cycloalkenyl is attached to the rest of the molecule by a single bond. Examples of monocyclic cycloalkenyl radicals include, but are not limited to, cyclopropenyl, cyclobutenyl, cyclopentenyl, cyclohexenyl, cycloheptenyl, cyclooctenyl, and the like. Unless otherwise specified, a cycloalkenyl group is optionally substituted.

As used herein, and unless otherwise specified, the term “cycloalkenylene” is a divalent cycloalkenyl group. Unless otherwise specified, a cycloalkenylene group is optionally substituted.

As used herein, and unless otherwise specified, the term “heterocyclyl” refers to a non-aromatic radical monocyclic or polycyclic moiety that contains one or more (e.g., one, one or two, one to three, or one to four)heteroatoms independently selected from nitrogen, oxygen, phosphorous, and sulfur. The heterocyclyl may be attached to the main structure at any heteroatom or carbon atom. A heterocyclyl group can be a monocyclic, bicyclic, tricyclic, tetracyclic, or other polycyclic ring system, wherein the polycyclic ring systems can be a fused, bridged or spiro ring system. Heterocyclyl polycyclic ring systems can include one or more heteroatoms in one or more rings. A heterocyclyl group can be saturated or partially unsaturated. Saturated heterocycloalkyl groups can be termed “heterocycloalkyl”. Partially unsaturated heterocycloalkyl groups can be termed “heterocycloalkenyl” if the heterocyclyl contains at least one double bond, or “heterocycloalkynyl” if the heterocyclyl contains at least one triple bond. In one embodiment, the heterocyclyl has, for example, 3 to 18 ring atoms (3- to 18-membered heterocyclyl), 4 to 18 ring atoms (4- to 18-membered heterocyclyl), 5 to 18 ring atoms (3- to 18-membered heterocyclyl), 4 to 8 ring atoms (4- to 8-membered heterocyclyl), or 5 to 8 ring atoms (5- to 8-membered heterocyclyl). Whenever it appears herein, a numerical range such as “3 to 18” refers to each integer in the given range; e.g., “3 to 18 ring atoms” means that the heterocyclyl group can consist of 3 ring atoms, 4 ring atoms, 5 ring atoms, 6 ring atoms, 7 ring atoms, 8 ring atoms, 9 ring atoms, 10 ring atoms, etc., up to and including 18 ring atoms. Examples of heterocyclyl groups include, but are not limited to, imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl. Unless otherwise specified, a heterocyclyl group is optionally substituted.

As used herein, and unless otherwise specified, the term “heterocyclylene” is a divalent heterocyclyl group. Unless otherwise specified, a heterocyclylene group is optionally substituted

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 18 ring carbon atoms (C₆-C₁₈ aryl), from 6 to 14 ring carbon atoms (C₆-C₁₄ aryl), or from 6 to 10 ring carbon atoms (C₆-C₁₀ aryl). Examples of aryl groups include, but are not limited to, phenyl, naphthyl, fluorenyl, azulenyl, anthryl, phenanthryl, pyrenyl, biphenyl, and terphenyl. The term “aryl” also refers to bicyclic, tricyclic, or other multicyclic hydrocarbon rings, where at least one of the rings is aromatic and the others of which may be saturated, partially unsaturated, or aromatic, for example, dihydronaphthyl, indenyl, indanyl, or tetrahydronaphthyl (tetralinyl). Unless otherwise specified, an aryl group is optionally substituted.

As used herein, and unless otherwise specified, the term “arylene” is a divalent aryl group. Unless otherwise specified, an arylene group is optionally substituted.

As used herein, and unless otherwise specified, the term “heteroaryl” refers to a monocyclic aromatic group and/or multicyclic aromatic group that contains at least one aromatic ring, wherein at least one aromatic ring contains one or more (e.g., one, one or two, one to three, or one to four) heteroatoms independently selected from O, S, and N. The heteroaryl may be attached to the main structure at any heteroatom or carbon atom. In certain embodiments, the heteroaryl has from 5 to 20, from 5 to 15, or from 5 to 10 ring atoms. The term “heteroaryl” also refers to bicyclic, tricyclic, or other multicyclic rings, where at least one of the rings is aromatic and the others of which may be saturated, partially unsaturated, or aromatic, wherein at least one aromatic ring contains one or more heteroatoms independently selected from O, S, and N. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. Unless otherwise specified, a heteroaryl group is optionally substituted.

As used herein, and unless otherwise specified, the term “heteroarylene” is a divalent heteroaryl group. Unless otherwise specified, a heteroarylene group is optionally substituted.

When the groups described herein are said to be “substituted,” they may be substituted with any appropriate substituent or substituents. Illustrative examples of substituents include, but are not limited to, those found in the exemplary compounds and embodiments provided herein, as well as: a halogen atom such as F, Cl, Br, or I; cyano; oxo (═O); hydroxyl (—OH); alkyl; alkenyl; alkynyl; cycloalkyl; aryl; —(C═O)OR′; —O(C═O)R′; —C(═O)R′; —OR′; —S(O)ₓR′; —S—SR′; —C(═O)SR′; —SC(═O)R′; —NR′R′; —NR′C(═O)R′; —C(═O)NR′R′; —NR′C(═O)NR′R′; —OC(═O)NR′R′; —NR′C(═O)OR′; —NR'S(O)ₓNR′R′; —NR'S(O)ₓR′; and —S(O)ₓNR′R′, wherein: R′ is, at each occurrence, independently H, C₁-C₁₅ alkyl or cycloalkyl, and x is 0, 1 or 2. In some embodiments the substituent is a C₁-C₁₂ alkyl group. In other embodiments, the substituent is a cycloalkyl group. In other embodiments, the substituent is a halo group, such as fluoro. In other embodiments, the substituent is an oxo group. In other embodiments, the substituent is a hydroxyl group. In other embodiments, the substituent is an alkoxy group (—OR′). In other embodiments, the substituent is a carboxyl group. In other embodiments, the substituent is an amino group (—NR′R′).

As used herein, and unless otherwise specified, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

As used herein, and unless otherwise specified, the term“prodrug” of a biologically active compound refers to a compound that may be converted under physiological conditions or by solvolysis to the biologically active compound. In one embodiment, the term “prodrug” refers to a metabolic precursor of the biologically active compound that is pharmaceutically acceptable. A prodrug may be inactive when administered to a subject in need thereof, but is converted in vivo to the biologically active compound. Prodrugs are typically rapidly transformed in vivo to yield the parent biologically active compound, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Dmg Design, Ed. Edward B. Roche, American Pharmaceutical Association and Pergamon Press, 1987.

In one embodiment, the term “prodrug” is also meant to include any covalently bonded carriers, which release the active compound in vivo when such prodrug is administered to a mammalian subject. Prodrugs of a compound may be prepared by modifying functional groups present in the compound in such a way that the modifications are cleaved, either in routine manipulation or in vivo, to the parent compound. Prodrugs include compounds wherein a hydroxyl, amino or mercapto group is bonded to any group that, when the prodrug of the compound is administered to a mammalian subject, cleaves to form a free hydroxyl, free amino or free mercapto group, respectively.

Examples of prodrugs include, but are not limited to, acetate, formate and benzoate derivatives of alcohol or amide derivatives of amine functional groups in the compounds provided herein.

As used herein, and unless otherwise specified, the term “pharmaceutically acceptable salt” includes both acid and base addition salts.

Examples of pharmaceutically acceptable acid addition salts include, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Examples of pharmaceutically acceptable base addition salt include, but are not limited to, salts prepared from addition of an inorganic base or an organic base to a free acid compound. Salts derived from inorganic bases include, but are not limited to, the sodium, potassium, lithium, ammonium, calcium, magnesium, iron, zinc, copper, manganese, aluminum salts and the like. In one embodiment, the inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In one embodiment, the organic bases are isopropylamine, diethylamine, ethanolamine, trimethylamine, dicyclohexylamine, choline and caffeine.

A compound provided herein may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. Unless otherwise specified, a compound provided herein is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution.

It should also be noted a compound described herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of a compound described herein, whether radioactive or not, are intended to be encompassed within the scope of the embodiments provided herein. In some embodiments, there are provided isotopologs of a compound described herein, for example, the isotopologs are deuterium, carbon-13, and/or nitrogen-15 enriched. As used herein, “deuterated”, means a compound wherein at least one hydrogen (H) has been replaced by deuterium (indicated by D or ²H), that is, the compound is enriched in deuterium in at least one position.

It should be noted that if there is a discrepancy between a depicted structure and a name for that structure, the depicted structure is to be accorded more weight.

As used herein, and unless otherwise specified, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The term “composition” is intended to encompass a product containing the specified ingredients (e.g., a mRNA molecule provided herein) in, optionally, the specified amounts.

The term “polynucleotide” or “nucleic acid,” as used interchangeably herein, refers to polymers of nucleotides of any length and includes, e.g., DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. Nucleic acid can be in either single- or double-stranded forms. As used herein and unless otherwise specified, “nucleic acid” also includes nucleic acid mimics such as locked nucleic acids (LNAs), peptide nucleic acids (PNAs), and morpholinos. “Oligonucleotide,” as used herein, refers to short synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, the term “non-naturally occurring” when used in reference to a nucleic acid molecule as described herein is intended to mean that the nucleic acid molecule is not found in nature. A non-naturally occurring nucleic acid encoding a viral peptide or protein contains at least one genetic alternation or chemical modification not normally found in a naturally occurring strain of the virus, including wild-type strains of the virus. Genetic alterations include, for example, modifications introducing expressible nucleic acid sequences encoding peptides or polypeptides heterologous to the virus, other nucleic acid additions, nucleic acid deletions, nucleic acid substitution, and/or other functional disruption of the vims' genetic material. Such modifications include, for example, modifications in the coding regions and functional fragments thereof, for heterologous, homologous or both heterologous and homologous polypeptides for the viral species. Additional modifications include, for example, modifications in non-coding regulatory regions in which the modifications alter expression of a gene or operon. Additional modifications also include, for example, incorporation of a nucleic acid sequence into a vector, such as a plasmid or an artificial chromosome. Chemical modifications include, for example, one or more functional nucleotide analog as described herein.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as an mRNA molecule, can be substantially free of other cellular material, or culture medium when produced by recombinant techniques, or substantially free of chemical precursors or other chemicals when chemically synthesized. In a specific embodiment, one or more nucleic acid molecules encoding an antigen as described herein are isolated or purified. The term embraces nucleic acid sequences that have been removed from their naturally occurring environment, and includes recombinant or cloned DNA or RNA isolates and chemically synthesized analogues or analogues biologically synthesized by heterologous systems. A substantially pure molecule may include isolated forms of the molecule.

The term “encoding nucleic acid” or grammatical equivalents thereof as it is used in reference to nucleic acid molecule encompasses (a) a nucleic acid molecule in its native state or when manipulated by methods well known to those skilled in the art that can be transcribed to produce mRNA which is then translated into a peptide and/or polypeptide, and (b) the mRNA molecule itself. The antisense strand is the complement of such a nucleic acid molecule, and the encoding sequence can be deduced therefrom. The term “coding region” refers to a portion in an encoding nucleic acid sequence that is translated into a peptide or polypeptide. The term “untranslated region” or “UTR” refers to the portion of an encoding nucleic acid that is not translated into a peptide or polypeptide. Depending on the orientation of a UTR with respect to the coding region of a nucleic acid molecule, a UTR is referred to as the 5′-UTR if located to the 5′-end of a coding region, and a UTR is referred to as the 3′-UTR if located to the 3′-end of a coding region.

The term “mRNA” as used herein refers to a message RNA molecule comprising one or more open reading frame (ORF) that can be translated by a cell or an organism provided with the mRNA to produce one or more peptide or protein product. The region containing the one or more ORFs is referred to as the coding region of the mRNA molecule. In certain embodiments, the mRNA molecule further comprises one or more untranslated regions (UTRs).

In certain embodiments, the mRNA is a monocistronic mRNA that comprises only one ORF. In certain embodiments, the monocistronic mRNA encodes a peptide or protein comprising at least one epitope of a selected antigen (e.g., a pathogenic antigen or a tumor associated antigen). In other embodiments, the mRNA is a multicistronic mRNA that comprises two or more ORFs. In certain embodiments, the multiecistronic mRNA encodes two or more peptides or proteins that can be the same or different from each other. In certain embodiments, each peptide or protein encoded by a multicistronic mRNA comprises at least one epitope of a selected antigen. In certain embodiments, different peptide or protein encoded by a multicistronic mRNA each comprises at least one epitope of different antigens. In any of the embodiments described herein, the at least one epitope can be at least 2, at least 3, at least 4, at least 5, at least 6, at least 7, at least 8, at least 9, or at least 10 epitopes of an antigen.

The term “nucleobases” encompasses purines and pyrimidines, including natural compounds adenine, thymine, guanine, cytosine, uracil, inosine, and natural or synthetic analogs or derivatives thereof.

The term “functional nucleotide analog” as used herein refers to a modified version of a canonical nucleotide A, G, C, U or T that (a) retains the base-pairing properties of the corresponding canonical nucleotide, and (b) contains at least one chemical modification to (i) the nucleobase, (ii) the sugar group, (iii) the phosphate group, or (iv) any combinations of (i) to (iii), of the corresponding natural nucleotide. As used herein, base pairing encompasses not only the canonical Watson-Crick adenine-thymine, adenine-uracil, or guanine-cytosine base pairs, but also base pairs formed between canonical nucleotides and functional nucleotide analogs or between a pair of functional nucleotide analogs, wherein the arrangement of hydrogen bond donors and hydrogen bond acceptors permits hydrogen bonding between a modified nucleobase and a canonical nucleobase or between two complementary modified nucleobase structures. For example, a functional analog of guanosine (G) retains the ability to base-pair with cytosine (C) or a functional analog of cytosine. One example of such non-canonical base pairing is the base pairing between the modified nucleotide inosine and adenine, cytosine, or uracil. As described herein, a functional nucleotide analog can be either naturally occurring or non-naturally occurring. Accordingly, a nucleic acid molecule containing a functional nucleotide analog can have at least one modified nucleobase, sugar group and/or internucleoside linkage. Exemplary chemical modifications to the nucleobases, sugar groups, or internucleoside linkages of a nucleic acid molecule are provided herein.

The terms “translational enhancer element,” “TEE” and “translational enhancers” as used herein refers to a region in a nucleic acid molecule that functions to promotes translation of a coding sequence of the nucleic acid into a protein or peptide product, such as via cap-dependent or cap-independent translation. A TEE typically locates in the UTR region of a nucleic acid molecule (e.g., mRNA) and enhance the translational level of a coding sequence located either upstream or downstream. For example, a TEE in a 5′-UTR of a nucleic acid molecule can locate between the promoter and the starting codon of the nucleic acid molecule. Various TEE sequences are known in the art (Wellensiek et al. Genome-wide profiling of human cap-independent translation-enhancing elements, Nature Methods, 2013 August; 10(8): 747-750; Chappell et al. PNAS Jun. 29, 2004 101 (26) 9590-9594). Some TEEs are known to be conserved across multiple species (Pánek et al. Nucleic Acids Research, Volume 41, Issue 16, 1 Sep. 2013, Pages 7625-7634).

As used herein, the term “stem-loop sequence” refers to a single-stranded polynucleotide sequence having at least two regions that are complementary or substantially complementary to each other when read in opposite directions, and thus capable of base-pairing with each other to form at least one double helix and an unpaired loop. The resulting structure is known as a stem-loop structure, a hairpin, or a hairpin loop, which is a secondary structure found in many RNA molecules.

The term “peptide” as used herein refers to a polymer containing between two and fifty (2-50) amino acid residues linked by one or more covalent peptide bond(s). The terms apply to naturally occurring amino acid polymers as well as amino acid polymers in which one or more amino acid residues is a non-naturally occurring amino acid (e.g., an amino acid analog or non-natural amino acid).

The terms “polypeptide” and “protein” are used interchangeably herein to refer to a polymer of greater than fifty (50) amino acid residues linked by covalent peptide bonds. That is, a description directed to a polypeptide applies equally to a description of a protein, and vice versa. The terms apply to naturally occurring amino acid polymers as well as amino acid polymers in which one or more amino acid residues is a non-naturally occurring amino acid (e.g., an amino acid analog). As used herein, the terms encompass amino acid chains of any length, including full length proteins (e.g., antigens).

In the context of a peptide or polypeptide, the term “derivative” as used herein refers to a peptide or polypeptide that comprises an amino acid sequence of the viral peptide or protein, or a fragment of a viral peptide or protein, which has been altered by the introduction of amino acid residue substitutions, deletions, or additions. The term “derivative” as used herein also refers to a viral peptide or protein, or a fragment of a viral peptide or protein, which has been chemically modified, e.g., by the covalent attachment of any type of molecule to the polypeptide. For example, but not by way of limitation, a viral peptide or protein or a fragment of the viral peptide or protein may be chemically modified, e.g., by glycosylation, acetylation, pegylation, phosphorylation, amidation, derivatization by known protecting/blocking groups, proteolytic cleavage, chemical cleavage, formulation, metabolic synthesis of tunicamycin, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the viral peptide or protein. Further, a derivative of a viral peptide or protein or a fragment of a viral peptide or protein may contain one or more non-classical amino acids. In specific embodiments, a derivative is a functional derivative of the native or unmodified peptide or polypeptide from which it was derived.

The term “functional derivative” refers to a derivative that retains one or more functions or activities of the naturally occurring or starting peptide or polypeptide from which it was derived. For example, a functional derivative of a VZV S protein may retain the ability to bind one or more of its receptors on a host cell. For example, a functional derivative of a VZV N protein may retain the ability to bind RNA or the package viral genome.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, fragments refers to polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the amino acid sequence of a polypeptide. In a specific embodiment, a fragment of a polypeptide retains at least 1, at least 2, at least 3, or more functions of the polypeptide.

The term “immunogenic fragment” as used herein in the context of a peptide or polypeptide (e.g., a protein), refers to a fragment of a peptide or polypeptide that retains the ability of the peptide or polypeptide in eliciting an immune response upon contacting the immune system of a mammal, including innate immune responses and/or adaptive immune responses. In some embodiments, an immunogenic fragment of a peptide or polypeptide can be an epitope.

The term “antigen” refers to a substance that can be recognized by the immune system of a subject (including by the adaptive immune system), and is capable of triggering an immune response after the subject is contacted with the antigen (including an antigen-specific immune response). In certain embodiments, the antigen is a protein associated with a diseased cell, such as a cell infected by a pathogen or a neoplastic cell (e.g., tumor associated antigen (TAA)).

An “epitope” is the site on the surface of an antigen molecule to which a single antibody molecule binds, such as a localized region on the surface of an antigen that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. In certain embodiments, an epitope is a three-dimensional surface feature of a polypeptide. In other embodiments, an epitope is linear feature of a polypeptide. Generally, an antigen has several or many different epitopes and may react with many different antibodies.

The term “heterologous” refers an entity not found in nature to be associated with (e.g., encoded by and/or expressed by the genome of) a naturally occurring VZV. The term “homologous” refers an entity found in nature to be associated with (e.g., encoded by and/or expressed by the genome of) a naturally occurring VZV.

The term “genetic vaccine” as used herein refers to a therapeutic or prophylactic composition comprising at least one nucleic acid molecule encoding an antigen associated with a target disease (e.g., an infectious disease or a neoplastic disease). Administration of the vaccine to a subject (“vaccination”) allows for the production of the encoded peptide or protein, thereby eliciting an immune response against the target disease in the subject. In certain embodiments, the immune response comprises adaptive immune response, such as the production of antibodies against the encoded antigen, and/or activation and proliferations of immune cells capable of specifically eliminating diseased cells expressing the antigen. In certain embodiments, the immune response further comprises innate immune response. According to the present disclosure, a vaccine can be administered to a subject either before or after the onset of clinical symptoms of the target disease. In some embodiments, vaccination of a healthy or asymptomatic subject renders the vaccinated subject immune or less susceptible to the development of the target disease. In some embodiments, vaccination of a subject showing symptoms of the disease improves the condition of, or treats, the disease in the vaccinated subject.

The term “vector” refers to a substance that is used to carry or include a nucleic acid sequence, including for example, a nucleic acid sequence encoding a viral peptide or protein as described herein, in order to introduce a nucleic acid sequence into a host cell, or serve as a transcription template to carry out in vitro transcription reaction in a cell-free system to produce mRNA. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate transcription or translation control sequences. Selectable marker genes that can be included, for example, provide resistance to antibiotics or toxins, complement auxotrophic deficiencies, or supply critical nutrients not in the culture media. Transcription or translation control sequences can include constitutive and inducible promoters, transcription enhancers, transcription terminators, and the like, which are well known in the art. When two or more nucleic acid molecules are to be co-transcribed or co-translated (e.g., nucleic acid molecules encoding two or more different viral peptides or proteins), both nucleic acid molecules can be inserted, for example, into a single expression vector or in separate expression vectors. For single vector transcription and/or translation, the encoding nucleic acids can be operationally linked to one common transcription or translation control sequence or linked to different transcription or translation control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a mRNA transcript of the nucleic acid as described herein), and it is further understood that expression levels can be optimized to obtain sufficient expression using methods well known in the art.

The terms “innate immune response” and “innate immunity” are recognized in the art, and refer to non-specific defense mechanism a body's immune system initiates upon recognition of pathogen-associated molecular patterns, which involves different forms of cellular activities, including cytokine production and cell death through various pathways. As used herein, innate immune responses include, without limitation, increased production of inflammation cytokines (e.g., type I interferon or IL-10 production), activation of the NFκB pathway, increased proliferation, maturation, differentiation and/or survival of immune cells, and in some cases, induction of cell apoptosis. Activation of the innate immunity can be detected using methods known in the art, such as measuring the (NF)-κB activation.

The terms “adaptive immune response” and “adaptive immunity” are recognized in the art, and refer to antigen-specific defense mechanism a body's immune system initiates upon recognition of a specific antigen, which include both humoral response and cell-mediated responses. As used herein, adaptive immune responses include cellular responses that is triggered and/or augmented by a vaccine composition, such as a genetic composition described herein. In some embodiments, the vaccine composition comprises an antigen that is the target of the antigen-specific adaptive immune response. In other embodiments, the vaccine composition, upon administration, allows the production in an immunized subject of an antigen that is the target of the antigen-specific adaptive immune response. Activation of an adaptive immune response can be detected using methods known in the art, such as measuring the antigen-specific antibody production, or the level of antigen-specific cell-mediated cytotoxicity.

“Antibody-dependent cell-mediated cytotoxicity” or “ADCC” refers to a form of cytotoxicity in which secreted immunoglobulin bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. NK cells, the primary cells for mediating ADCC, express FcγRIII only, whereas monocytes express FcγRI, FcγRII, and FcγRIII. FcR expression on hematopoietic cells is known (see, e.g., Ravetch and Kinet, 1991, Annu. Rev. Immunol. 9:457-92). To assess ADCC activity of a molecule of interest, an in vitro ADCC assay (see, e.g., U.S. Pat. Nos. 5,500,362 and 5,821,337) can be performed. Useful effector cells for such assays include peripheral blood mononuclear cells (PBMC) and Natural Killer (NK) cells. Alternatively or additionally, ADCC activity of the molecule of interest may be assessed in vivo, for example, in an animal model (see, e.g., Clynes et al., 1998, Proc. Natl. Acad. Sci. USA 95:652-56). Antibodies with little or no ADCC activity may be selected for use.

“Antibody-dependent cellular phagocytosis” or “ADCP” refers to the destruction of target cells via monocyte or macrophage-mediated phagocytosis when immunoglobulin bound onto Fc receptors (FcRs) present on certain phagocytotic cells (e.g., neutrophils, monocytes, and macrophages) enable these phagocytotic cells to bind specifically to an antigen-bearing target cell and subsequently kill the target cell. To assess ADCP activity of a molecule of interest, an in vitro ADCP assay (see, e.g., Bracher et al., 2007, J. Immunol. Methods 323:160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248:167-82). Antibodies with little or no ADCP activity may be selected for use. [00153]“Fc receptor” or “FcR” describes a receptor that binds to the Fc region of an antibody. An exemplary FcR is a native sequence human FcR. Moreover, an exemplary FcR is one that binds an IgG antibody (e.g., a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof (see, e.g., Daëron, 1997, Annu. Rev. Immunol. 15:203-34). Various FcRs are known (see, e.g., Ravetch and Kinet, 1991, Annu. Rev. Immunol. 9:457-92; Capel et al., 1994, Immunomethods 4:25-34; and de Haas et al., 1995, J. Lab. Clin. Med. 126:330-41). Other FcRs, including those to be identified in the future, are encompassed by the term “FcR” herein. The term also includes the neonatal receptor, FcRn, which is responsible for the transfer of maternal IgGs to the fetus (see, e.g., Guyer et al., 1976, J. Immunol. 117:587-93; and Kim et al., 1994, Eu. J. Immunol. 24:2429-34). Antibody variants with improved or diminished binding to FcRs have been described (see, e.g., WO 2000/42072; U.S. Pat. Nos. 7,183,387; 7,332,581; and 7,335,742; Shields et al. 2001, J. Biol. Chem. 9(2):6591-604).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay (see, e.g., Gazzano-Santoro et al., 1996, J. Immunol. Methods 202:163) may be performed. Polypeptide variants with altered Fc region amino acid sequences (polypeptides with a variant Fc region) and increased or decreased C1q binding capability have been described (see, e.g., U.S. Pat. No. 6,194,551; WO 1999/51642; Idusogie et al., 2000, J. Immunol. 164: 4178-84). Antibodies with little or no CDC activity may be selected for use.

The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3 d ed. 1997). In specific embodiments, the specific molecular antigen can be bound by an antibody provided herein, including a polypeptide, a fragment or an epitope thereof. Antibodies also include, but are not limited to, synthetic antibodies, recombinantly produced antibodies, camelized antibodies, intrabodies, anti-idiotypic (anti-Id) antibodies, and functional fragments of any of the above, which refers to a portion of an antibody heavy or light chain polypeptide that retains some or all of the binding activity of the antibody from which the fragment was derived. Non-limiting examples of functional fragments include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgAQ1, and IgA2) of immunoglobulin molecule.

The term “administer” or “administration” refers to the act of injecting or otherwise physically delivering a substance as it exists outside the body (e.g., a lipid nanoparticle composition as described herein) into a patient, such as by mucosal, intradermal, intravenous, intramuscular delivery, and/or any other method of physical delivery described herein or known in the art. When a disease, disorder, condition, or a symptom thereof, is being treated, administration of the substance typically occurs after the onset of the disease, disorder, condition, or symptoms thereof. When a disease, disorder, condition, or symptoms thereof, are being prevented, administration of the substance typically occurs before the onset of the disease, disorder, condition, or symptoms thereof.

“Chronic” administration refers to administration of the agent(s) in a continuous mode (e.g., for a period of time such as days, weeks, months, or years) as opposed to an acute mode, so as to maintain the initial therapeutic effect (activity) for an extended period of time. “Intermittent” administration is treatment that is not consecutively done without interruption, but rather is cyclic in nature.

The term “targeted delivery” or the verb form “target” as used herein refers to the process that promotes the arrival of a delivered agent (such as a therapeutic payload molecule in a lipid nanoparticle composition as described herein) at a specific organ, tissue, cell and/or intracellular compartment (referred to as the targeted location) more than any other organ, tissue, cell or intracellular compartment (referred to as the non-target location). Targeted delivery can be detected using methods known in the art, for example, by comparing the concentration of the delivered agent in a targeted cell population with the concentration of the delivered agent at a non-target cell population after systemic administration. In certain embodiments, targeted delivery results in at least 2 fold higher concentration at a targeted location as compared to a non-target location.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition, including for example, infection and neoplasia. In some embodiments, the effective amount is a therapeutically effective amount or a prophylactically effective amount.

The term “therapeutically effective amount” as used herein refers to the amount of an agent (e.g., a vaccine composition) that is sufficient to reduce and/or ameliorate the severity and/or duration of a given disease, disorder, or condition, and/or a symptom related thereto (e.g., an infectious disease such as caused by viral infection, or a neoplastic disease such as cancer). A “therapeutically effective amount” of a substance/molecule/agent of the present disclosure (e.g., the lipid nanoparticle composition as described herein) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a lipid nanoparticle composition as described herein or a therapeutic or prophylactic agent contained therein (e.g., a therapeutic mRNA) effective to “treat” a disease, disorder, or condition, in a subject or mammal.

A “prophylactically effective amount” is an amount of an agent or a pharmaceutical composition (e.g., a vaccine composition) that, when administered to a subject, will have the intended prophylactic effect, e.g., preventing, delaying, or reducing the likelihood of the onset (or reoccurrence) of a disease, disorder, condition, or associated symptom(s) (e.g., an infectious disease such as caused by viral infection, or a neoplastic disease such as cancer). In some embodiments, the term “prophylactically effective amount” refers to an amount of a lipid nanoparticle composition described herein or a prophylactic agent contained therein (e.g., a nucleic acid, such as, e.g., a nucleic acid described in Section 5.4 or 6, including an mRNA) effective to “prevent” a disease, disorder, or condition, in a subject or mammal. Typically, but not necessarily, since a prophylactic dose is used in subjects prior to or at an earlier stage of a disease, disorder, or condition, a prophylactically effective amount may be less than a therapeutically effective amount. The full therapeutic or prophylactic effect does not necessarily occur by administration of one dose, and may occur only after administration of a series of doses. Thus, a therapeutically or prophylactically effective amount may be administered in one or more administrations.

The terms “prevent,” “preventing,” and “prevention” refer to reducing the likelihood of the onset (or recurrence) of a disease, disorder, condition, or associated symptom(s)(e.g., an infectious disease such as caused by viral infection, or a neoplastic disease such as cancer).

The terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents, such as a lipid nanoparticle composition as described herein) to “manage” an infectious or neoplastic disease, one or more symptoms thereof, so as to prevent the progression or worsening of the disease.

The term “prophylactic agent” refers to any agent that can totally or partially inhibit the development, recurrence, onset, or spread of disease and/or symptom related thereto in a subject. In some embodiments, a prophylactic agent comprises or consists of a nucleic acid described herein (e.g., a nucleic acid described in Section 5.4 or Section 6). In some embodiments, a prophylactic agent comprises or consists of a protein described herein (e.g, a protein described in Section 5.3). In some embodiments, a prophylactic agent comprises or consists of a vector comprising a nucleic acid described herein.

The term “therapeutic agent” refers to any agent that can be used in treating preventing, or alleviating a disease, disorder, or condition, including in the treatment prevention, or alleviation of one or more symptoms of a disease, disorder, or condition and/or a symptom related thereto. In some embodiments, a therapeutic agent is a nucleic acid described herein (e.g., a nucleic acid described in Section 5.4 or Section 6). In some embodiments, a therapeutic agent is a protein described herein (e.g., a protein described in Section 5.3). In some embodiments, a prophylactic agent comprises or consists of a vector comprising a nucleic acid described herein.

The term “therapy” refers to any protocol, method, and/or agent that can be used in the prevention, management, treatment, and/or amelioration of a disease, disorder, or condition. In certain embodiments, the terms “therapies” and “therapy” refer to a biological therapy, supportive therapy, and/or other therapies useful in the prevention, management, treatment, and/or amelioration of a disease, disorder, or condition, known to one of skill in the art such as medical personnel.

As used herein, a “prophylactically effective serum titer” is the serum titer of an antibody in a subject (e.g., a human), that totally or partially inhibits the development recurrence, onset, or spread of a disease, disorder, or condition, and/or symptom related thereto in the subject.

In certain embodiments, a “therapeutically effective serum titer” is the serum titer of an antibody in a subject (e.g., a human), that reduces the severity, the duration, and/or the symptoms associated with a disease, disorder, or condition, in the subject.

The term “serum titer” refers to an average serum titer in a subject from multiple samples (e.g., at multiple time points) or in a population of at least 10, at least 20, at least 40 subjects, up to about 100, 1000, or more.

The term “side effects” encompasses unwanted and/or adverse effects of a therapy (e.g., a prophylactic or therapeutic agent). Unwanted effects are not necessarily adverse. An adverse effect from a therapy (e.g., a prophylactic or therapeutic agent) might be harmful, uncomfortable, or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, loss of appetite, rashes or swellings at the site of administration, flu-like symptoms such as fever, chills, and fatigue, digestive tract problems, and allergic reactions. Additional undesired effects experienced by patients are numerous and known in the art. Many are described in Physician's Desk Reference (68th ed. 2014).

The terms “subject” and “patient” may be used interchangeably. As used herein, in certain embodiments, a subject is a mammal, such as a non-primate (e.g., cow, pig horse, cat, dog, rat, etc.) or a primate (e.g., monkey and human). In specific embodiments, the subject is a human. In one embodiment, the subject is a mammal (e.g., a human) having an infectious disease or neoplastic disease. In another embodiment, the subject is a mammal (e.g., a human) at risk of developing an infectious disease or neoplastic disease.

The term “elderly human” refers to a human 65 years or older. The term “human adult” refers to a human that is 18 years or older. The term “human child” refers to a human that is 1 year to 18 years old. The term “human toddler” refers to a human that is 1 year to 3 years old. The term “human infant” refers to a newborn to 1 year old human.

The term “detectable probe” refers to a composition that provides a detectable signal. The term includes, without limitation, any fluorophore, chromophore, radiolabel, enzyme, antibody or antibody fragment, and the like, that provide a detectable signal via its activity.

The term “detectable agent” refers to a substance that can be used to ascertain the existence or presence of a desired molecule, such as an antigen encoded by an mRNA molecule as described herein, in a sample or subject. A detectable agent can be a substance that is capable of being visualized or a substance that is otherwise able to be determined and/or measured (e.g., by quantitation).

“Substantially all” refers to at least about 60%, at least about 65%, at least about 70%, at least about 75%, at least about 80%, at least about 85%, at least about 90%, at least about 95%, at least about 98%, at least about 99%, or about 100%.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, 0.05%, or less of a given value or range.

The singular terms “a,” “an,” and “the” as used herein include the plural reference unless the context clearly indicates otherwise.

All publications, patent applications, accession numbers, and other references cited in this specification are herein incorporated by reference in their entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section and examples are intended to illustrate but not limit the scope of invention described in the claims.

### 7.3 Proteins

VZV glycoproteins include glycoprotein E (gE), glycoprotein B (gB), glycoprotein H (gH), and glycoprotein L (gL) with gE being the most abundant VSV glycoprotein expressed by VZV-infected cells. VZV gE is generally 623 amino acid residues in length and includes a signal peptide (generally amino acid residues 1-30 of full length gE; e.g., the signal peptide of GenBank Accession No. AAG32558.1 consists of amino acid residues 1-30 of GenBank Accession No. AAG32558.1) and a transmembrane domain. Mature gE lacks the signal peptide. VZV gE forms a heterodimer with gI and the heterodimer gE/gI is required for cell-to-cell spread of the virus. In addition, the VZV gE/gI heterodimer interacts with the Fc region of IgG. VZV gE binds to insulin degrading enzyme (IDE). VZV gE is phosphorylated by the viral kinase encoded by ORF47. Exemplary VZV gE may be found in GenBank Accession Nos. AAG32558.1, AAF61669.1, AAK19946.1, AAK01056.1, AAK19955.1, ABF21641.1, ABF22006.1, ABF22152.1, ABF22152.1, ABF22225.1, ABF22298.1, AEW88044.1, AEW88116.1, AAY57677.1, AAY57748.1, Q9J3M8.1, CAA27951.1, QCA47220.1, AEW88980.1, WWU03079.1, and AEW88548.1, and Uniprot Nos. Q9J3M8, an P09259. In some embodiments, the VZV gE is the gE of the Dumas strain. In some embodiments, the VZV gE is the gE of the KPZ13-287 strain. In some embodiments, the VZV gE is the gE of the VZVi/Munich.GER/30.07/Z[3] strain. In some embodiments, the VZV gE is the gE of the NSYY3 strain. In some embodiments, the VZV gE is the gE of the 1002/2008 strain. In some embodiments, the VZV gE is the gE of the NSYY3 strain. In some embodiments, the VZV gE is the gE of the Oka strain.

In some embodiments, provided herein is fragment of a mature VZV gE, wherein the fragment comprises a truncation of at least one and at most 50, 49, 48, 47, 46, 45, 44, 43, or 42 amino acid residues from the C-terminal of the mature gE. In some embodiments, the truncation is a truncation of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues from the C-terminal of the mature gE. In some embodiments, the truncation is a truncation of 11, 12, 13, 14, 15, 16, 17, 18, 34, 35, 36, 37, 38, 39, 40, 41, 42, 43, or 44 amino acid residues from the C-terminal of the mature gE. In some embodiments, the truncation is a truncation of at most 49, 48, 47, 46, 45, 44, 43, or 42 amino acid residues from the C-terminal of the mature gE. In some embodiments, the truncation is a truncation of 14 or 37 amino acid residues from the C-terminal of the mature gE. In some embodiments, the truncation is a truncation of 37 amino acid residues from the C-terminal of the mature gE. In some embodiments, the truncation is a truncation of at most 41, 40, 39, 38, 37, 36, 35, 34, 33, 32, or 31 amino acid residues from the C-terminal of the mature gE. In some embodiments, the truncation is a truncation of at most 30 or 29 amino acid residues from the C-terminal of the mature gE. In some embodiments, the truncation is a truncation of at most 28 amino acid residues from the C-terminal of the mature gE. In some embodiments, the truncation is a truncation of at most 27 or 26 amino acid residues from the C-terminal of the mature gE. In some embodiments, the truncation is a truncation of at most 25, 24, 23, 22, 21, 20, 19, 18, 17, 16, 15, 14, 13, 12, 11, 10, 9, 8, 7, 6, 5, 4, 3, or 2 amino acid residues or at most one amino acid residue from the C-terminal of the mature gE. In some embodiments, the mature gE comprises the amino acid sequence set forth in SEQ ID NO: 1.

In some embodiments, the fragment of the mature gE further comprises one or more amino acid substitutions. In some embodiments, the fragment of the mature gE further comprises one, two, three, four, five, or all of the amino acid substitutions selected from Y569A, Y582G, S593A, S595A, T596A. and T598A, wherein amino acid residue positions 569, 582, 593, 595, 596, and 598 are the amino acid residue position numbers according to full length VZV gE. In some embodiments, the full length VZV gE comprises the amino acid sequence set forth in SEQ ID NO: 55.

In some embodiments, the fragment of the mature gE further comprises amino acid residue substitution Y569A, wherein amino acid residue position number 569 is the amino acid residue position number according to full length VZV gE. In some embodiments, the fragment of the mature gE further comprises the amino acid residue substitution Y582G, wherein amino acid residue position 582 is the amino acid residue position number according to full length VZV gE. In some embodiments, the fragment of the mature gE further comprises amino acid residue substitutions Y569A and Y582G, wherein amino acid residue position numbers 569 and 582 are the amino acid residue position numbers according to full length VZV gE. In some embodiments, the fragment of the mature gE further comprises amino acid residue substitutions Y569A, Y582G, and S593A, wherein amino acid residue position numbers 569, 582, and 593 are the amino acid residue position numbers according to full length VZV gE. In some embodiments, the fragment of the mature gE further comprises amino acid residue substitutions Y569A, Y582G, S595A wherein amino acid residue position numbers 569, 582, and 595 are the amino acid residue position numbers according to full length VZV gE. In some embodiments, the fragment of the mature gE further comprises amino acid residue substitutions Y569A, Y582G, and T596A, wherein amino acid residue position numbers 569, 582, and 596 are the amino acid residue position numbers according to full length VZV gE. In some embodiments, the fragment of the mature gE further comprises amino acid residue substitutions Y569A, Y582G, and T598A, wherein amino acid residue position numbers 569, 582, and 598 are the amino acid residue position numbers according to full length VZV gE. In some embodiments, the fragment of the mature gE further comprises amino acid residue substitutions Y569A, Y582G, S593A, and S595A, wherein amino acid residue position numbers 569, 582, 593, and 595 are the amino acid residue position numbers according to full length VZV gE. In some embodiments, the fragment of the mature gE further comprises amino acid residue substitutions Y569A, Y582G, S593A, S595A, T596A, wherein amino acid residue position numbers 569, 582, 593, 595, and 596 are the amino acid residue position numbers according to full length VZV gE. In some embodiments, the fragment of the mature gE further comprises amino acid residue substitutions Y569A, Y582G, S593A, S595A, T596A, and T598A, wherein amino acid residue position numbers 569, 582, 593, 595, 596, and 598 are the amino acid residue position numbers according to full length VZV gE. In some embodiments, the full length VZV gE comprises the amino acid sequence set forth in SEQ ID NO: 55.

In some embodiments, the fragment of the mature gE further comprises the amino acid residue substitution S593A, wherein amino acid residue position 593 is the amino acid residue position number according to full length VZV gE. In some embodiments, the fragment of the mature gE further comprises the amino acid residue substitution S595A wherein amino acid residue position 595 is the amino acid residue position number according to full length VZV gE. In some embodiments, the fragment of the mature gE further comprises the amino acid residue substitution T596A, wherein amino acid residue position 596 is the amino acid residue position number according to full length VZV gE. In some embodiments, the fragment of the mature gE further comprises the amino acid residue substitution T598A, wherein amino acid residue position 598 is the amino acid residue position number according to full length VZV gE. In some embodiments, the fragment of the mature gE further comprises the amino acid residue substitutions S593A and S595A, wherein amino acid residue positions 593 and 595 are the amino acid residue position numbers according to full length VZV gE. In some embodiments, the fragment of the mature gE further comprises the amino acid residue substitutions S593A and T596A, wherein amino acid residue positions 593 and 596 are the amino acid residue position numbers according to full length VZV gE. In some embodiments, the fragment of the mature gE further comprises the amino acid residue substitutions S593A and T598A, wherein amino acid residue positions 593 and 598 are the amino acid residue position numbers according to full length VZV gE. In some embodiments, the fragment of the mature gE further comprises the amino acid residue substitutions S595A and T596A, wherein amino acid residue positions 595 and 596 are the amino acid residue position numbers according to full length VZV gE. In some embodiments, the fragment of the mature gE further comprises the amino acid residue substitutions S595A and T598A, wherein amino acid residue positions 595 and 598 are the amino acid residue position numbers according to full length VZV gE. In some embodiments, the fragment of the mature gE further comprises the amino acid residue substitutions T596A and T598A, wherein amino acid residue positions 596 and 598 are the amino acid residue position numbers according to full length VZV gE. In some embodiments, the fragment of the mature gE further comprises the amino acid residue substitutions S593A, S595A, and T596A, wherein amino acid residue positions 593, 595, and 596 are the amino acid residue position numbers according to full length VZV gE. In some embodiments, the fragment of the mature gE further comprises the amino acid residue substitutions S593A, S595A, and T598A, wherein amino acid residue positions 593, 595, and 598 are the amino acid residue position numbers according to full length VZV gE. In some embodiments, the fragment of the mature gE further comprises the amino acid residue substitutions S595A, T596A. T598A, wherein amino acid residue positions 595, 596, and 598 are the amino acid residue position numbers according to full length VZV gE. In some embodiments, the fragment of the mature gE further comprises the amino acid residue substitutions S593A, S595A, T596A. T598A, wherein amino acid residue positions 593, 595, 596, and 598 are the amino acid residue position numbers according to full length VZV gE. In some embodiments, the fragment of the mature gE further comprises amino acid residue substitutions Y582G, S593A, S595A, T596A, and T598A, wherein amino acid residue position numbers 582, 593, 595, 596, and 598 are the amino acid residue position numbers according to full length VZV gE. In some embodiments, the full length VZV gE comprises the amino acid sequence set forth in SEQ ID NO: 55.

In some embodiments, the fragment of the mature gE further comprises the amino acid residue substitutions Y569A and S593A, wherein amino acid residue positions 569 and 593 are the amino acid residue position numbers according to full length VZV gE. In some embodiments, the fragment of the mature gE further comprises the amino acid residue substitutions Y569A and S595A, wherein amino acid residue positions 569 and 595 are the amino acid residue position numbers according to full length VZV gE. In some embodiments, the fragment of the mature gE further comprises the amino acid residue substitutions Y569A and T596A, wherein amino acid residue positions 569 and 596 are the amino acid residue position numbers according to full length VZV gE. In some embodiments, the fragment of the mature gE further comprises the amino acid residue substitutions Y569A and T598A, wherein amino acid residue positions 569 and 598 are the amino acid residue position numbers according to full length VZV gE. In some embodiments, the fragment of the mature gE further comprises the amino acid residue substitutions Y569A, S593A and S595A, wherein amino acid residue positions 569, 593 and 595 are the amino acid residue position numbers according to full length VZV gE. In some embodiments, the fragment of the mature gE further comprises the amino acid residue substitutions Y569A, S593A and T596A, wherein amino acid residue positions 569, 593 and 596 are the amino acid residue position numbers according to full length VZV gE. In some embodiments, the fragment of the mature gE further comprises the amino acid residue substitutions Y569A, S593A and T598A, wherein amino acid residue positions 569, 593 and 598 are the amino acid residue position numbers according to full length VZV gE. In some embodiments, the fragment of the mature gE further comprises the amino acid residue substitutions Y569A, S595A and T596A, wherein amino acid residue positions 569, 595 and 596 are the amino acid residue position numbers according to full length VZV gE. In some embodiments, the fragment of the mature gE further comprises the amino acid residue substitutions Y569A, S595A and T598A, wherein amino acid residue positions 569, 595 and 598 are the amino acid residue position numbers according to full length VZV gE. In some embodiments, the fragment of the mature gE further comprises the amino acid residue substitutions Y569A, T596A and T598A, wherein amino acid residue positions 569, 596 and 598 are the amino acid residue position numbers according to full length VZV gE. In some embodiments, the fragment of the mature gE further comprises the amino acid residue substitutions Y569A, S593A, S595A, and T596A, wherein amino acid residue positions 569, 593, 595, and 596 are the amino acid residue position numbers according to full length VZV gE. In some embodiments, the fragment of the mature gE further comprises the amino acid residue substitutions Y569A, S593A, S595A, and T598A, wherein amino acid residue positions 569, 593, 595, and 598 are the amino acid residue position numbers according to full length VZV gE. In some embodiments, the fragment of the mature gE further comprises the amino acid residue substitutions Y569A, S595A, T596A. T598A, wherein amino acid residue positions 569, 595, 596, and 598 are the amino acid residue position numbers according to full length VZV gE. In some embodiments, the fragment of the mature gE further comprises the amino acid residue substitutions Y569A, S593A, S595A, T596A. T598A, wherein amino acid residue positions 569, 593, 595, 596, and 598 are the amino acid residue position numbers according to full length VZV gE. In some embodiments, the full length VZV gE comprises the amino acid sequence set forth in SEQ ID NO: 55.

In some embodiments, provided herein is a fusion protein comprising the fragment of a mature gE of VZV, and a heterologous signal peptide, wherein the N-terminal of the fragment is fused to the C-terminal of the heterologous signal peptide. In some embodiments, the mature gE comprises the amino acid sequence of SEQ ID NO:1. In some embodiments, the heterologous signal peptide is a human tPA signal peptide. In some embodiments, the human tPA signal peptide comprises the amino acid sequence set forth in SEQ ID NO: 27. In some embodiments, the human tPA signal peptide comprises an amino acid sequence that is at least 90%, at least 910%, at least 92%, at least 93%, or at least 94% identical to the amino acid sequence set forth in SEQ ID NO: 27. In some embodiments, the human tPA signal peptide comprises an amino acid sequence that is at least 95%, at least 96%, at least 97%, at least 98%, or at least 99% identical to the amino acid sequence set forth in SEQ ID NO: 27. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises an amino acid sequence that is at least 90%, at least 91%, at least 92%, at least 93%, or at least 94% identical to the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23. In some embodiments, the fragment of the mature gE is a fragment described herein (e.g., a fragment of mature gE described in this Section or the Examples in Section 6). In some embodiments, the fragment of the mature gE comprises the amino acid sequence of SEQ ID NO: 3, 6, 8, 10, or 12. In specific embodiments, the fragment comprises the amino acid sequence of SEQ ID NO: 6. In some embodiments, the fragment of the mature gE comprises an amino acid sequence that is at least 80%, at least 81%, at least 82%, at least 83%, or at least 84% identical to SEQ ID NO: 3, 6, 8, 10, or 12. In some embodiments, the fragment of the mature gE comprises an amino acid sequence that is at least 85%, at least 86%, at least 87%, at least 88%, or at least 89% identical to SEQ ID NO: 3, 6, 8, 10, or 12. In some embodiments, the fragment of the mature gE comprises an amino acid sequence that is at least 90%, at least 91%, at least 92%, at least 93%, or at least 94% identical to SEQ ID NO: 3, 6, 8, 10, or 12. In some embodiments, the fragment of the mature gE comprises an amino acid sequence that is at least 95%, at least 96%, at least 97%, at least 98%, or at least 99% identical to SEQ ID NO: 3, 6, 8, 10, or 12.

In some embodiments, provided herein is a fusion protein comprising a fragment of a mature gE of VZV and a human IgE signal peptide, wherein the fragment comprises a truncation of 37 amino acid residues from the C-terminal of the mature gE, and the N-terminal of the fragment is fused to the C-terminal of the human IgE signal peptide. In some embodiments, provided herein is a fusion protein comprising a fragment of a mature gE of VZV and a human IgE signal peptide, wherein the fragment comprises a truncation of 37 amino acid residues from the C-terminal of the mature gE and amino acid residue substitutions Y569A and Y582G, wherein amino acid residue positions 569 and 582 are the amino acid residue position numbers according to full length VZV gE, and the N-terminal of the fragment is fused to the C-terminal of the human IgE signal peptide. In some embodiments, the human IgE signal peptide comprises the amino acid sequence set forth in SEQ ID NO: 23. In some embodiments, the human IgE signal peptide comprises an amino acid sequence that is at least 90%, at least 91%, at least 92%, at least 93%, or at least 94% identical to the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23.

In some embodiments, provided herein is a fusion protein comprising a fragment of a mature gE of VZV and a human IgE signal peptide, wherein the fragment comprises a truncation of 37 amino acid residues from the C-terminal of the mature gE and amino acid residue substitutions Y569A and Y582G, wherein amino acid residues 569 and 582 are amino acid residues position numbers according to full length VZV gE. In some embodiments, the mature gE comprises the amino acid sequence of SEQ ID NO: 1. In some embodiments, the full length VZV gE comprises the amino acid sequence set forth in SEQ ID NO: 55. In some embodiments, the fragment comprises the amino acid sequence set forth in SEQ ID NO: 6. In some embodiments, the human IgE signal peptide comprises the amino acid sequence set forth in SEQ ID NO: 23. In some embodiments, the human IgE signal peptide comprises an amino acid sequence that is at least 90%, at least 91%, at least 92%, at least 93%, or at least 94% identical to the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23. In some embodiments, the fusion protein comprises the amino acid sequence set forth in SEQ ID NO: 59. In some embodiments, the fusion protein comprises an amino acid sequences that is at least 90%, at least 91%, at least 92%, at least 93%, or at least 94% identical to the amino acid sequence of SEQ ID NO: 59. In some embodiments, the fusion protein comprises an amino acid sequences that is at least 95%, at least 96%, at least 97%, at least 98%, or at least 99% identical to the amino acid sequence set forth in SEQ ID NO: 59.

In some embodiments, provided herein is a fusion protein comprising a mutant of a mature gE of VZV and a human IgE signal peptide, wherein the mutant comprises amino acid residue substitutions Y569A, Y582G, S593A, S595A, T596A, and T598A, wherein amino acid residues 569, 582, 593, 595, 596, and 598 are the amino acid residues position numbers according to full length VZV gE. In some embodiments, the mature gE comprises the amino acid sequence of SEQ ID NO: 1. In some embodiments, the full length VZV gE comprises the amino acid sequence of SEQ ID NO: 55. In some embodiments, the mutant comprises the amino acid sequence of SEQ ID NO: 10. In some embodiments, the human IgE signal peptide comprises the amino acid sequence set forth in SEQ ID NO: 23. In some embodiments, the human IgE signal peptide comprises an amino acid sequence that is at least 90%, at least 91%, at least 92%, at least 93%, or at least 94% identical to the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23.

In some embodiments, provided herein is a fusion protein comprising a mutant of a mature gE of VZV and a human IgE signal peptide, wherein the mutant comprises amino acid residue substitutions Y582G, S593A, S595A, T596A, and T598A, wherein amino acid residues 582, 593, 595, 596, and 598 are the amino acid residues position numbers according to full length VZV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mutant comprises the amino acid sequence set forth in SEQ ID NO: 12. In some embodiments, the human IgE signal peptide comprises the amino acid sequence set forth in SEQ ID NO: 23. In some embodiments, the human IgE signal peptide comprises an amino acid sequence that is at least 90%, at least 910%, at least 92%, at least 93%, or at least 94% identical to the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23.

In some embodiments, provided herein is a fusion protein comprising a fragment of a mature gE of VZV and a human IgE signal peptide, wherein the fragment comprises a truncation of 50 amino acid residues from the C-terminal of the mature gE and amino acid residue substitution Y569A, wherein amino acid residue 569 is the amino acid residue position number according to full length VZV gE. In some embodiments, the full length VZV gE comprises the amino acid sequence set forth in SEQ ID NO: 55. In some embodiments, the mature gE comprises the amino acid sequence set forth in SEQ ID NO: 1. In some embodiments, the fragment comprises the amino acid sequence of SEQ ID NO: 3. In some embodiments, the human IgE signal peptide comprises the amino acid sequence set forth in SEQ ID NO: 23. In some embodiments, the human IgE signal peptide comprises an amino acid sequence that is at least 90%, at least 91%, at least 92%, at least 93%, or at least 94% identical to the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23.

In some embodiments, provided herein is a mutant of a full length gE of VZV, wherein the mutant comprises amino acid residue substitutions Y569A and Y582G relative to the full length VSV gE. In some embodiments, provided herein is a mutant of a full length gE of VZV, wherein the mutant comprises amino acid residue substitutions Y569A, Y582G, and S593A relative to the full length VSV gE. In some embodiments, provided herein is a mutant of a full length gE of VZV, wherein the mutant comprises amino acid residue substitutions Y569A, Y582G, and S595A relative to the full length VSV gE. In some embodiments, provided herein is a mutant of a full length gE of VZV, wherein the mutant comprises amino acid residue substitutions Y569A, Y582G, and T596A relative to the full length VSV gE. In some embodiments, provided herein is a mutant of a full length gE of VZV, wherein the mutant comprises amino acid residue substitutions Y569A, Y582G, and T598A relative to the full length VSV gE. In some embodiments, provided herein is a mutant of a full length gE of VZV, wherein the mutant comprises amino acid residue substitutions Y569A, Y582G, S593A, and S595A relative to the full length VSV gE. In some embodiments, provided herein is a mutant of a full length gE of VZV, wherein the mutant comprises amino acid residue substitutions Y569A, Y582G, S593A, and T596A relative to the full length VSV gE. In some embodiments, provided herein is a mutant of a full length gE of VZV, wherein the mutant comprises amino acid residue substitutions Y569A, Y582G, S593A, and T598A relative to the full length VSV gE. In some embodiments, provided herein is a mutant of a full length gE of VZV, wherein the mutant comprises amino acid residue substitutions Y569A, Y582G, S595A, and T596A relative to the full length VSV gE. In some embodiments, provided herein is a mutant of a full length gE of VZV, wherein the mutant comprises amino acid residue substitutions Y569A, Y582G, S595A, and T598A relative to the full length VSV gE. In some embodiments, provided herein is a mutant of a full length gE of VZV, wherein the mutant comprises amino acid residue substitutions Y569A, Y582G, T596A, and T598A relative to the full length VSV gE. In some embodiments, provided herein is a mutant of a full length gE of VZV, wherein the mutant comprises amino acid residue substitutions Y582G, S593A, S595A, T596A, and T598A relative to the full length VSV gE. In some embodiments, provided herein is a mutant of a full length gE of VZV, wherein the mutant comprises amino acid residue substitutions Y569A, Y582G, S593A, S595A, T596A, and T598A relative to the full length VSV gE. In some embodiments, the full length VZV gE comprises the amino acid sequence set forth in SEQ ID NO: 55.

In some embodiments, provided herein is a protein comprising a mutant of a mature gE of VZV, wherein the mutant comprises (i) a truncation of 37 amino acid residues from the C-terminus of the mature gE, and (ii) amino acid residue substitutions Y569A and Y582G, wherein amino acid residue positions 569 and 582 are the amino acid residue position numbers of full length VSV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mutant comprises the amino acid sequence set forth in SEQ ID NO: 6. In some embodiments, the amino acid sequence of the mutant consists of the amino acid sequence set forth in SEQ ID NO: 6. In some embodiments, the mutant comprises an amino acid sequence that is at least 80%, at least 81%, at least 82%, at least 83%, or at least 84% identical to the amino acid sequence set forth in SEQ ID NO: 6. In some embodiments, the mutant comprises an amino acid sequence that is at least 85%, at least 86%, at least 87%, at least 88%, or at least 89% identical to the amino acid sequence set forth in SEQ ID NO: 6. In some embodiments, the mutant comprises an amino acid sequence that is at least 90%, at least 91%, at least 92%, at least 930%, or at least 94% identical to the amino acid sequence set forth in SEQ ID NO: 6. In some embodiments, the mutant comprises an amino acid sequence that is at least 95%, at least 96%, at least 97%, at least 98%, or at least 99% identical to the amino acid sequence set forth in SEQ ID NO: 6. In some embodiments, the protein further comprises the signal peptide of the VZV gE. In some embodiments, the signal peptide of the VZV gE comprises the amino acid sequence set forth in SEQ ID NO: 18. In some embodiments, the signal peptide of the VZV gE comprises an amino acid sequence that is at least 95%, at least 96%, at least 97%, at least 98%, or at least 99% identical to the amino acid sequence set forth in SEQ ID NO: 18. In some embodiments, the protein further comprises a heterologous signal peptide, wherein the N-terminal of the mutant is fused to the C-terminal of the heterologous signal peptide. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23. In some embodiments, the amino acid sequence of the human IgE signal peptide consists of the amino acid sequence set forth in SEQ ID NO: 23. In some embodiments, the amino acid sequence of the mutant consists of the amino acid sequence set forth in SEQ ID NO: 6, and the amino acid sequence of the human IgE signal peptide consists of the amino acid sequence set forth in SEQ ID NO: 23. In some embodiments, the protein comprises the amino acid sequence set forth in SEQ ID NO: 59. In some embodiments, the amino acid sequence of the protein consists of the amino acid sequence set forth in SEQ ID NO: 59. In some embodiments, the heterologous signal peptide of the protein is a human tPA signal peptide. In some embodiments, the human tPA signal peptide comprises the amino acid sequence set forth in SEQ ID NO: 27. In some embodiments, the human tPA signal peptide comprises an amino acid sequence that is at least 95%, at least 96%, at least 97%, at least 98%, or at least 99% identical to the amino acid sequence set forth in SEQ ID NO: 28.

In some embodiments, provided herein is a protein comprising a mutant of a mature gE of VZV, wherein the mutant comprises amino acid residue substitutions Y569A and Y582G, wherein amino acid residue positions 569 and 582 are the amino acid reside position numbers of full length VSV gE. In some embodiments, the mature gE comprises the amino acid sequence of SEQ ID NO: 1. In some embodiments, the full length VZV gE comprises the amino acid sequence set forth in SEQ ID NO: 55. In some embodiments, the mutant comprises the amino acid sequence set forth in SEQ ID NO: 8. In some embodiments, the amino acid sequence of the mutant consists of the amino acid sequence set forth in SEQ ID NO: 8. In some embodiments, the mutant comprises an amino acid sequence that is at least 80%, at least 81%, at least 82%, at least 83%, or at least 84% identical to the amino acid sequence set forth in SEQ ID NO: 8. In some embodiments, the mutant comprises an amino acid sequence that is at least 85%, at least 86%, at least 87%, at least 88%, or at least 89% identical to the amino acid sequence set forth in SEQ ID NO: 8. In some embodiments, the mutant comprises an amino acid sequence that is at least 90%, at least 91%, at least 92%, at least 93%, or at least 94% identical to the amino acid sequence set forth in SEQ ID NO: 8. In some embodiments, the mutant comprises an amino acid sequence that is at least 95%, at least 96%, at least 97%, at least 98%, or at least 99% identical to the amino acid sequence set forth in SEQ ID NO: 8. In some embodiments, the protein further comprises the signal peptide of the VZV gE. In some embodiments, the signal peptide of the VZV gE comprises the amino acid sequence set forth in SEQ ID NO: 18. In some embodiments, the signal peptide of the VZV gE comprises an amino acid sequence that is at least 95%, at least 96%, at least 97%, at least 98%, or at least 99% identical to the amino acid sequence set forth in SEQ ID NO: 18. In some embodiments, the protein further comprises a heterologous signal peptide, wherein the N-terminal of the mutant is fused to the C-terminal of the heterologous signal peptide. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises: an amino acid sequence that is at least 95%, at least 96%, at least 97%, at least 98%, or at least 99% identical to the amino acid sequence set forth in SEQ ID NO: 23. In some embodiments, the amino acid sequence of the human IgE signal peptide consists of the amino acid sequence set forth in SEQ ID NO: 23. In some embodiments, the amino acid sequence of the mutant consists of the amino acid sequence set forth in SEQ ID NO: 8, and the amino acid sequence of the human IgE signal peptide consists of the amino acid sequence set forth in SEQ ID NO: 23. In some embodiments, the heterologous signal peptide of the protein is a human tPA signal peptide. In some embodiments, the human tPA signal peptide comprises the amino acid sequence set forth in SEQ ID NO: 27. In some embodiments, the human tPA signal peptide comprises an amino acid sequence that is at least 95%, at least 96%, at least 97%, at least 98%, or at least 99% identical to the amino acid sequence set forth in SEQ ID NO: 28.

In some embodiments, provided herein is a protein comprising a mutant of a mature gE of VZV, wherein the mutant comprises amino acid residue substitutions Y569A, Y582G, S593A, S595A, T596A, and T598A, wherein amino acid residue positions 569, 582, 593, 595, 596, and 598 are the amino acid reside position numbers of full length VSV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mutant comprises the amino acid sequence set forth in SEQ ID NO: 10. In some embodiments, the amino acid sequence of the mutant consists of the amino acid sequence set forth in SEQ ID NO: 10. In some embodiments, the mutant comprises an amino acid sequence that is at least 80%, at least 81%, at least 82%, at least 83%, or at least 84% identical to the amino acid sequence set forth in SEQ ID NO: 10. In some embodiments, the mutant comprises an amino acid sequence that is at least 85%, at least 86%, at least 87%, at least 88%, or at least 89% identical to the amino acid sequence set forth in SEQ ID NO: 10. In some embodiments, the mutant comprises an amino acid sequence that is at least 90%, at least 91%, at least 92%, at least 93%, or at least 94% identical to the amino acid sequence set forth in SEQ ID NO: 10. In some embodiments, the mutant comprises an amino acid sequence that is at least 95%, at least 96%, at least 97%, at least 98%, or at least 99% identical to the amino acid sequence set forth in SEQ ID NO: 10. In some embodiments, the protein further comprises the signal peptide of the VZV gE. In some embodiments, the signal peptide of the VZV gE comprises the amino acid sequence set forth in SEQ ID NO: 18. In some embodiments, the signal peptide of the VZV gE comprises an amino acid sequence that is at least 95%, at least 96%, at least 97%, at least 98%, or at least 99% identical to the amino acid sequence set forth in SEQ ID NO: 18. In some embodiments, the protein further comprises a heterologous signal peptide, wherein the N-terminal of the mutant is fused to the C-terminal of the heterologous signal peptide. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises: an amino acid sequence that is at least 95%, at least 96%, at least 97%, at least 98%, or at least 99% identical to the amino acid sequence set forth in SEQ ID NO: 23. In some embodiments, the amino acid sequence of the human IgE signal peptide consists of the amino acid sequence set forth in SEQ ID NO: 23. In some embodiments, the amino acid sequence of the mutant consists of the amino acid sequence set forth in SEQ ID NO: 10, and the amino acid sequence of the human IgE signal peptide consists of the amino acid sequence set forth in SEQ ID NO: 23. In some embodiments, the heterologous signal peptide of the protein is a human tPA signal peptide. In some embodiments, the human tPA signal peptide comprises the amino acid sequence set forth in SEQ ID NO: 27. In some embodiments, the human tPA signal peptide comprises an amino acid sequence that is at least 95%, at least 96%, at least 97%, at least 98%, or at least 99% identical to the amino acid sequence set forth in SEQ ID NO: 28.

In some embodiments, provided herein is a protein comprising a mutant of a mature gE of VZV, wherein the mutant comprises amino acid residue substitutions Y582G, S593A, S595A, T596A, and T598A, wherein amino acid residue positions 582, 593, 595, 596, and 598 are the amino acid reside position numbers of full length VSV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mutant comprises the amino acid sequence set forth in SEQ ID NO: 12. In some embodiments, the amino acid sequence of the mutant consists of the amino acid sequence set forth in SEQ ID NO: 12. In some embodiments, the mutant comprises an amino acid sequence that is at least 80%, at least 81%, at least 82%, at least 83%, or at least 84% identical to the amino acid sequence set forth in SEQ ID NO: 12. In some embodiments, the mutant comprises an amino acid sequence that is at least 85%, at least 86%, at least 87%, at least 88%, or at least 89% identical to the amino acid sequence set forth in SEQ ID NO: 12. In some embodiments, the mutant comprises an amino acid sequence that is at least 90%, at least 91%, at least 92%, at least 93%, or at least 94% identical to the amino acid sequence set forth in SEQ ID NO: 12. In some embodiments, the mutant comprises an amino acid sequence that is at least 95%, at least 96%, at least 97%, at least 98%, or at least 99% identical to the amino acid sequence set forth in SEQ ID NO: 12. In some embodiments, the protein further comprises the signal peptide of the VZV gE. In some embodiments, the signal peptide of the VZV gE comprises the amino acid sequence set forth in SEQ ID NO: 18. In some embodiments, the signal peptide of the VZV gE comprises an amino acid sequence that is at least 95%, at least 96%, at least 97%, at least 98%, or at least 99% identical to the amino acid sequence set forth in SEQ ID NO: 18. In some embodiments, the protein further comprises a heterologous signal peptide, wherein the N-terminal of the mutant is fused to the C-terminal of the heterologous signal peptide. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises: an amino acid sequence that is at least 95%, at least 96%, at least 97%, at least 98%, or at least 99% identical to the amino acid sequence set forth in SEQ ID NO: 23. In some embodiments, the amino acid sequence of the human IgE signal peptide consists of the amino acid sequence set forth in SEQ ID NO: 23. In some embodiments, the amino acid sequence of the mutant consists of the amino acid sequence set forth in SEQ ID NO: 12, and the amino acid sequence of the human IgE signal peptide consists of the amino acid sequence set forth in SEQ ID NO: 23. In some embodiments, the heterologous signal peptide of the protein is a human tPA signal peptide. In some embodiments, the human tPA signal peptide comprises the amino acid sequence set forth in SEQ ID NO: 27. In some embodiments, the human tPA signal peptide comprises an amino acid sequence that is at least 95%, at least 96%, at least 97%, at least 98%, or at least 99% identical to the amino acid sequence set forth in SEQ ID NO: 28.

In some embodiments, provided herein is a protein comprising a mutant of a mature gE of VZV, wherein the mutant comprises: (i) a truncation of 50 amino acid residues from the C-terminus of the mature gE protein, and (ii) amino acid residue substitution Y569A, wherein amino acid residue position 569 is the amino acid reside position of full length VSV gE. In some embodiments, the mature gE comprises the amino acid sequence set forth in SEQ ID NO: 1. In some embodiments, the full length VZV gE comprises the amino acid sequence set forth in SEQ ID NO: 55. In some embodiments, the mutant comprises the amino acid sequence set forth in SEQ ID NO: 3. In some embodiments, the amino acid sequence of the mutant consists of the amino acid sequence set forth in SEQ ID NO: 3. In some embodiments, the mutant comprises an amino acid sequence that is at least 80%, at least 81%, at least 82%, at least 830%, or at least 84% identical to the amino acid sequence set forth in SEQ ID NO: 3. In some embodiments, the mutant comprises an amino acid sequence that is at least 85%, at least 86%, at least 87%, at least 88%, or at least 89% identical to the amino acid sequence set forth in SEQ ID NO: 3. In some embodiments, the mutant comprises an amino acid sequence that is at least 90%, at least 91%, at least 92%, at least 93%, or at least 94% identical to the amino acid sequence set forth in SEQ ID NO: 3. In some embodiments, the mutant comprises an amino acid sequence that is at least 95%, at least 96%, at least 97%, at least 98%, or at least 99% identical to the amino acid sequence set forth in SEQ ID NO: 3. In some embodiments, the protein further comprises the signal peptide of the VZV gE that comprises the amino acid sequence set forth in SEQ ID NO: 18. In some embodiments, the signal peptide of the VZV gE comprises an amino acid sequence that is at least 95%, at least 96%, at least 97%, at least 98%, or at least 99% identical to the amino acid sequence set forth in SEQ ID NO: 18. In some embodiments, the protein further comprises a heterologous signal peptide, wherein the N-terminal of the mutant is fused to the C-terminal of the heterologous signal peptide. In some embodiments, the heterologous signal peptide is a human IgE signal peptide. In some embodiments, the human IgE signal peptide comprises the amino acid sequence set forth in SEQ ID NO: 23. In some embodiments, the human IgE signal peptide comprises an amino acid sequence that is at least 95%, at least 96%, at least 97%, at least 98%, or at least 99% identical to the amino acid sequence set forth in SEQ ID NO: 23. In some embodiments, the amino acid sequence of the human IgE signal peptide consists of the amino acid sequence set forth in SEQ ID NO: 23. In some embodiments, the amino acid sequence of the mutant consists of the amino acid sequence set forth in SEQ ID NO: 3, and the amino acid sequence of the human IgE signal peptide consists of the amino acid sequence set forth in SEQ ID NO: 23. In some embodiments, the heterologous signal peptide of the protein is a human tPA signal peptide. In some embodiments, the human tPA signal peptide comprises the amino acid sequence set forth in SEQ ID NO: 27. In some embodiments, the human tPA signal peptide comprises an amino acid sequence that is at least 95%, at least 96%, at least 97%, at least 98%, or at least 99% identical to the amino acid sequence set forth in SEQ ID NO: 28.

In some embodiments, provided herein is a protein comprising the amino acid sequence of a VZV gE mutein disclosed in Table 1. In some embodiments, provided herein is a protein comprising the amino acid sequence of VZV gE mutein-1 disclosed in Table 1. In some embodiments, provided herein is a protein comprising the amino acid sequence of VZV gE mutein-2 disclosed in Table 1. In some embodiments, provided herein is a protein comprising the amino acid sequence of VZV gE mutein-3 disclosed in Table 1. In some embodiments, provided herein is a protein comprising the amino acid sequence of VZV gE mutein-4 disclosed in Table 1. In some embodiments, provided herein is a protein comprising the amino acid sequence of VZV gE mutein-5 disclosed in Table 1.

In some embodiments, provided herein is a mutant of a VZV gE protein described in Section 6 below. In some embodiments, provided herein is a mutant of a VZV gE described in Section 6 below other than a control. In some embodiments, provided herein is mutant of a VZV gE encoded by a nucleic acid described in Section 6 below other than a control.

In some embodiments, a fragment described herein, mutant described herein, or fusion protein described herein has a similar structure as wild-type VZV gE as assessed by techniques known to one of skill in the art, such as, e.g., X-ray crystallography, nuclear magnetic resonance, or binding to antibodies specific for conformational epitopes of wild-type VZV gE. In some embodiments, a protein described herein comprising a mutant of mature gE has a similar structure as wild-type VZV gE as assessed by techniques known to one of skill in the art, such as, e.g., X-ray crystallography, nuclear magnetic resonance, or binding to antibodies specific for conformational epitopes of wild-type VZV gE.

In some embodiments, a fragment described herein, mutant described herein, or fusion protein described herein retains at least one activity or function of mature VZV gE. In some embodiments, a protein described herein comprising a mutant of mature gE retains at least one activity or function of mature VZV gE. For example, in some embodiments, the ability to form a heterodimer with gI is retained. In another example, in some embodiments, the ability to bind to Fc receptor is retained. In another example, in some embodiments, the ability to bind to insulin degrading enzyme (IDE) is retained. In another example, in some embodiments, the ability to be phosphorylated by the viral kinase encoded by ORF47 is retained.

### 7.4 Therapeutic Nucleic Acids

In one aspect, provided herein are therapeutic nucleic acid molecules for the management, prevention and treatment of VZV infection. In some embodiments, the therapeutic nucleic acid encodes a peptide or polypeptide, which upon administration into a subject in need thereof, is expressed by the cells in the subject to produce the encoded peptide or polypeptide. In some embodiments, the therapeutic nucleic acid molecules are DNA molecules. In other embodiments, the therapeutic nucleic acid molecules are RNA molecules. In particular embodiments, the therapeutic nucleic acid molecules are mRNA molecules.

In some embodiments, the therapeutic nucleic acid molecule is formulated in a vaccine composition. In some embodiments, the vaccine composition is a genetic vaccine as described herein. In some embodiments, the vaccine composition comprises an mRNA molecule as described herein.

In some embodiments, the mRNA molecule of the present disclosure encodes a peptide or polypeptide of interest, including any naturally or non-naturally occurring or otherwise modified polypeptide. A peptide or polypeptide encoded by an mRNA may be of any size and may have any secondary structure or activity. In some embodiments, the polypeptide encoded by an mRNA payload can have a therapeutic effect when expressed in a cell.

In some embodiment, the mRNA molecule of the present disclosure comprises at least one coding region encoding a peptide or polypeptide of interest (e.g., an open reading frame (ORF)). In some embodiments, the nucleic acid molecule further comprises at least one untranslated region (UTR). In particular embodiments, the untranslated region (UTR) is located upstream (to the 5′-end) of the coding region, and is referred to herein as the 5′-UTR In particular embodiments, the untranslated region (UTR) is located downstream (to the 3′-end) of the coding region, and is referred to herein as the 3′-UTR. In particular embodiments, the nucleic acid molecule comprises both a 5′-UTR and a 3′-UTR. In some embodiments, the 5′-UTR comprises a 5′-Cap structure. In some embodiments, the nucleic acid molecule comprises a Kozak sequence (e.g., in the 5′-UTR). In some embodiments, the nucleic acid molecule comprises a poly-A region (e.g., in the 3′-UTR). In some embodiments, the nucleic acid molecule comprises a polyadenylation signal (e.g., in the 3′-UTR). In some embodiments, the nucleic acid molecule comprises stabilizing region (e.g., in the 3′-UTR). In some embodiments, the nucleic acid molecule comprises a secondary structure. In some embodiments, the secondary structure is a stem-loop. In some embodiments, the nucleic acid molecule comprises a stem-loop sequence (e.g., in the 5′-UTR and/or the 3′-UTR). In some embodiments, the nucleic acid molecule comprises one or more intronic regions capable of being excised during splicing. In a specific embodiment, the nucleic acid molecule comprises one or more region selected from a 5′-UTR, and a coding region. In a specific embodiment the nucleic acid molecule comprises one or more region selected from a coding region and a 3′-UTR. In a specific embodiment, the nucleic acid molecule comprises one or more region selected from a 5′-UTR, a coding region, and a 3′-UTR.

### 7.4.1 Coding Region

In some embodiments, the nucleic acid molecule of the present disclosure comprises at least one coding region. In some embodiments, the coding region is an open reading frame (ORF) that encodes for a single peptide or protein. In some embodiments, the coding region comprises at least two ORFs, each encoding a peptide or protein. In those embodiments where the coding region comprises more than one ORFs, the encoded peptides and/or proteins can be the same as or different from each other. In some embodiments, the multiple ORFs in a coding region are separated by non-coding sequences. In specific embodiments, a non-coding sequence separating two ORFs comprises an internal ribosome entry site (IRES).

Without being bound by the theory, it is contemplated that an internal ribosome entry site (IRES) can act as the sole ribosome binding site, or serve as one of multiple ribosome binding sites of an mRNA. An mRNA molecule containing more than one functional ribosome binding site can encode several peptides or proteins that are translated independently by the ribosomes (e.g., multicistronic mRNA). Accordingly, in some embodiments, the nucleic acid molecule of the present disclosure (e.g., mRNA) comprises one or more internal ribosome entry sites (IRES). Examples of IRES sequences that can be used in connection with the present disclosure include, without limitation, those from picomaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In various embodiments, the nucleic acid molecule of the present disclosure encodes for at least 1, 2, 3, 4, 5, 6, 7, 8, 9, 10 or more than 10 peptides or proteins. Peptides and proteins encoded by a nucleic acid molecule can be the same or different. In some embodiments, the nucleic acid molecule of the present disclosure encodes a dipeptide (e.g., camosine and anserine). In some embodiments, the nucleic acid molecule encodes a tripeptide. In some embodiments, the nucleic acid molecule encodes a tetrapeptide. In some embodiments, the nucleic acid molecule encodes a pentapeptide. In some embodiments, the nucleic acid molecule encodes a hexapeptide. In some embodiments, the nucleic acid molecule encodes a heptapeptide. In some embodiments, the nucleic acid molecule encodes an octapeptide. In some embodiments, the nucleic acid molecule encodes a nonapeptide. In some embodiments, the nucleic acid molecule encodes a decapeptide. In some embodiments, the nucleic acid molecule encodes a peptide or polypeptide that has at least about 15 amino acids. In some embodiments, the nucleic acid molecule encodes a peptide or polypeptide that has at least about 50 amino acids. In some embodiments, the nucleic acid molecule encodes a peptide or polypeptide that has at least about 100 amino acids. In some embodiments, the nucleic acid molecule encodes a peptide or polypeptide that has at least about 150 amino acids. In some embodiments, the nucleic acid molecule encodes a peptide or polypeptide that has at least about 300 amino acids. In some embodiments, the nucleic acid molecule encodes a peptide or polypeptide that has at least about 500 amino acids. In some embodiments, the nucleic acid molecule encodes a peptide or polypeptide that has at least about 1000 amino acids.

In some embodiments, the nucleic acid molecule of the present disclosure is at least about 30 nucleotides (nt) in length. In some embodiments, the nucleic acid molecule is at least about 35 nt in length. In some embodiments, the nucleic acid molecule is at least about 40 nt in length. In some embodiments, the nucleic acid molecule is at least about 45 nt in length. In some embodiments the nucleic acid molecule is at least about 50 nt in length. In some embodiments, the nucleic acid molecule is at least about 55 nt in length. In some embodiments, the nucleic acid molecule is at least about 60 nt in length. In some embodiments, the nucleic acid molecule is at least about 65 nt in length. In some embodiments, the nucleic acid molecule is at least about 70 nt in length. In some embodiments, the nucleic acid molecule is at least about 75 nt in length. In some embodiments, the nucleic acid molecule is at least about 80 nt in length. In some embodiments the nucleic acid molecule is at least about 85 nt in length. In some embodiments, the nucleic acid molecule is at least about 90 nt in length. In some embodiments, the nucleic acid molecule is at least about 95 nt in length. In some embodiments, the nucleic acid molecule is at least about 100 nt in length. In some embodiments, the nucleic acid molecule is at least about 120 nt in length. In some embodiments, the nucleic acid molecule is at least about 140 nt in length. In some embodiments, the nucleic acid molecule is at least about 160 nt in length. In some embodiments, the nucleic acid molecule is at least about 180 nt in length. In some embodiments, the nucleic acid molecule is at least about 200 nt in length. In some embodiments, the nucleic acid molecule is at least about 250 nt in length. In some embodiments, the nucleic acid molecule is at least about 300 nt in length. In some embodiments, the nucleic acid molecule is at least about 400 nt in length. In some embodiments, the nucleic acid molecule is at least about 500 nt in length. In some embodiments, the nucleic acid molecule is at least about 600 nt in length. In some embodiments, the nucleic acid molecule is at least about 700 nt in length. In some embodiments, the nucleic acid molecule is at least about 800 nt in length. In some embodiments, the nucleic acid molecule is at least about 900 nt in length. In some embodiments, the nucleic acid molecule is at least about 1000 nt in length. In some embodiments, the nucleic acid molecule is at least about 1100 nt in length. In some embodiments, the nucleic acid molecule is at least about 1200 nt in length. In some embodiments, the nucleic acid molecule is at least about 1300 nt in length. In some embodiments, the nucleic acid molecule is at least about 1400 nt in length. In some embodiments, the nucleic acid molecule is at least about 1500 nt in length. In some embodiments, the nucleic acid molecule is at least about 1600 nt in length. In some embodiments, the nucleic acid molecule is at least about 1700 nt in length. In some embodiments, the nucleic acid molecule is at least about 1800 nt in length. In some embodiments, the nucleic acid molecule is at least about 1900 nt in length. In some embodiments, the nucleic acid molecule is at least about 2000 nt in length. In some embodiments, the nucleic acid molecule is at least about 2500 nt in length. In some embodiments, the nucleic acid molecule is at least about 3000 nt in length. In some embodiments, the nucleic acid molecule is at least about 3500 nt in length. In some embodiments, the nucleic acid molecule is at least about 4000 nt in length. In some embodiments, the nucleic acid molecule is at least about 4500 nt in length. In some embodiments, the nucleic acid molecule is at least about 5000 nt in length.

In specific embodiments, the therapeutic nucleic acid of the present disclosure is formulated as a vaccine composition (e.g., a genetic vaccine) as described herein. In some embodiments, the therapeutic nucleic acid encodes a peptide or protein capable of eliciting immunity against one or more target conditions or disease. In some embodiments, the target condition is related to or caused by infection by a pathogen, such as VZV. In some embodiments, the therapeutic nucleic acid sequence (e.g., mRNA) encoding a pathogenic protein characteristic for the pathogen, or an immunogenic fragment (e.g., epitope) or derivative thereof. The vaccine, upon administration to a vaccinated subject, allows for expression of the encoded pathogenic protein (or the immunogenic fragment or derivative thereof), thereby eliciting immunity in the subject against the pathogen.

In specific embodiments, provided herein are therapeutic compositions (e.g., vaccine compositions) for the management, prevention and treatment of a diseases or disorder caused by VZV or by infection with VZV.

Without being bound by the theory, it is contemplated that VZV, i.e., varicella zoster virus, also known as human herpesvirus type 3, is a double stranded DNA virus, belonging to alpha herpes virus. VZV has only one serotype. VZV has a genome comprising 71 genes and encoding 67 proteins, including 6 glycoproteins, which are now named gE, gB, gH, gI, gC, and gL. Glycoproteins gE, gB and gH are very abundant in infected cells, and also present in the envelop of virions. Antibodies induced by the three main glycoproteins can neutralize the virus. Specific humoral and cellular immunity and cytokines such as interferon play a major role in limiting the spread of VZV and in recovery, wherein specific cellular immunity is especially important.

Accordingly, in some embodiments, provided herein are therapeutic nucleic acids encoding a viral peptide or protein derived from VZV. In some embodiments, the nucleic acid encodes a viral peptide or protein derived from VZV, where the viral peptide or protein is one or more selected from (a) the gE protein, (b) the gB protein, (c) the gH protein, (d) the gI protein, (e) the gC protein, (f) the gL protein, (g) an immunogenic fragment of any one of (a) to (f), and (h) a functional derivative of any one of (a) to (g).

Accordingly, in some embodiments, the therapeutic nucleic acid of the present disclosure encodes the VZV gE protein, or an immunogenic fragment of the gE protein, or a functional derivative of the gE protein or the immunogenic fragment thereof. Table 1 shows, among others, exemplary VZV native antigen sequences.

TABLE 1
Exemplary sequences of VZV antigens.

| SEQUENCE | |
|---|---|
| NAME | |
| (SEQ ID NO:) | AMINO ACID OR NUCLEIC ACID SEQUENCE |
| VZV gE mature | SVLRYDDFHIDEDKLDTNSVYEPYYHSDHAESSWVNRGESSRKAYDHNS |
| protein amino | PYIWPRNDYDGFLENAHEHHGVYNQGRGIDSGERLMQPTQMSAQEDLG |
| acid sequence | DDTGIHVIPTLNGDDRHKIVNVDQRQYGDVFKGDLNPKPQGQRLIEVSVE |
| (SEQ ID NO: 1) | ENHPFTLRAPIQRIYGVRYTETWSFLPSLTCTGDAAPAIQHICLKHTTCFQD |
| | VVVDVDCAENTKEDQLAEISYRFQGKKEADQPWIVVNTSTLFDELELDPP |
| | EIEPGVLKVLRTEKQYLGVYIWNMRGSDGTSTYATFLVTWKGDEKTRNP |
| | TPAVTPQPRGAEFHMWNYHSHVFSVGDTFSLAMHLQYKIHEAPFDLLLE |
| | WLYVPIDPTCQPMRLYSTCLYHPNAPQCLSHMNSGCTFTSPHLAQRVAST |
| | VYQNCEHADNYTAYCLGISHMEPSFGLILHDGGTTLKFVDTPESLSGLYV |
| | FVVYFNGHVEAVAYTVVSTVDHFVNAIEERGFPPTAGQPPATTKPKEITP |
| | VNPGTSPLIRYAAWTGGLAAVVLLCLVIFLICTAKRMRVKAYRVDKSPY |
| | NQSMYYAGLPVDDFEDSESTDTEEEFGNAIGGSHGGSSYTVYIDKTR |
| VZV gE mature | AGCGTGCTGAGATACGACGACTTCCATATCGACGAGGACAAGCTGGA |
| protein coding | CACCAACAGCGTGTACGAGCCCTACTACCACAGCGACCACGCCGAGA |
| sequence (SEQ | GCAGCTGGGTGAACAGAGGCGAGAGCAGCAGAAAGGCCTACGACCAC |
| ID NO: 2) | AACAGCCCCTACATCTGGCCTAGAAACGACTACGACGGCTTCCTGGAG |
| | AACGCCCACGAGCACCACGGCGTGTACAACCAAGGCAGAGGCATCGA |
| | CAGCGGCGAGAGACTGATGCAGCCCACACAGATGAGCGCCCAAGAGG |
| | ACCTGGGCGACGACACCGGCATCCACGTGATCCCCACCCTGAACGGC |
| | GACGACAGACACAAGATCGTGAACGTGGATCAGAGACAGTACGGCGA |
| | CGTGTTCAAGGGCGACCTGAACCCCAAGCCCCAAGGGCAGAGACTGA |
| | TCGAGGTGAGCGTGGAGGAGAACCACCCCTTCACCCTGAGAGCCCCC |
| | ATTCAGAGAATCTACGGCGTGAGATACACCGAAACCTGGAGCTTCCTG |
| | CCTAGCCTGACCTGCACCGGCGACGCCGCCCCCGCCATTCAGCACATC |
| | TGCCTGAAGCACACCACCTGCTTCCAAGACGTGGTGGTGGACGTGGAC |
| | TGCGCCGAGAACACCAAGGAGGATCAGCTGGCCGAGATCAGCTACAG |
| | ATTCCAAGGCAAGAAGGAGGCCGATCAGCCCTGGATCGTGGTGAACA |
| | CAAGCACCCTGTTCGACGAGCTGGAGCTGGACCCCCCCGAGATCGAG |
| | CCCGGCGTGCTGAAGGTGCTGAGAACCGAGAAGCAGTACCTGGGCGT |
| | GTACATCTGGAACATGAGAGGCAGCGACGGCACAAGCACCTACGCCA |
| | CCTTCCTGGTGACCTGGAAGGGCGACGAGAAGACAAGAAACCCCACC |
| | CCCGCAGTGACGCCCCAACCTAGAGGCGCCGAGTTCCACATGTGGAA |
| | CTACCACAGCCACGTGTTCAGCGTGGGCGACACCTTCAGCCTGGCCAT |
| | GCACCTGCAGTACAAGATCCACGAGGCCCCCTTCGACCTGCTGCTGGA |
| | GTGGCTGTACGTGCCCATCGACCCCACCTGTCAGCCCATGAGACTGTA |
| | CAGCACCTGCCTGTACCACCCCAACGCCCCTCAGTGCCTTAGTCACAT |
| | GAACAGCGGCTGCACCTTCACAAGCCCCCACCTGGCTCAGAGAGTGG |
| | CTAGCACCGTGTATCAGAACTGCGAGCACGCCGACAACTACACCGCCT |
| | ACTGCCTGGGCATCAGCCACATGGAGCCTAGCTTCGGCCTGATCCTGC |
| | ATGACGGCGGCACGACCTTAAAGTTCGTGGACACCCCCGAGAGCCTG |
| | AGCGGCCTGTACGTGTTCGTGGTGTACTTCAACGGCCACGTGGAGGCC |
| | GTGGCCTACACCGTGGTGAGCACCGTGGACCACTTCGTGAACGCAATC |
| | GAGGAGCGCGGCTTTCCGCCGACTGCCGGTCAACCCCCCGCCACCACC |
| | AAGCCCAAGGAGATCACCCCCGTGAATCCGGGCACAAGCCCTCTTATC |
| | CGGTATGCCGCCTGGACCGGCGGCCTGGCCGCCGTGGTGCTGCTGTGC |
| | CTGGTGATCTTCCTGATCTGCACCGCCAAGAGAATGAGAGTGAAGGCC |
| | TACAGAGTGGACAAGAGCCCCTACAATCAGAGCATGTACTACGCCGG |
| | CCTGCCCGTGGACGACTTCGAGGACAGCGAGAGCACCGACACCGAGG |
| | AGGAGTTCGGCAACGCCATCGGCGGCAGCCACGGAGGCAGCAGCTAC |
| | ACCGTGTACATCGACAAGACAAGA |
| VZV gE full | MGTVNKPVVGVLMGFGIITGTLRITNPVRASVLRYDDFHIDEDKLDTNSVYE |
| length protein | PYYHSDHAESSWVNRGESSRKAYDHNSPYIWPRNDYDGFLENAHEHHG |
| amino acid | VYNQGRGIDSGERLMQPTQMSAQEDLGDDTGIHVIPTLNGDDRHKIVNV |
| sequence (SEQ | DQRQYGDVFKGDLNPKPQGQRLIEVSVEENHPFTLRAPIQRIYGVRYTET |
| ID NO: 55) | WSFLPSLTCTGDAAPAIQHICLKHTTCFQDVVVDVDCAENTKEDQLAEIS |
| | YRFQGKKEADQPWIVVNTSTLFDELELDPPEIEPGVLKVLRTEKQYLGVYI |
| | WNMRGSDGTSTYATFLVTWKGDEKTRNPTPAVTPQPRGAEFHMWNYHS |
| | HVFSVGDTFSLAMHLQYKIHEAPFDLLLEWLYVPIDPTCQPMRLYSTCLY |
| | HPNAPQCLSHMNSGCTFTSPHLAQRVASTVYQNCEHADNYTAYCLGISH |
| | MEPSFGLILHDGGTTLKFVDTPESLSGLYVFVVYFNGHVEAVAYTVVSTV |
| | DHFVNAIEERGFPPTAGQPPATTKPKEITPVNPGTSPLIRYAAWTGGLAAV |
| | VLLCLVIFLICTAKRMRVKAYRVDKSPYNQSMYYAGLPVDDFEDSESTDT |
| | EEEFGNAIGGSHGGSSYTVYIDKTR |
| VZV gE full | ATGGGCACCGTGAACAAGCCCGTGGTGGGCGTGCTGATGGGCTTCGGCAT |
| length protein | CATCACCGGCACCCTGAGAATCACCAACCCCGTGAGAGCTAGCGTGCTGA |
| coding sequence | GATACGACGACTTCCATATCGACGAGGACAAGCTGGACACCAACAGC |
| (SEQ ID NO: 56) | GTGTACGAGCCCTACTACCACAGCGACCACGCCGAGAGCAGCTGGGT |
| | GAACAGAGGCGAGAGCAGCAGAAAGGCCTACGACCACAACAGCCCCT |
| | ACATCTGGCCTAGAAACGACTACGACGGCTTCCTGGAGAACGCCCAC |
| | GAGCACCACGGCGTGTACAACCAAGGCAGAGGCATCGACAGCGGCGA |
| | GAGACTGATGCAGCCCACACAGATGAGCGCCCAAGAGGACCTGGGCG |
| | ACGACACCGGCATCCACGTGATCCCCACCCTGAACGGCGACGACAGA |
| | CACAAGATCGTGAACGTGGATCAGAGACAGTACGGCGACGTGTTCAA |
| | GGGCGACCTGAACCCCAAGCCCCAAGGGCAGAGACTGATCGAGGTGA |
| | GCGTGGAGGAGAACCACCCCTTCACCCTGAGAGCCCCCATTCAGAGA |
| | ATCTACGGCGTGAGATACACCGAAACCTGGAGCTTCCTGCCTAGCCTG |
| | ACCTGCACCGGCGACGCCGCCCCCGCCATTCAGCACATCTGCCTGAAG |
| | CACACCACCTGCTTCCAAGACGTGGTGGTGGACGTGGACTGCGCCGAG |
| | AACACCAAGGAGGATCAGCTGGCCGAGATCAGCTACAGATTCCAAGG |
| | CAAGAAGGAGGCCGATCAGCCCTGGATCGTGGTGAACACAAGCACCC |
| | TGTTCGACGAGCTGGAGCTGGACCCCCCCGAGATCGAGCCCGGCGTGC |
| | TGAAGGTGCTGAGAACCGAGAAGCAGTACCTGGGCGTGTACATCTGG |
| | AACATGAGAGGCAGCGACGGCACAAGCACCTACGCCACCTTCCTGGT |
| | GACCTGGAAGGGCGACGAGAAGACAAGAAACCCCACCCCCGCAGTGA |
| | CGCCCCAACCTAGAGGCGCCGAGTTCCACATGTGGAACTACCACAGCC |
| | ACGTGTTCAGCGTGGGCGACACCTTCAGCCTGGCCATGCACCTGCAGT |
| | ACAAGATCCACGAGGCCCCCTTCGACCTGCTGCTGGAGTGGCTGTACG |
| | TGCCCATCGACCCCACCTGTCAGCCCATGAGACTGTACAGCACCTGCC |
| | TGTACCACCCCAACGCCCCTCAGTGCCTTAGTCACATGAACAGCGGCT |
| | GCACCTTCACAAGCCCCCACCTGGCTCAGAGAGTGGCTAGCACCGTGT |
| | ATCAGAACTGCGAGCACGCCGACAACTACACCGCCTACTGCCTGGGC |
| | ATCAGCCACATGGAGCCTAGCTTCGGCCTGATCCTGCATGACGGCGGC |
| | ACGACCTTAAAGTTCGTGGACACCCCCGAGAGCCTGAGCGGCCTGTAC |
| | GTGTTCGTGGTGTACTTCAACGGCCACGTGGAGGCCGTGGCCTACACC |
| | GTGGTGAGCACCGTGGACCACTTCGTGAACGCAATCGAGGAGCGCGG |
| | CTTTCCGCCGACTGCCGGTCAACCCCCCGCCACCACCAAGCCCAAGGA |
| | GATCACCCCCGTGAATCCGGGCACAAGCCCTCTTATCCGGTATGCCGC |
| | CTGGACCGGCGGCCTGGCCGCCGTGGTGCTGCTGTGCCTGGTGATCTT |
| | CCTGATCTGCACCGCCAAGAGAATGAGAGTGAAGGCCTACAGAGTGG |
| | ACAAGAGCCCCTACAATCAGAGCATGTACTACGCCGGCCTGCCCGTGG |
| | ACGACTTCGAGGACAGCGAGAGCACCGACACCGAGGAGGAGTTCGGC |
| | AACGCCATCGGCGGCAGCCACGGAGGCAGCAGCTACACCGTGTACAT |
| | CGACAAGACAAGA |
| VZV gE mutein-1 | SVLRYDDFHIDEDKLDTNSVYEPYYHSDHAESSWVNRGESSRKAYDHNS |
| amino acid | PYIWPRNDYDGFLENAHEHHGVYNQGRGIDSGERLMQPTQMSAQEDLG |
| sequence (SEQ | DDTGIHVIPTLNGDDRHKIVNVDQRQYGDVFKGDLNPKPQGQRLIEVSVE |
| ID NO: 3) | ENHPFTLRAPIQRIYGVRYTETWSFLPSLTCTGDAAPAIQHICLKHTTCFQD |
| 31-573 aa, Y569A | VVVDVDCAENTKEDQLAEISYRFQGKKEADQPWIVVNTSTLFDELELDPP |
| | EIEPGVLKVLRTEKQYLGVYIWNMRGSDGTSTYATFLVTWKGDEKTRNP |
| | TP AVTPQPRGAEFHMWNYHSHVFSVGDTFSLAMHLQYKIHEAPFDLLLE |
| | WLYVPIDPTCQPMRLYSTCLYHPNAPQCLSHMNSGCTFTSPHLAQRVAST |
| | VYQNCEHADNYTAYCLGISHMEPSFGLILHDGGTTLKFVDTPESLSGLYV |
| | FVVYFNGHVEAVAYTVVSTVDHFVNAIEERGFPPTAGQPPATTKPKEITP |
| | VNPGTSPLIRYAAWTGGLAAVVLLCLVIFLICTAKRMRVKAARVDK |
| VZV gE mutein-1 | AGCGTGCTGAGATACGACGACTTCCATATCGACGAGGACAAGCTGGA |
| coding sequence | CACCAACAGCGTGTACGAGCCCTACTACCACAGCGACCACGCCGAGA |
| 1 (SEQ ID NO: 4) | GCAGCTGGGTGAACAGAGGCGAGAGCAGCAGAAAGGCCTACGACCAC |
| | AACAGCCCCTACATCTGGCCTAGAAACGACTACGACGGCTTCCTGGAG |
| | AACGCCCACGAGCACCACGGCGTGTACAACCAAGGCAGAGGCATCGA |
| | CAGCGGCGAGAGACTGATGCAGCCCACACAGATGAGCGCCCAAGAGG |
| | ACCTGGGCGACGACACCGGCATCCACGTGATCCCCACCCTGAACGGC |
| | GACGACAGACACAAGATCGTGAACGTGGATCAGAGACAGTACGGCGA |
| | CGTGTTCAAGGGCGACCTGAACCCCAAGCCCCAAGGGCAGAGACTGA |
| | TCGAGGTGAGCGTGGAGGAGAACCACCCCTTCACCCTGAGAGCCCCC |
| | ATTCAGAGAATCTACGGCGTGAGATACACCGAAACCTGGAGCTTCCTG |
| | CCTAGCCTGACCTGCACCGGCGACGCCGCCCCCGCCATTCAGCACATC |
| | TGCCTGAAGCACACCACCTGCTTCCAAGACGTGGTGGTGGACGTGGAC |
| | TGCGCCGAGAACACCAAGGAGGATCAGCTGGCCGAGATCAGCTACAG |
| | ATTCCAAGGCAAGAAGGAGGCCGATCAGCCCTGGATCGTGGTGAACA |
| | CAAGCACCCTGTTCGACGAGCTGGAGCTGGACCCCCCCGAGATCGAG |
| | CCCGGCGTGCTGAAGGTGCTGAGAACCGAGAAGCAGTACCTGGGCGT |
| | GTACATCTGGAACATGAGAGGCAGCGACGGCACAAGCACCTACGCCA |
| | CCTTCCTGGTGACCTGGAAGGGCGACGAGAAGACAAGAAACCCCACC |
| | CCCGCAGTGACGCCCCAACCTAGAGGCGCCGAGTTCCACATGTGGAA |
| | CTACCACAGCCACGTGTTCAGCGTGGGCGACACCTTCAGCCTGGCCAT |
| | GCACCTGCAGTACAAGATCCACGAGGCCCCCTTCGACCTGCTGCTGGA |
| | GTGGCTGTACGTGCCCATCGACCCCACCTGTCAGCCCATGAGACTGTA |
| | CAGCACCTGCCTGTACCACCCCAACGCCCCTCAGTGCCTTAGTCACAT |
| | GAACAGCGGCTGCACCTTCACAAGCCCCCACCTGGCTCAGAGAGTGG |
| | CTAGCACCGTGTATCAGAACTGCGAGCACGCCGACAACTACACCGCCT |
| | ACTGCCTGGGCATCAGCCACATGGAGCCTAGCTTCGGCCTGATCCTGC |
| | ATGACGGCGGCACGACCTTAAAGTTCGTGGACACCCCCGAGAGCCTG |
| | AGCGGCCTGTACGTGTTCGTGGTGTACTTCAACGGCCACGTGGAGGCC |
| | GTGGCCTACACCGTGGTGAGCACCGTGGACCACTTCGTGAACGCAATC |
| | GAGGAGCGCGGCTTTCCGCCGACTGCCGGTCAACCCCCCGCCACCACC |
| | AAGCCCAAGGAGATCACCCCCGTGAATCCGGGCACAAGCCCTCTTATC |
| | CGGTATGCCGCCTGGACCGGCGGCCTGGCCGCCGTGGTGCTGCTGTGC |
| | CTGGTGATCTTCCTGATCTGCACCGCCAAGAGAATGAGAGTGAAGGCC |
| | GCCAGAGTGGACAAG |
| VZV gE mutein-1 | AGCGTGCTGCGGTACGACGACTTCCACATCGACGAGGATAAGCTGGA |
| coding sequence | TACCAACTCTGTGTACGAGCCCTACTACCATTCTGATCATGCCGAAAG |
| 2 (SEQ ID NO: 5) | CAGCTGGGTCAATAGAGGCGAGTCTAGCAGAAAGGCCTATGACCACA |
| | ACAGCCCTTACATCTGGCCACGGAACGACTACGACGGCTTTCTGGAAA |
| | ACGCCCACGAGCACCACGGCGTTTACAACCAGGGCAGAGGAATCGAT |
| | AGCGGAGAAAGACTGATGCAGCCTACACAAATGAGCGCCCAGGAGGA |
| | CCTCGGCGACGATACCGGCATCCACGTGATTCCTACACTGAATGGAGA |
| | TGATAGACACAAGATCGTGAATGTGGATCAGAGACAGTACGGCGATG |
| | TGTTCAAGGGCGACCTGAACCCTAAGCCCCAGGGCCAGAGACTGATT |
| | GAAGTGTCCGTCGAGGAAAACCACCCCTTCACCCTGAGAGCCCCCATC |
| | CAAAGAATCTACGGCGTGCGGTATACAGAGACATGGTCCTTCCTGCCT |
| | AGCCTGACCTGTACCGGCGACGCCGCTCCTGCCATCCAGCACATCTGC |
| | CTGAAACACACCACCTGCTTCCAGGACGTGGTGGTCGATGTGGACTGC |
| | GCCGAGAATACCAAAGAGGACCAGCTGGCTGAGATCAGCTACAGATT |
| | CCAAGGCAAGAAAGAGGCGGATCAGCCTTGGATCGTGGTGAACACCA |
| | GCACACTGTTTGATGAACTGGAACTGGACCCCCCCGAGATCGAGCCTG |
| | GCGTGCTGAAAGTGCTCCGCACCGAGAAGCAGTACCTGGGCGTGTAT |
| | ATCTGGAACATGCGGGGCAGCGACGGAACCAGCACCTACGCCACATT |
| | CCTGGTGACCTGGAAGGGCGACGAGAAGACCAGAAACCCCACCCCTG |
| | CCGTGACACCCCAGCCCAGAGGCGCCGAGTTCCACATGTGGAATTACC |
| | ACAGCCACGTGTTCAGCGTGGGCGACACATTTAGCCTGGCCATGCACC |
| | TGCAGTACAAGATCCACGAGGCCCCTTTTGACCTGCTGCTGGAATGGC |
| | TGTACGTGCCAATCGACCCCACCTGTCAGCCTATGAGGCTGTACAGCA |
| | CCTGTCTGTACCACCCTAACGCTCCTCAGTGCCTGTCCCACATGAACA |
| | GCGGATGTACCTTCACCAGCCCCCACCTGGCCCAGCGGGTGGCCTCTA |
| | CCGTGTACCAGAACTGCGAGCACGCCGATAACTACACAGCCTACTGCC |
| | TGGGCATCAGCCACATGGAACCTAGCTTCGGCCTGATCCTGCACGACG |
| | GCGGCACAACACTGAAGTTCGTGGACACCCCTGAGTCACTGTCTGGCC |
| | TGTACGTGTTCGTGGTATACTTCAACGGCCACGTGGAAGCCGTGGCCT |
| | ACACCGTCGTTTCCACCGTGGACCATTICGTGAACGCCATCGAGGAAA |
| | GAGGATTTCCTCCTACAGCCGGCCAGCCTCCAGCTACAACCAAGCCTA |
| | AAGAAATCACACCTGTGAACCCTGGAACATCCCCACTGATTAGATACG |
| | CCGCTTGGACCGGCGGGCTGGCCGCCGTGGTCCTGCTGTGCCTGGTGA |
| | TCTTCCTTATCTGCACCGCAAAGCGGATGCGGGTTAAGGCCGCCAGAG |
| | TGGACAAG |
| VZV gE mutein-2 | SVLRYDDFHIDEDKLDTNSVYEPYYHSDHAESSWVNRGESSRKAYDHNS |
| amino acid | PYIWPRNDYDGFLENAHEHHGVYNQGRGIDSGERLMQPTQMSAQEDLG |
| sequence (SEQ | DDTGIHVIPTLNGDDRHKIVNVDQRQYGDVFKGDLNPKPQGQRLIEVSVE |
| ID NO: 6) | ENHPFTLRAPIQRIYGVRYTETWSFLPSLTCTGDAAPAIQHICLKHTTCFQD |
| 31-586 aa, Y569A | VVVDVDCAENTKEDQLAEISYRFQGKKEADQPWIVVNTSTLFDELELDPP |
| Y582G | EIEPGVLKVLRTEKQYLGVYIWNMRGSDGTSTYATFLVTWKGDEKTRNP |
| | TPAVTPQPRGAEFHMWNYHSHVFSVGDTFSLAMHLQYKIHEAPFDLLLE |
| | WLYVPIDPTCQPMRLYSTCLYHPNAPQCLSHMNSGCTFTSPHLAQRVAST |
| | VYQNCEHADNYTAYCLGISHMEPSFGLILHDGGTTLKFVDTPESLSGLYV |
| | FVVYFNGHVEAVAYTVVSTVDHFVNAIEERGFPPTAGQPPATTKPKEITP |
| | VNPGTSPLIRYAAWTGGLAAVVLLCLVIFLICTAKRMRVKAARVDKSPY |
| | NQSMYGAGLP |
| VZV gE mutein-2 | AGCGTGCTGAGATACGACGACTTCCATATCGACGAGGACAAGCTGGA |
| coding sequence | CACCAACAGCGTGTACGAGCCCTACTACCACAGCGACCACGCCGAGA |
| (SEQ ID NO: 7) | GCAGCTGGGTGAACAGAGGCGAGAGCAGCAGAAAGGCCTACGACCAC |
| | AACAGCCCCTACATCTGGCCTAGAAACGACTACGACGGCTTCCTGGAG |
| | AACGCCCACGAGCACCACGGCGTGTACAACCAAGGCAGAGGCATCGA |
| | CAGCGGCGAGAGACTGATGCAGCCCACACAGATGAGCGCCCAAGAGG |
| | ACCTGGGCGACGACACCGGCATCCACGTGATCCCCACCCTGAACGGC |
| | GACGACAGACACAAGATCGTGAACGTGGATCAGAGACAGTACGGCGA |
| | CGTGTTCAAGGGCGACCTGAACCCCAAGCCCCAAGGGCAGAGACTGA |
| | TCGAGGTGAGCGTGGAGGAGAACCACCCCTTCACCCTGAGAGCCCCC |
| | ATTCAGAGAATCTACGGCGTGAGATACACCGAAACCTGGAGCTTCCTG |
| | CCTAGCCTGACCTGCACCGGCGACGCCGCCCCCGCCATTCAGCACATC |
| | TGCCTGAAGCACACCACCTGCTTCCAAGACGTGGTGGTGGACGTGGAC |
| | TGCGCCGAGAACACCAAGGAGGATCAGCTGGCCGAGATCAGCTACAG |
| | ATTCCAAGGCAAGAAGGAGGCCGATCAGCCCTGGATCGTGGTGAACA |
| | CAAGCACCCTGTTCGACGAGCTGGAGCTGGACCCCCCCGAGATCGAG |
| | CCCGGCGTGCTGAAGGTGCTGAGAACCGAGAAGCAGTACCTGGGCGT |
| | GTACATCTGGAACATGAGAGGCAGCGACGGCACAAGCACCTACGCCA |
| | CCTTCCTGGTGACCTGGAAGGGCGACGAGAAGACAAGAAACCCCACC |
| | CCCGCAGTGACGCCCCAACCTAGAGGCGCCGAGTTCCACATGTGGAA |
| | CTACCACAGCCACGTGTTCAGCGTGGGCGACACCTTCAGCCTGGCCAT |
| | GCACCTGCAGTACAAGATCCACGAGGCCCCCTTCGACCTGCTGCTGGA |
| | GTGGCTGTACGTGCCCATCGACCCCACCTGTCAGCCCATGAGACTGTA |
| | CAGCACCTGCCTGTACCACCCCAACGCCCCTCAGTGCCTTAGTCACAT |
| | GAACAGCGGCTGCACCTTCACAAGCCCCCACCTGGCTCAGAGAGTGG |
| | CTAGCACCGTGTATCAGAACTGCGAGCACGCCGACAACTACACCGCCT |
| | ACTGCCTGGGCATCAGCCACATGGAGCCTAGCTTCGGCCTGATCCTGC |
| | ATGACGGCGGCACGACCTTAAAGTTCGTGGACACCCCCGAGAGCCTG |
| | AGCGGCCTGTACGTGTTCGTGGTGTACTTCAACGGCCACGTGGAGGCC |
| | GTGGCCTACACCGTGGTGAGCACCGTGGACCACTTCGTGAACGCAATC |
| | GAGGAGCGCGGCTTTCCGCCGACTGCCGGTCAACCCCCCGCCACCACC |
| | AAGCCCAAGGAGATCACCCCCGTGAATCCGGGCACAAGCCCTCTTATC |
| | CGGTATGCCGCCTGGACCGGCGGCCTGGCCGCCGTGGTGCTGCTGTGC |
| | CTGGTGATCTTCCTGATCTGCACCGCCAAGAGAATGAGAGTGAAGGCC |
| | GCCAGAGTGGACAAGAGCCCCTACAATCAGAGCATGTACGGCGCCGG |
| | CCTGCCC |
| VZV gE mutein-3 | SVLRYDDFHIDEDKLDTNSVYEPYYHSDHAESSWVNRGESSRKAYDHNS |
| amino acid | PYIWPRNDYDGFLENAHEHHGVYNQGRGIDSGERLMQPTQMSAQEDLG |
| sequence (SEQ | DDTGIHVIPTLNGDDRHKIVNVDQRQYGDVFKGDLNPKPQGQRLIEVSVE |
| ID NO: 8) | ENHPFTLRAPIQRIYGVRYTETWSFLPSLTCTGDAAPAIQHICLKHTTCFQD |
| 31-623 aa, Y569A | VVVDVDCAENTKEDQLAEISYRFQGKKEADQPWIVVNTSTLFDELELDPP |
| Y582G | EIEPGVLKVLRTEKQYLGVYIWNMRGSDGTSTYATFLVTWKGDEKTRNP |
| | TPAVTPQPRGAEFHMWNYHSHVFSVGDTFSLAMHLQYKIHEAPFDLLLE |
| | WLYVPIDPTCQPMRLYSTCLYHPNAPQCLSHMNSGCTFTSPHLAQRVAST |
| | VYQNCEHADNYTAYCLGISHMEPSFGLILHDGGTTLKFVDTPESLSGLYV |
| | FVVYFNGHVEAVAYTVVSTVDHFVNAIEERGFPPTAGQPPATTKPKEITP |
| | VNPGTSPLIRYAAWTGGLAAVVLLCLVIFLICTAKRMRVKAARVDKSPY |
| | NQSMYGAGLPVDDFEDSESTDTEEEFGNAIGGSHGGSSYTVYIDKTR |
| VZV gE mutein-3 | AGCGTGCTGAGATACGACGACTTCCATATCGACGAGGACAAGCTGGA |
| coding sequence | CACCAACAGCGTGTACGAGCCCTACTACCACAGCGACCACGCCGAGA |
| (SEQ ID NO: 9) | GCAGCTGGGTGAACAGAGGCGAGAGCAGCAGAAAGGCCTACGACCAC |
| | AACAGCCCCTACATCTGGCCTAGAAACGACTACGACGGCTTCCTGGAG |
| | AACGCCCACGAGCACCACGGCGTGTACAACCAAGGCAGAGGCATCGA |
| | CAGCGGCGAGAGACTGATGCAGCCCACACAGATGAGCGCCCAAGAGG |
| | ACCTGGGCGACGACACCGGCATCCACGTGATCCCCACCCTGAACGGC |
| | GACGACAGACACAAGATCGTGAACGTGGATCAGAGACAGTACGGCGA |
| | CGTGTTCAAGGGCGACCTGAACCCCAAGCCCCAAGGGCAGAGACTGA |
| | TCGAGGTGAGCGTGGAGGAGAACCACCCCTTCACCCTGAGAGCCCCC |
| | ATTCAGAGAATCTACGGCGTGAGATACACCGAAACCTGGAGCTTCCTG |
| | CCTAGCCTGACCTGCACCGGCGACGCCGCCCCCGCCATTCAGCACATC |
| | TGCCTGAAGCACACCACCTGCTTCCAAGACGTGGTGGTGGACGTGGAC |
| | TGCGCCGAGAACACCAAGGAGGATCAGCTGGCCGAGATCAGCTACAG |
| | ATTCCAAGGCAAGAAGGAGGCCGATCAGCCCTGGATCGTGGTGAACA |
| | CAAGCACCCTGTTCGACGAGCTGGAGCTGGACCCCCCCGAGATCGAG |
| | CCCGGCGTGCTGAAGGTGCTGAGAACCGAGAAGCAGTACCTGGGCGT |
| | GTACATCTGGAACATGAGAGGCAGCGACGGCACAAGCACCTACGCCA |
| | CCTTCCTGGTGACCTGGAAGGGCGACGAGAAGACAAGAAACCCCACC |
| | CCCGCAGTGACGCCCCAACCTAGAGGCGCCGAGTTCCACATGTGGAA |
| | CTACCACAGCCACGTGTTCAGCGTGGGCGACACCTTCAGCCTGGCCAT |
| | GCACCTGCAGTACAAGATCCACGAGGCCCCCTTCGACCTGCTGCTGGA |
| | GTGGCTGTACGTGCCCATCGACCCCACCTGTCAGCCCATGAGACTGTA |
| | CAGCACCTGCCTGTACCACCCCAACGCCCCTCAGTGCCTTAGTCACAT |
| | GAACAGCGGCTGCACCTTCACAAGCCCCCACCTGGCTCAGAGAGTGG |
| | CTAGCACCGTGTATCAGAACTGCGAGCACGCCGACAACTACACCGCCT |
| | ACTGCCTGGGCATCAGCCACATGGAGCCTAGCTTCGGCCTGATCCTGC |
| | ATGACGGCGGCACGACCTTAAAGTTCGTGGACACCCCCGAGAGCCTG |
| | AGCGGCCTGTACGTGTTCGTGGTGTACTTCAACGGCCACGTGGAGGCC |
| | GTGGCCTACACCGTGGTGAGCACCGTGGACCACTTCGTGAACGCAATC |
| | GAGGAGCGCGGCTTTCCGCCGACTGCCGGTCAACCCCCCGCCACCACC |
| | AAGCCCAAGGAGATCACCCCCGTGAATCCGGGCACAAGCCCTCTTATC |
| | CGGTATGCCGCCTGGACCGGCGGCCTGGCCGCCGTGGTGCTGCTGTGC |
| | CTGGTGATCTTCCTGATCTGCACCGCCAAGAGAATGAGAGTGAAGGCC |
| | GCCAGAGTGGACAAGAGCCCCTACAATCAGAGCATGTACGGCGCCGG |
| | CCTGCCCGTGGACGACTTCGAGGACAGCGAGAGCACCGACACCGAGG |
| | AGGAGTTCGGCAACGCCATCGGCGGCAGCCACGGAGGCAGCAGCTAC |
| | ACCGTGTACATCGACAAGACAAGA |
| VZV gE mutein-4 | SVLRYDDFHIDEDKLDTNSVYEPYYHSDHAESSWVNRGESSRKAYDHNS |
| amino acid | PYIWPRNDYDGFLENAHEHHGVYNQGRGIDSGERLMQPTQMSAQEDLG |
| sequence (SEQ | DDTGIHVIPTLNGDDRHKIVNVDQRQYGDVFKGDLNPKPQGQRLIEVSVE |
| ID NO: 10) | ENHPFTLRAPIQRIYGVRYTETWSFLPSLTCTGDAAPAIQHICLKHTTCFQD |
| 31-623 aa, | VVVDVDCAENTKEDQLAEISYRFQGKKEADQPWIVVNTSTLFDELELDPP |
| Y569A, Y582G, | EIEPGVLKVLRTEKQYLGVYIWNMRGSDGTSTYATFLVTWKGDEKTRNP |
| S593A, S595A, | TP AVTPQPRGAEFHMWNYHSHVFSVGDTFSLAMHLQYKIHEAPFDLLLE |
| T596A, T598A | WLYVPIDPTCQPMRLYSTCLYHPNAPQCLSHMNSGCTFTSPHLAQRVAST |
| | VYQNCEHADNYTAYCLGISHMEPSFGLILHDGGTTLKFVDTPESLSGLYV |
| | FVVYFNGHVEAVAYTVVSTVDHFVNAIEERGFPPTAGQPPATTKPKEITP |
| | VNPGTSPLIRYAAWTGGLAAVVLLCLVIFLICTAKRMRVKAARVDKSPY |
| | NQSMYGAGLPVDDFEDAEAADAEEEFGNAIGGSHGGSSYTVYIDKTR |
| VZV gE mutein-4 | AGCGTGCTGAGATACGACGACTTCCATATCGACGAGGACAAGCTGGA |
| coding sequence | CACCAACAGCGTGTACGAGCCCTACTACCACAGCGACCACGCCGAGA |
| (SEQ ID NO: 11) | GCAGCTGGGTGAACAGAGGCGAGAGCAGCAGAAAGGCCTACGACCAC |
| | AACAGCCCCTACATCTGGCCTAGAAACGACTACGACGGCTTCCTGGAG |
| | AACGCCCACGAGCACCACGGCGTGTACAACCAAGGCAGAGGCATCGA |
| | CAGCGGCGAGAGACTGATGCAGCCCACACAGATGAGCGCCCAAGAGG |
| | ACCTGGGCGACGACACCGGCATCCACGTGATCCCCACCCTGAACGGC |
| | GACGACAGACACAAGATCGTGAACGTGGATCAGAGACAGTACGGCGA |
| | CGTGTTCAAGGGCGACCTGAACCCCAAGCCCCAAGGGCAGAGACTGA |
| | TCGAGGTGAGCGTGGAGGAGAACCACCCCTTCACCCTGAGAGCCCCC |
| | ATTCAGAGAATCTACGGCGTGAGATACACCGAAACCTGGAGCTTCCTG |
| | CCTAGCCTGACCTGCACCGGCGACGCCGCCCCCGCCATTCAGCACATC |
| | TGCCTGAAGCACACCACCTGCTTCCAAGACGTGGTGGTGGACGTGGAC |
| | TGCGCCGAGAACACCAAGGAGGATCAGCTGGCCGAGATCAGCTACAG |
| | ATTCCAAGGCAAGAAGGAGGCCGATCAGCCCTGGATCGTGGTGAACA |
| | CAAGCACCCTGTTCGACGAGCTGGAGCTGGACCCaCCCGAGATCGAGC |
| | CCGGCGTGCTGAAGGTGCTGAGAACCGAGAAGCAGTACCTGGGCGTG |
| | TACATCTGGAACATGAGAGGCAGCGACGGCACAAGCACCTACGCCAC |
| | CTTCCTGGTGACCTGGAAGGGCGACGAGAAGACAAGAAACCCCACCC |
| | CCGCAGTGACGCCCCAACCTAGAGGCGCCGAGTTCCACATGTGGAACT |
| | ACCACAGCCACGTGTTCAGCGTGGGCGACACCTTCAGCCTGGCCATGC |
| | ACCTGCAGTACAAGATCCACGAGGCCCCCTTCGACCTGCTGCTGGAGT |
| | GGCTGTACGTGCCCATCGACCCCACCTGTCAGCCCATGAGACTGTACA |
| | GCACCTGCCTGTACCACCCaAACGCCCCTCAGTGCCTTAGTCACATGA |
| | ACAGCGGCTGCACCTTCACAAGCCCCCACCTGGCTCAGAGAGTGGCTA |
| | GCACCGTGTATCAGAACTGCGAGCACGCCGACAACTACACCGCCTACT |
| | GCCTGGGCATCAGCCACATGGAGCCTAGCTTCGGCCTGATCCTGCATG |
| | ACGGCGGCACGACCTTAAAGTTCGTGGACACCCCCGAGAGCCTGAGC |
| | GGCCTGTACGTGTTCGTGGTGTACTTCAACGGCCACGTGGAGGCCGTG |
| | GCCTACACCGTGGTGAGCACCGTGGACCACTTCGTGAACGCAATCGAG |
| | GAGCGCGGCTTTCCGCCGACTGCCGGTCAACCCCCCGCCACCACCAAG |
| | CCCAAGGAGATCACCCCCGTGAATCCGGGCACAAGCCCTCTTATCCGG |
| | TATGCCGCCTGGACCGGCGGCCTGGCCGCCGTGGTGCTGCTGTGCCTG |
| | GTGATCTTCCTGATCTGCACCGCCAAGAGAATGAGAGTGAAGGCCGCC |
| | AGAGTGGACAAGAGCCCCTACAATCAGAGCATGTACGGCGCCGGCCT |
| | GCCCGTGGACGACTTCGAGGACGCCGAGGCCGCCGACGCCGAGGAGG |
| | AGTTCGGCAACGCCATCGGCGGCAGCCACGGAGGCAGCAGCTACACC |
| | GTGTACATCGACAAGACAAGA |
| VZV gE mutein-5 | SVLRYDDFHIDEDKLDTNSVYEPYYHSDHAESSWVNRGESSRKAYDHNS |
| amino acid | PYIWPRNDYDGFLENAHEHHGVYNQGRGIDSGERLMQPTQMSAQEDLG |
| sequence (SEQ | DDTGIHVIPTLNGDDRHKIVNVDQRQYGDVFKGDLNPKPQGQRLIEVSVE |
| ID NO: 12) | ENHPFTLRAPIQRIYGVRYTETWSFLPSLTCTGDAAPAIQHICLKHTTCFQD |
| 31-623 aa Y582G, | VVVDVDCAENTKEDQLAEISYRFQGKKEADQPWIVVNTSTLFDELELDPP |
| S593A, S595A, | EIEPGVLKVLRTEKQYLGVYIWNMRGSDGTSTYATFLVTWKGDEKTRNP |
| T596A, T598A | TPAVTPQPRGAEFHMWNYHSHVFSVGDTFSLAMHLQYKIHEAPFDLLLE |
| | WLYVPIDPTCQPMRLYSTCLYHPNAPQCLSHMNSGCTFTSPHLAQRVAST |
| | VYQNCEHADNYTAYCLGISHMEPSFGLILHDGGTTLKFVDTPESLSGLYV |
| | FVVYFNGHVEAVAYTVVSTVDHFVNAIEERGFPPTAGQPPATTKPKEITP |
| | VNPGTSPLIRYAAWTGGLAAVVLLCLVIFLICTAKRMRVKAYRVDKSPY |
| | NQSMYGAGLPVDDFEDAEAADAEEEFGNAIGGSHGGSSYTVYIDKTR |
| VZV gE mutein-5 | AGCGTGCTGAGATACGACGACTTCCATATCGACGAGGACAAGCTGGA |
| coding sequence | CACCAACAGCGTGTACGAGCCCTACTACCACAGCGACCACGCCGAGA |
| (SEQ ID NO: 13) | GCAGCTGGGTGAACAGAGGCGAGAGCAGCAGAAAGGCCTACGACCAC |
| | AACAGCCCCTACATCTGGCCTAGAAACGACTACGACGGCTTCCTGGAG |
| | AACGCCCACGAGCACCACGGCGTGTACAACCAAGGCAGAGGCATCGA |
| | CAGCGGCGAGAGACTGATGCAGCCCACACAGATGAGCGCCCAAGAGG |
| | ACCTGGGCGACGACACCGGCATCCACGTGATCCCCACCCTGAACGGC |
| | GACGACAGACACAAGATCGTGAACGTGGATCAGAGACAGTACGGCGA |
| | CGTGTTCAAGGGCGACCTGAACCCCAAGCCCCAAGGGCAGAGACTGA |
| | TCGAGGTGAGCGTGGAGGAGAACCACCCCTTCACCCTGAGAGCCCCC |
| | ATTCAGAGAATCTACGGCGTGAGATACACCGAAACCTGGAGCTTCCTG |
| | CCTAGCCTGACCTGCACCGGCGACGCCGCCCCCGCCATTCAGCACATC |
| | TGCCTGAAGCACACCACCTGCTTCCAAGACGTGGTGGTGGACGTGGAC |
| | TGCGCCGAGAACACCAAGGAGGATCAGCTGGCCGAGATCAGCTACAG |
| | ATTCCAAGGCAAGAAGGAGGCCGATCAGCCCTGGATCGTGGTGAACA |
| | CAAGCACCCTGTTCGACGAGCTGGAGCTGGACCCCCCCGAGATCGAG |
| | CCCGGCGTGCTGAAGGTGCTGAGAACCGAGAAGCAGTACCTGGGCGT |
| | GTACATCTGGAACATGAGAGGCAGCGACGGCACAAGCACCTACGCCA |
| | CCTTCCTGGTGACCTGGAAGGGCGACGAGAAGACAAGAAACCCCACC |
| | CCCGCAGTGACGCCCCAACCTAGAGGCGCCGAGTTCCACATGTGGAA |
| | CTACCACAGCCACGTGTTCAGCGTGGGCGACACCTTCAGCCTGGCCAT |
| | GCACCTGCAGTACAAGATCCACGAGGCCCCCTTCGACCTGCTGCTGGA |
| | GTGGCTGTACGTGCCCATCGACCCCACCTGTCAGCCCATGAGACTGTA |
| | CAGCACCTGCCTGTACCACCCCAACGCCCCTCAGTGCCTTAGTCACAT |
| | GAACAGCGGCTGCACCTTCACAAGCCCCCACCTGGCTCAGAGAGTGG |
| | CTAGCACCGTGTATCAGAACTGCGAGCACGCCGACAACTACACCGCCT |
| | ACTGCCTGGGCATCAGCCACATGGAGCCTAGCTTCGGCCTGATCCTGC |
| | ATGACGGCGGCACGACCTTAAAGTTCGTGGACACCCCCGAGAGCCTG |
| | AGCGGCCTGTACGTGTTCGTGGTGTACTTCAACGGCCACGTGGAGGCC |
| | GTGGCCTACACCGTGGTGAGCACCGTGGACCACTTCGTGAACGCAATC |
| | GAGGAGCGCGGCTTTCCGCCGACTGCCGGTCAACCCCCCGCCACCACC |
| | AAGCCCAAGGAGATCACCCCCGTGAATCCGGGCACAAGCCCTCTTATC |
| | CGGTATGCCGCCTGGACCGGCGGCCTGGCCGCCGTGGTGCTGCTGTGC |
| | CTGGTGATCTTCCTGATCTGCACCGCCAAGAGAATGAGAGTGAAGGCC |
| | TACAGAGTGGACAAGAGCCCCTACAATCAGAGCATGTACGGCGCCGG |
| | CCTGCCCGTGGACGACTTCGAGGACGCCGAGGCCGCCGACGCCGAGG |
| | AGGAGTTCGGCAACGCCATCGGCGGCAGCCACGGAGGCAGCAGCTAC |
| | ACCGTGTACATCGACAAGACAAGA |

Note:
sequence in italic (in case of full length protein) is signal peptide.

In particular embodiments, the therapeutic nucleic acid of the present disclosure encodes the mature gE protein of VZV, wherein the mature gE protein has an amino acid sequence of SEQ ID NO:1. In particular embodiments, the therapeutic nucleic acid of the present disclosure encodes the mature gE protein of VZV, wherein the therapeutic nucleic acid comprises a DNA coding sequence of SEQ ID NO:2. In particular embodiments, the therapeutic nucleic acid of the present disclosure encodes the mature gE protein of VZV, wherein the therapeutic nucleic acid comprises a RNA sequence transcribed from the DNA coding sequence of SEQ ID NO:2. In particular embodiments, the therapeutic nucleic acid of the present disclosure encodes the full length gE protein of VZV, wherein the full length gE protein has an amino acid sequence of SEQ ID NO:55. In particular embodiments, the therapeutic nucleic acid of the present disclosure encodes the full length gE protein of VZV, wherein the therapeutic nucleic acid comprises a DNA coding sequence of SEQ ID NO:56. In particular embodiments, the therapeutic nucleic acid of the present disclosure encodes the full length gE protein of VZV, wherein the therapeutic nucleic acid comprises a RNA sequence transcribed from the DNA coding sequence of SEQ ID NO:56. In some embodiments, the RNA sequence is in vitro transcribed. In particular embodiments, the nucleic acid molecule is an mRNA molecule.

In particular embodiments, the therapeutic nucleic acid of the present disclosure encodes an immunogenic fragment of the gE protein of VZV. In particular embodiments, the therapeutic nucleic acid of the present disclosure encodes a functional derivative of the gE protein of VZV. In particular embodiments, the therapeutic nucleic acid of the present disclosure encodes an immunogenic fragment of the gE protein of VZV, wherein the immunogenic fragment of the gE protein comprises a truncation of at least 1 amino acid residue and at most 49 amino acid residues from the C-terminal as compared with the mature gE protein. In particular embodiments, the therapeutic nucleic acid of the present disclosure encodes an immunogenic fragment of the gE protein of VZV, wherein the immunogenic fragment of the gE protein comprises a truncation of 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acid residue(s) from the C-terminal as compared with the mature gE protein. In particular embodiments, the therapeutic nucleic acid of the present disclosure encodes an immunogenic fragment of the gE protein of VZV, wherein the immunogenic fragment of the gE protein comprises a truncation of 11-18 (e.g., 11, 12, 13, 14, 15, 16, 17, or 18) or 34-44 (e.g., 34, 35, 36, 37, 38, 39, 40, 41, 42, 43, or 44) amino acid residues from the C-terminal as compared with the mature gE protein. In particular embodiments, the therapeutic nucleic acid of the present disclosure encodes an immunogenic fragment of the gE protein of VZV, wherein the immunogenic fragment of the gE protein comprises a truncation of 12-16 (e.g., 12, 13, 14, 15, or 16) or 35-39 (e.g., 35, 36, 37, 38, or 39) amino acid residues from the C-terminal as compared with the mature gE protein. In particular embodiments, the therapeutic nucleic acid of the present disclosure encodes an immunogenic fragment of the gE protein of VZV, wherein the immunogenic fragment of the gE protein comprises a truncation of 14 or 37 amino acid residues from the C-terminal as compared with the mature gE protein. In some embodiments, the RNA sequence is in vitro transcribed. In particular embodiments, the nucleic acid molecule is an mRNA molecule.

In particular embodiments, the therapeutic nucleic acid of the present disclosure encodes a mutant of the gE protein of VZV. In particular embodiments, the therapeutic nucleic acid of the present disclosure encodes a mutant of the gE protein of VZV, wherein the mutant comprises the substitution Y569A. In particular embodiments, the therapeutic nucleic acid of the present disclosure encodes a mutant of the gE protein of VZV, wherein the mutant comprises the substitution Y582G. In particular embodiments, the therapeutic nucleic acid of the present disclosure encodes a mutant of the gE protein of VZV, wherein the mutant comprises the substitutions Y569A and Y582G. In particular embodiments, the therapeutic nucleic acid of the present disclosure encodes a mutant of the gE protein of VZV, wherein the mutant comprises the substitution S593A. In particular embodiments, the therapeutic nucleic acid of the present disclosure encodes a mutant of the gE protein of VZV, wherein the mutant comprises the substitution S595A. In particular embodiments, the therapeutic nucleic acid of the present disclosure encodes a mutant of the gE protein of VZV, wherein the mutant comprises the substitution T596A. In particular embodiments, the therapeutic nucleic acid of the present disclosure encodes a mutant of the gE protein of VZV, wherein the mutant comprises the substitution T598A. In particular embodiments, the therapeutic nucleic acid of the present disclosure encodes a mutant of the gE protein of VZV, wherein the mutant comprises the substitutions S593A, S595A, T596A, and T598A. In particular embodiments, the therapeutic nucleic acid of the present disclosure encodes a mutant of the gE protein of VZV, wherein the mutant comprises the substitutions Y569A, Y582G, S593A, S595A, T596A, and T598A. In such embodiments, the amino acid positions are numbered on the basis of the full length gE protein. In some embodiments, the RNA sequence is in vitro transcribed. In particular embodiments, the nucleic acid molecule is an mRNA molecule.

In particular embodiments, the therapeutic nucleic acid of the present disclosure encodes a mutated fragment of the gE protein of VZV. In particular embodiments, the therapeutic nucleic acid of the present disclosure encodes a mutated fragment of the gE protein of VZV, wherein the mutated fragment comprises a truncation of 49, 48, 47, 46, 45, 44, 43, or 42 amino acid residues from the C-terminal as compared with the mature gE protein. In such embodiments, the mutated fragment optionally comprises the substitution Y569A. In particular embodiments, the therapeutic nucleic acid of the present disclosure encodes a mutated fragment of the gE protein of VZV, wherein the mutated fragment comprises a truncation of 41, 40, 39, 38, 37, 36, 35, 34, 33, 32, or 31 amino acid residues from the C-terminal as compared with the mature gE protein. In such embodiments, the mutated fragment optionally comprises the substitution Y582G, with or without the substitution Y569A. In such embodiments, the mutated fragment comprises the substitutions Y569A and Y582G. In particular embodiments, the therapeutic nucleic acid of the present disclosure encodes a mutated fragment of the gE protein of VZV, wherein the mutated fragment comprises a truncation of 30 or 29 amino acid residues from the C-terminal as compared with the mature gE protein. In such embodiments, the mutated fragment optionally comprises the substitution S593A, with or without the substitutions Y569A and/or Y582G. In such embodiments, the mutated fragment comprises the substitutions Y569A, Y582G and S593A. In particular embodiments, the therapeutic nucleic acid of the present disclosure encodes a mutated fragment of the gE protein of VZV, wherein the mutated fragment comprises a truncation of 28 amino acid residues from the C-terminal as compared with the mature gE protein. In such embodiments, the mutated fragment optionally comprises the substitution S595A, with or without the substitutions Y569A and/or Y582G and/or S593A. In such embodiments, the mutated fragment comprises the substitutions Y569A, Y582G, S593A, and S595A. In particular embodiments, the therapeutic nucleic acid of the present disclosure encodes a mutated fragment of the gE protein of VZV, wherein the mutated fragment comprises a truncation of 27 or 26 amino acid residues from the C-terminal as compared with the mature gE protein. In such embodiments, the mutated fragment optionally comprises the substitution T596A, with or without the substitutions Y569A and/or Y582G and/or S593A and/or S595A. In such embodiments, the mutated fragment comprises the substitutions Y569A, Y582G, S593A, S595A, and T596A. In particular embodiments, the therapeutic nucleic acid of the present disclosure encodes a mutated fragment of the gE protein of VZV, wherein the mutated fragment comprises a truncation of 25, 24, 23, 22, 21, 20, 19, 18, 17, 16, 15, 14, 13, 12, 11, 10, 9, 8, 7, 6, 5, 4, 3, or 2 amino acid residues or 1 amino acid residue from the C-terminal as compared with the mature gE protein. In such embodiments, the mutated fragment optionally comprises the substitution T598A, with or without the substitutions Y569A and/or Y582G and/or S593A and/or S595A and/or T596A. In such embodiments, the mutated fragment comprises the substitutions Y569A, Y582G, S593A, S595A, T596A, and T598A. In particular embodiments, the therapeutic nucleic acid of the present disclosure encodes a mutated fragment of the gE protein of VZV, wherein the mutated fragment has an amino acid sequence of SEQ ID NO:3. In particular embodiments, the therapeutic nucleic acid of the present disclosure encodes a mutated fragment of the gE protein of VZV, wherein the therapeutic nucleic acid comprises a DNA coding sequence of SEQ ID NO:4 or 5. In particular embodiments, the therapeutic nucleic acid of the present disclosure encodes a mutated fragment of the gE protein of VZV, wherein the therapeutic nucleic acid comprises a RNA sequence transcribed from the DNA coding sequence of SEQ ID NO:4 or 5. In particular embodiments, the therapeutic nucleic acid of the present disclosure encodes a mutated fragment of the gE protein of VZV, wherein the mutated fragment has an amino acid sequence of SEQ ID NO:6. In particular embodiments, the therapeutic nucleic acid of the present disclosure encodes a mutated fragment of the gE protein of VZV, wherein the therapeutic nucleic acid comprises a DNA coding sequence of SEQ ID NO:7. In particular embodiments, the therapeutic nucleic acid of the present disclosure encodes a mutated fragment of the gE protein of VZV, wherein the therapeutic nucleic acid comprises a RNA sequence transcribed from the DNA coding sequence of SEQ ID NO:7. In particular embodiments, the therapeutic nucleic acid of the present disclosure encodes a mutated fragment of the gE protein of VZV, wherein the mutated fragment has an amino acid sequence of SEQ ID NO:8. In particular embodiments, the therapeutic nucleic acid of the present disclosure encodes a mutated fragment of the gE protein of VZV, wherein the therapeutic nucleic acid comprises a DNA coding sequence of SEQ ID NO:9. In particular embodiments, the therapeutic nucleic acid of the present disclosure encodes a mutated fragment of the gE protein of VZV, wherein the therapeutic nucleic acid comprises a RNA sequence transcribed from the DNA coding sequence of SEQ ID NO:9. In particular embodiments, the therapeutic nucleic acid of the present disclosure encodes a mutated fragment of the gE protein of VZV, wherein the mutated fragment has an amino acid sequence of SEQ ID NO:10. In particular embodiments, the therapeutic nucleic acid of the present disclosure encodes a mutated fragment of the gE protein of VZV, wherein the therapeutic nucleic acid comprises a DNA coding sequence of SEQ ID NO:11. In particular embodiments, the therapeutic nucleic acid of the present disclosure encodes a mutated fragment of the gE protein of VZV, wherein the therapeutic nucleic acid comprises a RNA sequence transcribed from the DNA coding sequence of SEQ ID NO:11. In particular embodiments, the therapeutic nucleic acid of the present disclosure encodes a mutated fragment of the gE protein of VZV, wherein the mutated fragment has an amino acid sequence of SEQ ID NO:12. In particular embodiments, the therapeutic nucleic acid of the present disclosure encodes a mutated fragment of the gE protein of VZV, wherein the therapeutic nucleic acid comprises a DNA coding sequence of SEQ ID NO:13. In particular embodiments, the therapeutic nucleic acid of the present disclosure encodes a mutated fragment of the gE protein of VZV, wherein the therapeutic nucleic acid comprises a RNA sequence transcribed from the DNA coding sequence of SEQ ID NO:13. In some embodiments, the RNA sequence is in vitro transcribed. In particular embodiments, the nucleic acid molecule is an mRNA molecule.

Without being bound by the theory, it is contemplated that in some embodiments, the therapeutic nucleic acid of the present disclosure encodes a fusion protein comprising the VZV gE protein or a fragment thereof fused to a trimerization peptide, such that the fusion protein is capable of forming a trimeric complex comprising three copies of the gE protein or fragment thereof. In some embodiments, the gE protein or fragment thereof is fused to a trimerization peptide via a peptidic linker.

Table 2 shows exemplary trimerization peptide and linker peptide that can be used in connection with the present disclosure, and sequences of fusion proteins.

TABLE 2
Exemplary sequences of linker peptides and trimerization peptides.

| SEQUENCE NAME | |
|---|---|
| (SEQ ID NO:) | AMINO ACID OR NUCLEIC ACID SEQUENCE |
| (G3S)2 linker peptide | GGGSGGGS |
| amino acid sequence | |
| (SEQ ID NO: 14) | |
| (G3S)2 linker peptide | GGAGGAGGAAGTGGAGGAGGAAGT |
| coding sequence (SEQ | |
| ID NO: 15) | |
| Trimmerization | GYIPEAPRDGQAYVRKDGEWVLLSTFLG |
| peptide amino acid | |
| sequence (SEQ ID | |
| NO: 16) | |
| Trimmerization | GGCTATATTCCGGAAGCGCCGCGCGATGGCCAGGCGTATGTGCGCAA |
| peptide coding | AGATGGCGAATGGGTGCTGCTGAGCACCTTTCTGGGC |
| sequence (SEQ ID | |
| NO: 17) | |

In some embodiments, the therapeutic nucleic acid encodes a fusion protein comprising the gE protein of VZV or a functional derivative thereof fused to a trimerization peptide. In some embodiments, the fusion between the gE protein and the trimerization peptide is via a peptide linker. In specific embodiments, the peptide linker comprises the amino acid sequence of SEQ ID NO:14. In some embodiments, the trimerization peptide comprises the amino acid sequence of SEQ ID NO:16.

In particular embodiments, the therapeutic nucleic acid encodes a fusion protein comprising the gE protein of VZV fused to a trimerization peptide, wherein the nucleic acid comprises a DNA coding sequence. In particular embodiments, the therapeutic nucleic acid encodes a fusion protein comprising the gE protein of VZV fused to a trimerization peptide, wherein the nucleic acid comprises an RNA sequence transcribed from the DNA coding sequence. In some embodiments, the RNA sequence is in vitro transcribed. In particular embodiments, the nucleic acid molecule is an mRNA molecule.

Without being bound by the theory, it is contemplated that a fusion protein comprising a viral peptide or polypeptide fused to an immunoglobulin Fc region can enhance immunogenicity of the viral peptide or polypeptide. Accordingly, in some embodiments, the therapeutic nucleic acid molecule of the present disclosure encodes a fusion protein comprising a viral peptide or protein derived from VZV fused with an Fc region of an immunoglobulin. In particular embodiments, the viral peptide or protein is one or more selected from (a) the gE protein, (b) the gB protein, (c) the gH protein, (d) the gI protein, (e) the gC protein, (f) the gL protein, (g) an immunogenic fragment of any one of (a) to (f), and (h) a functional derivative of any one of (a) to (g). In particular embodiments, the immunoglobulin is human immunoglobulin (Ig). In particular embodiments, the immunoglobulin is human IgG, IgA, IgD, IgE, or IgM. In particular embodiments, the immunoglobulin is human IgG1, IgG2, IgG3 or IgG4. In some embodiments, the immunoglobulin Fc is fused to the N terminus of the viral peptide or polypeptide. In other embodiments, the immunoglobulin Fc is fused to the C terminus of the viral peptide or polypeptide.

Without being bound by theory, it is contemplated that a signal peptide can mediate transportation of a polypeptide fused thereto to particular locations of a cell. Accordingly, in some embodiments, the therapeutic nucleic acid molecule of the present disclosure encodes a fusion protein comprising a viral peptide or protein fused to a signal peptide. In particular embodiments, the viral peptide or protein is one or more selected from (a) the gE protein, (b) the gB protein, (c) the gH protein, (d) the gI protein, (e) the gC protein, (f) the gL protein, (g) an immunogenic fragment of any one of (a) to (f), and (h) a functional derivative of any one of (a) to (g). In some embodiments, the signal peptide is fused to the N terminus of the viral peptide or polypeptide. In other embodiments, the signal peptide is fused to the C terminus of the viral peptide or polypeptide. Table 3 shows exemplary sequences for signal peptides that can be use in connection with the present disclosure, and exemplary VZV antigenic sequences comprising the signal peptides.

TABLE 3
Exemplary sequences of signal peptides.

| SEQUENCE | |
|---|---|
| NAME | |
| (SEQ ID NO:) | AMINO ACID OR NUCLEIC ACID SEQUENCE |
| VZV gE protein | MGTVNKPVVGVLMGFGIITGTLRITNPVRA |
| native signal peptide | |
| amino acid sequence | |
| (SEQ ID NO: 18) | |
| VZV gE protein | ATGGGCACCGTGAACAAGCCCGTGGTGGGCGTGCTGATGGGCTTCGG |
| native signal peptide | CATCATCACCGGCACCCTGAGAATCACCAACCCCGTGAGAGCT |
| coding sequence 1 | |
| (SEQ ID NO: 19) | |
| VZV gE protein | ATGGGGACAGTTAATAAACCTGTGGTGGGGGTATTGATGGGGTTCGG |
| native signal peptide | AATTATCACGGGAACGTTGCGTATAACGAATCCGGTCAGAGCA |
| coding sequence 2 | |
| (SEQ ID NO: 20) | |
| VZV gE protein | ATGGGAACCGTGAACAAGCCTGTGGTTGGAGTTCTGATGGGCTTTGG |
| native signal peptide | CATCATCACAGGAACACTCAGAATCACCAACCCAGTGCGGGCC |
| coding sequence 3 | |
| (SEQ ID NO: 21) | |
| VZV gE protein | ATGGGCACGGTGAACAAGCCCGTGGTCGGGGTGCTGATGGGGTTTGG |
| native signal peptide | GATCATCACAGGGACTCTGCGTATCACGAACCCAGTGCGTGCC |
| coding sequence 4 | |
| (SEQ ID NO: 22) | |
| Human IgE signal | MDWTWILFLVAAATRVHS |
| peptide amino acid | |
| sequence (SEQ ID | |
| NO: 23) | |
| Human IgE signal | ATGGACTGGACCTGGATTCTCTTCTTGGTGGCAGCAGCCACGCGAGT |
| peptide coding | CCACTCC |
| sequence 1 (SEQ ID | |
| NO: 24) | |
| Human IgE signal | ATGGACTGGACCTGGATTCTGTTCCTGGTGGCCGCCGCCACAAGAGT |
| peptide coding | GCACAGC |
| sequence 2 (SEQ ID | |
| NO: 25) | |
| Human IgE signal | ATGGACTGGACCTGGATTCTGTTCCTGGTGGCTGCCGCTACCCGGGTG |
| peptide coding | CACAGC |
| sequence 3 (SEQ ID | |
| NO: 26) | |
| Human tPA signal | MDAMKRGLCCVLLLCGAVFVSNS |
| peptide amino acid | |
| sequence (SEQ ID | |
| NO: 27) | |
| Human tPA signal | ATGGACGCCATGAAGAGAGGCCTGTGCTGCGTGCTGCTGCTGTGCGG |
| peptide coding | CGCCGTGTTCGTGAGCAACAGC |
| sequence (SEQ ID | |
| NO: 28) | |

In particular embodiments, the signal peptide is encoded by a gene of the VZV from which the viral peptide or polypeptide is derived. In particular embodiments, a signal peptide encoded by a gene of VZV is fused to a viral peptide or polypeptide encoded by a different gene of VZV. In other embodiments, a signal peptide encoded by a gene of VZV is fused to a viral peptide or polypeptide encoded by the same gene of VZV. For example, in some embodiments, a signal peptide having amino acid sequence of MGTVNKPVVGVLMGFGIITGTLRITNPVRA (SEQ ID NO:18) is fused to the viral peptide or polypeptide encoded by the nucleic acid molecule of the present disclosure. In various embodiments, the viral peptide or protein is one or more selected from (a) the E protein, (b) the B protein, (c) the H protein, (d) the I protein, (e) the C protein, (f) the L protein, (g) an immunogenic fragment of any one of (a) to (f), and (h) a functional derivative of any one of (a) to (g).

In particular embodiments, the therapeutic nucleic acid of the present disclosure encodes the gE protein or fragment of VZV without the native signal peptide. In particular embodiments, the encoded gE protein or fragment comprises a signal peptide having an amino acid sequence of SEQ ID NO:23 or 27. In particular embodiments, the therapeutic nucleic acid of the present disclosure encodes the gE protein or fragment of VZV having a signal peptide, and wherein the therapeutic nucleic acid comprises a DNA coding sequence of SEQ ID NO:24, 25, 26 or 28. In particular embodiments, the therapeutic nucleic acid of the present disclosure encodes the gE protein or fragment of VZV having a signal peptide, and wherein the therapeutic nucleic acid comprises a RNA sequence transcribed from the DNA coding sequence of SEQ ID NO:24, 25, 26 or 28. In some embodiments, the RNA sequence is in vitro transcribed. In particular embodiments, the nucleic acid molecule is an mRNA molecule.

In other embodiments, the signal peptide is encoded by an exogenous gene sequence that does not present in VZV from which the viral peptide or polypeptide is derived. In some embodiments, a heterologous signal peptide replaces a homologous signal peptide in the fusion protein encoded by the nucleic acid molecule of the present disclosure. In specific embodiments, the signal peptide is encoded by a mammalian gene. In specific embodiments, the signal peptide is encoded by human Immunoglobulin gene. In specific embodiments, the signal peptide is encoded by human IgE gene. For example, in some embodiments, a signal peptide having amino acid sequence of MDWTWILFLVAAATRVHS (SEQ ID NO:23) is fused to the viral peptide or polypeptide encoded by the nucleic acid molecule of the present disclosure. In various embodiments, the viral peptide or protein is one or more selected from (a) the E protein, (b) the B protein, (c) the H protein, (d) the I protein, (e) the C protein, (f) the L protein, (g) an immunogenic fragment of any one of (a) to (f), and (h) a functional derivative of any one of (a) to (g).

In some embodiments, provided herein is a nucleic acid encoding a fragment of a mature gE described in Section 5.3. In some embodiments, provided herein is a nucleic acid encoding a fusion protein described in Section 5.3. In some embodiments, provided herein is a nucleic acid encoding a mutant of full length VZV gE described in Section 5.3. In some embodiments, provided herein is a nucleic acid encoding a protein described in Section 5.3. In some embodiments, provided herein is a nucleic acid comprising a nucleotide sequence set forth in SEQ ID NO: 7, 9, 11, 13, 4, or 5. In some embodiments, provided herein is a nucleic acid comprising a nucleotide sequence having at least 80%, 85%, 90%, 91%, 92%, 93%, 94%, 95%, 96%, 97%, 98%, or 99% identity with the nucleotide sequence set forth in SEQ ID NO: 7, 9, 11, 13, 4, or 5. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g, one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g., one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid encoding a protein or fusion protein described in Section 5.3, comprising a mutant or fragment of mature gE and a human IgE signal peptide, wherein the nucleotide sequence encoding the IgE signal peptide comprises the nucleotide sequence set forth in SEQ ID NO: 24, 25, or 26. In some embodiments, provided herein is a nucleic acid encoding a protein or fusion protein described in Section 5.3, comprising a mutant or fragment of mature gE and a human IgE signal peptide, wherein the nucleotide sequence encoding the IgE signal peptide comprises a nucleotide sequence that is at least 80%, 85%, 90%, 91%, 92%, 93%, 94%, 95%, 96%, 97%, 98%, or 99% identical to the nucleotide sequence set forth in SEQ ID NO: 24, 25, or 26. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g, one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid encoding a protein described in Section 5.3, comprising a mutant of mature gE and a signal peptide, wherein the nucleotide sequence encoding the signal peptide comprises the nucleotide sequence set forth in SEQ ID NO: 19, 20, 21, or 22. In some embodiments, provided herein is a nucleic acid encoding a protein described in Section 5.3, comprising a mutant of mature gE and a signal peptide, wherein the nucleotide sequence encoding the signal peptide is at least 80%, 85%, 90%, 91%, 92%, 93%, 94%, 95%, 96%, 97%, 98%, or 99% identical to the nucleotide sequence set forth in SEQ ID NO: 19, 20, 21, or 22. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g., one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid encoding a protein or the fusion protein described in Section 5.3, comprising a mutant or fragment of mature gE and a human tPA signal peptide, wherein the nucleotide sequence encoding the human tPA signal peptide comprises the nucleotide sequence set forth in SEQ ID NO: 28. In some embodiments, provided herein is a nucleic acid encoding a protein or the fusion protein described in Section 5.3, comprising a mutant or fragment of mature gE and a human tPA signal peptide, wherein the nucleotide sequence encoding the human tPA signal peptide comprises a nucleotide sequence that is at least 80%, 85%, 90%, 91%, 92%, 93%, 94%, 95%, 96%, 97%, 98%, or 99% identical to the nucleotide sequence set forth in SEQ ID NO: 28. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g, one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid comprising the nucleotide sequence set forth in SEQ ID NO: 63, 7, 51, 60, 62, 62, 64, 9, 11, 13, 52, 53, 54, 58, 49, 50, 4, or 5. In some embodiments, provided herein is a nucleic acid comprising the nucleotide sequence set forth in SEQ ID NO: 63. In some embodiments, provided herein is a nucleic acid consisting of, consisting essentially of, or comprising the nucleotide sequence set forth in SEQ ID NO: 63, 7, 51, 60, 62, 62, 64, 9, 11, 13, 52, 53, 54, 58, 49, 50, 4, or 5. In some embodiments, provided herein is a nucleic acid consisting of, consisting essentially of, or comprising the nucleotide sequence set forth in SEQ ID NO: 63. In some embodiments, provided herein is a nucleic acid consisting of, consisting essentially of, or comprising a nucleotide sequence with at least 80%, at least 81%, at least 82%, at least 83%, or at least 84% identity to the nucleotide sequence set forth in SEQ ID NO: 63, 7, 51, 60, 62, 62, 64, 9, 11, 13, 52, 53, 54, 58, 49, 50, 4, or 5. In some embodiments, provided herein is a nucleic acid consisting of, consisting essentially of, or comprising a nucleotide sequence with at least 85%, at least 86%, at least 87%, at least 88%, or at least 89% identity to the nucleotide sequence set forth in SEQ ID NO: 63, 7, 51, 60, 62, 62, 64, 9, 11, 13, 52, 53, 54, 58, 49, 50, 4, or 5. In some embodiments, provided herein is a nucleic acid consisting of, consisting essentially of, or comprising a nucleotide sequence with at least 90%, at least 910%, at least 92%, at least 93%, or at least 94% identity to the nucleotide sequence set forth in SEQ ID NO: 63, 7, 51, 60, 62, 62, 64, 9, 11, 13, 52, 53, 54, 58, 49, 50, 4, or 5. In some embodiments, provided herein is a nucleic acid consisting of, consisting essentially of, or comprising a nucleotide sequence with at least 95%, at least 96%, at least 97%, at least 98%, or at least 99% identity to the nucleotide sequence set forth in SEQ ID NO: 63, 7, 51, 60, 62, 62, 64, 9, 11, 13, 52, 53, 54, 58, 49, 50, 4, or 5. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g., one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid encoding the amino acid sequence set forth in SEQ ID NO: 6. In some embodiments, the nucleic acid comprises the nucleotide sequence set forth in SEQ IN NO: 7. In some embodiments, provided herein is a nucleic acid encoding the amino acid sequence set forth in SEQ ID NO: 8. In some embodiments, the nucleic acid comprises the nucleotide sequence set forth in SEQ IN NO: 9. In some embodiments, provided herein is a nucleic acid encoding the amino acid sequence set forth in SEQ ID NO: 10. In some embodiments, the nucleic acid comprises the nucleotide sequence set forth in SEQ IN NO: 11. In some embodiments, provided herein is a nucleic acid encoding the amino acid sequence set forth in SEQ ID NO: 12. In some embodiments, the nucleic acid comprises the nucleotide sequence set forth in SEQ IN NO: 13. In some embodiments, provided herein is a nucleic acid encoding the amino acid sequence set forth in SEQ ID NO: 3. In some embodiments, the nucleic acid comprises the nucleotide sequence set forth in SEQ IN NO: 4 or 5. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g., one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid encoding the amino acid sequence of SEQ ID NO: 59. In some embodiments, the nucleic acid comprises the nucleotide sequence set forth in SEQ ID NO: 51, 60, 61, 62, 63, or 64. In some embodiments, the nucleic acid comprises the nucleotide sequence set forth in SEQ ID NO: 63. In some embodiments, the nucleic acid consists of the nucleotide sequence set forth in SEQ ID NO: 51, 60, 61, 62, 63, or 64. In some embodiments, the nucleic acid consists of the nucleotide sequence set forth in SEQ ID NO: 63. In some embodiments, the nucleic acid comprises a nucleotide sequence that is at least 80%, at least 81%, at least 82%, at least 83%, or at least 84% identical to the nucleotide sequence set forth in SEQ ID NO: 51, 60, 61, 62, 63, or 64. In some embodiments, the nucleic acid comprises a nucleotide sequence that is at least 85%, at least 86%, at least 87%, at least 88%, or at least 89% identical to the nucleotide sequence set forth in SEQ ID NO: 51, 60, 61, 62, 63, or 64. In some embodiments, the nucleic acid comprises a nucleotide sequence that is at least 90%, at least 91%, at least 92%, at least 92%, at least 93%, or at least 94% identical to the nucleotide sequence set forth in SEQ ID NO: 51, 60, 61, 62, 63, or 64. In some embodiments, the nucleic acid comprises a nucleotide sequence that is at least 95%, at least 96%, at least 97%, at least 98%, or at least 99% identical to the nucleotide sequence set forth in SEQ ID NO: 51, 60, 61, 62, 63, or 64. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g, one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid comprising the nucleotide sequence set forth in SEQ ID NO: 51, 60, 61, 62, 63, or 64. In some embodiments, provided herein is a nucleic acid consisting of the nucleotide sequence set forth in SEQ ID NO: 51, 60, 61, 62, 63, or 64. In some embodiments, provided herein is a nucleic acid consisting of, consisting essentially of, or comprising a nucleotide sequence with at least 80%, at least 81%, at least 82%, at least 83%, or at least 84% identity to the nucleotide sequence set forth in SEQ ID NO: 51, 60, 61, 62, 63, or 64. In some embodiments, provided herein is a nucleic acid consisting of, consisting essentially of, or comprising a nucleotide sequence with at least 85%, at least 86%, at least 87%, at least 88%, or at least 89% identity to the nucleotide sequence set forth in SEQ ID NO: 51, 60, 61, 62, 63, or 64. In some embodiments, provided herein is a nucleic acid consisting of, consisting essentially of, or comprising a nucleotide sequence with at least 90%, at least 910%, at least 92%, at least 930%, or at least 94% identity to the nucleotide sequence set forth in SEQ ID NO: 51, 60, 61, 62, 63, or 64. In some embodiments, provided herein is a nucleic acid consisting of, consisting essentially of, or comprising a nucleotide sequence with at least 95%, at least 96%, at least 97%, at least 98%, or at least 99% identity to the nucleotide sequence set forth in SEQ ID NO: 51, 60, 61, 62, 63, or 64. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g, one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid comprising the nucleotide sequence set forth in SEQ ID NO: 63. In some embodiments, provided herein is a nucleic acid consisting of the nucleotide sequence set forth in SEQ ID NO: 63. In some embodiments, provided herein is a nucleic acid consisting of, consisting essentially of, or comprising a nucleotide sequence with at least 80%, at least 81%, at least 82%, at least 83%, or at least 84% identity to the nucleotide sequence set forth in SEQ ID NO: 63. In some embodiments, provided herein is a nucleic acid consisting of, consisting essentially of, or comprising a nucleotide sequence with at least 85%, at least 86%, at least 87%, at least 88%, or at least 89% identity to the nucleotide sequence set forth in SEQ ID NO: 63. In some embodiments, provided herein is a nucleic acid consisting of, consisting essentially of, or comprising a nucleotide sequence with at least 90%, at least 91%, at least 92%, at least 93%, or at least 94% identity to the nucleotide sequence set forth in SEQ ID NO: 63. In some embodiments, provided herein is a nucleic acid consisting of, consisting essentially of, or comprising a nucleotide sequence with at least 95%, at least 96%, at least 97%, at least 98%, or at least 99% identity to the nucleotide sequence set forth in SEQ ID NO: 63. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g, one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid comprising the nucleotide sequence set forth in SEQ ID NO: 49, 50, 52, 53, or 54. In some embodiments, provided herein is a nucleic acid consisting of the nucleotide sequence set forth in SEQ ID NO: 49, 50, 52, 53, or 54. In some embodiments, provided herein is a nucleic acid consisting of, consisting essentially of, or comprising a nucleotide sequence with at least 80%, at least 81%, at least 82%, at least 83%, or at least 84% identity to the nucleotide sequence set forth in SEQ ID NO: 49, 50, 52, 53, or 54. In some embodiments, provided herein is a nucleic acid consisting of, consisting essentially of, or comprising a nucleotide sequence with at least 85%, at least 86%, at least 87%, at least 88%, or at least 89% identity to the nucleotide sequence set forth in SEQ ID NO: 49, 50, 52, 53, or 54. In some embodiments, provided herein is a nucleic acid consisting of, consisting essentially of, or comprising a nucleotide sequence with at least 90%, at least 91%, at least 92%, at least 93%, 49, 50, 52, 53, or 5460, 61, 62, 63, or 64. In some embodiments, provided herein is a nucleic acid consisting of, consisting essentially of, or comprising a nucleotide sequence with at least 95%, at least 96%, at least 97%, at least 98%, or at least 99% identity to the nucleotide sequence set forth in SEQ ID NO: 49, 50, 52, 53, or 54. In some embodiments, the nucleic acid is non-naturally occurring. In some embodiments, the nucleic acid is DNA. In some embodiments, the nucleic acid is mRNA. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g., one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In some embodiments, provided herein is a nucleic acid set forth in Table 1 or an mRNA of a nucleic acid set forth in Table 1. In some embodiments, provided herein is a nucleic acid set forth in Table 1 other than SEQ ID NOs: 2 and 56, or an mRNA of a nucleic acid set forth in Table 1 other than SEQ ID NOs: 2 and 56. In some embodiments, provided herein is a nucleic acid comprising a VZV gE mutein coding sequence set forth in Table 1, or an mRNA of a nucleic acid of a VZV gE mutein coding sequence set forth in Table 1. In some embodiments, provided herein is a nucleic acid consisting of a VZV gE mutein coding sequence set forth in Table 1, or an mRNA of a nucleic acid of a VZV gE mutein coding sequence set forth in Table 1. In some embodiments, provided herein is a nucleic acid comprising a VZV gE mutein-1 coding sequence set forth in Table 1, or an mRNA of a nucleic acid of a VZV gE mutein-1 coding sequence set forth in Table 1. In some embodiments, provided herein is a nucleic acid comprising a VZV gE mutein-2 coding sequence set forth in Table 1, or an mRNA of a nucleic acid of a VZV gE mutein-2 coding sequence set forth in Table 1. In some embodiments, provided herein is a nucleic acid comprising a VZV gE mutein-3 coding sequence set forth in Table 1, or an mRNA of a nucleic acid of a VZV gE mutein-3 coding sequence set forth in Table 1. In some embodiments, provided herein is a nucleic acid comprising a VZV gE mutein-4 coding sequence set forth in Table 1, or an mRNA of a nucleic acid of a VZV gE mutein-4 coding sequence set forth in Table 1. In some embodiments, provided herein is a nucleic acid comprising a VZV gE mutein-5 coding sequence set forth in Table 1, or an mRNA of a nucleic acid of a VZV gE mutein-5 coding sequence set forth in Table 1. In some embodiments, the nucleic acid comprises one or more functional nucleotide analogs (e.g., one or more functional nucleotide analogs described in Section 5.4.6, 5.4.7, and/or 5.4.8). In some embodiments, the nucleic acid is mRNA and the nucleic acid comprises one or more functional nucleotide analogs that are selected from pseudouridine, 1-methyl-pseudouridine and 5-methylcytosine. In some embodiments, the nucleic acid comprises one or more modified internucleoside linkages (e.g., one or more modified internucleoside linkages described in Section 5.4.9). In some embodiments, the nucleic acid further comprises a 5′-cap structure, such as, e.g., described in Section 5.4.2. In some embodiments, the nucleic acid further comprises a 5′-cap structure, such as, e.g., described in Section 5.4.2. In some embodiments, the nucleic acid further comprises a 5′ untranslated region (5′-UTR), such as, e.g., described in Section 5.4.3. In some embodiments, the nucleic acid further comprises a 3′ untranslated region (3′-UTR), such as, e.g., described in Section 5.4.3. In some embodiments, the nucleic acid further comprises a 5′ untranslated region (5′-UTR), such as, e.g., described in Section 5.4.3, and a 3′ untranslated region (3′-UTR), such as, e.g., described in Section 5.4.3. In some embodiments, the 5′-UTR comprises the nucleotide sequence set forth in any one of SEQ ID NOS: 29-38. In some embodiments, the 3′-UTR comprises the nucleotide sequence set forth in any one of SEQ ID NOS: 39-46. In some embodiments, the nucleic acid further comprises a 5′-UTR and a 3′-UTR, wherein the 5′-UTR comprises the nucleotide sequence set forth in any one of SEQ ID NOS: 29-38 and the 3′-UTR comprises the nucleotide sequence set forth in any one of SEQ ID NOS: 39-46. In some embodiments, the 3′-UTR further comprises a poly-A tail or a polyadenylation signal, such as, e.g., described in Section 5.4.4.

In specific embodiments, the coding nucleotide sequence of a nucleic acid (e.g, a non-naturally occurring nucleic acid) described herein has been codon optimized for expression in cells of a subject, optionally, wherein the subject is a non-human mammal or a human.

In specific embodiments, a nucleic acid (e.g., a non-naturally occurring nucleic acid) described herein is mRNA, and wherein thymines (e.g., in the corresponding DNA sequence) are substituted for uracils or a functional analog thereof in the nucleic acid.

In specific embodiments, provided herein is a nucleic acid described in Section 6 below. In specific embodiments, provided herein is a codon optimized nucleic acid described in Section 6 below. In specific embodiments, provided herein is a non-naturally occurring nucleic acid described in Section 6 below. In some embodiments, provided herein is a nucleic acid encoding a mutant of a VZV gE described in Section 6 below other than a control.

### 7.4.2 5′-Cap Structure

Without being bound by the theory, it is contemplated that, a 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The 5′-cap structure further assists the removal of 5′-proximal introns removal during mRNA splicing. Accordingly, in some embodiments, the nucleic acid molecules of the present disclosure comprise a 5′-cap structure.

Nucleic acid molecules may be 5′-end capped by the endogenous transcription machinery of a cell to generate a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a nucleic acid molecule, such as an mRNA molecule, for degradation.

In some embodiments, the nucleic acid molecules of the present disclosure comprise one or more alterations to the natural 5′-cap structure generated by the endogenous process. Without being bound by the theory, a modification on the 5′-cap may increase the stability of polynucleotide, increase the half-life of the polynucleotide, and could increase the polynucleotide translational efficiency.

Exemplary alterations to the natural 5′-Cap structure include generation of a non-hydrolyzable cap structure preventing decapping and thus increasing polynucleotide half-life. In some embodiments, because cap structure hydrolysis requires cleavage of 5′-ppp-5′ phosphorodiester linkages, in some embodiments, modified nucleotides may be used during the capping reaction. For example, in some embodiments, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used, such as α-methyl-phosphonate and seleno-phosphate nucleotides.

Additional exemplary alterations to the natural 5′-Cap structure also include modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

Additional exemplary alterations to the natural 5′-cap structure include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of a polynucleotide, such as an mRNA molecule. Additional exemplary 5′-Cap structures that can be used in connection with the present disclosure further include those described in International Patent Publication Nos. WO 2008/127688, WO 2008/016473, and WO 2011/015347, the entire contents of each of which are incorporated herein by reference.

In various embodiments, 5′-terminal caps can include cap analogs. Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide.

For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7,3′-O-dimethyl-guanosine-5′-triphosphate-5′-guanosine, m7G-3′mppp-G, which may equivalently be designated 3′ O-Me-m7G(5′)ppp(5′)G). The 3′-O atom of the other, unaltered, guanosine becomes linked to the 5′-terminal nucleotide of the capped polynucleotide (e.g., an mRNA). The N7- and 3′-O-methlyated guanosine provides the terminal moiety of the capped polynucleotide (e.g., mRNA). Another exemplary cap structure is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, a cap analog can be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the entire content of which is herein incorporated by reference in its entirety.

In some embodiments, a cap analog can be a N7-(4-chlorophenoxyethyl) substituted dinucleotide cap analog known in the art and/or described herein. Non-limiting examples of N7-(4-chlorophenoxyethyl) substituted dinucleotide cap analogs include a N7-(4-chlorophenoxyethyl)-G(5′)ppp(5′)G and a N7-(4-chlorophenoxyethyl)-m3′-OG(5′)ppp(5′)G cap analog (see, e.g., the various cap analogs and the methods of synthesizing cap analogs described in Kore et al. Bioorganic & Medicinal Chemistry 2013 21:4570-4574; the entire content of which is herein incorporated by reference). In other embodiments, a cap analog useful in connection with the nucleic acid molecules of the present disclosure is a 4-chloro/bromophenoxyethyl analog.

In various embodiments, a cap analog can include a guanosine analog. Useful guanosine analogs include but are not limited to inosine, N1-methyl-guanosine, 2′-fluoro-guanosine, 7-deaza-guanosine, 8-oxo-guanosine, 2-amino-guanosine, LNA-guanosine, and 2-azido-guanosine.

Without being bound by the theory, it is contemplated that while cap analogs allow for the concomitant capping of a polynucleotide in an in vitro transcription reaction, up to 20% of transcripts remain uncapped. This, as well as the structural differences of a cap analog from the natural 5′-cap structures of polynucleotides produced by the endogenous transcription machinery of a cell, may lead to reduced translational competency and reduced cellular stability.

Accordingly, in some embodiments, a nucleic acid molecule of the present disclosure can also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in connection with the nucleic acid molecules of the present disclosure are those which, among other things, have enhanced binding of cap binding proteins, increased half-life, reduced susceptibility to 5′-endonucleases, and/or reduced 5′-decapping as compared to synthetic 5′-cap structures known in the art (or to a wild-type, natural or physiological 5′-cap structure). For example, in some embodiments, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Cap 1 structure. This cap results in a higher translational-competency, cellular stability, and a reduced activation of cellular pro-inflammatory cytokines, as compared, e.g., to other 5′cap analog structures known in the art. Other exemplary cap structures include 7mG(5′)ppp(5′)N, pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4). In some embodiments, the cap structure comprises m⁷ GpppAmpU.

Without being bound by the theory, it is contemplated that the nucleic acid molecules of the present disclosure can be capped post-transcriptionally, and because this process is more efficient, nearly 100% of the nucleic acid molecules may be capped.

In some embodiments, provided herein is anon-naturally occurring nucleic acid consisting of, consisting essentially of, or comprising (1) the nucleotide sequence set forth in SEQ ID NO: 63, or (2) a nucleotide sequence that is at least 80%, 85%, 90%, 91%, 92%, 93%, 94%, 95%, 96%, 97%, 98%, or 99% identical to the nucleotide sequence set forth in SEQ ID NO: 63; except that all thymines (T) are substituted for uracil (U) or N1-methylpseudouridine, and/or the first nucleotide G is substituted for m⁷GpppAmpU. In some embodiments, provided herein is a nucleic acid consisting of, consisting essentially of, or comprising: (1) the nucleotide sequence set forth in SEQ ID NO: 63, or (2) a nucleotide sequence that is at least 80%, 85%, 90%, 91%, 92%, 93%, 94%, 95%, 96%, 97%, 98%, or 99% identical to the nucleotide sequence set forth in SEQ ID NO: 63; except that all thymines (T) are substituted for uracil (U) or N1-methylpseudouridine, and/or the first nucleotide G is substituted for m⁷ GpppAmpU. In some embodiments, provided herein is a nucleic acid consisting of, consisting essentially of, or comprising the nucleotide sequence set forth in SEQ ID NO: 63, except that all thymines (T) are substituted for uracil (U) or N1-methylpseudouridine, and/or the first nucleotide G is substituted for m⁷ GpppAmpU. In some embodiments, provided herein is a nucleic acid consisting of, consisting essentially of, or comprising the nucleotide sequence set forth in SEQ ID NO: 63, except that all thymines (T) are substituted for N1-methylpseudouridine, and the first nucleotide G is substituted for m⁷ GpppAmpU.

### 7.4.3 Untranslated Regions (UTRs)

In some embodiments, the nucleic acid molecules of the present disclosure comprise one or more untranslated regions (UTRs). In some embodiments, an UTR is positioned upstream to a coding region in the nucleic acid molecule, and is termed 5′-UTR In some embodiments, an UTR is positioned downstream to a coding region in the nucleic acid molecule, and is termed 3′-UTR. The sequence of an UTR can be homologous or heterologous to the sequence of the coding region found in a nucleic acid molecule. Multiple UTRs can be included in a nucleic acid molecule and can be of the same or different sequences, and/or genetic origin. According to the present disclosure, any portion of UTRs in a nucleic acid molecule (including none) can be codon optimized and any may independently contain one or more different structural or chemical modification, before and/or after codon optimization.

In some embodiments, a nucleic acid molecule of the present disclosure (e.g., mRNA) comprises UTRs and coding regions that are homologous with respect to each other. In other embodiments, a nucleic acid molecule of the present disclosure (e.g., mRNA) comprises UTRs and coding regions that are heterologous with respect to each other. In some embodiments, to monitor the activity of a UTR sequence, a nucleic acid molecule comprising the UTR and a coding sequence of a detectable probe can be administered in vitro (e.g., cell or tissue culture) or in vivo (e.g., to a subject), and an effect of the UTR sequence (e.g., modulation on the expression level, cellular localization of the encoded product, or half-life of the encoded product) can be measured using methods known in the art.

In some embodiments, the UTR of a nucleic acid molecule of the present disclosure (e.g., mRNA) comprises at least one translation enhancer element (TEE) that functions to increase the amount of polypeptide or protein produced from the nucleic acid molecule. In some embodiments, the TEE is located in the 5′-UTR of the nucleic acid molecule. In other embodiments, the TEE is located at the 3′-UTR of the nucleic acid molecule. In yet other embodiments, at least two TEE are located at the 5′-UTR and 3′-UTR of the nucleic acid molecule respectively. In some embodiments, a nucleic acid molecule of the present disclosure (e.g., mRNA) can comprise one or more copies of a TEE sequence or comprise more than one different TEE sequences. In some embodiments, different TEE sequences that are present in a nucleic acid molecule of the present disclosure can be homologues or heterologous with respect to one another.

Various TEE sequences that are known in the art and can be used in connection with the present disclosure. For example, in some embodiments, the TEE can be an internal ribosome entry site (IRES), HCV-IRES or an IRES element. Chappell et al. Proc. Natl. Acad. Sci. USA 101:9590-9594, 2004; Zhou et al. Proc. Natl. Acad. Sci. 102:6273-6278, 2005. Additional internal ribosome entry site (IRES) that can be used in connection with the present disclosure include but are not limited to those described in U.S. Pat. No. 7,468,275, U.S. Patent Publication No. 2007/0048776 and U.S. Patent Publication No. 2011/0124100 and International Patent Publication No. WO 2007/025008 and International Patent Publication No. WO 2001/055369, the content of each of which is enclosed herein by reference in its entirety. In some embodiments, the TEE can be those described in Supplemental Table 1 and in Supplemental Table 2 of Wellensiek et al Genome-wide profiling of human cap-independent translation-enhancing elements, Nature Methods, 2013 August; 10(8): 747-750; the content of which is incorporated by reference in its entirety.

Additional exemplary TEEs that can be used in connection with the present disclosure include but are not limited to the TEE sequences disclosed in U.S. Pat. Nos. 6,310,197, 6,849,405, 7,456,273, 7,183,395, U.S. Patent Publication No. 2009/0226470, U.S. Patent Publication No. 2013/0177581, U.S. Patent Publication No. 2007/0048776, U.S. Patent Publication No. 2011/0124100, U.S. Patent Publication No. 2009/0093049, International Patent Publication No. WO 2009/075886, International Patent Publication No. WO 2012/009644, and International Patent Publication No. WO 1999/024595, International Patent Publication No. WO 2007/025008, International Patent Publication No. WO 2001/055371, European Patent No. 2610341, European Patent No. 2610340, the content of each of which is enclosed herein by reference in its entirety.

In various embodiments, a nucleic acid molecule of the present disclosure (e.g., mRNA) comprises at least one UTR that comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some embodiments, the TEE sequences in the UTR of a nucleic acid molecule are copies of the same TEE sequence. In other embodiments, at least two TEE sequences in the UTR of a nucleic acid molecule are of different TEE sequences. In some embodiments, multiple different TEE sequences are arranged in one or more repeating patterns in the UTR region of a nucleic acid molecule. For illustrating purpose only, a repeating pattern can be, for example, ABABAB, AABBAABBAABB, ABCABCABC, or the like, where in these exemplary patterns, each capitalized letter (A, B, or C) represents a different TEE sequence. In some embodiments, at least two TEE sequences are consecutive with one another (i.e., no spacer sequence in between) in a UTR of a nucleic acid molecule. In other embodiments, at least two TEE sequences are separated by a spacer sequence. In some embodiments, a UTR can comprise a TEE sequence-spacer sequence module that is repeated at least once, at least twice, at least 3 times, at least 4 times, at least 5 times, at least 6 times, at least 7 times, at least 8 times, at least 9 times, or more than 9 times in the UTR. In any of the embodiments described in this paragraph, the UTR can be a 5′-UTR, a 3′-UTR or both 5′-UTR and 3′-UTR of a nucleic acid molecule.

In some embodiments, the UTR of a nucleic acid molecule of the present disclosure (e.g., mRNA) comprises at least one translation suppressing element that functions to decrease the amount of polypeptide or protein produced from the nucleic acid molecule. In some embodiments, the UTR of the nucleic acid molecule comprises one or more miR sequences or fragment thereof (e.g., miR seed sequences) that are recognized by one or more microRNA. In some embodiments, the UTR of the nucleic acid molecule comprises one or more stem-loop structure that downregulates translational activity of the nucleic acid molecule. Other mechanisms for suppressing translational activities associated with nucleic acid molecules are known in the art. In any of the embodiments described in this paragraph, the UTR can be a 5′-UTR, a 3′-UTR or both 5′-UTR and 3′-UTR of a nucleic acid molecule. Table 4 shows exemplary 5′-UTR and 3′-UTR sequences that can be used in connection with the present disclosure.

TABLE 4
Exemplary Untranslated Region (UTR) Sequences.

| SEQUENCE | |
|---|---|
| NAME | |
| (SEQ ID NO:) | AMINO ACID OR NUCLEIC ACID SEQUENCE |
| 5′-UTR DNA | GAAATAAGAGAGAAAAGAAGAGTAAGAAGAAATATAAGA |
| Sequence (SEQ ID | |
| NO: 29) | |
| 5′-UTR RNA | GAAAUAAGAGAGAAAAGAAGAGUAAGAAGAAAUAUAAGA |
| Sequence (SEQ ID | |
| NO: 30) | |
| 5′-UTR DNA | CTTGTTCTTTTTGCAGAAGCTCAGAATAAACGCTCAACTITGG |
| Sequence (SEQ ID | |
| NO: 31) | |
| 5′-UTR RNA | CUUGUUCUUUUUGCAGAAGCUCAGAAUAAACGCUCAACUUUGG |
| Sequence (SEQ ID | |
| NO: 32) | |
| 5′-UTR DNA | GCAGGAGCCAGGGCTGGGCATAAAAGTCAGGGCAGAGCCATCTATT |
| Sequence (SEQ ID | GCTTACATTTGCTTCTGACACAACTGTGTTCACTAGCAACCTCAAACA |
| NO: 33) | GACACC |
| 5′-UTR RNA | GCAGGAGCCAGGGCUGGGCAUAAAAGUCAGGGCAGAGCCAUCUAU |
| Sequence (SEQ ID | UGCUUACAUUUGCUUCUGACACAACUGUGUUCACUAGCAACCUCAA |
| NO: 34) | ACAGACACC |
| 5′-UTR DNA | AGCTCTCAACACAACATATACAAAACAAACGAATCTCAAGCAATCAA |
| Sequence (SEQ ID | GCATTCTACTTCTATTGCAGCAATTTAAATCATTTCTTTTAAAGCAAA |
| NO: 35) | AGCAATTTTCTGAAAATTTTCACCATTTACGAACGATAGCCC |
| 5′-UTR RNA | AGCUCUCAACACAACAUAUACAAAACAAACGAAUCUCAAGCAAUCA |
| Sequence (SEQ ID | AGCAUUCUACUUCUAUUGCAGCAAUUUAAAUCAUUUCUUUUAAAG |
| NO: 36) | CAAAAGCAAUUUUCUGAAAAUUUUCACCAUUUACGAACGAUAGCC |
| | C |
| 5′-UTR DNA | CTTGTTCTTTTTGCAGAAGCTCAGAATAAACGCTCAACTTTGGC |
| Sequence (SEQ ID | |
| NO: 37) | |
| 5′-UTR RNA | CUUGUUCUUUUUGCAGAAGCUCAGAAUAAACGCUCAACUUUGGC |
| Sequence (SEQ ID | |
| NO: 38) | |
| 3′-UTR DNA | GCTGGAGCCTCGGTGGCCATGCTTCTTGCCCCTTGGGCCTCCCCCCAG |
| Sequence (SEQ ID | CCCCTCCTCCCCTTCCTGCACCCGTACCCCCGTGGTCTTTGAATAAAG |
| NO: 39) | TCTGAGTGGGCGGC |
| 3′-UTR RNA | GCUGGAGCCUCGGUGGCCAUGCUUCUUGCCCCUUGGGCCUCCCCCC |
| Sequence (SEQ ID | AGCCCCUCCUCCCCUUCCUGCACCCGUACCCCCGUGGUCUUUGAAU |
| NO: 40) | AAAGUCUGAGUGGGCGGC |
| 3′-UTR DNA | GCTCGCTTTCTTGCTGTCCAATTTCTATTAAAGGTTCCTTTGTTCCCTA |
| Sequence (SEQ ID | AGTCCAACTACTAAACTGGGGGATATTATGAAGGGCCTTGAGCATCT |
| NO: 41) | GGATTCTGCCTAATAAAAAACATITATTTTCATTGC |
| 3′-UTR RNA | GCUCGCUUUCUUGCUGUCCAAUUUCUAUUAAAGGUUCCUUUGUUCC |
| Sequence (SEQ ID | CUAAGUCCAACUACUAAACUGGGGGAUAUUAUGAAGGGCCUUGAG |
| NO: 42) | CAUCUGGAUUCUGCCUAAUAAAAAACAUUUAUUUUCAUUGC |
| 3′-UTR DNA | CTGGTACTGCATGCACGCAATGCTAGCTGCCCCTTTCCCGTCCTGGGT |
| Sequence (SEQ ID | ACCCCGAGTCTCCCCCGACCTCGGGTCCCAGGTATGCTCCCACCTCCA |
| NO: 43) | CCTGCCCCACTCACCACCTCTGCTAGTTCCAGACACCTCCCAAGCACG |
| | CAGCAATGCAGCTCAAAACGCTTAGCCTAGCCACACCCCCACGGGAA |
| | ACAGCAGTGATTAACCTTTAGCAATAAACGAAAGTITAACTAAGCTA |
| | TACTAACCCCAGGGTTGGTCAATTTCGTGCCAGCCACACC |
| 3′-UTR RNA | CUGGUACUGCAUGCACGCAAUGCUAGCUGCCCCUUUCCCGUCCUGG |
| Sequence (SEQ ID | GUACCCCGAGUCUCCCCCGACCUCGGGUCCCAGGUAUGCUCCCACC |
| NO: 44) | UCCACCUGCCCCACUCACCACCUCUGCUAGUUCCAGACACCUCCCA |
| | AGCACGCAGCAAUGCAGCUCAAAACGCUUAGCCUAGCCACACCCCC |
| | ACGGGAAACAGCAGUGAUUAACCUUUAGCAAUAAACGAAAGUUUA |
| | ACUAAGCUAUACUAACCCCAGGGUUGGUCAAUUUCGUGCCAGCCAC |
| | ACC |
| 3′-UTR DNA | GCTGGAGCCTCGGTGGCCATGCTTCTTGCCCCTTGGGCCTCCCCCaAG |
| Sequence (SEQ ID | CCCCTCCTCCCCTTCCTGCACCCGTACCCCCGTGGTCTTTGAATAAAGT |
| NO: 45) | CTGAGTGGGCGGC |
| 3′-UTR RNA | GCUGGAGCCUCGGUGGCCAUGCUUCUUGCCCCUUGGGCCUCCCCCa |
| Sequence (SEQ ID | AGCCCCUCCUCCCCUUCCUGCACCCGUACCCCCGUGGUCUUUGAAU |
| NO: 46) | AAAGUCUGAGUGGGGGC |

In specific embodiments, the nucleic acid molecule of the present disclose comprises a 5′-UTR selected from any one of SEQ ID NOS:29-38. In specific embodiments, the nucleic acid molecule of the present disclose comprises a 3′-UTR selected from any one of SEQ ID NOS:39-46. In specific embodiments, the nucleic acid molecule of the present disclose comprises a 5′-UTR selected from any one of SEQ ID NOS:29-38 and a 3′-UTR selected from any one of SEQ ID NOS:39-46. In any of the embodiments described in this paragraph, the nucleic acid molecule may further comprise a coding region having a sequence as described herein, such as any of the DNA coding sequences in Tables 1 to 4 or equivalent RNA sequences thereof. In particular embodiments, the nucleic acid molecules described in this paragraph can be RNA molecules in vitro transcribed.

TABLE 5
Exemplary DNA constructs

| name | Sequence |
|---|---|
| ABOC-115S-1 | GGATCCAAGCTTAGCTTG |
| (SEQ ID NO: 49) | CTTGTTCTTTTTGCAGAAGCTCAGAATAAACGCTCAACTTTGGC |
| Encoding human | GCCGCCACC |
| IgE SP fused with | ATGGACTGGACCTGGATTCTGTTCCTGGTGGCCGCCGCCACAAGA |
| gE mutein-1 | GTGCACAGC |
| | AGCGTGCTGAGATACGACGACTTCCATATCGACGAGGACAAGCTG |
| | GACACCAACAGCGTGTACGAGCCCTACTACCACAGCGACCACGCC |
| | GAGAGCAGCTGGGTGAACAGAGGCGAGAGCAGCAGAAAGGCCTA |
| | CGACCACAACAGCCCCTACATCTGGCCTAGAAACGACTACGACGG |
| | CTTCCTGGAGAACGCCCACGAGCACCACGGCGTGTACAACCAAGG |
| | CAGAGGCATCGACAGCGGCGAGAGACTGATGCAGCCCACACAGAT |
| | GAGCGCCCAAGAGGACCTGGGCGACGACACCGGCATCCACGTGAT |
| | CCCCACCCTGAACGGCGACGACAGACACAAGATCGTGAACGTGGA |
| | TCAGAGACAGTACGGCGACGTGTTCAAGGGCGACCTGAACCCCAA |
| | GCCCCAAGGGCAGAGACTGATCGAGGTGAGCGTGGAGGAGAACC |
| | ACCCCTTCACCCTGAGAGCCCCCATTCAGAGAATCTACGGCGTGA |
| | GATACACCGAAACCTGGAGCTTCCTGCCTAGCCTGACCTGCACCG |
| | GCGACGCCGCCCCCGCCATTCAGCACATCTGCCTGAAGCACACCA |
| | CCTGCTTCCAAGACGTGGTGGTGGACGTGGACTGCGCCGAGAACA |
| | CCAAGGAGGATCAGCTGGCCGAGATCAGCTACAGATTCCAAGGCA |
| | AGAAGGAGGCCGATCAGCCCTGGATCGTGGTGAACACAAGCACCC |
| | TGTTCGACGAGCTGGAGCTGGACCCCCCCGAGATCGAGCCCGGCG |
| | TGCTGAAGGTGCTGAGAACCGAGAAGCAGTACCTGGGCGTGTACA |
| | TCTGGAACATGAGAGGCAGCGACGGCACAAGCACCTACGCCACCT |
| | TCCTGGTGACCTGGAAGGGCGACGAGAAGACAAGAAACCCCACCC |
| | CCGCAGTGACGCCCCAACCTAGAGGCGCCGAGTTCCACATGTGGA |
| | ACTACCACAGCCACGTGTTCAGCGTGGGCGACACCTTCAGCCTGG |
| | CCATGCACCTGCAGTACAAGATCCACGAGGCCCCCTTCGACCTGCT |
| | GCTGGAGTGGCTGTACGTGCCCATCGACCCCACCTGTCAGCCCATG |
| | AGACTGTACAGCACCTGCCTGTACCACCCCAACGCCCCTCAGTGCC |
| | TTAGTCACATGAACAGCGGCTGCACCTTCACAAGCCCCCACCTGGC |
| | TCAGAGAGTGGCTAGCACCGTGTATCAGAACTGCGAGCACGCCGA |
| | CAACTACACCGCCTACTGCCTGGGCATCAGCCACATGGAGCCTAG |
| | CTTCGGCCTGATCCTGCATGACGGCGGCACGACCTTAAAGTTCGTG |
| | GACACCCCCGAGAGCCTGAGCGGCCTGTACGTGTTCGTGGTGTACT |
| | TCAACGGCCACGTGGAGGCCGTGGCCTACACCGTGGTGAGCACCG |
| | TGGACCACTTCGTGAACGCAATCGAGGAGCGCGGCTTTCCGCCGA |
| | CTGCCGGTCAACCCCCCGCCACCACCAAGCCCAAGGAGATCACCC |
| | CCGTGAATCCGGGCACAAGCCCTCTTATCCGGTATGCCGCCTGGAC |
| | CGGCGGCCTGGCCGCCGTGGTGCTGCTGTGCCTGGTGATCTTCCTG |
| | ATCTGCACCGCCAAGAGAATGAGAGTGAAGGCCgccAGAGTGGAC |
| | AAG |
| | TGAtaa |
| | GAATTCTAG |
| | GCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAAT |
| | AAAGTCTGAGTGGGCGGC |
| | ACTAGT |
| | Aₙ |
| ABOC-115S-1-1 | GGATCCAAGCTTAGCTTG |
| (SEQ ID NO: 50) | CTTGTTCTTTTTGCAGAAGCTCAGAATAAACGCTCAACTTTGGC |
| Encoding human | GCCGCCACC |
| IgE SP fused with | ATGGACTGGACCTGGATTCTGTTCCTGGTGGCTGCCGCTACCCGGG |
| mutein-1 | TGCACAGC |
| | AGCGTGCTGCGGTACGACGACTTCCACATCGACGAGGATAAGCTG |
| | GATACCAACTCTGTGTACGAGCCCTACTACCATTCTGATCATGCCG |
| | AAAGCAGCTGGGTCAATAGAGGCGAGTCTAGCAGAAAGGCCTATG |
| | ACCACAACAGCCCTTACATCTGGCCACGGAACGACTACGACGGCT |
| | TTCTGGAAAACGCCCACGAGCACCACGGCGTTTACAACCAGGGCA |
| | GAGGAATCGATAGCGGAGAAAGACTGATGCAGCCTACACAAATG |
| | AGCGCCCAGGAGGACCTCGGCGACGATACCGGCATCCACGTGATT |
| | CCTACACTGAATGGAGATGATAGACACAAGATCGTGAATGTGGAT |
| | CAGAGACAGTACGGCGATGTGTTCAAGGGCGACCTGAACCCTAAG |
| | CCCCAGGGCCAGAGACTGATTGAAGTGTCCGTCGAGGAAAACCAC |
| | CCCTTCACCCTGAGAGCCCCCATCCAAAGAATCTACGGCGTGCGGT |
| | ATACAGAGACATGGTCCTTCCTGCCTAGCCTGACCTGTACCGGCGA |
| | CGCCGCTCCTGCCATCCAGCACATCTGCCTGAAACACACCACCTGC |
| | TTCCAGGACGTGGTGGTCGATGTGGACTGCGCCGAGAATACCAAA |
| | GAGGACCAGCTGGCTGAGATCAGCTACAGATTCCAAGGCAAGAAA |
| | GAGGCGGATCAGCCTTGGATCGTGGTGAACACCAGCACACTGTTT |
| | GATGAACTGGAACTGGACCCCCCCGAGATCGAGCCTGGCGTGCTG |
| | AAAGTGCTCCGCACCGAGAAGCAGTACCTGGGCGTGTATATCTGG |
| | AACATGCGGGGCAGCGACGGAACCAGCACCTACGCCACATTCCTG |
| | GTGACCTGGAAGGGCGACGAGAAGACCAGAAACCCCACCCCTGCC |
| | GTGACACCCCAGCCCAGAGGCGCCGAGTTCCACATGTGGAATTAC |
| | CACAGCCACGTGTTCAGCGTGGGCGACACATTTAGCCTGGCCATG |
| | CACCTGCAGTACAAGATCCACGAGGCCCCTTTTGACCTGCTGCTGG |
| | AATGGCTGTACGTGCCAATCGACCCCACCTGTCAGCCTATGAGGCT |
| | GTACAGCACCTGTCTGTACCACCCTAACGCTCCTCAGTGCCTGTCC |
| | CACATGAACAGCGGATGTACCTTCACCAGCCCCCACCTGGCCCAG |
| | CGGGTGGCCTCTACCGTGTACCAGAACTGCGAGCACGCCGATAAC |
| | TACACAGCCTACTGCCTGGGCATCAGCCACATGGAACCTAGCTTCG |
| | GCCTGATCCTGCACGACGGCGGCACAACACTGAAGTTCGTGGACA |
| | CCCCTGAGTCACTGTCTGGCCTGTACGTGTTCGTGGTATACTTCAA |
| | CGGCCACGTGGAAGCCGTGGCCTACACCGTCGTTTCCACCGTGGA |
| | CCATTTCGTGAACGCCATCGAGGAAAGAGGATTTCCTCCTACAGCC |
| | GGCCAGCCTCCAGCTACAACCAAGCCTAAAGAAATCACACCTGTG |
| | AACCCTGGAACATCCCCACTGATTAGATACGCCGCTTGGACCGGC |
| | GGGCTGGCCGCCGTGGTCCTGCTGTGCCTGGTGATCTTCCTTATCT |
| | GCACCGCAAAGCGGATGCGGGTTAAGGCCgccAGAGTGGACAAG |
| | TGATAA |
| | GAATTCTAG |
| | GCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAAT |
| | AAAGTCTGAGTGGGCGGC |
| | ACTAGT |
| | Aₙ |
| ABOC-115S-5 | GGATCCAAGCTTAGCTTG |
| (SEQ ID NO: 51) | CTTGTTCTTTTTGCAGAAGCTCAGAATAAACGCTCAACTTTGGC |
| Encoding human | GCCGCCACC |
| IgE SP fused with | ATGGACTGGACCTGGATTCTGTTCCTGGTGGCCGCCGCCACAAGA |
| mutein-2 | GTGCACAGC |
| | AGCGTGCTGAGATACGACGACTTCCATATCGACGAGGACAAGCTG |
| | GACACCAACAGCGTGTACGAGCCCTACTACCACAGCGACCACGCC |
| | GAGAGCAGCTGGGTGAACAGAGGCGAGAGCAGCAGAAAGGCCTA |
| | CGACCACAACAGCCCCTACATCTGGCCTAGAAACGACTACGACGG |
| | CTTCCTGGAGAACGCCCACGAGCCCACGGCGTTGTACAACCAAGG |
| | CAGAGGCATCGACAGCGGCGAGAGACTGATGCAGCCCACACAGAT |
| | GAGCGCCCAAGAGGACCTGGGCGACGACACCGGCATCCACGTGAT |
| | CCCCACCCTGAACGGCGACGACAGACACAAGATCGTGAACGTGGA |
| | TCAGAGACAGTACGGCGACGTGTTCAAGGGCGACCTGAACCCCAA |
| | GCCCCAAGGGCAGAGACTGATCGAGGTGAGCGTGGAGGAGAACC |
| | ACCCCTTCACCCTGAGAGCCCCCATTCAGAGAATCTACGGCGTGA |
| | GATACACCGAAACCTGGAGCTTCCTGCCTAGCCTGACCTGCACCG |
| | GCGACGCCGCCCCCGCCATTCAGCACATCTGCCTGAAGCACACCA |
| | CCTGCTTCCAAGACGTGGTGGTGGACGTGGACTGCGCCGAGAACA |
| | CCAAGGAGGATCAGCTGGCCGAGATCAGCTACAGATTCCAAGGCA |
| | AGAAGGAGGCCGATCAGCCCTGGATCGTGGTGAACACAAGCACCC |
| | TGTTCGACGAGCTGGAGCTGGACCCCCCCGAGATCGAGCCCGGCG |
| | TGCTGAAGGTGCTGAGAACCGAGAAGCAGTACCTGGGCGTGTACA |
| | TCTGGAACATGAGAGGCAGCGACGGCACAGCACCTACGGCCACCT |
| | TCCTGGTGACCTGGAAGGGCGACGAGAAGACAAGAAACCCCACCC |
| | CCGCAGTGACGCCCCAACCTAGAGGCGCCGAGTTCCACATGTGGA |
| | ACTACCACAGCCACGTGTTCAGCGTGGGCGACACCTTCAGCCTGCT |
| | CCATGCACCTGCAGTACAAGATCCACGAGGCCCCCTTCGACCTGCT |
| | GCTGGAGTGGCTGTACGTGCCCATCGACCCCACCTGTCAGCCCATG |
| | AGACTGTACAGCACCTGCCTGTACCACCCCAACGCCCCTCAGTGCC |
| | TTAGTCACATGAACAGCGGCTGCACCTTCACAAGCCCCCACCTGGC |
| | TCAGAGAGTGGCTAGCACCGTGTATCAGAACTGCGAGCACGCCGA |
| | CAACTACACCGCCTACTGCCTGGGCATCAGCCACATGGAGCCTAG |
| | CTTCGGCCTGATCCTGCATGACGGCGGCACGACCTTAAAGTTCGTG |
| | GACACCCCCGAGAGCCTGAGCGGCCTGTACGTGTTCGTGGTGTACT |
| | TCAACGGCCACGTGGAGGCCGTGGCCTACACCGTGGTGAGCACCG |
| | TGGACCAACTTCGTGAACGCAATCGAGGAGCGCGGCTTTCCGCCGA |
| | CTGCCGGTCAACCCCCCGCCACCACCAAGCCCAAGGAGATCACCC |
| | CCGTGAATCCGGGCACAAGCCCTCTTATCCGGTATGCCGCCTGGAC |
| | CGGCGGCCTGGCCGCCGTGGTGCTGCTGTGCCTGGTGATCTTCCTG |
| | ATCTGCACCGCCAAGAGAATGAGAGTGAAGGCCgccAGAGTGGAC |
| | AAGAGCCCCTACAATCAGAGCATGTACggcGCCGGCCTGCCC |
| | TGATAA |
| | GAATTCTAG |
| | GCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAAT |
| | AAAGTCTGAGTGGGCGGC |
| | ACTAGT |
| | Aₙ |
| ABOC-115S-4 | GGATCCAAGCTTAGCTTG |
| (SEQ ID NO: 52) | CTTGTTCTTTTTGCAGAAGCTCAGAATAAACGCTCAACTTTGGC |
| Encoding human | GCCGCCACC |
| IgE SP fused with | ATGGACTGGACCTGGATTCTGTTCCTGGTGGCCGCCGCCACAAGA |
| gE mutein-3 | GTGCACAGC |
| | AGCGTGCTGAGATACGACGACTTCCATATCGACGAGGACAAGCTG |
| | GACACCAACAGCGTGTACGAGCCCTACTACCACAGCGACCACGCC |
| | GAGAGCAGCTGGGTGAACAGAGGCGAGAGCAGCAGAAAGGCCTA |
| | CGACCACAACAGCCCCTACATCTGGCCTAGAAACGACTACGACGG |
| | CTTCCTGGAGAACGCCCACGAGCACCACGGCGTGTACAACCAAGG |
| | CAGAGGCATCGACAGCGGCGAGAGACTGATGCAGCCCACACAGAT |
| | GAGCGCCCAAGAGGACCTGGGCGACGACACCGGCATCCACGTGAT |
| | CCCCACCCTGAACGGCGACGACAGACACAAGATCGTGAACGTGGA |
| | TCAGAGACAGTACGGCGACGTGTTCAAGGGCGACCTGAACCCCAA |
| | GCCCCAAGGGCAGAGACTGATCGAGGTGAGCGTGGAGGAGAACC |
| | ACCCCTTCACCCTGAGAGCCCCCATTCAGAGAATCTACGGCGTGA |
| | GATACACCGAAACCTGGAGCTTCCTGCCTAGCCTGACCTGCACCG |
| | GCGACGCCGCCCCCGCCATTCAGCACATCTGCCTGAAGCACACCA |
| | CCTGCTTCCAAGACGTGGTGGTGGACGTGGACTGCGCCGAGAACA |
| | CCAAGGAGGATCAGCTGGCCGAGATCAGCTACAGATTCCAAGGCA |
| | AGAAGGAGGCCGATCAGCCCTGGATCGTGGTGAACACAAGCACCC |
| | TGTTCGACGAGCTGGAGCTGGACCCCCCCGAGATCGAGCCCGGCG |
| | TGCTGAAGGTGCTGAGAACCGAGAAGCAGTACCTGGGCGTGTACA |
| | TCTGGAACATGAGAGGCAGCGACGGCACAAGCACCTACGCCACCT |
| | TCCTGGTGACCTGGAAGGGCGACGAGAAGACAAGAAACCCCACCC |
| | CCGCAGTGACGCCCCAACCTAGAGGCGCCGAGTTCCACATGTGGA |
| | ACTACCACAGCCACGTGTTCAGCGTGGGCGACACCTTCAGCCTGG |
| | CCATGCACCTGCAGTACAAGATCCACGAGGCCCCCTTCGACCTGCT |
| | GCTGGAGTGGCTGTACGTGCCCATCGACCCCACCTGTCAGCCCATG |
| | AGACTGTACAGCACCTGCCTGTACCACCCCAACGCCCCCTCAGTGCC |
| | TTAGTCACATGAACAGCGGCTGCACCTTCACAAGCCCCCACCTGGC |
| | TCAGAGAGTGGCTAGCACCGTGTATCAGAACTGCGAGCACGCCGA |
| | CAACTACACCGCCTACTGCCTGGGCATCAGCCACATGGAGCCTAG |
| | CTTCGGCCTGATCCTGCATGACGGCGGCACGACCTTAAAGTTCGTG |
| | GACACCCCCGAGAGCCTGAGCGGCCTGTACGTGTTCGTGGTGTACT |
| | TCAACGGCCACGTGGAGGCCGTGGCCTACACCGTGGTGAGCACCG |
| | TGGACCCTTCGTGAACGCAATCGAGGAGCGCGGCTTTCCGCCGA |
| | CTGCCGGTCAACCCCCCGCCACCACCAAGCCCAAGGAGATCACCC |
| | CCGTGAATCCGGGCACAAGCCCTCTTATCCGGTATGCCGCCTGGAC |
| | CGGCGGCCTGGCCGCCGTGGTGCTGCTGTGCCTGGTGATCTTCCTG |
| | ATCTGCACCGCCAAGAGAATGAGAGTGAAGGCCgccAGAGTGGAC |
| | AAGAGCCCCTACAATCAGAGCATGTACggcGCCGGCCTGCCCGTGG |
| | ACGACTTCGAGGACAGCGAGAGCACCGACACCGAGGAGGAGTTC |
| | GGCAACGCCATCGGCGGCAGCCACGGAGGCAGCAGCTACACCGTG |
| | TACATCGACAAGACAAGA |
| | TGATAA |
| | GAATTCTAG |
| | GCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAAT |
| | AAAGTCTGAGTGGGCGGC |
| | ACTAGT |
| | Aₙ |
| ABOC-115S-2-2 | GGATCCAAGCTTAGCTTG |
| (SEQ ID NO: 53) | CTTGTTCTTTTTGCAGAAGCTCAGAATAAACGCTCAACTTTGGC |
| Encoding human | GCCGCCACC |
| IgE SP fused with | ATGGACTGGACCTGGATTCTGTTCCTGGTGGCCGCCGCCACAAGA |
| gE mutein-4 | GTGCACAGC |
| | AGCGTGCTGAGATACGACGACTTCCATATCGACGAGGACAAGCTG |
| | GACACCAACAGCGTGTACGAGCCCTACTACCACAGCGACCACGCC |
| | GAGAGCAGCTGGGTGAACAGAGGCGAGAGCAGCAGAAAGGCCTA |
| | CGACCACAACAGCCCCTACATCTGGCCTAGAAACGACTACGACGG |
| | CTTCCTGGAGAACGCCCACGAGCACCACGGCGTGTACAACCAAGG |
| | CAGAGGCATCGACAGCGGCGAGAGACTGATGCAGCCCACACAGAT |
| | GAGCGCCCAAGAGGACCTGGGCGACGACACCGGCATCCACGTGAT |
| | CCCCACCCTGAACGGCGACGACAGACACAAGATCGTGAACGTGGA |
| | TCAGAGACAGTACGGCGACGTGTTCAAGGGCGACCTGAACCCCAA |
| | GCCCCAAGGGCAGAGACTGATCGAGGTGAGCGTGGAGGAGAACC |
| | ACCCCTTCACCCTGAGAGCCCCCATTCAGAGAATCTACGGCGTGA |
| | GATACACCGAAACCTGGAGCTTCCTGCCTAGCCTGACCTGCACCG |
| | GCGACGCCGCCCCCGCCATTCAGCACATCTGCCTGAAGCACACCA |
| | CCTGCTTCCAAGACGTGGTGGTGGACGTGGACTGCGCCGAGAACA |
| | CCAAGGAGGATCAGCTGGCCGAGATCAGCTACAGATTCCAAGGCA |
| | AGAAGGAGGCCGATCAGCCCTGGATCGTGGTGAACACAAGCACCC |
| | TGTTCGACGAGCTGGAGCTGGACCCaCCCGAGATCGAGCCCGGCGT |
| | GCTGAAGGTGCTGAGAACCGAGAAGCAGTACCTGGGCGTGTACAT |
| | CTGGAACATGAGAGGCAGCGACGGCACAAGCACCTACGCCACCTT |
| | CCTGGTGACCTGGAAGGGCGACGAGAAGACAAGAAACCCCACCCC |
| | CGCAGTGACGCCCCAACCTAGAGGCGCCGAGTTCCACATGTGGAA |
| | CTACCACAGCCACGTGTTCAGCGTGGGCGACACCTTCAGCCTGGCC |
| | ATGCACCTGCAGTACAAGATCCACGAGGCCCCCTTCGACCTGCTGC |
| | TGGAGTGGCTGTACGTGCCCATCGACCCCACCTGTCAGCCCATGAG |
| | ACTGTACAGCACCTGCCTGTACCACCCaAACGCCCCTCAGTGCCTT |
| | AGTCACATGAACAGCGGCTGCACCTTCACAAGCCCCCACCTGGCT |
| | CAGAGAGTGGCTAGCACCGTGTATCAGAACTGCGAGCACGCCGAC |
| | AACTACACCGCCTACTGCCTGGGCATCAGCCACATGGAGCCTAGC |
| | TTCGGCCTGATCCTGCATGACGGCGGCACGACCTTAAAGTTCGTGG |
| | ACACCCCCGAGAGCCTGAGCGGCCTGTACGTGTTCGTGGTGTACTT |
| | CAACGGCCACGTGGAGGCCGTGGCCTACACCGTGGTGAGCACCGT |
| | GGACCACTTCGTGAACGCAATCGAGGAGCGCGGCTTTCCGCCGAC |
| | TGCCGGTCAACCCCCCGCCACCACCAAGCCCAAGGAGATCACCCC |
| | CGTGAATCCGGGCACAAGCCCTCTTATCCGGTATGCCGCCTGGACC |
| | GGCGGCCTGGCCGCCGTGGTGCTGCTGTGCCTGGTGATCTTCCTGA |
| | TCTGCACCGCCAAGAGAATGAGAGTGAAGGCCgccAGAGTGGACA |
| | AGAGCCCCTACAATCAGAGCATGTACggcGCCGGCCTGGCCCGTGGA |
| | CGACTTCGAGGACgccGAGgccgccGACgccGAGGAGGAGTTCGGCAA |
| | CGCCATCGGCGGCAGCCACGGAGGCAGCAGCTACACCGTGTACAT |
| | CGACAAGACAAGA |
| | TGAtaa |
| | gaattcTAG |
| | GCTGGAGCCTCGGTGGCCATGCTTCTTGCCCCTTGGGCCTCCCCCa |
| | AGCCCCTCCTCCCCTTCCTGCACCCGTACCCCCGTGGTCTTTGAAT |
| | AAAGTCTGAGTGGGCGGC |
| | ACTAGT |
| | Aₙ |
| ABOC-115S-3 | GGATCCAAGCTTAGCTTG |
| (SEQ ID NO: 54) | CTTGTTCTTTTTGCAGAAGCTCAGAATAAACGCTCAACTTTGGC |
| Encoding human | GCCGCCACC |
| IgE SP fused with | ATGGACTGGACCTGGATTCTGTTCCTGGTGGCCGCCGCCACAAGA |
| mutein-5 | GTGCACAGC |
| | AGCGTGCTGAGATACGACGACTTCCATATCGACGAGGACAAGCTG |
| | GACACCAACAGCGTGTACGAGCCCTACTACCACAGCGACCACGCC |
| | GAGAGCAGCTGGGTGAACAGAGGCGAGAGCAGCAGAAAGGCCTA |
| | CGACCACAACAGCCCCTACATCTGGCCTAGAAACGACTACGACGG |
| | CTTCCTGGAGAACGCCCACGAGCACCACGGCGTGTACAACCAAGG |
| | CAGAGGCATCGACAGCGGCGAGAGACTGATGCAGCCCACACAGAT |
| | GAGCGCCCAAGAGGACCTGGGCGACGACACCGGCATCCACGTGAT |
| | CCCCACCCTGAACGGCGACGACAGACACAAGATCGTGAACGTGGA |
| | TCAGAGACAGTACGGCGACGTGTTCAAGGGCGACCTGAACCCCAA |
| | GCCCCAAGGGCAGAGACTGATCGAGGTGAGCGTGGAGGAGAACC |
| | ACCCCTTCACCCTGAGAGCCCCCATTCAGAGAATCTACGGCGTGA |
| | GATACACCGAAACCTGGAGCTTCCTGCCTAGCCTGACCTGCACCG |
| | GCGACGCCGCCCCCGCCATTCAGCACATCTGCCTGAAGCACACCA |
| | CCTGCTTCCAAGACGTGGTGGTGGACGTGGACTGCGCCGAGAACA |
| | CCAAGGAGGATCAGCTGGCCGAGATCAGCTACAGATTCCAAGGCA |
| | AGAAGGAGGCCGATCAGCCCTGGATCGTGGTGAACACAAGCACCC |
| | TGTTCGACGAGCTGGAGCTGGACCCCCCCGAGATCGAGCCCGGCG |
| | TGCTGAAGGTGCTGAGAACCGAGAAGCAGTACCTGGGCGTGTACA |
| | TCTGGAACATGAGAGGCAGCGACGGCACAAGCACCTACGCCACCT |
| | TCCTGGTGACCTGGAAGGGCGACGAGAAGACAAGAAACCCCACCC |
| | CCGCAGTGACGCCCCAACCTAGAGGCGCCGAGTTCCACATGTGGA |
| | ACTACCACAGCCACGTGTTCAGCGTGGGCGACACCTTCAGCCTGG |
| | CCATGCACCTGCAGTACAAGATCCACGAGGCCCCCTTCGACCTGCT |
| | GCTGGAGTGGCTGTACGTGCCCATCGACCCCACCTGTCAGCCCATG |
| | AGACTGTACAGCACCTGCCTGTACCACCCCAACGCCCCTCAGTGCC |
| | TTAGTCACATGAACAGCGGCTGCACCTTCACAAGCCCCCACCTGGC |
| | TCAGAGAGTGGCTAGCACCGTGTATCAGAACTGCGAGCACGCCGA |
| | CAACTACACCGCCTACTGCCTGGGCATCAGCCACATGGAGCCTAG |
| | CTTCGGCCTGATCCTGCATGACGGCGGCACGACCTTAAAGTTCGTG |
| | GACACCCCCGAGAGCCTGAGCGGCCTGTACGTGTTCGTGGTGTACT |
| | TCAACGGCCACGTGGAGGCCGTGGCCTACACCGTGGTGAGCACCG |
| | TGGACCACTTCGTGAACGCAATCGAGGAGCGCGGCTTTCCGCCGA |
| | CTGCCGGTCAACCCCCCGCCACCACCAAGCCCAAGGAGATCACCC |
| | CCGTGAATCCGGGCACAAGCCCTCTTATCCGGTATGCCGCCTGGAC |
| | CGGCGGCCTGGCCGCCGTGGTGCTGCTGTGCCTGGTGATCTTCCTG |
| | ATCTGCACCGCCAAGAGAATGAGAGTGAAGGCCTACAGAGTGGAC |
| | AAGAGCCCCTACAATCAGAGCATGTACggcGCCGGCCTGCCCGTGG |
| | ACGACTTCGAGGACgccGAGgccgccGACgccGAGGAGGAGTTCGGCA |
| | ACGCCATCGGCGGCAGCCACGGAGGCAGCAGCTACACCGTGTACA |
| | TCGACAAGACAAGA |
| | TGAtaa |
| | GAATTCTAG |
| | GCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAAT |
| | AAAGTCTGAGTGGGCGGC |
| | ACTAGT |
| | Aₙ |
| ABOC-115S | MDWTWILFLVAAATRVHSSVLRYDDFHIDEDKLDTNSVYEPYYHSDHA |
| (wild type) | ESSWVNRGESSRKAYDHNSPYIWPRNDYDGFLENAHEHHGVYNQGR |
| (SEQ ID NO: 57) | GIDSGERLMQPTQMSAQEDLGDDTGIHVIPTLNGDDRHKIVNVDQRQ |
| Human IgE SP | YGDVFKGDLNPKPQGQRLIEVSVEENHPFTLRAPIQRIYGVRYTETWS |
| fused with WT gE | FLPSLTCTGDAAPAIQHICLKHTTCFQDVVVDVDCAENTKEDQLAEIS |
| | YRFQGKKEADQPWIVVNTSTLFDELELDPPEIEPGVLKVLRTEKQYLG |
| | VYIWNMRGSDGTSTYATFLVTWKGDEKTRNPTPAVTPQPRGAEFHM |
| | WNYHSHVFSVGDTFSLAMHLQYKIHEAPFDLLLEWLYVPIDPTCQPM |
| | RLYSTCLYHPNAPQCLSHMNSGCTFTSPHLAQRVASTVYQNCEHADN |
| | YTAYCLGISHMEPSFGLILHDGGTTLKFVDTPESLSGLYVFVVYFNGH |
| | VEAVAYTVVSTVDHFVNAIEERGFPPTAGQPPATTKPKEITPVNPGTSP |
| | LIRYAAWTGGLAAVVLLCLVIFLICTAKRMRVKAYRVDKSPYNQSM |
| | YYAGLPVDDFEDSESTDTEEEFGNAIGGSHGGSSYTVYIDKTR |
| ABOC-115S | GGGATCCAAGCTTAGCTTGCTTGTTCTTTTTGCAGAAGCTCAGAAT |
| (wild type) | AAACGCTCAACTTTGGCGCCGCCACCATGGACTGGACCTGGATTCT |
| (SEQ ID NO: 58) | GTTCCTGGTGGCCGCCGCCACAAGAGTGCACAGCAGCGTGCTGAG |
| Encoding human | ATACGACGACTTCCATATCGACGAGGACAAGCTGGACACCAACAG |
| IgE SP fused with | CGTGTACGAGCCCTACTACCACAGCGACCACGCCGAGAGCAGCTG |
| WT gE | GGTGAACAGAGGCGAGAGCAGCAGAAAGGCCTACGACCACAACA |
| | GCCCCTACATCTGGCCTAGAAACGACTACGACGGCTTCCTGGAGA |
| | ACGCCCACGAGCACCACGGCGTGTACAACCAAGGCAGAGGCATCG |
| | ACAGCGGCGAGAGACTGATGCAGCCCAACACAGATGAGCGCCCAA |
| | GAGGACCTGGGCGACGACACCGGCATCCACGTGATCCCCACCCTG |
| | AACGGCGACGACAGACACAAGATCGTGAACGTGGATCAGAGACA |
| | GTACGGCGACGTGTTCAAGGGCGACCTGAACCCCAAGCCCCAAGG |
| | GCAGAGACTGATCGAGGTGAGCGTGGAGGAGAACCACCCCTTCAC |
| | CCTGAGAGCCCCCATTCAGAGAATCTACGGCGTGAGATACACCGA |
| | AACCTGGAGCTTCCTGCCTAGCCTGACCTGCACCGGCGACGCCGC |
| | CCCCGCCATTCAAGCACATCTGCCTGAAGCACACCACCTGCTTCCAA |
| | GACGTGGTGGTGGACGTGGACTGCGCCGAGAACACCAAGGAGGAT |
| | CAGCTGGCCGAGATCAGCTACAGATTCCAAGGCAAGAAGGAGGCC |
| | GATCAGCCCTGGATCGTGGTGAACACAAGCACCCTGTTCGACGAG |
| | CTGGAGCTGGACCCCCCCGAGATCGAGCCCGGCGTGCTGAAGGTG |
| | CTGAGAACCGAGAAGCAGTACCTGGGCGTGTACATCTGGAACATG |
| | AGAGGCAGCGACGGCACAAGCACCTACGCCACCTTCCTGGTGACC |
| | TGGAAGGGCGACGAGAAGACAAGAAACCCCACCCCCGCAGTGAC |
| | GCCCCAACCTAGAGGCGCCGAGTTCCACATGTGGAACTACCACAG |
| | CCACGTGTTCAGCGTGGGCGACACCTTCAGCCTGGCCATGCACCTG |
| | CAGTACAAGATCCACGAGGCCCCCTTCGACCTGCTGCTGGAGTGG |
| | CTGTACGTGCCCATCGACCCCACCTGTCAGCCCATGAGACTGTACA |
| | GCACCTGCCTGTACCACCCCAACGCCCCTCAGTGCCTTAGTCACAT |
| | GAACAGCGGCTGCACCTTCACAAGCCCCCACCTGGCTCAGAGAGT |
| | GGCTAGCACCGTGTATCAGAACTGCGAGCACGCCGACAACTACAC |
| | CGCCTACTGCCTGGGCATCAGCCACATGGAGCCTAGCTTCGGCCTG |
| | ATCCTGCATGACGGCGGCACGACCTTAAAGTTCGTGGACACCCCC |
| | GAGAGCCTGAGCGGCCTGTACGTGTTCGTGGTGTACTTCAACGGCC |
| | ACGTGGAGGCCGTGGCCTACACCGTGGTGAGCACCGTGGACCACT |
| | TCGTGAACGCAATCGAGGAGCGCGGCTTTCCGCCGACTGCCGGTC |
| | AACCCCCCGCCACCACCAAGCCCAAGGAGATCACCCCCGTGAATC |
| | CGGGCACAAGCCCTCTTATCCGGTATGCCGCCTGGACCGGCGGCCT |
| | GGCCGCCGTGGTGCTGCTGTGCCTGGTGATCTTCCTGATCTGCACC |
| | GCCAAGAGAATGAGAGTGAAGGCCTACAGAGTGGACAAGAGCCC |
| | CTACAATCAGAGCATGTACTACGCCGGCCTGCCCGTGGACGACTTC |
| | GAGGACAGCGAGAGCACCGACACCGAGGAGGAGTTCGGCAACGC |
| | CATCGGCGGCAGCCACGGAGGCAGCAGCTACACCGTGTACATCGA |
| | CAAGACAAGATGATAAGAATTCTAGGCTGGAGCCTCGGTGGCCAT |
| | GCTTCTTGCCCCTTGGGCCTCCCCCCAGCCCCTCCTCCCCTTCCTGC |
| | ACCCGTACCCCCGTGGTCTTTGAATAAAGTCTGAGTGGGCGGCACT |
| | AGT |
| | Aₙ |
| ABOC-115S-5 | MDWTWILFLVAAATRVHSSVLRYDDFHIDEDKLDTNSVYEPYYHSDHA |
| (SEQ ID NO: 59) | ESSWVNRGESSRKAYDHNSPYIWPRNDYDGFLENAHEHHGVYNQGR |
| Human IgE SP | GIDSGERLMQPTQMSAQEDLGDDTGIHVIPTLNGDDRHKIVNVDQRQ |
| fused with | YGDVFKGDLNPKPQGQRLIEVSVEENHPFTLRAPIQRIYGVRYTETWS |
| mutein-2 | FLPSLTCTGDAAPAIQHICLKHTTCFQDVVVDVDCAENTKEDQLAEIS |
| | YRFQGKKEADQPWIVVNTSTLFDELELDPPEIEPGVLKVLRTEKQYLG |
| | VYIWNMRGSDGTSTYATFLVTWKGDEKTRNPTPAVTPQPRGAEFHM |
| | WNYHSHVFSVGDTFSLAMHLQYKIHEAPFDLLLEWLYVPIDPTCQPM |
| | RLYSTCLYHPNAPQCLSHMNSGCTFTSPHLAQRVASTVYQNCEHADN |
| | YTAYCLGISHMEPSFGLILHDGGTTLKFVDTPESLSGLYVFVVYFNGH |
| | VEAVAYTVVSTVDHFVNAIEERGFPPTAGQPPATTKPKEITPVNPGTSP |
| | LIRYAAWTGGLAAVVLLCLVIFLICTAKRMRVKAARVDKSPYNQSM |
| | YGAGLP |
| ABOC-115S-5-2 | GGGGGATCCAAGCTTAGCTTGCTTGTTCTTTTTGCAGAAGCTCAGA |
| (SEQ ID NO: 60) | ATAAACGCTCAACTTTGGCGCCGCCAccATGGACTGGACCTGGATT |
| Encoding human | CTGTTCCTGGTGGCGGCCGCCACAAGGGTGCACAGCAGCGTCCTG |
| IgE SP fused with | CGCTATGATGACTTTCACATAGACGAGGACAAGCTGGATACCAAC |
| mutein-2 | TCTGTGTACGAGCCCTACTACCACAGCGACCACGCGGAGAGTAGC |
| | TGGGTGAACAGGGGGGAGAGCAGCAGGAAGGCCTACGACCACAA |
| | TTCCCCCTACATCTGGCCCCGGAACGACTACGACGGCTTCCTGGAG |
| | AACGCCCACGAGCACCACGGGGTGTACAACCAGGGCCGCGGGATC |
| | GATTCCGGGGAGAGGCTGATGCAGCCTACCCAGATGAGCGCTCAG |
| | GAGGACCTGGGGGATGACACTGGCATCCATGTCATCCCCACCCTG |
| | AACGGGGACGATCGGCACAAGATCGTGAATGTGGATCAGAGGCA |
| | GTATGGGGACGTGTTCAAGGGCGACCTGAATCCCAAGCCCCAGGG |
| | GCAGCGCCTGATCGAGGTGAGCGTGGAGGAGAACCATCCCTTCAC |
| | GCTGAGGGCCCCGATCCAGCGCATCTACGGGGTGCGGTACACGGA |
| | GACTTGGAGCTTCCTGCCAAGTCTCACGTGTACCGGCGACGCCGCC |
| | CCCGCCATCCAGCACATCTGCCTGAAGCACACCACGTGTTTTCAGG |
| | ACGTGGTGGTGGACGTGGACTGCGCTGAGAATACCAAGGAGGATC |
| | AGCTGGCCGAGATCTCCTATAGGTTTCAGGGTAAGAAGGAGGCTG |
| | ACCAGCCGTGGATCGTGGTGAACACCTCTACCCTGTTCGATGAGCT |
| | GGAGCTCGACCCTCCGGAGATCGAGCCCGGCGTGCTGAAGGTGCT |
| | CCGCACGGAGAAGCAGTACCTGGGAGTCTACATATGGAACATGCG |
| | GGGGTCGGACGGGACTTCTACGTACGCCACCTTCCTGGTGACCTGG |
| | AAGGGCGACGAGAAGACCCGGAATCCGACCCCCGCCGTGACGCCC |
| | CAGCCTCGGGGCGCCGAGTTCCATATGTGGAACTACCACAGTCAC |
| | GTCTTCTCCGTGGGAGACACCTTCAGCCTGGCCATGCACCTCCAGT |
| | ACAAGATCCACGAGGCCCCCTTTGATCTGCTGCTGGAGTGGCTGTA |
| | TGTGCCCATCGACCCTACCTGTCAGCCTATGAGGCTGTACAGCACG |
| | TGCCTGTACCACCCGAACGCCCCGCAGTGTCTGTCCCACATGAACA |
| | GCGGGTGCACGTTCACCAGCCCGCATCTGGCCCAGAGGGTGGCCT |
| | CCACCGTGTACCAGAACTGTGAGCATGCTGACAATTACACTGCTTA |
| | CTGTCTGGGGATCAGCCACATGGAGCCCAGCTTCGGGCTGATCCTG |
| | CACGACGGGGGGACCACCCTGAAGTTCGTGGACACTCCTGAGAGC |
| | CTGAGCGGGCTGTACGTGTTCGTGGTGTACTTCAACGGGCACGTGG |
| | AGGCGGTGGCCTACACCGTGGTGTCCACGGTGGATCACTTCGTGA |
| | ATGCCATCGAGGAGAGAGGGTTCCCACCCACGGCTGGTCAGCCTC |
| | CTGCTACCACGAAGCCTAAGGAGATCACGCCAGTGAATCCTGGCA |
| | CCTCCCCTCTGATCAGGTACGCTGCTTGGACAGGGGGGCTGGCCGC |
| | CGTGGTGCTCCTGTGCCTGGTGATCTTTCTGATCTGCACCGCCAAG |
| | CGGATGCGGGTCAAGGCAGCCCGCGTGGACAAGAGCCCGTACAAT |
| | CAGAGCATGTACGGCGCCGGGCTGCCCTGATAAGAATTCTAGGCT |
| | GGAGCCTCGGTGGCCATGCTTCTTGCCCCTTGGGCCTCCCCCCAGC |
| | CCCTCCTCCCCTTCCTGCACCCGTACCCCCGTGGTCTTTGAATAAA |
| | GTCTGAGTGGGCGGCACTAGT |
| | Aₙ |
| ABOC-115S-5-3 | GGGGGATCCAAGCTTAGCTTGCTTGTTCTTTTTGCAGAAGCTCAGA |
| (SEQ ID NO: 61) | ATAAACGCTCAACTTTGGCGCCGCCAccATGGACTGGACCTGGATT |
| Encoding human | CTGTTCCTGGTGGCGGCCGCCACAAGGGTGCACAGCAGCGTCCTG |
| IgE SP fused with | CGGTATGACGACTTTCATATCGACGAGGACAAGCTGGATACCAAC |
| mutein-2 | TCTGTGTACGAGCCCTACTACCACAGCGACCACGCGGAGAGTAGC |
| | TGGGTGAACAGGGGGGAGTCTTCCAGGAAGGCCTACGACCACAAT |
| | TCCCCCTACATCTGGCCCCGGAACGACTACGACGGCTTCCTGGAGA |
| | ACGCCCACGAGCACCACGGGGTGTACAACCAGGGCCGCGGGATCG |
| | ATTCCGGGGAGAGGCTGATGCAGCCTACCCAGATGAGCGCTCAGG |
| | AGGACCTGGGGGATGACACTGGCATCCATGTCATCCCCACCCTGA |
| | ACGGGGACGATCGGCACAAGATCGTGAATGTGGATCAGAGGCAGT |
| | ACGGGGACGTGTTCAAGGGGGATTTGAACCCAAAGCCCCAGGGGC |
| | AGAGGCTGATTGAGGTGAGCGTGGAGGAGAACCACCCTTTCACTC |
| | TGAGGGCCCCCATCCAGAGGATCTACGGGGTGCGGTACACTGAAA |
| | CCTGGAGCTTCCTGCCCAGCCTGACCTGTACCGGCGACGCCGCTCC |
| | CGCCATCCAGCACATCTGCCTGAAGCACACCACGTGTTTTCAGGAC |
| | GTGGTGGTGGACGTGGACTGCGCCGAGAATACCAAGGAGGATCAG |
| | CTGGCAGAGATCAGCTACCGGTTTCAGGGGAAGAAGGAGGCCGAC |
| | CAGCCCTGGATCGTGGTGAACACCAGCACCCTCTTCGATGAGCTG |
| | GAGCTCGATCCTCCCGAGATCGAGCCCGGCGTGCTGAAGGTGCTC |
| | CGCACGGAGAAGCAGTACCTGGGAGTCTACATATGGAACATGCGG |
| | GGGAGCGACGGGACTTCTACGTACGCCACCTTCCTGGTGACCTGG |
| | AAGGGCGACGAGAAGACCCGGAATCCTACCCCCGCCGTGACGCCC |
| | CAGCCTCGGGGCGCCGAGTTCCATATGTGGAACTACCACAGTCAC |
| | GTCTTCTCCGTGGGAGACACCTTCAGCCTGGCCATGCACCTCCAGT |
| | ACAAGATCCACGAGGCCCCCTTTGATCTGCTGCTGGAGTGGCTGTA |
| | TGTGCCCATCGATCCTACCTGTCAGCCTATGAGGCTGTACAGCACC |
| | TGCCTGTACCACCCCAACGCCCCACAGTGCCTGTCACACATGAAC |
| | AGCGGGTGCACCTTCACCAGCCCGCATCTGGCCCAGAGGGTGGCC |
| | TCCACCGTGTACCAGAACTGTGAGCATGCTGACAATTACACTGCTT |
| | ACTGTCTGGGGATCAGCCACATGGAGCCCAGCTTCGGGCTGATCCT |
| | GCACGACGGGGGGACCACCCTGAAGTTCGTGGACACTCCTGAGAG |
| | CCTGAGCGGGCTGTACGTGTTCGTGGTGTACTTTAACGGGCACGTG |
| | GAGGCCGTGGCCTACACCGTGGTGAGCACCGTGGATCACTTTGTG |
| | AATGCCATCGAGGAGAGGGGGTTCCCTCCCACCGCCGGGCAGCCT |
| | CCAGCCACGACCAAGCCCAAGGAGATCACTCCTGTGAACCCCGGC |
| | ACCAGCCCCCTGATCCGGTACGCTGCCTGGACAGGCGGGCTGGCT |
| | GCCGTGGTGTTGCTGTGTCTGGTGATCTTCCTGATCTGCACCGCCA |
| | AGCGGATGCGGGTGAAGGCTGCCCGCGTGGACAAGTCTCCCTACA |
| | ATCAGTCCATGTATGGCGCCGGGCTCCCCTGATAAGAATTCTAGGC |
| | TGGAGCCTCGGTGGCCATGCTTCTTGCCCCTTGGGCCTCCCCCCAG |
| | CCCCTCCTCCCCTTCCTGCACCCGTACCCCCGTGGTCTTTGAATAA |
| | AGTCTGAGTGGGCGGCACTAGT |
| | Aₙ |
| ABOC-115S-5-4 | GGGGGATCCAAGCTTAGCTTGCTTGTTCTTTTTGCAGAAGCTCAGA |
| (SEQ ID NO: 62) | ATAAACGCTCAACTTTGGCGCCGCCAccATGGACTGGACCTGGATT |
| Encoding human | CTGTTCCTGGTGGCCGCTGCCACCAGGGTGCACAGCTCCGTGCTGC |
| IgE SP fused with | GGTACGATGATTTTCACATCGATGAGGACAAGCTCGACACCAACA |
| mutein-2 | GCGTGTACGAGCCTTACTACCACTCTGACCACGCCGAGTCCTCCTG |
| | GGTGAATAGGGGCGAGTCTTCCAGGAAGGCCTATGACCACAATAG |
| | CCCCTACATCTGGCCCCGGAACGACTACGACGGCTTCCTGGAGAA |
| | CGCCCACGAGCACCAACGGGGTGTACAACCAGGGCCGCGGGATCGA |
| | TTCCGGGGAGAGGCTGATGCAGCCTACCCAGATGTCCGCCCAGGA |
| | GGACCTGGGCGACGACACTGGCATCCACGTGATCCCCACCCTGAA |
| | TGGGGACGATCGCCACAAGATCGTGAACGTGGATCAGAGGCAGTA |
| | CGGAGACGTGTTTAAGGGCGACCTGAACCCTAAGCCCCAGGGGCA |
| | GCGGCTGATCGAGGTGAGCGTGGAGGAGAACCATCCTTTCACCCT |
| | GCGGGCCCCCATTCAGAGGATCTACGGGGTGCGGTACACCGAAAC |
| | CTGGAGCTTCCTGCCCAGCCTGACCTGTACCGGCGACGCCGCTCCC |
| | GCCATCCAGCACATCTGCCTGAAGCATACCACCTGCTTCCAGGACG |
| | TGGTGGTGGACGTGGACTGCGCCGAGAATACCAAGGAGGATCAGC |
| | TGGCAGAGATCAGCTACCGGTTTCAGGGGAAGAAGGAGGCTGACC |
| | AGCCTTGGATCGTGGTGAACACCAGCACACTGTTCGATGAGCTGG |
| | AGCTGGACCCACCCGAGATCGAGCCAGGCGTGCTGAAGGTGCTGC |
| | GCACTGAGAAGCAGTATCTGGGGGTGTACATCTGGAACATGCGGG |
| | GGAGCGATGGCACCAGCACATACGCCACCTTCCTGGTGACCTGGA |
| | AGGGGGACGAGAAGACTCGGAACCCCACCCCTGCTGTGACCCCAC |
| | AGCCCAGGGGCGCTGAGTTCCACATGTGGAACTACCATAGCCACG |
| | TGTTCAGCGTGGGGGACACCTTCTCCCTGGCTATGCACCTGCAGTA |
| | CAAGATCCACGAGGCCCCCTTTGATCTGCTGCTGGAGTGGCTGTAC |
| | GTGCCCATCGACCCCACCTGTCAGCCTATGAGGCTGTACAGCACCT |
| | GCCTGTACCACCCCAATGCTCCTCAGTGCCTGAGCCACATGAACAG |
| | TGGCTGCACCTTCACCAGCCCCCATCTGGCCCAGAGGGTGGCCTCC |
| | ACCGTGTACCAGAATTGCGAGCACGCCGACAACTACACCGCTTAC |
| | TGTCTGGGGATCAGCCACATGGAGCCCAGCTTCGGGCTGATCCTGC |
| | ACGACGGGGGCACCACCCTGAAGTTCGTGGACACTCCCGAGTCCC |
| | TGAGCGGCCTGTACGTGTTCGTGGTGTACTTCAACGGACACGTGGA |
| | GGCCGTGGCCTACACCGTGGTGAGCACCGTGGATCACTTCGTGAA |
| | TGCCATCGAGGAGAGGGGGTTTCCCCCTACCGCTGGCCAGCCTCC |
| | AGCCACAACCAAGCCCAAGGAGATCACCCCCGTGAACCCCGGCAC |
| | CAGCCCCCTGATCCGGTACGCTGCCTGGACAGGCGGGCTGGCTGC |
| | CGTGGTGCTGCTGTGCCTGGTGATCTTCCTGATCTGCACCGCCAAG |
| | CGGATGAGGGTGAAGGCCGCCAGGGTGGACAAGAGCCCCTACAAT |
| | CAGAGCATGTACGGGGCCGGGCTCCCCTGATAAGAATTCGAATC |
| | TAGGCTGGAGCCTCGGTGGCCATGCTTCTTGCCCCTTGGGCCTCCC |
| | CCCAGCCCCTCCTCCCCTTCCTGCACCCGTACCCCCGTGGTCTTTG |
| | AATAAAGTCTGAGTGGGCGGCACTAGT |
| | Aₙ |
| ABOC-115S-5-5 | GGGGGATCCCTGAACTGGGTTAGGTGCCGCTGTTGCTGCTCGTGTT |
| (SEQ ID NO: 63) | GAATCTAGAACCGTAGCCAGACGCCACCATGGACTGGACCTGGAT |
| Encoding human | TCTGTTCCTGGTGGCCGCCGCCACCAGGGTGCACAGCTCTGTCCTG |
| IgE SP fused with | AGGTACGACGACTTCCACATCGATGAGGACAAGCTGGACACCAAC |
| mutein-2 | AGCGTGTACGAGCCCTACTACCACAGCGACCACGCCGAGTCCTCC |
| | TGGGTGAATAGGGGCGAGTCTTCCAGGAAGGCCTATGACCACAAT |
| | AGCCCCTACATCTGGCCCCGGAACGACTACGACGGCTTCCTGGAG |
| | AACGCCCACGAGCACCACGGGGTGTACAACCAGGGCCGCGGGATC |
| | GATTCCGGGGAGAGGCTGATGCAGCCTACCCAGATGTCCGCCCAG |
| | GAGGACCTGGGCGACGATACCGGCATTCACGTGATCCCCACCCTG |
| | AATGGGGACGATCGGCACAAGATCGTGAATGTGGATCAGCGCCAG |
| | TACGGCGATGTGTTCAAGGGCGACCTGAATCCCAAGCCCCAGGGC |
| | CAGCGGCTGATCGAGGTGAGCGTGGAGGAGAATCACCCTTTTACC |
| | CTGCGGGCCCCCATTCAGAGGATCTACGGGGTGCGGTACACCGAA |
| | ACCTGGAGCTTCCTGCCCAGCCTGACCTGTACCGGCGACGCCGCCC |
| | CCGCCATCCAGCACATCTGCCTGAAGCATACCACCTGCTTCCAGGA |
| | CGTGGTGGTGGACGTGGACTGCGCCGAGAATACCAAGGAGGATCA |
| | GCTGGCAGAGATCAGCTACCGGTTTCAGGGGAAGAAGGAGGCCGA |
| | CCAGCCTTGGATCGTGGTGAACACCAGCACCCTGTTCGACGAGCT |
| | GGAGCTGGACCCTCCCGAGATCGAGCCCGGCGTGCTGAAGGTGCT |
| | GAGGACCGAGAAGCAGTACCTGGGGGTGTACATCTGGAACATGCG |
| | GGGGAGCGATGGCACCAGCACATACGCCACCTTCCTGGTGACCTG |
| | GAAGGGGGACGAGAAGACTCGGAACCCCACCCCTGCTGTGACCCC |
| | ACAGCCCAGGGGCGCTGAGTTCCACATGTGGAACTACCATAGCCA |
| | CGTGTTCAGCGTGGGGGACACCTTCTCCCTGGCTATGCACCTGCAG |
| | TACAAGATCCACGAGGCCCCCTTTGATCTGCTGCTGGAGTGGCTGT |
| | ACGTGCCCATCGACCCCACCTGTCAGCCTATGAGGCTGTACAGCAC |
| | CTGCCTGTACCACCCCAACGCCCCACAGTGCCTGTCCCACATGAAC |
| | AGCGGGTGCACCTTCACCAGCCCCCACCTGGCCCAGAGGGTGGCC |
| | TCCACCGTGTACCAGAATTGCGAGCACGCCGACAATTACACCGCC |
| | TACTGTCTGGGGATCAGCCACATGGAGCCCAGCTTCGGGCTGATCC |
| | TGCACGACGGGGGGACCACCCTGAAGTTCGTGGACACCCCTGAGA |
| | GCCTGAGCGGGCTGTACGTGTTCGTGGTGTACTTTAACGGGCACGT |
| | GGAGGCCGTGGCCTACACCGTGGTGTCCACCGTGGACCACTTCGT |
| | GAACGCCATCGAGGAGAGGGGCTTCCCTCCTACCGCCGGGCAGCC |
| | CCCTGCCACCACCAAGCCCAAGGAGATCACCCCCGTGAACCCCGG |
| | CACCAGCCCCCTGATCCGGTACGCCGCCTGGACAGGCGGGCTGGC |
| | TGCCGTGGTGCTGCTGTGCCTGGTGATCTTCCTGATCTGCACCGCC |
| | AAGCGGATGCGGGTGAAGGCCGCCAGGGTGGACAAGAGCCCCTA |
| | CAATCAGAGCATGTACGGGGCTGGGCTGCCCTGATAAGAATTCTA |
| | GGCTGGAGCCTCGGTGGCCATGCTTCTTGCCCCTTGGGCCTCCCCC |
| | CAGCCCCTCCTCCCCTTCCTGCACCCGTACCCCCGTGGTCTTTGAA |
| | TAAAGTCTGAGTGGGCGGCACTAGT |
| | Aₙ |
| ABOC-115S-5-6 | GGGGGATCCGCTGGTTTTGGTGATTTTTGTCTGATTGCAGCCACCA |
| (SEQ ID NO: 64) | TGGACTGGACCTGGATTCTGTTCCTGGTGGCCGCCGCCACCAGGGT |
| Encoding human | GCACAGCTCTGTGCTGCGGTACGACGACTTCCAACATCGACGAGGA |
| IgE SP fused with | CAAGCTGGACACCAACTCAGTGTACGAGCCCTATTACCACAGCGA |
| mutein-2 | CCACGCGGAGAGTAGCTGGGTGAACAGGGGGGAGAGCAGCAGGA |
| | AGGCCTACGACCACAATTCCCCCTACATCTGGCCCCGGAACGACT |
| | ACGACGGCTTCCTGGAGAACGCCCACGAGCACCACGGGGTGTACA |
| | ACCAGGGCCGCGGGATCGATTCCGGGGAGAGGCTGATGCAGCCTA |
| | CCCAGATGAGCGCTCAGGAGGACCTGGGGGATGACCACTGGCATCC |
| | ATGTCATCCCCACCCTGAACGGGGACGATCGGCACAAGATCGTGA |
| | ATGTGGATCAGAGGCAGTATGGGGACGTGTTCAAGGGGGATCTGA |
| | ATCCAAAGCCCCAGGGGCAGCGGCTGATCGAGGTGTCCGTGGAGG |
| | AGAACCACCCTTTCACTCTGAGGGCCCCCATCCAGAGGATCTACG |
| | GGGTGCGGTACACTGAAACCTGGAGCTTCCTGCCCAGCCTGACCT |
| | GTACCGGCGACGCCGCTCCCGCCATCCAGCACATCTGCCTGAAGC |
| | ACACCACGTGTTTTCAGGACGTGGTGGTGGACGTGGACTGCGCCG |
| | AGAATACCAAGGAGGATCAGCTGGCAGAGATCAGCTACCGGTTTC |
| | AGGGGAAGAAGGAGGCCGACCAGCCCTGGATCGTGGTGAACACC |
| | AGCACCCTCTTCGATGAGCTGGAGCTCGATCCTCCCGAGATCGAGC |
| | CCGGCGTGCTGAAGGTGCTCCGCACAGAGAAGCAGTACCTGGGAG |
| | TCTACATCTGGAACATGCGGGGGAGCGACGGGACTTCTACATACG |
| | CCACCTTCCTGGTGACCTGGAAGGGCGATGAGAAGACCCGGAATC |
| | CTACCCCCGCCGTGACCCCTCAGCCTCGGGGGGCCGAGTTCCACAT |
| | GTGGAACTACCAACAGTCACGTCTTCTCTGTGGGAGACACCTTCAGC |
| | CTGGCCATGCACCTCCAGTACAAGATCCACGAGGCCCCCTTTGATC |
| | TGCTGCTGGAGTGGCTGTATGTGCCCATCGATCCTACCTGTCAGCC |
| | TATGAGGCTGTACAGCACCTGCCTGTACCACCCCAACGCCCCACA |
| | GTGCCTGTCACACATGAACAGCGGGTGCACCTTCACCAGCCCGCA |
| | TCTGGCCCAGAGGGTGGCCTCCACCGTGTACCAGAACTGTGAGCA |
| | TGCTGACAATTACACTGCTTACTGTCTGGGGATCAGCCACATGGAG |
| | CCCAGCTTCGGGCTGATCCTGCACGACGGGGGGACCACCCTGAAG |
| | TTCGTGGACACTCCTGAGAGCCTGAGCGGGCTGTACGTGTTCGTGG |
| | TGTACTTTAACGGGCACGTGGAGGCCGTGGCCTACACCGTGGTGA |
| | GCACCGTGGATCACTTTGTGAATGCCATCGAGGAGAGGGGGTTCC |
| | CTCCCACAGCCGGGCAGCCCCCTGCCACCACCAAGCCCAAGGAGA |
| | TCACCCCCGTGAACCCCGGCACCAGCCCCCTGATCCGGTACGCTGC |
| | CTGGACAGGCGGGCTGGCTGCCGTGGTGCTGCTGTGCCTGGTGATC |
| | TTCCTGATCTGCACCGCCAAGCGGATGCGGGTCAAGGCCGCCAGG |
| | GTGGACAAGAGCCCCTACAATCAGAGCATGTACGGGGCTGGGCTG |
| | CCCTGATAAGAATTCTAGGCTGGAGCCTCGGTGGCCATGCTTCTTG |
| | CCCCTTGGGCCTCCCCCCAGCCCCTCCTCCCCTTCCTGCACCCGTA |
| | CCCCCGTGGTCTTTGAATAAAGTCTGAGTGGGCGGCACTAGT |
| | Aₙ |
| ABOC-115S-5-5 | m1ψ refers to N1-methylpseudouridine modification, in which all uridine |
| (m1ψ, cap | triphosphates of RNA are substituted for N1-methylpseudouridine |
| analog:m7GpppA | except for the capping structure. |
| mpU) | m⁷ GpppAmpU refers to the formula: |
| Encoding human IgE SP fused with mutein-2 | 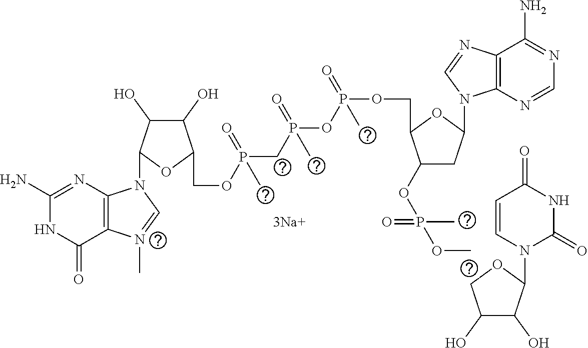 |
| | in which m⁷ GpppAm represents N7-methylguanosine-5′- |
| | triphosphate-5′-adenosine, wherein Am represents 2′-O-methylated A, and |
| | pU represents the concentration of U by a phosphate bond. |
| | This construct refers to SEQ ID NO: 63, in which when in vitro |
| | transcription, UTP was replaced with m1ψ in the rNTP mixture, and the |
| | capping is performed by addition of m7 GpppAmpU, i.e., the first nucleotide |
| | G is substituted for m⁷ GpppAmpU. |
| gE (1-573aa | MGTVNKPVVGVLMGFGIITGTLRITNPVRASVLRYDDFHIDEDKLDTNS |
| Y569A) | VYEPYYHSDHAESSWVNRGESSRKAYDHNSPYIWPRNDYDGFLENA |
| (SEQ ID NO: 65) | HEHHGVYNQGRGIDSGERLMQPTQMSAQEDLGDDTGIHVIPTLNGDD |
| Based on gE of | RHKIVNVDQRQYGDVFKGDLNPKPQGQRLIEVSVEENHPFTLRAPIQR |
| Oka strain | IYGVRYTETWSFLPSLTCTGDAAPAIQHICLKHTTCFQDVVVDVDCAE |
| | NTKEDQLAEISYRFQGKKEADQPWIVVNTSTLFDELELDPPEIEPGVL |
| | KVLRTEKQYLGVYIWNMRGSDGTSTYATFLVTWKGDEKTRNPTPAV |
| | TPQPRGAEFHMWNYHSHVFSVGDTFSLAMHLQYKIHEAPFDLLLEW |
| | LYVPIDPTCQPMRLYSTCLYHPNAPQCLSHMNSGCTFTSPHLAQRVAS |
| | TVYQNCEHADNYTAYCLGISHMEPSFGLILHDGGTTLKFVDTPESLSG |
| | LYVFVVYFNGHVEAVAYTVVSTVDHFVNAIEERGFPPTAGQPPATTK |
| | PKEITPVNPGTSPLLRYAAWTGGLAAVVLLCLVIFLICTAKRMRVKAA |
| | RVDK |
| gE(1-573aa | GGGAAATAAGAGAGAAAAGAAGAGTAAGAAGAAATATAAGAGCC |
| Y569A, codon | ACCATGGGGACAGTTAATAAACCTGTGGTGGGGGTATTGATGGGG |
| optimization 1) | TTCGGAATTATCACGGGAACGTTGCGTATAACGAATCCGGTCAGA |
| (SEQ ID NO: 66) | GCATCCGTCTTGCGATACGATGATTTTCACATCGATGAAGACAAA |
| | TGGATACAAACTCCGTATATGAGCCTTACTACCATTCAGATCATGC |
| | GGAGTCTTCATGGGTAAATCGGGGAGAGTCTTCGCGAAAAGCGTA |
| | CGATCATAACTCACCTTATATATGGCCACGTAATGATTATGATGGA |
| | TTTAGAGAACGCACACGAACACCATGGGGTGTATAATCAGGGC |
| | CGTGGTATCGATAGCGGGGAACGGTTAATGCAACCCACACAAATG |
| | TCTGCACAGGAGGATCTTGGGGACGATACGGGCATCCACGTTATC |
| | CCTACGTTAAACGGCGATGACAGACATAAAATTGTAAATGTGGAC |
| | CAACGTCAATACGGTGACGTGTTTAAAGGAGATCTTAATCCAAAA |
| | CCCCAAGGCCAAAGACTCATTGAGGTGTCAGTGGAAGAAAATCAC |
| | CCGTTTACTTTACGCGCACCGATTCAGCGGATTTATGGAGTCCGGT |
| | ACACCGAGACTTGGAGCTTTTTGCCGTCATTAACCTGTACGGGAGA |
| | CGCAGCGCCCGCCATCCAGCATATATGTTTAAAACATACAACATG |
| | CTTTCAAGACGTGGTGGTGGATGTGGATTGCGCGGAAAATACTAA |
| | AGAGGATCAGTTGGCCGAAATCAGTTACCGTTTTCAAGGTAAGAA |
| | GGAAGCGGACCAACCGTGGATTGTTGTAAACACGAGCACACTGTT |
| | TGATGAACTCGAATTAGACCCCCCCGAGATTGAACCGGGTGTCTTG |
| | AAAGTACTTCGGACAGAAAAACAATACTTGGGTGTGTACATTTGG |
| | AACATGCGCGGCTCCGATGGTACGTCTACCTACGCCACGTTTTTGG |
| | TCACCTGGAAAGGGGATGAAAAAACAAGAAACCCTACGCCCGCA |
| | GTAACTCCTCAACCAAGAGGGGCTGAGTTTCATATGTGGAATTACC |
| | ACTCGCATGTATTTTCAGTTGGTGATACGTTTAGCTTGGCAATGCA |
| | TCTTCAGTATAAGATACATGAAGCGCCATTTGATTTGCTGGTTAGAG |
| | TGGTTGTATGTCCCCATCGATCCTACATGTCAACCAATGCGGTTAT |
| | ATTCTACGTGTTTGTATCATCCCAACGCACCCCAATGCCTCTCTCAT |
| | ATGAATTCCGGTTGTACATTTACCTCGCCACATTTAGCCCAGCGTG |
| | TTGCAAGCACAGTGTATCAAAATTGTGAACATGCAGATAACTACA |
| | CCGCATATTGTCTGGGAATATCTCATATGGAGCCTAGCTTTGGTCT |
| | AATCTTACACGACGGGGGCACCACGTTAAAGTTTGTAGATACACC |
| | CGAGAGTTTGTCGGGATTATACGTTTTTGTGGTGTATTTTAACGGG |
| | CATGTTGAAGCCGTAGCATACACTGTTGTATCCACAGTAGATCATT |
| | TTGTAAACGCAATTGAAGAGCGTGGATTTCCGCCAACGGCCGGTC |
| | AGCCACCGGCGACTACTAAACCCAAGGAAATTACCCCCGTAAACC |
| | CCGGAACGTCACCACTTCTACGATATGCCGCATGGACCGGAGGGC |
| | TTGCAGCAGTAGTACTTTTATGTCTCGTAATATTTTTAATCTGTACG |
| | GCTAAACGAATGAGGGTTAAAGCCGCCAGGGTAGACAAGTGATGA |
| | TAATAGGCTGGAGCCTCGGTGGCCATGCTTCTTGCCCCTTGGGCCT |
| | CCCCCCAGCCCCTCCTCCCCTTCCTGCACCCGTACCCCCGTGGTCTT |
| | TGAATAAAGTCTGAGTGGGCGGCACTAGT |
| | Aₙ |
| gE(1-573aa | GGGAAATAAGAGAGAAAAGAAGAGTAAGAAGAAATATAAGAGCC |
| Y569A, codon | ACCATGGGGACAGTTAATAAACCTGTGGTGGGGGTATTGATGGGG |
| optimization 2) | TTCGGAATTATCACGGGAACGTTGCGTATAACGAATCCGGTCAGA |
| (SEQ ID NO: 67) | GCATCCGTCTTGCGATACGATGATTTTCACATCGATGAAGACAAAC |
| | TGGATACAAACTCCGTATATGAGCCTTACTACCATTCAGATCATGC |
| | GGAGTCTTCATGGGTAAATCGGGGAGAGTCTTCGCGAAAAGCGTA |
| | CGATCATAACTCACCTTATATATGGCCACGTAATGATTATGATGGA |
| | TTTTTAGAGAACGCACACGAACACCATGGGGTGTATAATCAGGGC |
| | CGTGGTATCGATAGCGGGGAACGGTTAATGCAACCCACACAAATG |
| | TCTGCACAGGAGGATCTTGGGGACGATACGGGCATCCACGTTATC |
| | CCTACGTTAAACGGCGATGACAGACATAAAATTGTAAATGTGGAC |
| | CAACGTCAATACGGTGACGTGTTTAAAGGAGATCTTAATCCAAAA |
| | CCCCAAGGCCAAAGACTCATTGAGGTGTCAGTGGAAGAAAATCAC |
| | CCGTTTACTTTACGCGCACCGATTCAGCGGATTTATGGAGTCCGGT |
| | ACACCGAGACTTGGAGCTTTTTGCCGTCATTAACCTGTACGGGAGA |
| | CGCAGCGCCCGCCATCCAGCATATATGTTTAAAGCATACAACATG |
| | CTTTCAAGACGTGGTGGTGGATGTGGATTGCGCGGAAAATACTAA |
| | AGAGGATCAGTTGGCCGAAATCAGTTACCGTTTTCAAGGTAAGAA |
| | GGAAGCGGACCAACCGTGGATTGTTGTAAACACGAGCACACTGTT |
| | TGATGAACTCGAATTAGACCCACCCGAGATTGAACCGGGTGTCTT |
| | GAAAGTACTTCGGACAGAGAAACAATACTTGGGTGTGTACATTTG |
| | GAACATGCGCGGCTCCGATGGTACGTCTACCTACGCCACGTTTTTG |
| | GTCACCTGGAAAGGGGATGAGAAGACAAGAAACCCTACGCCCGC |
| | AGTAACTCCTCAACCAAGAGGGGCTGAGTTTCATATGTGGAATTA |
| | CCACTCGCATGTATTTTCAGTTGGTGATACGTTTAGCTTGGCAATG |
| | CATCTTCAGTATAAGATACATGAAGCGCCATTTGATTTGCTGTTAG |
| | AGTGGTTGTATGTCCCCATCGATCCTACATGTCAACCAATGCGGTT |
| | ATATTCTACGTGTTTGTATCATCCCAACGCACCCCAATGCCTCTCTC |
| | ATATGAATTCCGGTTGTACATTTACCTCGCCACATTTAGCCCAGCG |
| | TGTTGCAAGCACAGTGTATCAGAATTGTGAACATGCAGATAAACTA |
| | CACCGCATATTGTCTGGGAATATCTCATATGGAGCCTAGCTTTGGT |
| | CTAATCTTACACGACGGGGGCACCACGTTAAAGTTTGTAGATACA |
| | CCCGAGAGTTTGTCGGGATTATACGTTTTTGTGGTGTATTTTAACG |
| | GGCATGTTGAAGCCGTAGCATACACTGTTGTATCCACAGTAGATCA |
| | TTTTGTAAACGCAATTGAAGAGCGTGGATTTCCGCCAACGGCCGGT |
| | CAGCCACCGGCGACTACTAAACCCAAGGAAATTACCCCCGTAAAC |
| | CCCGGACGTCACCACTTCTACGATATGCCGCATGGACCGGAGGG |
| | CTTGCAGCAGTAGTACTTTTATGTCTCGTAATATTTTTAATCTGTAC |
| | GGCTAAACGAATGAGGGTTAAAGCCGCCAGGGTAGACAAGTGATA |
| | ATAGGCTGGAGCCTCGGTGGCCATGCTTCTTGCCCCTTGGGCCTCC |
| | CCCCAGCCCCTCCTCCCCTTCCTGCACCCGTACCCCCGTGGTCTTTG |
| | AATAAAGTCTGAGTGGGCGGCACTAGT |
| | Aₙ |

Aₙ = 120mer of A in the sequences in table 5. More generally, Aₙ = 30~3000mer of A, for example.

### 7.4.4 The Polyadenylation (Poly-A) Regions

During natural RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Without being bound by the theory, it is contemplated that a poly-A region can confer various advantages to the nucleic acid molecule of the present disclosure.

Accordingly, in some embodiments, a nucleic acid molecule of the present disclosure (e.g., an mRNA) comprises a polyadenylation signal. In some embodiments, a nucleic acid molecule of the present disclosure (e.g., an mRNA) comprises one or more polyadenylation (poly-A) regions. In some embodiments, a poly-A region is composed entirely of adenine nucleotides or functional analogs thereof. In some embodiments, the nucleic acid molecule comprises at least one poly-A region at its 3′-end. In some embodiments, the nucleic acid molecule comprises at least one poly-A region at its 5′-end. In some embodiments, the nucleic acid molecule comprises at least one poly-A region at its 5′-end and at least one poly-A region at its 3′-end.

According to the present disclosure, the poly-A region can have varied lengths in different embodiments. Particularly, in some embodiments, the poly-A region of a nucleic acid molecule of the present disclosure is at least 30 nucleotides in length. In some embodiments, the poly-A region of a nucleic acid molecule of the present disclosure is at least 35 nucleotides in length. In some embodiments, the poly-A region of a nucleic acid molecule of the present disclosure is at least 40 nucleotides in length. In some embodiments, the poly-A region of a nucleic acid molecule of the present disclosure is at least 45 nucleotides in length. In some embodiments, the poly-A region of a nucleic acid molecule of the present disclosure is at least 50 nucleotides in length. In some embodiments, the poly-A region of a nucleic acid molecule of the present disclosure is at least 55 nucleotides in length. In some embodiments, the poly-A region of a nucleic acid molecule of the present disclosure is at least 60 nucleotides in length. In some embodiments, the poly-A region of a nucleic acid molecule of the present disclosure is at least 65 nucleotides in length. In some embodiments, the poly-A region of a nucleic acid molecule of the present disclosure is at least 70 nucleotides in length. In some embodiments, the poly-A region of a nucleic acid molecule of the present disclosure is at least 75 nucleotides in length. In some embodiments, the poly-A region of a nucleic acid molecule of the present disclosure is at least 80 nucleotides in length. In some embodiments, the poly-A region of a nucleic acid molecule of the present disclosure is at least 85 nucleotides in length. In some embodiments, the poly-A region of a nucleic acid molecule of the present disclosure is at least 90 nucleotides in length. In some embodiments, the poly-A region of a nucleic acid molecule of the present disclosure is at least 95 nucleotides in length. In some embodiments, the poly-A region of a nucleic acid molecule of the present disclosure is at least 100 nucleotides in length. In some embodiments, the poly-A region of a nucleic acid molecule of the present disclosure is at least 110 nucleotides in length. In some embodiments, the poly-A region of a nucleic acid molecule of the present disclosure is at least 120 nucleotides in length. In some embodiments, the poly-A region of a nucleic acid molecule of the present disclosure is at least 130 nucleotides in length. In some embodiments, the poly-A region of a nucleic acid molecule of the present disclosure is at least 140 nucleotides in length. In some embodiments, the poly-A region of a nucleic acid molecule of the present disclosure is at least 150 nucleotides in length. In some embodiments, the poly-A region of a nucleic acid molecule of the present disclosure is at least 160 nucleotides in length. In some embodiments, the poly-A region of a nucleic acid molecule of the present disclosure is at least 170 nucleotides in length. In some embodiments, the poly-A region of a nucleic acid molecule of the present disclosure is at least 180 nucleotides in length. In some embodiments, the poly-A region of a nucleic acid molecule of the present disclosure is at least 190 nucleotides in length. In some embodiments, the poly-A region of a nucleic acid molecule of the present disclosure is at least 200 nucleotides in length. In some embodiments, the poly-A region of a nucleic acid molecule of the present disclosure is at least 225 nucleotides in length. In some embodiments, the poly-A region of a nucleic acid molecule of the present disclosure is at least 250 nucleotides in length. In some embodiments, the poly-A region of a nucleic acid molecule of the present disclosure is at least 275 nucleotides in length. In some embodiments, the poly-A region of a nucleic acid molecule of the present disclosure is at least 300 nucleotides in length. In some embodiments, the poly-A region of a nucleic acid molecule of the present disclosure is at least 350 nucleotides in length. In some embodiments, the poly-A region of a nucleic acid molecule of the present disclosure is at least 400 nucleotides in length. In some embodiments, the poly-A region of a nucleic acid molecule of the present disclosure is at least 450 nucleotides in length. In some embodiments, the poly-A region of a nucleic acid molecule of the present disclosure is at least 500 nucleotides in length. In some embodiments, the poly-A region of a nucleic acid molecule of the present disclosure is at least 600 nucleotides in length. In some embodiments, the poly-A region of a nucleic acid molecule of the present disclosure is at least 700 nucleotides in length. In some embodiments, the poly-A region of a nucleic acid molecule of the present disclosure is at least 800 nucleotides in length. In some embodiments, the poly-A region of a nucleic acid molecule of the present disclosure is at least 900 nucleotides in length. In some embodiments, the poly-A region of a nucleic acid molecule of the present disclosure is at least 1000 nucleotides in length. In some embodiments, the poly-A region of a nucleic acid molecule of the present disclosure is at least 1100 nucleotides in length. In some embodiments, the poly-A region of a nucleic acid molecule of the present disclosure is at least 1200 nucleotides in length. In some embodiments, the poly-A region of a nucleic acid molecule of the present disclosure is at least 1300 nucleotides in length. In some embodiments, the poly-A region of a nucleic acid molecule of the present disclosure is at least 1400 nucleotides in length. In some embodiments, the poly-A region of a nucleic acid molecule of the present disclosure is at least 1500 nucleotides in length. In some embodiments, the poly-A region of a nucleic acid molecule of the present disclosure is at least 1600 nucleotides in length. In some embodiments, the poly-A region of a nucleic acid molecule of the present disclosure is at least 1700 nucleotides in length. In some embodiments, the poly-A region of a nucleic acid molecule of the present disclosure is at least 1800 nucleotides in length. In some embodiments, the poly-A region of a nucleic acid molecule of the present disclosure is at least 1900 nucleotides in length. In some embodiments, the poly-A region of a nucleic acid molecule of the present disclosure is at least 2000 nucleotides in length. In some embodiments, the poly-A region of a nucleic acid molecule of the present disclosure is at least 2250 nucleotides in length. In some embodiments, the poly-A region of a nucleic acid molecule of the present disclosure is at least 2500 nucleotides in length. In some embodiments, the poly-A region of a nucleic acid molecule of the present disclosure is at least 2750 nucleotides in length. In some embodiments, the poly-A region of a nucleic acid molecule of the present disclosure is at least 3000 nucleotides in length.

In some embodiments, length of a poly-A region in a nucleic acid molecule can be selected based on the overall length of the nucleic acid molecule, or a portion thereof (such as the length of the coding region or the length of an open reading frame of the nucleic acid molecule, etc.). For example, in some embodiments, the poly-A region accounts for about 5%, 10%, 15%, 20%, 25%, 30%, 35%, 40%, 45%, 50%, 55%, 60%, 65%, 70%, 75%, 80%, 85%, 90%, 95% or more of the total length of nucleic acid molecule containing the poly-A region.

Without being bound by the theory, itis contemplated that certain RNA-binding proteins can bind to the poly-A region located at the 3′-end of an mRNA molecule. These poly-A binding proteins (PABP) can modulate mRNA expression, such as interacting with translation initiation machinery in a cell and/or protecting the 3′-poly-A tails from degradation. Accordingly, in some embodiments, in some embodiments, the nucleic acid molecule of the present disclosure (e.g., mRNA) comprises at least one binding site for poly-A binding protein (PABP). In other embodiments, the nucleic acid molecule is conjugated or complex with a PABP before loaded into a delivery vehicle (e.g., lipid nanoparticles).

In some embodiments, the nucleic acid molecule of the present disclosure (e.g., mRNA) comprises a poly-A-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant polynucleotides (e.g., mRNA) may be assayed for stability, protein production and other parameters including half-life at various time points. It has been discovered that the polyA-G quartet structure results in protein production equivalent to at least 75% of that seen using a poly-A region of 120 nucleotides alone.

In some embodiments, the nucleic acid molecule of the present disclosure (e.g., mRNA) may include a poly-A region and may be stabilized by the addition of a 3′-stabilizing region. In some embodiments, the 3′-stabilizing region which may be used to stabilize a nucleic acid molecule (e.g., mRNA) including the poly-A or poly-A-G Quartet structures as described in International Patent Publication No. WO 2013/103659, the content of which is incorporated herein by reference in its entirety.

In other embodiments, the 3′-stabilizing region which may be used in connection with the nucleic acid molecules of the present disclosure include a chain termination nucleoside such as but is not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

### 7.4.5 Secondary Structure

Without being bound by the theory, itis contemplated that a stem-loop structure can direct RNA folding, protect structural stability of a nucleic acid molecule (e.g., mRNA), provide recognition sites for RNA binding proteins, and serve as a substrate for enzymatic reactions. For example, the incorporation of a miR sequence and/or a TEE sequence changes the shape of the stem loop region which may increase and/or decrease translation (Kedde et al. A Pumilio-induced RNA structure switch in p27-3′UTR controls miR-221 and miR-222 accessibility. Nat Cell Biol., 2010 October; 12(10):1014-20, the content of which is herein incorporated by reference in its entirety).

Accordingly, in some embodiments, the nucleic acid molecules as described herein (e.g., mRNA) or a portion thereof may assume a stem-loop structure, such as but is not limited to a histone stem loop. In some embodiments, the stem-loop structure is formed from a stem-loop sequence that is about 25 or about 26 nucleotides in length such as, but not limited to, those as described in International Patent Publication No. WO 2013/103659, the content of which is incorporated herein by reference in its entirety. Additional examples of stem-loop sequences include those described in International Patent Publication No. WO 2012/019780 and International Patent Publication No. WO 2015/02667, the contents of which are incorporated herein by reference. In some embodiments, the step-loop sequence comprises a TEE as described herein. In some embodiments, the step-loop sequence comprises a miR sequence as described herein. In specific embodiments, the stem loop sequence may include a miR-122 seed sequence. In specific embodiments, the nucleic acid molecule comprises the stem-loop sequence CAAAGGCTCTTTTCAGAGCCACCA (SEQ ID NO:47). In other embodiments, the nucleic acid molecule comprises the stem-loop sequence CAAAGGCUCUUUUCAGAGCCACCA (SEQ ID NO:48).

In some embodiments, the nucleic acid molecule of the present disclosure (e.g., mRNA) comprises a stem-loop sequence located upstream (to the 5′-end) of the coding region in a nucleic acid molecule. In some embodiments, the stem-loop sequence is located within the 5′-UTR of the nucleic acid molecule. In some embodiments, the nucleic acid molecule of the present disclosure (e.g., mRNA) comprises a stem-loop sequence located downstream (to the 3′-end) of the coding region in a nucleic acid molecule. In some embodiments, the stem-loop sequence is located within the 3′-UTR of the nucleic acid molecule. In some cases, a nucleic acid molecule can contain more than one stem-loop sequences. In some embodiment, the nucleic acid molecule comprises at least one stem-loop sequence in the 5′-UTR, and at least one stem-loop sequence in the 3′-UTR.

In some embodiments, a nucleic acid molecule comprising a stem-loop structure further comprises a stabilization region. In some embodiment, the stabilization region comprises at least one chain terminating nucleoside that functions to slow down degradation and thus increases the half-life of the nucleic acid molecule. Exemplary chain terminating nucleoside that can be used in connection with the present disclosure include but are not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein. In other embodiments, a stem-loop structure may be stabilized by an alteration to the 3′-region of the polynucleotide that can prevent and/or inhibit the addition of oligio(U) (International Patent Publication No. WO 2013/103659, incorporated herein by reference in its entirety).

In some embodiments, a nucleic acid molecule of the present disclosure comprises at least one stem-loop sequence and a poly-A region or polyadenylation signal. Non-limiting examples of polynucleotide sequences comprising at least one stem-loop sequence and a poly-A region or a polyadenylation signal include those described in International Patent Publication No. WO 2013/120497, International Patent Publication No. WO 2013/120629, International Patent Publication No. WO 2013/120500, International Patent Publication No. WO 2013/120627, International Patent Publication No. WO 2013/120498, International Patent Publication No. WO 2013/120626, International Patent Publication No. WO 2013/120499 and International Patent Publication No. WO 2013/120628, the content of each of which is incorporated herein by reference in its entirety.

In some embodiments, the nucleic acid molecule comprising a stem-loop sequence and a poly-A region or a polyadenylation signal can encode for a pathogen antigen or fragment thereof such as the polynucleotide sequences described in International Patent Publication No. WO 2013/120499 and International Patent Publication No. WO 2013/120628, the content of each of which is incorporated herein by reference in its entirety.

In some embodiments, the nucleic acid molecule comprising a stem-loop sequence and a poly-A region or a polyadenylation signal can encode for a therapeutic protein such as the polynucleotide sequences described in International Patent Publication No. WO 2013/120497 and International Patent Publication No. WO 2013/120629, the content of each of which is incorporated herein by reference in its entirety.

In some embodiments, the nucleic acid molecule comprising a stem-loop sequence and a poly-A region or a polyadenylation signal can encode for a tumor antigen or fragment thereof such as the polynucleotide sequences described in International Patent Publication No. WO 2013/120500 and International Patent Publication No. WO 2013/120627, the content of each of which is incorporated herein by reference in its entirety.

In some embodiments, the nucleic acid molecule comprising a stem-loop sequence and a poly-A region or a polyadenylation signal can code for an allergenic antigen or an autoimmune self-antigen such as the polynucleotide sequences described in International Patent Publication No. WO 2013/120498 and International Patent Publication No. WO 2013/120626, the content of each of which is incorporated herein by reference in its entirety.

### 7.4.6 Functional Nucleotide Analogs

In some embodiments, a payload nucleic acid molecule described herein contains only canonical nucleotides selected from A (adenosine), G (guanosine), C (cytosine), U (uridine), and T (thymidine). Without being bound by the theory, it is contemplated that certain functional nucleotide analogs can confer useful properties to a nucleic acid molecule. Examples of such as useful properties in the context of the present disclosure include but are not limited to increased stability of the nucleic acid molecule, reduced immunogenicity of the nucleic acid molecule in inducing innate immune responses, enhanced production of protein encoded by the nucleic acid molecule, increased intracellular delivery and/or retention of the nucleic acid molecule, and/or reduced cellular toxicity of the nucleic acid molecule, etc.

Accordingly, in some embodiments, a payload nucleic acid molecule comprises at least one functional nucleotide analog as described herein. In some embodiments, the functional nucleotide analog contains at least one chemical modification to the nucleobase, the sugar group and/or the phosphate group. Accordingly, a payload nucleic acid molecule comprising at least one functional nucleotide analog contains at least one chemical modification to the nucleobases, the sugar groups, and/or the internucleoside linkage. Exemplary chemical modifications to the nucleobases, sugar groups, or internucleoside linkages of a nucleic acid molecule are provided herein.

As described herein, ranging from 0% to 100% of all nucleotides in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 500%, from about 20% to about 60%, from about 20% to about 70%, from about 20% to about 800%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of all nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In some embodiments, a single nucleic acid molecule can contain different sugar modifications, different nucleobase modifications, and/or different types internucleoside linkages (e.g., backbone structures).

As described herein, ranging from 0% to 100% of all nucleotides of a kind (e.g., all purine-containing nucleotides as a kind, or all pyrimidine-containing nucleotides as a kind, or all A, G, C, T or U as a kind) in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of a kind of nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In some embodiments, a single nucleic acid molecule can contain different sugar modifications, different nucleobase modifications, and/or different types internucleoside linkages (e.g., backbone structures).

### 7.4.7 Modification to Nucleobases

In some embodiments, a functional nucleotide analog contains a non-canonical nucleobase. In some embodiments, canonical nucleobases (e.g., adenine, guanine, uracil, thymine, and cytosine) in a nucleotide can be modified or replaced to provide one or more functional analogs of the nucleotide. Exemplary modification to nucleobases include but are not limited to one or more substitutions or modifications including but not limited to alkyl, aryl, halo, oxo, hydroxyl, alkyloxy, and/or thio substitutions; one or more fused or open rings, oxidation, and/or reduction.

In some embodiments, the non-canonical nucleobase is a modified uracil. Exemplary nucleobases and nucleosides having an modified uracil include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (τm⁵U), 1-taurinomethyl-pseudouridine, 5-taurinomethyl-2-thio-uracil(τm⁵5s²U), 1-taurinomethyl-4-thio-pseudouridine, 5-methyl-uracil (m⁵U, i.e., having the nucleobase deoxythymine), 1-methyl-pseudouridine (m¹ψ), 1-ethyl-pseudouridine (Et¹ψ), 5-methyl-2-thio-uracil (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ψ), 5-(isopentenylaminomethyl)uracil (m⁵U), 5-(isopentenylaminomethyl)-2-thio-uracil (m⁵s²U), 5,2′-O-dimethyl-uridine (m⁵Um), 2-thio-2′-O-methyl-uridine (s²Um), 5-methoxycarbonylmethyl-2′-O-methyl-uridine (mcm³Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the non-canonical nucleobase is a modified cytosine. Exemplary nucleobases and nucleosides having a modified cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (fSCm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the non-canonical nucleobase is a modified adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the non-canonical nucleobase is a modified guanine. Exemplary nucleobases and nucleosides having a modified guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine.

In some embodiments, the non-canonical nucleobase of a functional nucleotide analog can be independently a purine, a pyrimidine, a purine or pyrimidine analog. For example, in some embodiments, the non-canonical nucleobase can be modified adenine, cytosine, guanine, uracil, or hypoxanthine. In other embodiments, the non-canonical nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

### 7.4.8 Modification to the Sugar

In some embodiments, a functional nucleotide analog contains a non-canonical sugar group. In various embodiments, the non-canonical sugar group can be a 5-carbon or 6-carbon sugar (such as pentose, ribose, arabinose, xylose, glucose, galactose, or a deoxy derivative thereof) with one or more substitutions, such as a halo group, a hydroxy group, a thiol group, an alkyl group, an alkoxy group, an alkenyloxy group, an alkynyloxy group, an cycloalkyl group, an aminoalkoxy group, an alkoxyalkoxy group, an hydroxyalkoxy group, an amino group, an azido group, an aryl group, an aminoalkyl group, an aminoalkenyl group, an aminoalkynyl group, etc.

Generally, RNA molecules contains the ribose sugar group, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone).

In some embodiments, the sugar group contains one or more carbons that possess the opposite stereochemical configuration of the corresponding carbon in ribose. Thus, a nucleic acid molecule can include nucleotides containing, e.g., arabinose or L-ribose, as the sugar. In some embodiments, the nucleic acid molecule includes at least one nucleoside wherein the sugar is L-ribose, 2′-O-methyl-ribose, 2′-fluoro-ribose, arabinose, hexitol, an LNA, or a PNA.

### 7.4.9 Modifications to the Internucleoside Linkage

In some embodiments, the payload nucleic acid molecule of the present disclosure can contain one or more modified internucleoside linkage (e.g., phosphate backbone). Backbone phosphate groups can be altered by replacing one or more of the oxygen atoms with a different substituent.

In some embodiments, the functional nucleotide analogs can include the replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl, and/or methoxy. As a non-limiting example, two non-bridging oxygens at the same position (e.g., the alpha (α), beta (β) or gamma (γ) position) can be replaced with a sulfur (thio) and a methoxy. The replacement of one or more of the oxygen atoms at the position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other internucleoside linkages that may be employed according to the present disclosure, including internucleoside linkages which do not contain a phosphorous atom, are described herein.

Additional examples of nucleic acid molecules (e.g., mRNA), compositions, formulations and/or methods associated therewith that can be used in connection with the present disclosure further include those described in WO 2002/098443, WO 2003/051401, WO 2008/052770, WO 2009/127230, WO 2006/122828, WO 2008/083949, WO 2010/088927, WO 2010/037539, WO 2004/004743, WO 2005/016376, WO 2006/024518, WO 2007/095976, WO 2008/014979, WO 2008/077592, WO 2009/030481, WO 2009/095226, WO 2011069586, WO 2011026641, WO 2011/144358, WO 2012019780, WO 2012013326, WO 2012/089338, WO 2012/113513, WO 2012/116811, WO 2012/116810, WO 2013/113502, WO 2013/113501, WO 2013/113736, WO 2013/143698, WO 2013/143699, WO 2013/143700, WO 2013/120626, WO 2013120627, WO 2013/120628, WO 2013/120629, WO 2013/174409, WO 2014/127917, WO 2015/024669, WO 2015/024668, WO 2015/024667, WO 2015/024665, WO 2015/024666, WO 2015/024664, WO 2015/101415, WO 2015/101414, WO 2015/024667, WO 2015/062738, WO 2015/101416, the content of each of which is incorporated herein in its entirety.

Therapeutic nucleic acid molecules as described herein can by isolated or synthesized using methods known in the art. In some embodiments, DNA or RNA molecules to be used in connection with the present disclosure are chemically synthesized. In other embodiments, DNA or RNA molecules to be used in connection with the present disclosure are isolated from a natural source.

In some embodiments, mRNA molecules to be used in connection with the present disclosure are biosynthesized using a host cell. In particular embodiments, an mRNA is produced by transcribing a corresponding DNA sequencing using a host cell. In some embodiments, a DNA sequence encoding an mRNA sequence is incorporated into an expression vector, which vector is then introduced into a host cell (e.g., E. coli) using methods known in the art. The host cell is then cultured under a suitable condition to produce mRNA transcripts. Other methods for producing an mRNA molecule from an encoding DNA are known in the art. For example, in some embodiments, a cell-free (in vitro) transcription system comprising enzymes of the transcription machinery of a host cell can be used to produce mRNA transcripts. An exemplary cell-free transcription reaction system is described in the present disclosure.

### 7.4.10 Vectors and Host Cells

In one aspect, provided herein is a vector comprising a nucleic acid described herein (e.g., in Section 5.4 or 6). In specific embodiments, the nucleic acid is non-naturally occurring. In some embodiments, the vector may be a viral or non-viral vector (e.g., phage, transposon, cosmid, bacmid, or mini-plasmid). In specific embodiments, the vector is an IVT (In Vitro Transcription) plasmid.

In another aspect, provided herein is a host cell (e.g., an in vivo or an in vitro host cell) comprising a nucleic acid described herein (e.g., a nucleic acid described in Section 5.4 or 6), or a vector described herein. A host cell may be in the form of a single cell, a population of similar or different cells, for example in the form of a culture (such as a liquid culture or a culture on a solid substrate), an organism or part thereof. The term “host cell” is intended to include a particular subject cell transformed, transduced, or transfected with a nucleic acid described herein, or vector described herein and the progeny or potential progeny of such a cell. Progeny of such a cell may not be identical to the parent cell transformed, transduced, or transfected with the nucleic acid or vector due to mutations or environmental influences that may occur in succeeding generations or integration of the nucleic acid into the cell genome. The “host cell” can be any type of cell, e.g., a primary cell, a cell in culture, or a cell from a cell line. Any cell which allows for replication of a nucleic acid or vector and/or production of the protein encoded by the nucleic acid or vector, and which can be maintained in culture is a part of the present disclosure. In some embodiments, the host cell is a cultured cell. In some embodiments, the host cell is an in vitro or ex vivo cell. In specific embodiments, the host cells are suitable for use in expressing a recombinant gene or protein. It is not intended that the present disclosure be limited to any particular type of cell. Indeed, it is contemplated that any suitable cell will find use herein. In some embodiments, the cell or host cell is isolated.

A host cell of the disclosure can be, for example, a bacterial, a yeast, an insect, or a mammalian cell, or a human cell. Examples of insect cells are High Five, Sf9, Se301, SeIZD2109, SeUCR1, Sf900+, Sf21, BTI-TN-5B1-4, MG-1, Tn368, HzAm1, BM-N, Ha2302, Hz2E5, or Ao38. Examples of mammalian cells are HEK293, HeLa, CHO, NS0, SP2/0, PER.C6, Vero, RD, BHK, HT 1080, A549, Cos-7, ARPE-19, or MRC-5 cells. The host cell may be one described herein. In some embodiments, a host cell is from a mouse. In specific embodiments, the host cell is a non-human mammalian cell. In other embodiments, the host cell is a bacterial, yeast or insect cell. In some embodiments, the host cell is a human cell. In specific embodiments, the human cell is a primary cell isolated from a human subject. In some embodiments, the host cell is from a cell line. In some embodiments, the host cell is in vitro or in cell culture (i.e., a cultured cell). In other embodiments, the host cell is in vivo. In specific embodiments, a host cell(s) is isolated from a tissue.

### 7.5 Nanoparticle Compositions

In one aspect, nucleic acid molecules described herein are formulated for in vitro and in vivo delivery. Particularly, in some embodiments the nucleic acid molecule is formulated into a lipid-containing composition. In some embodiments, the lipid-containing composition forms lipid nanoparticles enclosing the nucleic acid molecule within a lipid shell. In some embodiments, the lipid shells protect the nucleic acid molecules from degradation. In some embodiments, the lipid nanoparticles also facilitate transportation of the enclosed nucleic acid molecules into intracellular compartments and/or machinery to exert an intended therapeutic of prophylactic function. In certain embodiments, nucleic acids, when present in the lipid nanoparticles, are resistant in aqueous solution to degradation with a nuclease. Lipid nanoparticles comprising nucleic acids and their method of preparation are known in the art; such as those disclosed in, e.g., U.S. Patent Publication No. 2004/0142025, U.S. Patent Publication No. 2007/0042031, PCT Publication No. WO 2017/004143, PCT Publication No. WO 2015/199952, PCT Publication No. WO 2013/016058, and PCT Publication No. WO 2013/086373, the full disclosures of each of which are herein incorporated by reference in their entirety for all purposes.

In some embodiments, the largest dimension of a nanoparticle composition provided herein is 1 μm or shorter (e.g., ≤1 μm, ≤900 nm, ≤800 nm, ≤700 nm, ≤600 nm, ≤500 nm, ≤400 nm, ≤300 nm, ≤200 nm, ≤175 nm, ≤150 nm, ≤125 nm, ≤100 nm, ≤75 nm, ≤50 nm, or shorter), such as when measured by dynamic light scattering (DLS), transmission electron microscopy, scanning electron microscopy, or another method. In one embodiment, the lipid nanoparticle provided herein has at least one dimension that is in the range of from about 40 to about 200 nm. In one embodiment, the at least one dimension is in the range of from about 40 to about 100 nm.

Nanoparticle compositions that can be used in connection with the present disclosure include, for example, lipid nanoparticles (LNPs), nano lipoprotein particles, liposomes, lipid vesicles, and lipoplexes. In some embodiments, nanoparticle compositions are vesicles including one or more lipid bilayers. In some embodiments, a nanoparticle composition includes two or more concentric bilayers separated by aqueous compartments. Lipid bilayers may be functionalized and/or crosslinked to one another. Lipid bilayers may include one or more ligands, proteins, or channels.

In some embodiments, a nanoparticle composition comprises one or more lipid components described in Section 6 below. In some embodiments, a nanoparticle composition comprises one or more lipid components described in International Patent Application Publication No. WO 2021/204175. In some embodiments, a nanoparticle composition comprises one or more lipid components described in International Patent Application Publication No. WO 2022/152109. In some embodiments, a nanoparticle composition comprises one or more lipid components described in International Patent Application Publication No. WO 2022/247755. In some embodiments, a nanoparticle composition is one described in Section 6 below. In some embodiments, a nanoparticle composition is produced as described in Section 6. In some embodiments, a lipid (e.g., a compound according to one of Series 01, 02, 03, and 04 of lipids, such as e.g., a compound according to one of Formula (01-I), (01-II), (02-I), (03-I), and (04-I) (and sub-formulas thereof)) is produced as described in Section 6.

In some embodiments, nanoparticle compositions as described comprise a lipid component including at least one lipid, such as a compound according to one of Series 01, 02, 03, and 04 of lipids, e.g., a compound according to one of Formula (01-I), (01-II), (02-I), (03-I), and (04-I) (and sub-formulas thereof) as described herein. For example, in some embodiments, a nanoparticle composition may include a lipid component including one of compounds provided herein. Nanoparticle compositions may also include one or more other lipid or non-lipid components as described below.

### 7.5.1 Cationic Lipids

Cationic lipids include the following Series 01-04 of lipids (and sub-formulas thereof).

### Series 01 of Lipids

In one embodiment, provided herein is a compound of Formula (01-I):

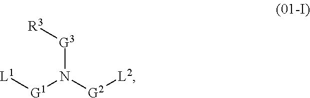

or a pharmaceutically acceptable salt, prodrug or stereoisomer thereof, wherein:
   G¹ and G² are each independently a bond, C₂-C₁₂ alkylene, or C₂-C₁₂ alkenylene, wherein one or more —CH₂— in the alkylene or alkenylene is optionally replaced by —O—;
   L¹ is —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
   L² is —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR), —(C₆-C₁₀ arylene)-R², -(6- to 10-membered heteroarylene)-R², or R²;
   R¹ and R² are each independently C₆-C₃₂ alkyl or C₆-C₃₂ alkenyl;
   R^{a}, R^{b}, R^{d}, and R^{e} are each independently H, C₁-C₂₄ alkyl, or C₂-C₂₄ alkenyl;
   R^{e} and R^{f} are each independently C₁-C₃₂ alkyl or C₂-C₃₂ alkenyl;
   G³ is C₂-C₂₄ alkylene, C₂-C₂₄ alkenylene, C₃-C₈ cycloalkylene, or C₃-C₈ cycloalkenylene;
   R³ is —N(R⁴)R⁵;
   R⁴ is C₃-C₈ cycloalkyl, C₃-C₈ cycloalkenyl, 4- to 8-membered heterocyclyl, or C₆-C₁₀ aryl; or R⁴, G³ or part of G³, together with the nitrogen to which they are attached form a cyclic moiety;
   R⁵ is C₁-C₁₂ alkyl or C₃-C₈ cycloalkyl; or R⁴, R⁵, together with the nitrogen to which they are attached form a cyclic moiety;
   x is 0, 1 or 2; and
   wherein each alkyl, alkenyl, cycloalkyl, cycloalkenyl, heterocyclyl, aryl, alkylene, alkenylene, cycloalkylene, cycloalkenylene, arylene, heteroarylene, and cyclic moiety is independently optionally substituted.

In one embodiment, provided herein is a compound of Formula (01-II):

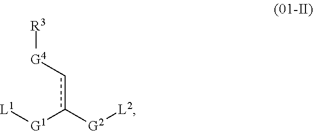

or a pharmaceutically acceptable salt, prodrug or stereoisomer thereof, wherein: [symbol] is a single bond or a double bond;
   G¹ and G² are each independently a bond, C₂-C₁₂ alkylene, or C₂-C₁₂ alkenylene, wherein one or more —CH₂— in the alkylene or alkenylene is optionally replaced by —O—;
   L¹ is —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
   L² is —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR), —(C₆-C₁₀ arylene)-R², -(6- to 10-membered heteroarylene)-R², or R²;
   R¹ and R² are each independently C₆-C₃₂ alkyl or C₆-C₃₂ alkenyl;
   R^{a}, R^{b}, R^{d}, and R^{e} are each independently H, C₁-C₂₄ alkyl, or C₂-C₂₄ alkenyl;
   R^{c} and R^{f} are each independently C₁-C₃₂ alkyl or C₂-C₃₂ alkenyl;
   G⁴ is a bond, C₁-C₂₃ alkylene, C₂-C₂₃ alkenylene, C₃-C₈ cycloalkylene, or C₃-C₈ cycloalkenylene;
   R³ is —N(R⁴)R⁵;
   R⁴ is C₁-C₁₂ alkyl, C₃-C₈ cycloalkyl, C₃-C₈ cycloalkenyl, 4- to 8-membered heterocyclyl, or C₆-C₁₀ aryl; or R⁴, G³ or part of G³, together with the nitrogen to which they are attached form a cyclic moiety;
   R⁵ is C₁-C₁₂ alkyl or C₃-C₅ cycloalkyl; or R⁴, R⁵, together with the nitrogen to which they are attached form a cyclic moiety;
   x is 0, 1 or 2; and
   wherein each alkyl, alkenyl, cycloalkyl, cycloalkenyl, heterocyclyl, aryl, alkylene, alkenylene, cycloalkylene, cycloalkenylene, arylene, heteroarylene, and cyclic moiety is independently optionally substituted.

In one embodiment, the compound is a compound in Table 01-1, or a pharmaceutically acceptable salt, prodrug or stereoisomer thereof.

TABLE 01-1 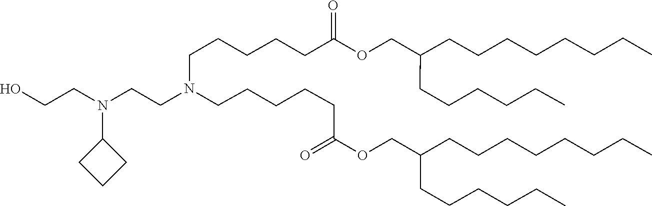 [SMILES: CCCCCCCCC(CCCCCC)COC(=O)CCCCCN(CCCCCC(=O)OCC(CCCCCC)CCCCCCCC)CCN(CCO)C1CCC1]
Compound 01-1 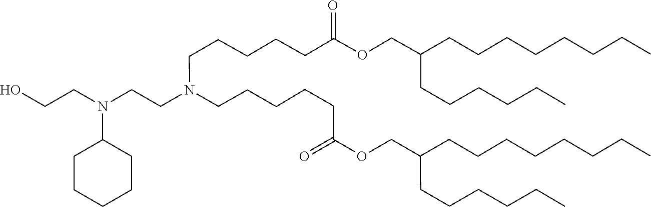 [SMILES: CCCCCCCCC(CCCCCC)COC(=O)CCCCCN(CCCCCC(=O)OCC(CCCCCC)CCCCCCCC)CCN(CCO)C1CCCCC1]
Compound 01-2 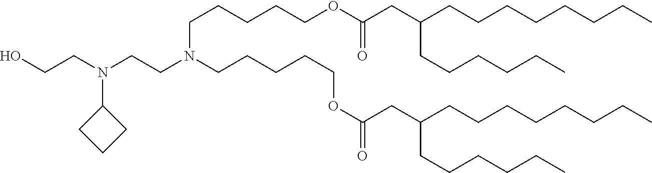 [SMILES: CCCCCCCCC(CCCCCC)CC(=O)OCCCCCN(CCCCCOC(=O)CC(CCCCCC)CCCCCCCC)CCN(CCO)C1CCC1]
Compound 01-20 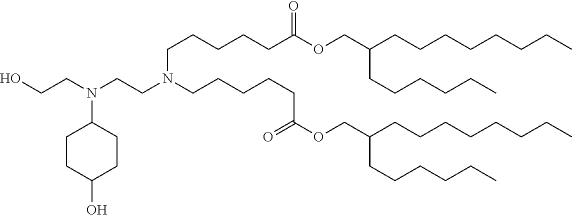 [SMILES: CCCCCCCCC(CCCCCC)COC(=O)CCCCCN(CCCCCC(=O)OCC(CCCCCC)CCCCCCCC)CCN(CCO)C1CCC(O)CC1]
Compound 01-21 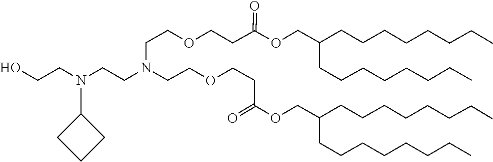 [SMILES: CCCCCCCCC(CCCCCCCC)COC(=O)CCOCCN(CCOCCC(=O)OCC(CCCCCCCC)CCCCCCCC)CCN(CCO)C1CCC1]
Compound 01-102 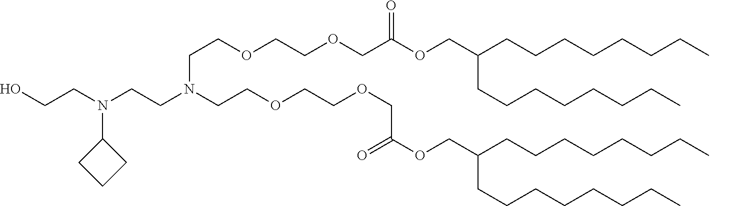
Compound 01-103 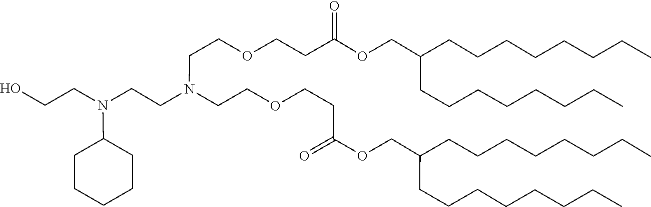 [SMILES: CCCCCCCCC(CCCCCCCC)COC(=O)CCOCCN(CCOCCC(=O)OCC(CCCCCCCC)CCCCCCCC)CCN(CCO)C1CCCCC1]
Compound 01-104 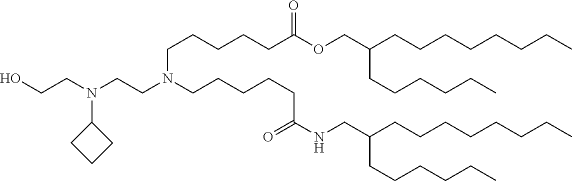 [SMILES: CCCCCCCCC(CCCCCC)CNC(=O)CCCCCN(CCCCCC(=O)OCC(CCCCCC)CCCCCCCC)CCN(CCO)C1CCC1]
Compound 01-106 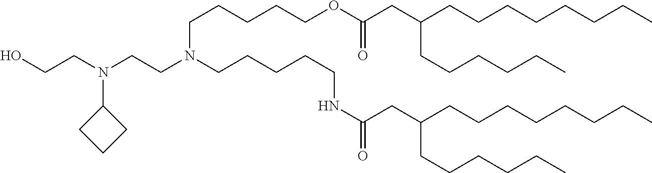 [SMILES: CCCCCCCCC(CCCCCC)CC(=O)NCCCCCN(CCCCCOC(=O)CC(CCCCCC)CCCCCCCC)CCN(CCO)C1CCC1]
Compound 01-108 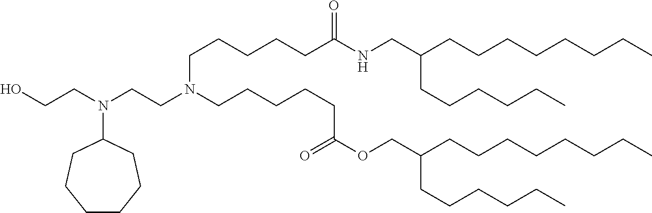 [SMILES: CCCCCCCCC(CCCCCC)CNC(=O)CCCCCN(CCCCCC(=O)OCC(CCCCCC)CCCCCCCC)CCN(CCO)C1CCCCCC1]
Compound 01-128 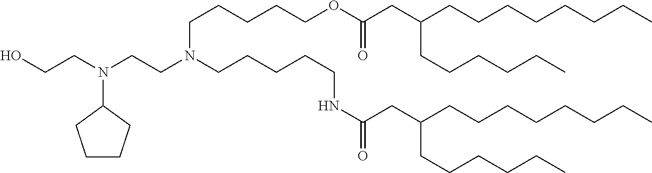 [SMILES: CCCCCCCCC(CCCCCC)CC(=O)NCCCCCN(CCCCCOC(=O)CC(CCCCCC)CCCCCCCC)CCN(CCO)C1CCCC1]
Compound 01-130 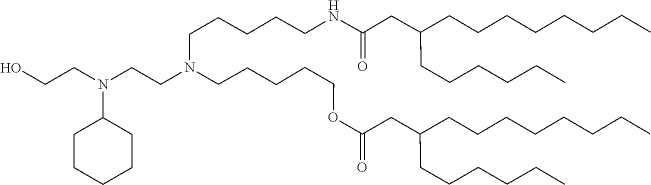 [SMILES: CCCCCCCCC(CCCCCC)CC(=O)NCCCCCN(CCCCCOC(=O)CC(CCCCCC)CCCCCCCC)CCN(CCO)C1CCCCC1]
Compound 01-131 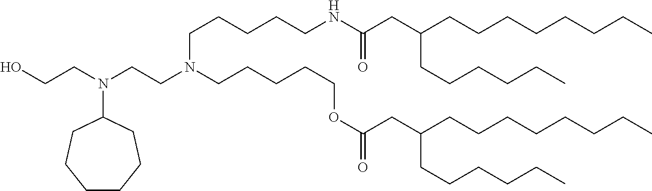 [SMILES: CCCCCCCCC(CCCCCC)CC(=O)NCCCCCN(CCCCCOC(=O)CC(CCCCCC)CCCCCCCC)CCN(CCO)C1CCCCCC1]
Compound 01-132 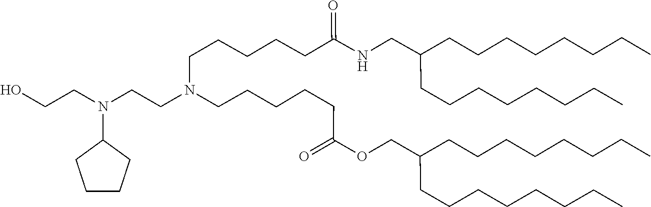 [SMILES: CCCCCCCCC(CCCCCCCC)CNC(=O)CCCCCN(CCCCCC(=O)OCC(CCCCCCCC)CCCCCCCC)CCN(CCO)C1CCCC1]
Compound 01-136

### Series 02 of Lipids

In one embodiment, provided herein is a compound of Formula (02-I):

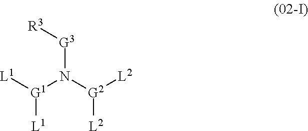

or a pharmaceutically acceptable salt, prodrug or stereoisomer thereof, wherein:
   G¹ and G² are each independently C₂-C₁₂ alkylene or C₂-C₁₂ alkenylene, wherein one or more —CH₂— in G¹ and G² is optionally replaced by —O—, —C(═O)O—, or —OC(═O)—;
   each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c});
   each L² is independently —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —NR^{d}P(═O)(OR^{e})(OR);
   R¹ and R² are each independently C₆-C₂₄ alkyl or C₆-C₂₄ alkenyl;
   R^{a}, R^{b}, R^{d}, and R^{e} are each independently H, C₁-C₂₄ alkyl, or C₂-C₂₄ alkenyl;
   R^{c} and R^{f} are each independently C₁-C₂₄ alkyl or C₂-C₂₄ alkenyl;
   G³ is C₂-C₁₂ alkylene or C₂-C₁₂ alkenylene, wherein part or all of alkylene or alkenylene is optionally replaced by a C₃-C₈ cycloalkylene or C₃-C₈ cycloalkenylene; R³ is —N(R⁴)R⁵, —OR⁶, or —SR⁶;
   R⁴ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
   R⁵ is H, C₁-C₁₂ alkyl, C₃-C₈ cycloalkyl, C₃-C₈ cycloalkenyl, C₆-C₁₀ aryl, or 4- to 8-membered heterocycloalkyl;
   R⁶ is hydrogen, C₁-C₁₂ alkyl, C₃-C₈ cycloalkyl, C₃-C₈ cycloalkenyl, or C₆-C₁₀ aryl;
   x is 0, 1, or 2; and
   wherein each alkyl, alkenyl, cycloalkyl, cycloalkenyl, heterocycloalkyl, aryl, alkylene, alkenylene, cycloalkylene, and cycloalkenylene is independently optionally substituted.

In one embodiment, the compound is a compound in Table 02-1, or a pharmaceutically acceptable salt, prodrug or stereoisomer thereof.

TABLE 02-1 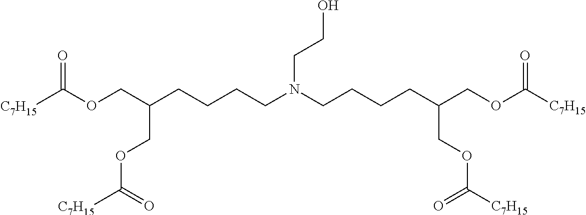
Compound 02-1 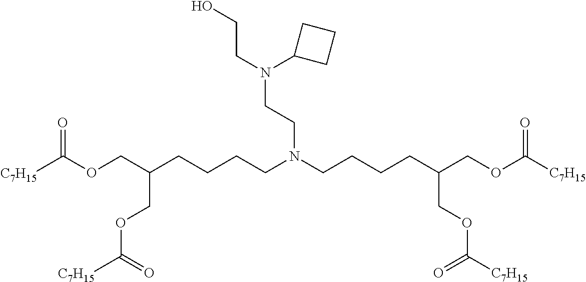
Compound 02-2 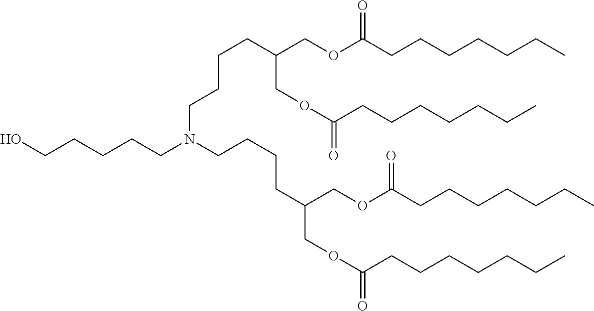 [SMILES: CCCCCCCC(=O)OCC(CCCCN(CCCCCO)CCCCC(COC(=O)CCCCCCC)COC(=O)CCCCCCC)COC(=O)CCCCCCC]
Compound 02-3 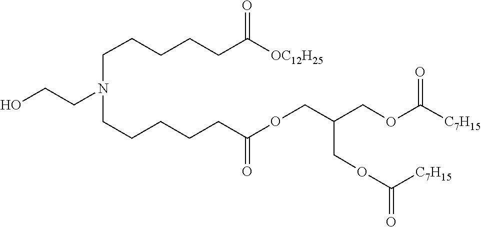
Compound 02-4 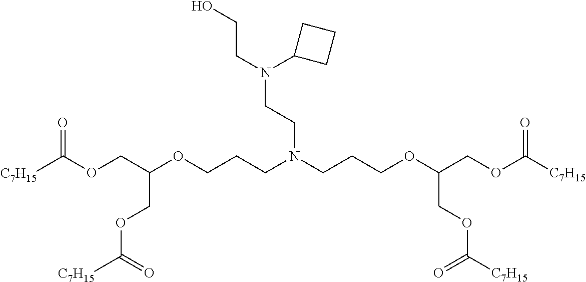
Compound 02-9 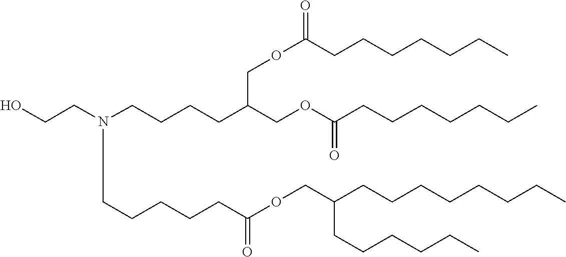 [SMILES: CCCCCCCCC(CCCCCC)COC(=O)CCCCCN(CCO)CCCCC(COC(=O)CCCCCCC)COC(=O)CCCCCCC]
Compound 02-10 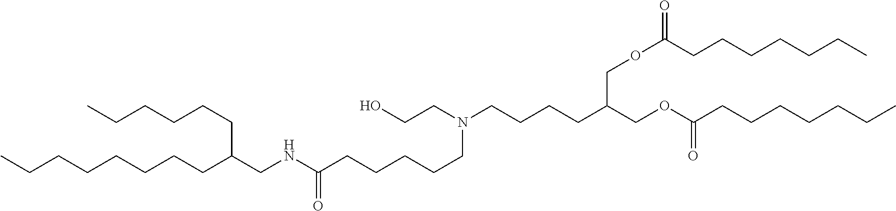 [SMILES: CCCCCCCCC(CCCCCC)CNC(=O)CCCCCN(CCO)CCCCC(COC(=O)CCCCCCC)COC(=O)CCCCCCC]
Compound 02-11 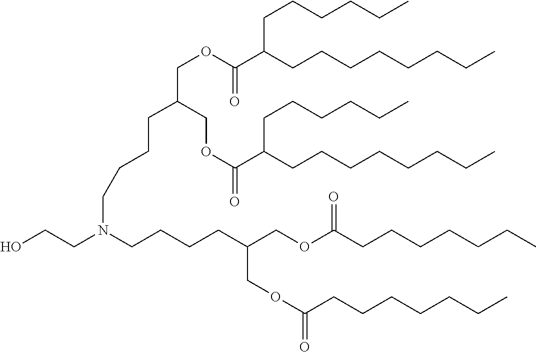 [SMILES: CCCCCCCCC(CCCCCC)C(=O)OCC(CCCCN(CCO)CCCCC(COC(=O)CCCCCCC)COC(=O)CCCCCCC)COC(=O)C(CCCCCC)CCCCCCCC]
Compound 02-12 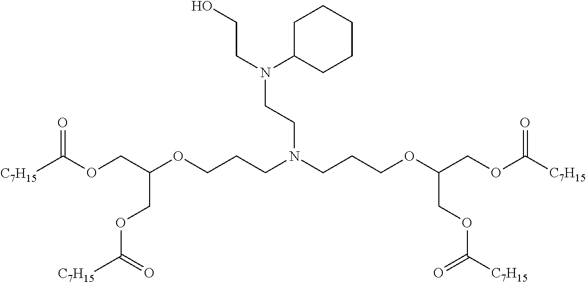
Compound 02-14 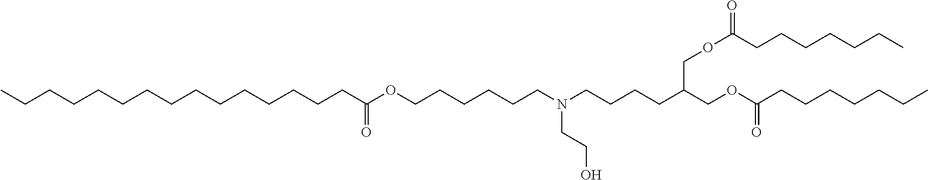 [SMILES: CCCCCCCCCCCCCCCC(=O)OCCCCCCN(CCO)CCCCC(COC(=O)CCCCCCC)COC(=O)CCCCCCC]
Compound 02-20 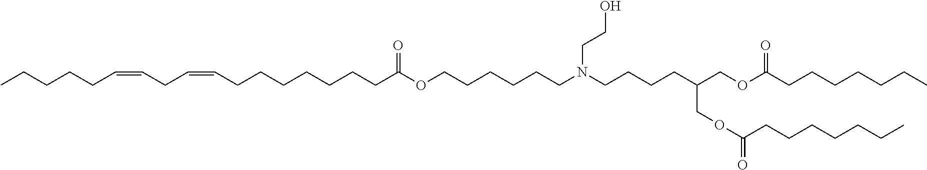 [SMILES: CCCCC/C=C\C/C=C\CCCCCCCC(=O)OCCCCCCN(CCO)CCCCC(COC(=O)CCCCCCC)COC(=O)CCCCCCC]
Compound 02-22

### Series 03 of Lipids

In one embodiment, provided herein is a compound of Formula (03-I):

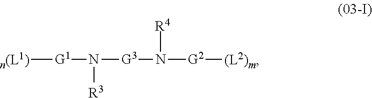

or a pharmaceutically acceptable salt, prodrug or stereoisomer thereof, wherein:
   G¹ and G² are each independently a bond, C₂-C₁₂ alkylene, or C₂-C₁₂ alkenylene, wherein one or more —CH₂— in G¹ and G² is optionally replaced by —O—;
   each L¹ is independently —OC(═O)R¹, —C(═O)OR¹, —OC(═O)OR¹, —C(═R)R¹, —OR¹, —S(O)R¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c}), —(C₆-C₁₀ arylene)-R¹, -(6- to 10-membered heteroarylene)-R¹, -(4- to 8-membered heterocyclylene)-R¹, or R¹;
   each L² is independently —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —NR^{d}P(═O)(OR^{e})(OR^{f}), —(C₆-C₁₀ arylene)-R², -(6- to 10-membered heteroarylene)-R², -(4- to 8-membered heterocyclylene)-R², or R²;
   R¹ and R² are each independently C₆-C₂₄ alkyl or C₆-C₂₄ alkenyl;
   R^{a}, R^{b}, R^{d}, and R^{e} are each independently H, C₁-C₂₄ alkyl, or C₂-C₂₄ alkenyl;
   R^{e} and R^{f} are each independently C₁-C₂₄ alkyl or C₂-C₂₄ alkenyl;
   G³ is C₂-C₁₂ alkylene or C₂-C₁₂ alkenylene, wherein part or all of alkylene or alkenylene is optionally replaced by C₃-C₈ cycloalkylene, C₃-C₈ cycloalkenylene, C₃-C₈ cycloalkynylene, 4- to 8-membered heterocyclylene, C₆-C₁₀ arylene, or 5- to 10-membered heteroarylene;
   R³ is hydrogen, C₁-C₁₂ alkyl, C₂-C₁₂ alkenyl, C₂-C₁₂ alkynyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl; or R³, G¹ or part of G¹, together with the nitrogen to which they are attached form a cyclic moiety; or R³, G³ or part of G³, together with the nitrogen to which they are attached form a cyclic moiety;
   R⁴ is C₁-C₁₂ alkyl or C₃-C₈ cycloalkyl;
   x is 0, 1, or 2;
   n is 1 or 2;
   m is 1 or 2; and
   wherein each alkyl, alkenyl, alkynyl, cycloalkyl, cycloalkenyl, cycloalkynyl, heterocyclyl, aryl, heteroaryl, alkylene, alkenylene, cycloalkylene, cycloalkenylene, cycloalkynylene, heterocyclylene, arylene, heteroarylene, and cyclic moiety is independently optionally substituted.

In one embodiment, the compound is a compound in Table 03-1, or a pharmaceutically acceptable salt, prodrug or stereoisomer thereof.

TABLE 03-1 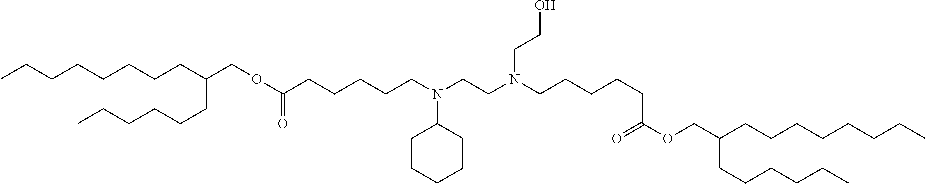 [SMILES: CCCCCCCCC(CCCCCC)COC(=O)CCCCCN(CCO)CCN(CCCCCC(=O)OCC(CCCCCC)CCCCCCCC)C1CCCCC1]
Compound 03-1 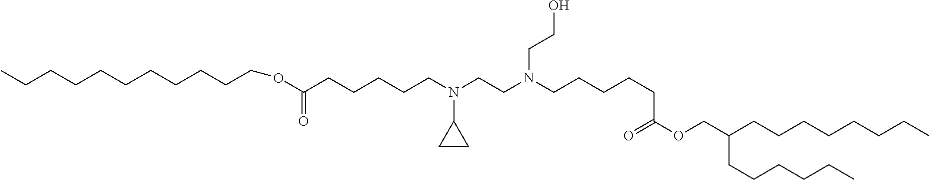 [SMILES: CCCCCCCCCCCOC(=O)CCCCCN(CCN(CCO)CCCCCC(=O)OCC(CCCCCC)CCCCCCCC)C1CC1]
Compound 03-3 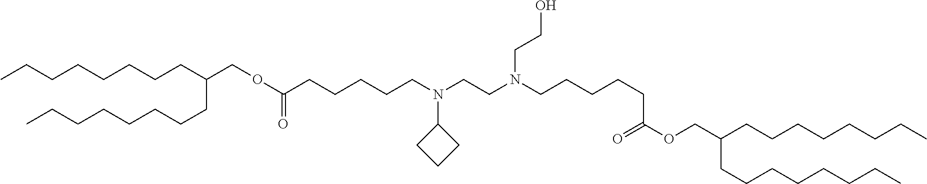 [SMILES: CCCCCCCCC(CCCCCCCC)COC(=O)CCCCCN(CCO)CCN(CCCCCC(=O)OCC(CCCCCCCC)CCCCCCCC)C1CCC1]
Compound 03-10 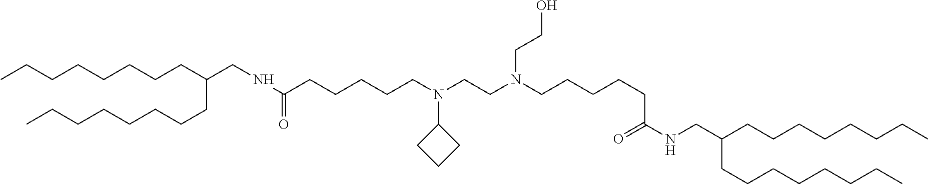 [SMILES: CCCCCCCCC(CCCCCCCC)CNC(=O)CCCCCN(CCO)CCN(CCCCCC(=O)NCC(CCCCCCCC)CCCCCCCC)C1CCC1]
Compound 03-11 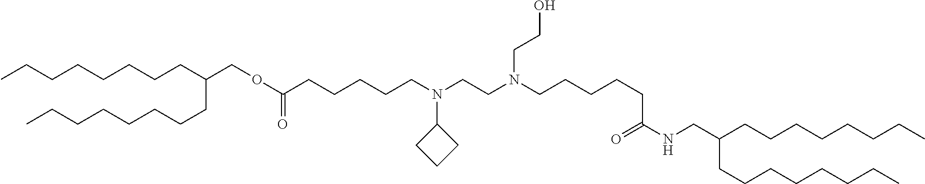 [SMILES: CCCCCCCCC(CCCCCCCC)CNC(=O)CCCCCN(CCO)CCN(CCCCCC(=O)OCC(CCCCCCCC)CCCCCCCC)C1CCC1]
Compound 03-15 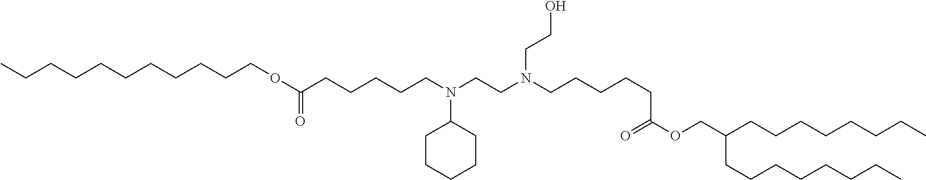 [SMILES: CCCCCCCCCCCOC(=O)CCCCCN(CCN(CCO)CCCCCC(=O)OCC(CCCCCCCC)CCCCCCCC)C1CCCCC1]
Compound 03-21 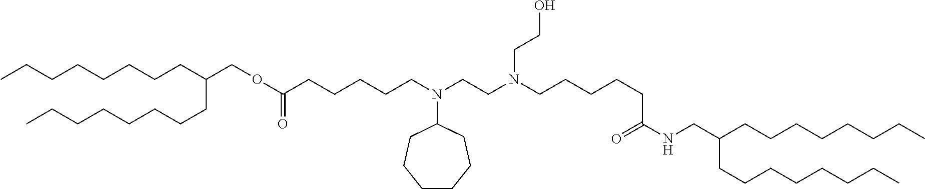 [SMILES: CCCCCCCCC(CCCCCCCC)CNC(=O)CCCCCN(CCO)CCN(CCCCCC(=O)OCC(CCCCCCCC)CCCCCCCC)C1CCCCCC1]
Compound 03-17 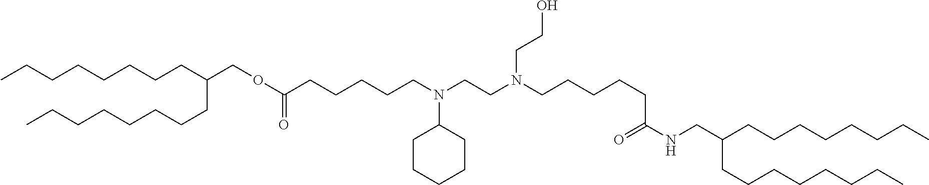 [SMILES: CCCCCCCCC(CCCCCCCC)CNC(=O)CCCCCN(CCO)CCN(CCCCCC(=O)OCC(CCCCCCCC)CCCCCCCC)C1CCCCC1]
Compound 03-23 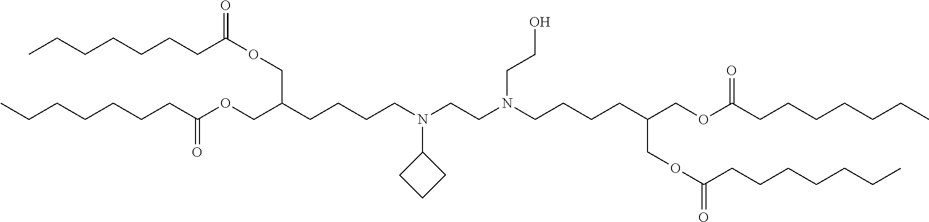 [SMILES: CCCCCCCC(=O)OCC(CCCCN(CCO)CCN(CCCCC(COC(=O)CCCCCCC)COC(=O)CCCCCCC)C1CCC1)COC(=O)CCCCCCC]
Compound 03-71 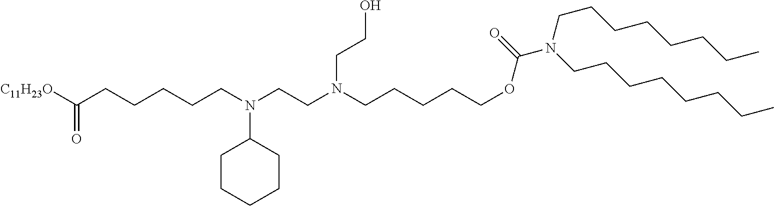
Compound 03-99 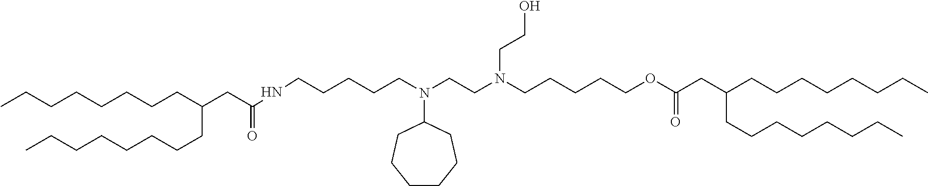 [SMILES: CCCCCCCCC(CCCCCCCC)CC(=O)NCCCCCN(CCN(CCO)CCCCCOC(=O)CC(CCCCCCCC)CCCCCCCC)C1CCCCCC1]
Compound 03-74 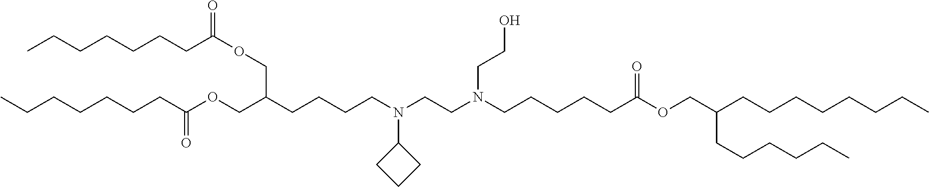 [SMILES: CCCCCCCCC(CCCCCC)COC(=O)CCCCCN(CCO)CCN(CCCCC(COC(=O)CCCCCCC)COC(=O)CCCCCCC)C1CCC1]
Compound 03-135 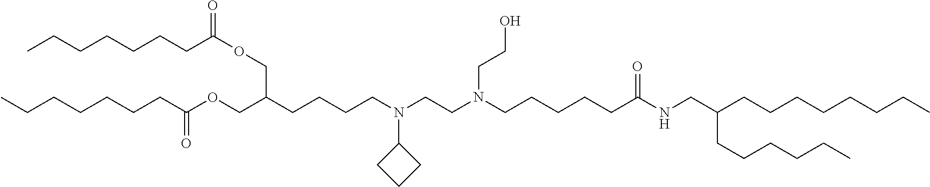 [SMILES: CCCCCCCCC(CCCCCC)CNC(=O)CCCCCN(CCO)CCN(CCCCC(COC(=O)CCCCCCC)COC(=O)CCCCCCC)C1CCC1]
Compound 03-136 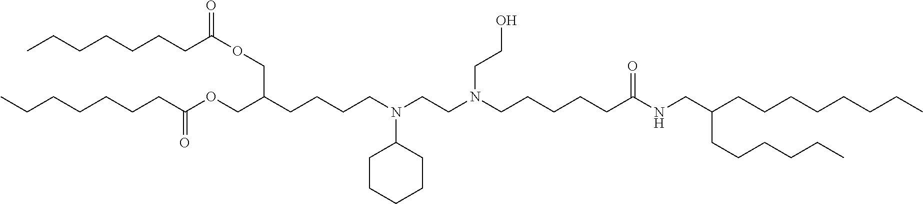 [SMILES: CCCCCCCCC(CCCCCC)CNC(=O)CCCCCN(CCO)CCN(CCCCC(COC(=O)CCCCCCC)COC(=O)CCCCCCC)C1CCCCC1]
Compound 03-140 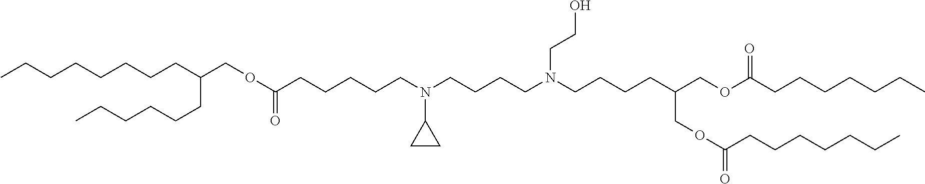 [SMILES: CCCCCCCCC(CCCCCC)COC(=O)CCCCCN(CCCCN(CCO)CCCCC(COC(=O)CCCCCCC)COC(=O)CCCCCCC)C1CC1]
Compound 03-190 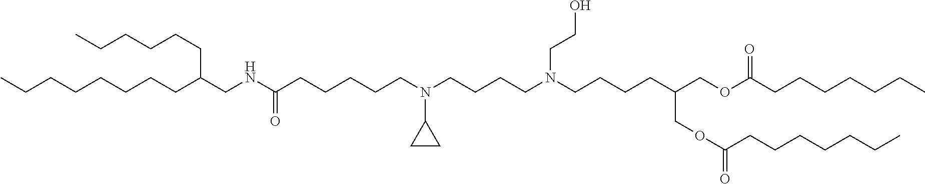 [SMILES: CCCCCCCCC(CCCCCC)CNC(=O)CCCCCN(CCCCN(CCO)CCCCC(COC(=O)CCCCCCC)COC(=O)CCCCCCC)C1CC1]
Compound 03-195 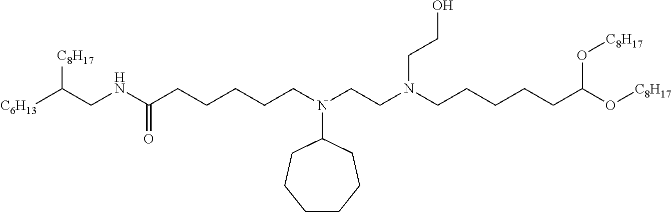
Compound 03-202 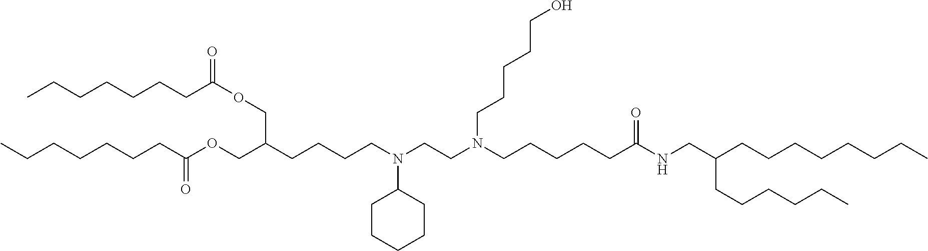 [SMILES: CCCCCCCCC(CCCCCC)CNC(=O)CCCCCN(CCCCCO)CCN(CCCCC(COC(=O)CCCCCCC)COC(=O)CCCCCCC)C1CCCCC1]
Compound 03-209

### Series 04 of Lipids

In one embodiment, provided herein is a compound of Formula (04-I):

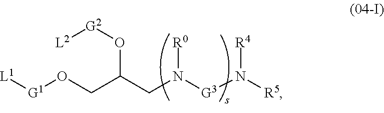

or a pharmaceutically acceptable salt, prodrug or stereoisomer thereof, wherein:
   G¹ and G² are each independently a bond, C₂-C₁₂ alkylene, or C₂-C₁₂ alkenylene;
   L¹ is —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
   L² is —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR), —(C₆-C₁₀ arylene)-R², -(6- to 10-membered heteroarylene)-R², or R²;
   R¹ and R² are each independently C₅-C₃₂ alkyl or C₅-C₃₂ alkenyl;
   R^{a}, R^{b}, R^{d}, and R^{e} are each independently H, C₁-C₂₄ alkyl, or C₂-C₂₄ alkenyl;
   R^{e} and R^{f} are each independently C₁-C₃₂ alkyl or C₂-C₃₂ alkenyl;
   R⁰ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
   G³ is C₂-C₁₂ alkylene or C₂-C₁₂ alkenylene;
   R⁴ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
   R⁵ is C₁-C₁₂ alkyl, C₃-C₈ cycloalkyl, C₃-C₈ cycloalkenyl, C₆-C₁₀ aryl, or 4- to 8-membered heterocycloalkyl;
   x is 0, 1, or 2;
   s is 0 or 1; and
   wherein each alkyl, alkenyl, cycloalkyl, cycloalkenyl, heterocycloalkyl, aryl, alkylene, alkenylene, arylene, and heteroarylene, is independently optionally substituted.

In one embodiment, the compound is a compound in Table 04-1, or a pharmaceutically acceptable salt, prodrug or stereoisomer thereof.

TABLE 04-1 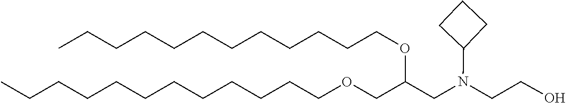 [SMILES: CCCCCCCCCCCCOCC(CN(CCO)C1CCC1)OCCCCCCCCCCCC]
Compound 04-1 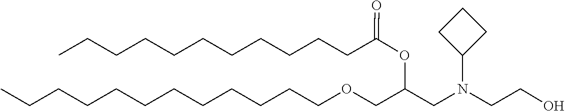 [SMILES: CCCCCCCCCCCCOCC(CN(CCO)C1CCC1)OC(=O)CCCCCCCCCCC]
Compound 04-2 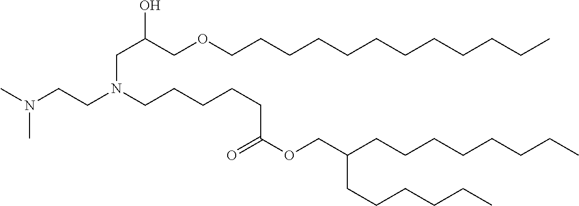 [SMILES: CCCCCCCCCCCCOCC(O)CN(CCCCCC(=O)OCC(CCCCCC)CCCCCCCC)CCN(C)C]
Compound 04-7 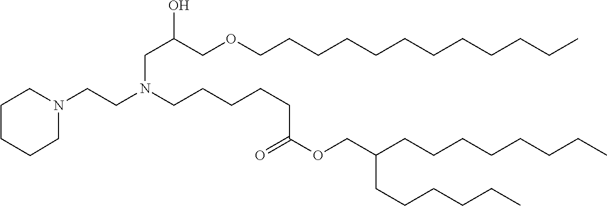 [SMILES: CCCCCCCCCCCCOCC(O)CN(CCCCCC(=O)OCC(CCCCCC)CCCCCCCC)CCN1CCCCC1]
Compound 04-8 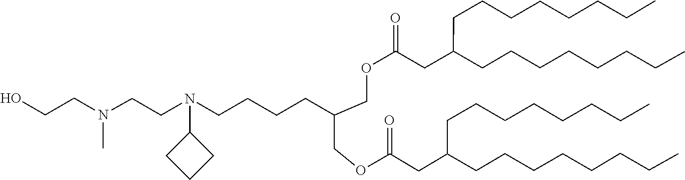 [SMILES: CCCCCCCCC(CCCCCCCC)CC(=O)OCC(CCCCN(CCN(C)CCO)C1CCC1)COC(=O)CC(CCCCCCCC)CCCCCCCC]
Compound 04-65 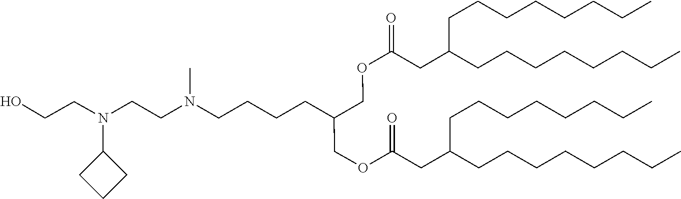 [SMILES: CCCCCCCCC(CCCCCCCC)CC(=O)OCC(CCCCN(C)CCN(CCO)C1CCC1)COC(=O)CC(CCCCCCCC)CCCCCCCC]
Compound 04-66 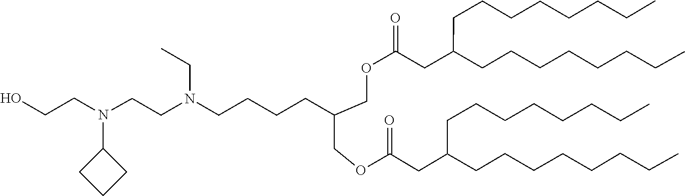 [SMILES: CCCCCCCCC(CCCCCCCC)CC(=O)OCC(CCCCN(CC)CCN(CCO)C1CCC1)COC(=O)CC(CCCCCCCC)CCCCCCCC]
Compound 04-67 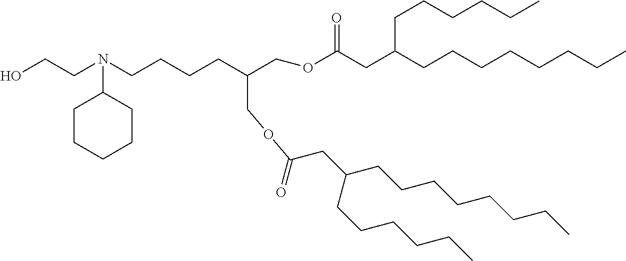 [SMILES: CCCCCCCCC(CCCCCC)CC(=O)OCC(CCCCN(CCO)C1CCCCC1)COC(=O)CC(CCCCCC)CCCCCCCC]
Compound 04-68 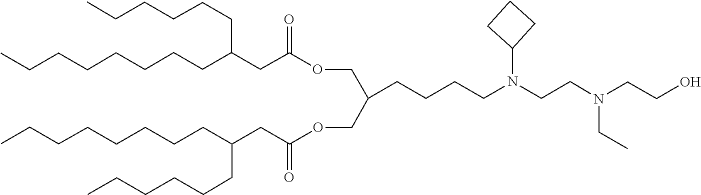 [SMILES: CCCCCCCCC(CCCCCC)CC(=O)OCC(CCCCN(CCN(CC)CCO)C1CCC1)COC(=O)CC(CCCCCC)CCCCCCCC]
Compound 04-69 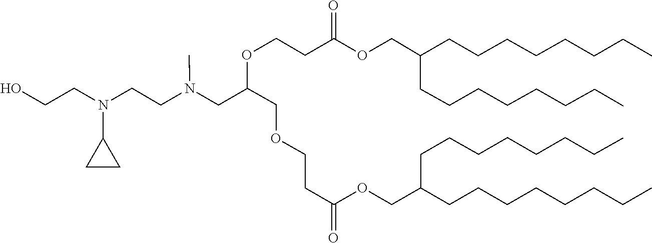 [SMILES: CCCCCCCCC(CCCCCCCC)COC(=O)CCOCC(CN(C)CCN(CCO)C1CC1)OCCC(=O)OCC(CCCCCCCC)CCCCCCCC]
Compound 04-70 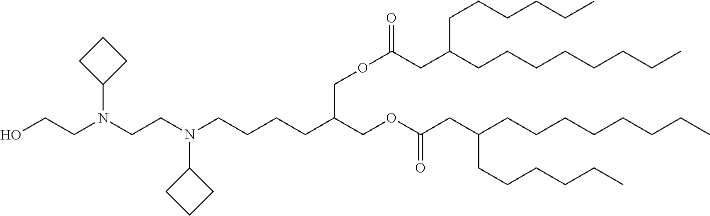 [SMILES: CCCCCCCCC(CCCCCC)CC(=O)OCC(CCCCN(CCN(CCO)C1CCC1)C1CCC1)COC(=O)CC(CCCCCC)CCCCCCCC]
Compound 04-79 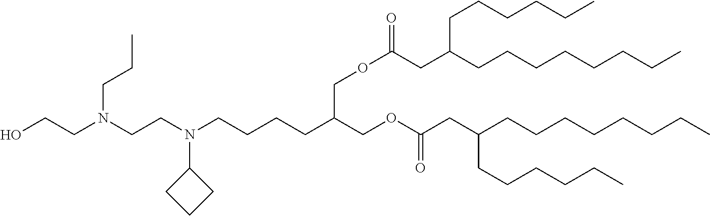 [SMILES: CCCCCCCCC(CCCCCC)CC(=O)OCC(CCCCN(CCN(CCC)CCO)C1CCC1)COC(=O)CC(CCCCCC)CCCCCCCC]
Compound 04-80

It is understood that any embodiment of the compounds provided herein, as set forth above, and any specific substituent and/or variable in the compound provided herein, as set forth above, may be independently combined with other embodiments and/or substituents and/or variables of the compounds to form embodiments not specifically set forth above. In addition, in the event that a list of substituents and/or variables is listed for any particular group or variable, it is understood that each individual substituent and/or variable may be deleted from the particular embodiment and/or claim and that the remaining list of substituents and/or variables will be considered to be within the scope of embodiments provided herein.

It is understood that in the present description, combinations of substituents and/or variables of the depicted formulae are permissible only if such contributions result in stable compounds.

### 7.5.2 Other Ionizable Lipids

As described herein, in some embodiments, a nanoparticle composition provided herein comprises one or more charged or ionizable lipids in addition to a compound according to Series 01, 02, 03, and 04, e.g., a lipid according to Formulae (01-I), (01-II), (02-I), (03-I), and (04-I) (and sub-formulas thereof). Without being bound by the theory, it is contemplated that certain charged or zwitterionic lipid components of a nanoparticle composition resembles the lipid component in the cell membrane, thereby can improve cellular uptake of the nanoparticle. Exemplary charged or ionizable lipids that can form part of the present nanoparticle composition include but are not limited to 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLinDMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3 [(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)—-octadeca-9,12-dien-1-yloxy]propan-1-amine (Octyl-CLinDMA (2R)), (2S)-2-({8-[(3β)-cholest-5-en-3-yloxy]octyl}oxy)-N,N-dimethyl-3-[(9Z-, 12Z)-octadeca-9,12-dien-1-yloxy]propan-1-amine (Octyl-CLinDMA (2S)), (12Z,15Z)—N,N-dimethyl-2-nonylhenicosa-12,15-den-1-amine, N,N-dimethyl-1-{(1S,2R)-2-octylcyclopropyl}heptadecan-8-amine. Additional exemplary charged or ionizable lipids that can form part of the present nanoparticle composition include the lipids (e.g., lipid 5) described in Sabnis et al. “A Novel Amino Lipid Series for mRNA Delivery: Improved Endosomal Escape and Sustained Pharmacology and Safety in Non-human Primates”, Molecular Therapy Vol. 26 No 6, 2018, the entirety of which is incorporated herein by reference.

In some embodiments, suitable cationic lipids include N-[1-(2,3-dioleyloxy)propyl]-N,N,N-trimethylammonium chloride (DOTMA); N-[1-(2,3-dioleoyloxy)propyl]-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1); N1-[2-((1S)-1-[(3-aminopropyl)amino]-4-[di(3-amino-propyl)amino]butylcarboxamido)ethyl]-3,4-di[oleyloxy]-benzamide (MVL5); dioctadecylamido-glycylspermine (DOGS); 3b-[N—(N′,N′-dimethylaminoethyl)carbamoyl]cholesterol (DC-Chol); dioctadecyl dimethylammonium bromide (DDAB); SAINT-2, N-methyl-4-(dioleyl)methylpyridinium; 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); di-alkylated amino acid (DILA²) (e.g., C18:1-norArg-C16); dioleyldimethylammoniumchloride (DODAC); 1-palmitoyl-2-oleoyl-sn-glycero-3-ethylphosphocholine (POEPC); 1,2-dimyristoleoyl-sn-glycero-3-ethylphosphocholine (MOEPC); (R)-5-(dimethylamino)pentane-1,2-diyl dioleate hydrochloride (DODAPen-Cl); (R)-5-guanidinopentane-1,2-diyl dioleate hydrochloride (DOPen-G); and (R)—N,N,N-trimethyl-4,5-bis(oleoyloxy)pentan-1-aminium chloride (DOTAPen). Also suitable are cationic lipids with headgroups that are charged at physiological pH, such as primary amines (e.g., DODAG N′,N′-dioctadecyl-N-4,8-diaza-10-aminodecanoylglycine amide) and guanidinium head groups (e.g., bis-guanidinium-spermidine-cholesterol (BGSC), bis-guanidiniumtren-cholesterol (BGTC), PONA, and (R)-5-guanidinopentane-1,2-diyl dioleate hydrochloride (DOPen-G)). Yet another suitable cationic lipid is (R)-5-(dimethylamino) pentane-1,2-diyl dioleate hydrochloride (DODAPen-Cl). In certain embodiments, the cationic lipid is a particular enantiomer or the racemic form, and includes the various salt forms of a cationic lipid as above (e.g., chloride or sulfate). For example, in some embodiments, the cationic lipid is N-[1-(2,3-dioleoyloxy)propyl]-N,N,N-trimethylammonium chloride (DOTAP-Cl) or N-[1-(2,3-dioleoyloxy)propyl]-N,N,N-trimethylammonium sulfate (DOTAP-Sulfate). In some embodiments, the cationic lipid is an ionizable cationic lipid such as, e.g., dioctadecyl dimethylammonium bromide (DDAB); 1,2-dilinoleyloxy-3-dimethylaminopropane (DLinDMA); 2,2-dilinoleyl-4-(2dimethylaminoethyl)-[1,3]-dioxolane (DLin-KC2-DMA); heptatriaconta-6,9,28,31-tetraen-19-yl 4-(dimethylamino)butanoate (DLin-MC3-DMA); 1,2-dioleoyloxy-3-dimethylaminopropane (DODAP); 1,2-dioleyloxy-3-dimethylaminopropane (DODMA); and morpholino cholesterol (Mo-CHOL). In certain embodiments, a lipid nanoparticle includes a combination or two or more cationic lipids (e.g., two or more cationic lipids as above).

Additionally, in some embodiments, the charged or ionizable lipid that can form part of the present nanoparticle composition is a lipid including a cyclic amine group. Additional cationic lipids that are suitable for the formulations and methods disclosed herein include those described in WO 2015/199952, WO 2016/176330, and WO 2015/011633, the entire contents of each of which are hereby incorporated by reference in their entireties. Additionally, in some embodiments, the charged or ionizable lipid that can form part of the present nanoparticle composition is a lipid including a cyclic amine group. Additional cationic lipids that are suitable for the formulations and methods disclosed herein include those described in WO 2015/199952, WO 2016/176330, and WO 2015/011633, the entire contents of each of which are hereby incorporated by reference in their entireties.

### 7.5.3 Polymer Conjugated Lipids

In some embodiments, the lipid component of a nanoparticle composition can include one or more polymer conjugated lipids, such as PEGylated lipids (PEG lipids). Without being bound by the theory, it is contemplated that a polymer conjugated lipid component in a nanoparticle composition can improve of colloidal stability and/or reduce protein absorption of the nanoparticles. Exemplary cationic lipids that can be used in connection with the present disclosure include but are not limited to PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DSPE, Ceramide-PEG2000, or Chol-PEG2000.

In one embodiment, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O—(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In one embodiment, the polymer conjugated lipid is present in a concentration ranging from 1.0 to 2.5 molar percent. In one embodiment, the polymer conjugated lipid is present in a concentration of about 1.7 molar percent. In one embodiment, the polymer conjugated lipid is present in a concentration of about 1.5 molar percent.

In one embodiment, the molar ratio of cationic lipid to the polymer conjugated lipid ranges from about 35:1 to about 25:1. In one embodiment, the molar ratio of cationic lipid to polymer conjugated lipid ranges from about 100:1 to about 20:1.

In one embodiment, the molar ratio of cationic lipid to the polymer conjugated lipid ranges from about 35:1 to about 25:1. In one embodiment, the molar ratio of cationic lipid to polymer conjugated lipid ranges from about 100:1 to about 20:1.

In one embodiment, the pegylated lipid has the following Formula:

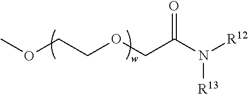

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and
   w has a mean value ranging from 30 to 60. In one embodiment, R12 and R13 are each independently straight, saturated alkyl chains containing from 12 to 16 carbon atoms. In other embodiments, the average w ranges from 42 to 55, for example, the average w is 42, 43, 44, 45, 46, 47, 48, 49, 50, 51, 52, 53, 54 or 55. In some specific embodiments, the average w is about 49.

In one embodiment, the pegylated lipid has the following Formula:

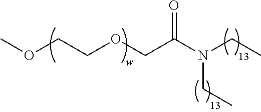

wherein the average w is about 49.

### Series 05 of Lipids

In one embodiment, provided herein is a compound of Formula (05-I):

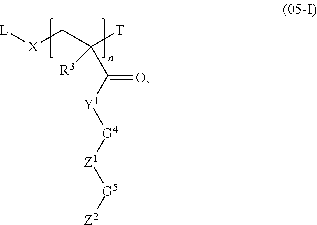

or a pharmaceutically acceptable salt or stereoisomer thereof, wherein:
L is a lipid;
X is a linker;
each R³ is independently H or C₁-C₆ alkyl;
each Y¹ is independently a bond, O, S, or NR^{a};
each G⁴ is independently a bond or C₁-C₁₂ alkylene, wherein one or more —CH₂— is independently optionally replaced by —O—, —S—, or —NR^{a}—;
each G⁵ is independently a bond or C₁-C₁₂ alkylene, wherein one or more —CH₂— is independently optionally replaced by —O—, —S—, or —NR^{a}—;
each R^{a} is independently H, C₁-C₁₂ alkyl, or C₂-C₁₂ alkenyl;
one of Z¹ and Z² is a positively charged moiety and the other of Z¹ and Z² is a negatively charged moiety;
n is an integer from 2 to 100;
T is hydrogen, halogen, alkyl, alkenyl, —OR″, —SR″, —COOR″, —OCOR″, —NR″R″, —N⁺(R″)₃, —P⁺(R″)₃, —S—C(═S)—S—R″, —S—C(═S)—O—R″, —S—C(═S)—NR″R″, —S—C(═S)-aryl, cyano, azido, aryl, heteroaryl, or a targeting group, wherein each occurrence of R″ is independently hydrogen or alkyl; and wherein each alkyl, alkenyl, alkylene, aryl, and heteroaryl is independently optionally substituted.

### 7.5.4 Structural Lipids

In some embodiments, the lipid component of a nanoparticle composition can include one or more structural lipids. Without being bound by the theory, it is contemplated that structural lipids can stabilize the amphiphilic structure of a nanoparticle, such as but not limited to the lipid bilayer structure of a nanoparticle. Exemplary structural lipids that can be used in connection with the present disclosure include but are not limited to cholesterol, fecosterol, sitosterol, ergosterol, campesterol, stigmasterol, brassicasterol, tomatidine, tomatine, ursolic acid, alpha-tocopherol, and mixtures thereof. In certain embodiments, the structural lipid is cholesterol. In some embodiments, the structural lipid includes cholesterol and a corticosteroid (such as prednisolone, dexamethasone, prednisone, and hydrocortisone), or a combination thereof.

In one embodiment, the lipid nanoparticles provided herein comprise a steroid or steroid analogue. In one embodiment, the steroid or steroid analogue is cholesterol. In one embodiment, the steroid is present in a concentration ranging from 39 to 49 molar percent; 40 to 46 molar percent, from 40 to 44 molar percent, from 40 to 42 molar percent, from 42 to 44 molar percent, or from 44 to 46 molar percent. In one embodiment, the steroid is present in a concentration of 40, 41, 42, 43, 44, 45, or 46 molar percent.

In one embodiment, the molar ratio of cationic lipid to the steroid ranges from 1.0:0.9 to 1.0:1.2, or from 1.0:1.0 to 1.0:1.2. In one embodiment, the molar ratio of cationic lipid to cholesterol ranges from about 5:1 to 1:1. In one embodiment, the steroid is present in a concentration ranging from 32 to 40 mol percent of the steroid.

In one embodiment, the molar ratio of cationic lipid to the steroid ranges from 1.0:0.9 to 1.0:1.2, or from 1.0:1.0 to 1.0:1.2. In one embodiment, the molar ratio of cationic lipid to cholesterol ranges from about 5:1 to 1:1. In one embodiment, the steroid is present in a concentration ranging from 32 to 40 mol percent of the steroid.

### 7.5.5 Phospholipids

In some embodiments, the lipid component of a nanoparticle composition can include one or more phospholipids, such as one or more (poly)unsaturated lipids. Without being bound by the theory, it is contemplated that phospholipids may assemble into one or more lipid bilayers structures. Exemplary phospholipids that can form part of the present nanoparticle composition include but are not limited to 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16LysoPC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In certain embodiments, a nanoparticle composition includes DSPC. In certain embodiments, a nanoparticle composition includes DOPE. In some embodiments, a nanoparticle composition includes both DSPC and DOPE.

Additional exemplary neutral lipids include, for example, dipalmitoylphosphatidylglycerol (DPPG), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC). In one embodiment, the neutral lipid is selected from DSPC, DPPC, DMPC, DOPC, POPC, DOPE and SM.

In one embodiment, the neutral lipid is phosphatidylcholine (PC), phosphatidylethanolamine (PE) phosphatidylserine (PS), phosphatidic acid (PA), or phosphatidylglycerol (PG).

Additionally, phospholipids that can form part of the present nanoparticle composition also include those described in WO 2017/112865, the entire content of which is hereby incorporated by reference in its entirety.

### 7.5.6 Formulation

According to the present disclosure, nanoparticle compositions described herein can include at least one lipid component and one or more additional components, such as a therapeutic and/or prophylactic agent (e.g., the therapeutic nucleic acid described herein). In specific embodiments, a nanoparticle composition described herein comprises at least one lipid component (e.g., a lipid component described herein, e.g., in Section 5.5 or 6) and a nucleic acid described herein (e.g., a nucleic acid described in Section 5.4 or 6). A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a lipid according to one of Formula 01-I, 01-II, 02-I, 02-II, 03-I, 03-II-A, 03-II-B, 03-II-C, 03-II-D, 04-I, 04-III, 04-IV (and sub-formulas thereof) described herein, a phospholipid (such as an unsaturated lipid, e.g., DOPE or DSPC), a polymer conjugated lipid (such as a PEG lipid), and a structural lipid (such as a steroid). The elements of the lipid component may be provided in specific fractions.

In one embodiment, provided herein is a nanoparticle composition comprising a cationic or ionizable lipid compound provided herein, a therapeutic agent, and one or more excipients. In one embodiment, cationic or ionizable lipid compound comprises a compound according to one of Formula 01-I, 01-II, 02-I, 02-II, 03-I, 03-II-A, 03-II-B, 03-II-C, 03-II-D, 04-I, 04-III, 04-IV (and sub-formulas thereof) as described herein, and optionally one or more additional ionizable lipid compounds. In one embodiment, the one or more excipients are selected from neutral lipids, phospholipids, steroids, and polymer conjugated lipids. In one embodiment, the therapeutic agent is encapsulated within or associated with the lipid nanoparticle.

In one embodiment, provided herein is a nanoparticle composition (lipid nanoparticle) comprising:
i) from about 40 to about 50 mol percent of a cationic lipid;
ii) a neutral lipid;
iii) a steroid;
iv) a polymer conjugated lipid; and
v) a therapeutic agent.

In one embodiment, provided herein is a nanoparticle composition (lipid nanoparticle) comprising:
i) from about 20 to about 65 mol percent of a cationic lipid;
ii) from about 5 to about 40 mol percent of a phospholipid;
iii) from about 20 to about 50 mol percent of a steroid;
iv) a polymer conjugated lipid; and
v) a therapeutic agent.

In some embodiment, the nanoparticle composition (lipid nanoparticle) comprising:
i) from about 40 to about 55 mol percent of a cationic lipid;
ii) from about 5 to about 15 mol percent of a phospholipid;
iii) from about 35 to about 50 mol percent of a steroid; and
iv) from about 2 to about 10 mol percent of a polymer conjugated lipid.

In one embodiment, the nanoparticle composition (lipid nanoparticle) comprising:
i) from about 45 to about 55 mol percent of a cationic lipid;
ii) from about 6 to about 10 mol percent of a phospholipid;
iii) from about 40 to about 48 mol percent of a steroid; and
iv) from about 1 to about 2.5 mol percent of a polymer conjugated lipid.

As used herein, “mol percent” refers to a component's molar percentage relative to total mols of all lipid components in the LNP (i.e., total mols of cationic lipid(s), the neutral lipid, the steroid and the polymer conjugated lipid).

In one embodiment, the therapeutic agent is a nucleic acid molecule according to the present disclosure. In some embodiments, the therapeutic agent comprises any one or more nucleic acid sequences described in Section 5.3 (Therapeutic Nucleic Acids) herein.

In one embodiment, the lipid nanoparticle comprises from 40 to 50 mol percent from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, or from 47.2 to 47.8 mol percent of the cationic lipid. In one embodiment, the lipid nanoparticle comprises about 47.0, 47.1, 47.2, 47.3, 47.4, 47.5, 47.6, 47.7, 47.8, 47.9, or 48.0 mol percent of the cationic lipid. In one embodiment, the lipid nanoparticle comprises about 50 mol percent of the cationic lipid.

In one embodiment, the neutral lipid is present in a concentration ranging from 5 to 15 mol percent, 7 to 13 mol percent, or 9 to 11 mol percent. In one embodiment, the neutral lipid is present in a concentration of about 9.5, 10 or 10.5 mol percent. In one embodiment, the molar ratio of the cationic lipid to the neutral lipid ranges from about 4.1:1.0 to about 4.9:1.0, from about 4.5:1.0 to about 4.8:1.0, or from about 4.7:1.0 to 4.8:1.0.

In one embodiment, the steroid is present in a concentration ranging from 38 to 49 molar percent, 40 to 46 molar percent, from 40 to 44 molar percent, from 40 to 42 molar percent, from 42 to 44 molar percent, or from 44 to 46 molar percent. In one embodiment the steroid is present in a concentration of 40, 41, 42, 43, 44, 45, or 46 molar percent. In one embodiment, the steroid is present in a concentration of 38.0, 38.1, 38.2, 38.3, 38.4, 38.5, 38.6, 38.7, 38.8, or 38.9 molar percent. In one embodiment, the molar ratio of cationic lipid to the steroid ranges from 1.0:0.9 to 1.0:1.2, or from 1.0:1.0 to 1.0:1.2. In one embodiment, the steroid is cholesterol.

In one embodiment, the lipid nanoparticle comprises about 50 mol percent of the cationic lipid, and about 38.5 mol percent of steroid. In some embodiments, the molar ratio of cationic lipid to the steroid is 1.3:1.0. In one embodiment, the steroid is cholesterol.

In one embodiment, the therapeutic agent to lipid ratio in the LNP (i.e., N/P, were N represents the moles of cationic lipid and P represents the moles of phosphate present as part of the nucleic acid backbone) range from 2:1 to 30:1, for example 3:1 to 22:1. In one embodiment, N/P ranges from 6:1 to 20:1 or 2:1 to 12:1. Exemplary N/P ranges include about 3:1. About 6:1, about 12:1 and about 22:1.

In one embodiment, provided herein is a lipid nanoparticle comprising:
i) a cationic lipid having an effective pKa greater than 6.0; ii) from 5 to 15 mol percent of a neutral lipid;
iii) from 1 to 15 mol percent of an anionic lipid;
iv) from 30 to 45 mol percent of a steroid;
v) a polymer conjugated lipid; and
vi) a therapeutic agent, or a pharmaceutically acceptable salt or prodrug thereof,
wherein the mol percent is determined based on total mol of lipid present in the lipid nanoparticle.

In one embodiment, the cationic lipid can be any of a number of lipid species which carry a net positive charge at a selected pH, such as physiological pH. Exemplary cationic lipids are described herein below. In one embodiment, the cationic lipid has a pKa greater than 6.25. In one embodiment, the cationic lipid has a pKa greater than 6.5. In one embodiment, the cationic lipid has a pKa greater than 6.1, greater than 6.2, greater than 6.3, greater than 6.35, greater than 6.4, greater than 6.45, greater than 6.55, greater than 6.6, greater than 6.65, or greater than 6.7.

In one embodiment, the lipid nanoparticle comprises from 40 to 45 mol percent of the cationic lipid. In one embodiment, the lipid nanoparticle comprises from 45 to 50 mole percent of the cationic lipid.

In one embodiment, the molar ratio of the cationic lipid to the neutral lipid ranges from about 2:1 to about 8:1. In one embodiment, the lipid nanoparticle comprises from 5 to 10 mol percent of the neutral lipid.

Exemplary anionic lipids include, but are not limited to, phosphatidylglycerol, dioleoylphosphatidylglycerol (DOPG), dipalmitoylphosphatidylglycerol (DPPG) or 1,2-distearoyl-sn-glycero-3-phospho-(1′-rac-glycerol) (DSPG).

In one embodiment, the lipid nanoparticle comprises from 1 to 10 mole percent of the anionic lipid. In one embodiment, the lipid nanoparticle comprises from 1 to 5 mole percent of the anionic lipid. In one embodiment, the lipid nanoparticle comprises from 1 to 9 mole percent, from 1 to 8 mole percent, from 1 to 7 mole percent, or from 1 to 6 mole percent of the anionic lipid. In one embodiment, the mol ratio of anionic lipid to neutral lipid ranges from 1:1 to 1:10.

In one embodiment, the steroid cholesterol. In one embodiment, the molar ratio of the cationic lipid to cholesterol ranges from about 5:1 to 1:1. In one embodiment, the lipid nanoparticle comprises from 32 to 40 mol percent of the steroid.

In one embodiment, the sum of the mol percent of neutral lipid and mol percent of anionic lipid ranges from 5 to 15 mol percent. In one embodiment, wherein the sum of the mol percent of neutral lipid and mol percent of anionic lipid ranges from 7 to 12 mol percent.

In one embodiment, the mol ratio of anionic lipid to neutral lipid ranges from 1:1 to 1:10. In one embodiment, the sum of the mol percent of neutral lipid and mol percent steroid ranges from 35 to 45 mol percent.

In one embodiment, the lipid nanoparticle comprises:
i) from 45 to 55 mol percent of the cationic lipid;
ii) from 5 to 10 mol percent of the neutral lipid;
iii) from 1 to 5 mol percent of the anionic lipid; and
iv) from 32 to 40 mol percent of the steroid.

In one embodiment, the lipid nanoparticle comprises from 1.0 to 2.5 mol percent of the conjugated lipid. In one embodiment, the polymer conjugated lipid is present in a concentration of about 1.5 mol percent.

In one embodiment, the neutral lipid is present in a concentration ranging from 5 to 15 mol percent, 7 to 13 mol percent, or 9 to 11 mol percent. In one embodiment, the neutral lipid is present in a concentration of about 9.5, 10 or 10.5 mol percent. In one embodiment, the molar ratio of the cationic lipid to the neutral lipid ranges from about 4.1:1.0 to about 4.9:1.0, from about 4.5:1.0 to about 4.8:1.0, or from about 4.7:1.0 to 4.8:1.0.

In one embodiment, the steroid is cholesterol. In some embodiments, the steroid is present in a concentration ranging from 39 to 49 molar percent, 40 to 46 molar percent from 40 to 44 molar percent, from 40 to 42 molar percent, from 42 to 44 molar percent, or from 44 to 46 molar percent. In one embodiment, the steroid is present in a concentration of 40, 41, 42, 43, 44, 45, or 46 molar percent. In certain embodiments, the molar ratio of cationic lipid to the steroid ranges from 1.0:0.9 to 1.0:1.2, or from 1.0:1.0 to 1.0:1.2.

In one embodiment, the molar ratio of cationic lipid to steroid ranges from 5:1 to 1:1.

In one embodiment, the lipid nanoparticle comprises from 1.0 to 2.5 mol percent of the conjugated lipid. In one embodiment, the polymer conjugated lipid is present in a concentration of about 1.5 mol percent.

In one embodiment, the molar ratio of cationic lipid to polymer conjugated lipid ranges from about 100:1 to about 20:1. In one embodiment, the molar ratio of cationic lipid to the polymer conjugated lipid ranges from about 35:1 to about 25:1.

In one embodiment, the molar ratio of cationic lipid to polymer conjugated lipid ranges from about 100:1 to about 20:1. In one embodiment, the molar ratio of cationic lipid to the polymer conjugated lipid ranges from about 35:1 to about 25:1.

In one embodiment, the lipid nanoparticle has a mean diameter ranging from 50 nm to 100 nm, or from 60 nm to 85 nm.

In one embodiment, the composition comprises a cationic lipid provided herein, DSPC, cholesterol, and PEG-lipid, and mRNA. In one embodiment, a cationic lipid provided herein, DSPC, cholesterol, and PEG-lipid are at a molar ratio of about 50:10:38.5:1.5.

Nanoparticle compositions can be designed for one or more specific applications or targets. For example, a nanoparticle composition can be designed to deliver a therapeutic and/or prophylactic agent such as an RNA to a particular cell, tissue, organ, or system or group thereof in a mammal's body. Physiochemical properties of nanoparticle compositions can be altered in order to increase selectivity for particular bodily targets. For instance, particle sizes can be adjusted based on the fenestration sizes of different organs. The therapeutic and/or prophylactic agent included in a nanoparticle composition can also be selected based on the desired delivery target or targets. For example, a therapeutic and/or prophylactic agent can be selected for a particular indication, condition, disease, or disorder and/or for delivery to a particular cell, tissue, organ, or system or group thereof (e.g., localized or specific delivery). In certain embodiments, a nanoparticle composition can include an mRNA encoding a polypeptide of interest capable of being translated within a cell to produce the polypeptide of interest. Such a composition can be designed to be specifically delivered to a particular organ. In certain embodiments, a composition can be designed to be specifically delivered to a mammalian liver.

The amount of a therapeutic and/or prophylactic agent in a nanoparticle composition can depend on the size, composition, desired target and/or application, or other properties of the nanoparticle composition as well as on the properties of the therapeutic and/or prophylactic agent. For example, the amount of an RNA useful in a nanoparticle composition can depend on the size, sequence, and other characteristics of the RNA. The relative amounts of a therapeutic and/or prophylactic agent and other elements (e.g., lipids) in a nanoparticle composition can also vary. In some embodiments, the wt/wt ratio of the lipid component to a therapeutic and/or prophylactic agent in a nanoparticle composition can be from about 5:1 to about 60:1, such as about 5:1, 6:1, 7:1, 8:1, 9:1, 10:1, 11:1, 12:1, 13:1, 14:1, 15:1, 16:1, 17:1, 18:1, 19:1, 20:1, 22:1, 25:1, 30:1, 35:1, 40:1, 45:1, 50:1, and 60:1. For example, the wt/wt ratio of the lipid component to a therapeutic and/or prophylactic agent can be from about 10:1 to about 40:1. In certain embodiments, the wt/wt ratio is about 20:1. The amount of a therapeutic and/or prophylactic agent in a nanoparticle composition can, for example, be measured using absorption spectroscopy (e.g., ultraviolet-visible spectroscopy).

In some embodiments, a nanoparticle composition includes one or more RNAs, and the one or more RNAs, lipids, and amounts thereof can be selected to provide a specific N:P ratio. The N:P ratio of the composition refers to the molar ratio of nitrogen atoms in one or more lipids to the number of phosphate groups in an RNA. In some embodiments, a lower N:P ratio is selected. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

The physical properties of a nanoparticle composition can depend on the components thereof. For example, a nanoparticle composition including cholesterol as a structural lipid can have different characteristics compared to a nanoparticle composition that includes a different structural lipid. Similarly, the characteristics of a nanoparticle composition can depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvem Instruments Ltd, Malvem, Worcestershire, UK) may also be used to measure multiple characteristics of a nanoparticle composition, such as particle size, polydispersity index, and zeta potential.

In various embodiments, the mean size of a nanoparticle composition can be between 10s of nm and 100s of nm. For example, the mean size can be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition can be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition can be from about 70 nm to about 100 nm. In some embodiments, the mean size can be about 80 nm. In other embodiments, the mean size can be about 100 nm.

A nanoparticle composition can be relatively homogenous. A polydispersity index can be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition can be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic agent (e.g, a nucleic acid described herein) describes the amount of therapeutic and/or prophylactic agent that is encapsulated or otherwise associated with a nanoparticle composition after preparation, relative to the initial amount provided. The encapsulation efficiency is desirably high (e.g., close to 100%). The encapsulation efficiency can be measured, for example, by comparing the amount of therapeutic and/or prophylactic agent in a solution containing the nanoparticle composition before and after breaking up the nanoparticle composition with one or more organic solvents or detergents. Fluorescence can be used to measure the amount of free therapeutic and/or prophylactic agent (e.g., RNA) in a solution. For the nanoparticle compositions described herein, the encapsulation efficiency of a therapeutic and/or prophylactic agent can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

A nanoparticle composition can optionally comprise one or more coatings. For example, a nanoparticle composition can be formulated in a capsule, film, or tablet having a coating. A capsule, film, or tablet including a composition described herein can have any useful size, tensile strength, hardness, or density.

### 7.5.7 Pharmaceutical Compositions

In one aspect, provided herein are compositions comprising a nanoparticle composition described herein (e.g., a nanoparticle composition described in Section 5.5 or 6), a nucleic acid described herein (e.g., a nucleic acid described in, e.g., Section 5.4 or 6), a vector comprising a nucleic acid described herein (e.g., a vector described in, e.g., Section 5.4 or 6), or a protein, fusion protein, fragment, mutant described herein (e.g., a protein, fusion protein, fragment, mutant described in e.g., Section 5.3 or 6). In some embodiments, provided herein are pharmaceutical compositions comprising a nanoparticle composition described herein (e.g, a nanoparticle composition described in Section 5.5 or 6), a nucleic acid described herein (e.g, a nucleic acid described in, e.g., Section 5.4 or 6), a vector comprising a nucleic acid described herein (e.g., a vector described in, e.g., Section 5.4 or 6), or a protein, fusion protein, fragment mutant described herein (e.g., a protein, fusion protein, fragment, mutant described in e.g., Section 5.3 or 6), and a pharmaceutically acceptable carrier, diluent or excipient.

According to the present disclosure, nanoparticle compositions can be formulated in whole or in part as pharmaceutical compositions. Pharmaceutical compositions can include one or more nanoparticle compositions disclosed herein. For example, a pharmaceutical composition can include one or more nanoparticle compositions including one or more different therapeutic and/or prophylactic agents (e.g., a nucleic acid described herein, such as, e.g., in Section 5.4 or 6). Pharmaceutical compositions can further include one or more pharmaceutically acceptable excipients or accessory ingredients such as those described herein. In some embodiments, provided herein is a pharmaceutical composition comprising a nanoparticle composition described herein (e.g., a nanoparticle composition described in Section 5.5 or 6), and a pharmaceutically acceptable carrier, diluent or excipient. In some embodiments, provided herein is a pharmaceutical composition comprising a nanoparticle composition described herein which includes a nucleic acid described herein (e.g., a nucleic acid described in Section 5.4 or 6), and pharmaceutically acceptable carrier, diluent or excipient. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients can be used in any pharmaceutical composition, except insofar as any conventional excipient or accessory ingredient can be incompatible with one or more components of a nanoparticle composition. An excipient or accessory ingredient can be incompatible with a component of a nanoparticle composition if its combination with the component can result in any undesirable biological effector otherwise deleterious effect.

In some embodiments, one or more excipients or accessory ingredients can make up greater than 50% of the total mass or volume of a pharmaceutical composition including a nanoparticle composition. For example, the one or more excipients or accessory ingredients can make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition can comprise between 0.1% and 100% (wt/wt) of one or more nanoparticle compositions.

In certain embodiments, the nanoparticle compositions and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising a compound according to Series 01, 02, 03, and 04, e.g., a compound of any of Formulae (01-I), (01-II), (02-I), (03-I), and (04-I) (and sub-formulas thereof) is a solution that is refrigerated for storage and/or shipment at, for example, about −20° C., −30° C., −40° C., −50° C., −60° C., −70° C., or −80° C. In certain embodiments, the disclosure also relates to a method of increasing stability of the nanoparticle compositions and/or pharmaceutical compositions comprising a compound according to Series 01, 02, 03, and 04, e.g., a compound of any of Formulae (01-I), (01-II), (02-I), (03-I), and (04-I) (and sub-formulas thereof) by storing the nanoparticle compositions and/or pharmaceutical compositions at a temperature of 4° C. or lower, such as a temperature between about −150° C. and about 0° C. or between about-80° C. and about-20° C., e.g., about-5° C., −10° C., −15° C., −20° C., −25° C., −30° C., −40° C., −50° C., −60° C., −70° C., −80° C., −90° C., −130° C. or −150° C.). For example, the nanoparticle compositions and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.). In one embodiment the formulation is stabilized for at least 4 weeks at about 4° C.

In certain embodiments, the pharmaceutical composition of the disclosure comprises a nanoparticle composition disclosed herein, and a pharmaceutically acceptable carrier selected from one or more of Tris, an acetate (e.g., sodium acetate), a citrate (e.g., sodium citrate), saline, PBS, and sucrose. In certain embodiments, the pharmaceutical composition of the disclosure has a pH value between about 7 and 8 (e.g., 6.8, 6.9, 7.0, 7.1, 7.2, 7.3, 7.4, 7.5, 7.6, 7.7, 7.8, 7.9 or 8.0, or between 7.5 and 8 or between 7 and 7.8). In certain embodiments, a pharmaceutical composition of the disclosure comprises a nanoparticle composition disclosed herein, Tris, saline and sucrose, and has a pH of about 7.5-8, which is suitable for storage and/or shipment at, for example, about −20° C. In certain embodiments, a pharmaceutical composition of the disclosure comprises a nanoparticle composition disclosed herein and PBS and has a pH of about 7-7.8, suitable for storage and/or shipment at, for example, about 4° C. or lower. “Stability,” “stabilized,” and“stable” in the context of the present disclosure refers to the resistance of nanoparticle compositions and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

In certain embodiments, the pharmaceutical composition of the disclosure (e.g, an aqueous composition or a frozen composition) comprises a nanoparticle composition disclosed herein and a buffer. Representative buffers include, but not limited to, acetic acid, adipic acid, ammonia solution, ammonium phosphate, ammonium sulfate, arginine, asparagine, boric acid, calcium carbonate, calcium lactate, tricalcium phosphate, citric acid monohydrate, potassium phosphate dibasic, sodium phosphate dibasic, diethanolamine, glycine, histidine, hydroxyethylpiperazine ethanesulfonic acid, lysine acetate, lysine hydrochloride, maleic acid, malic acid, meglumine, methionine, sodium phosphate monobasic, monoethanolamine, monosodium glutamate, phosphoric acid, potassium citrate, potassium metaphosphate, sodium acetate, sodium bicarbonate, sodium borate, sodium carbonate, sodium citrate, sodium lactate, triethanolamine, tromethamine, phosphate buffer (e.g., sodium phosphate monobasic monohydrate, anhydrous sodium phosphate dibasic), acetate buffer, citrate buffer, borate buffer, and HBSS (Hank's Balanced Salt Solution), trisaminomethane buffer, trisaminomethane hydrochloride buffer, tris(hydroxymethyl)-aminomethane (Tris) buffer, borate buffer and diethylamine buffer and the like.

In certain embodiments, the pharmaceutical composition of the disclosure (e.g, an aqueous composition or a frozen composition) comprises a nanoparticle composition disclosed herein and a buffer, and further comprises one or more cryoprotecants including but not limited to a saccharide, a surfactant, a polyol, an amino acid, a peptide, a protein and/or the like.

Representative saccharides include monosaccharides, disaccharides and polysaccharides. Representative monosaccharides include glucose, fructose, galactose, arabinose, xylose, mannose, lactulose, allose, altrose, gulose, idose, talose, ribose, lyxose and the like. Representative disaccharides include sucrose, trehalose, lactose, maltose, isomaltose, cellobiose and the like. Representative polysaccharides include starch and starch derivatives, cellulose and cellulose derivatives (e.g., methylcellulose, ethylcellulose, hydroxycellulose, hydroxypropylcellulose, hydroxypropylmethylcellulose), chitosan and derivatives thereof, chitin, inulin, chitin, glycogen, pectin, and hyaluronic acid.

Surfactants include non-ionic surfactants, specifically polyoxyethylene fatty alcohol ethers (Brij), polysorbates (e.g., Tween 80, Tween 60, Tween 20, Tween 40), polyoxyethylene fatty acid esters (OEO), polyoxyethylene castor oil derivatives, polyoxyethylene polypropylene glycol copolymers, sucrose fatty acid esters, polyethylene glycol fatty acid esters, polyoxyethylene sorbitan monofatty acid esters, polyoxyethylene sorbitan difatty acid esters, polyoxyethylene glycerol monofatty acid esters, polyoxyethylene glycerol difatty acid esters, polyglycerol fatty acid esters, polypropylene glycol monoesters, arylalkyl polyether alcohols, polyoxyethylene-polyoxypropylene copolymers (poloxamers), polyvinyl alcohol (PVA) derivatives, and polyvinylpyrrolidone (PVP).

Polyols include sugar alcohol, polymeric polyol, low molecular weight polyol and the like. Representative sugar alcohols include maltitol, sorbitol, xylitol, erythritol, isomalt, maltitiol and the like, mannitol, polyglycitol and the like. Representative polymeric polyols include: polyvinyl alcohol, polyethylene glycol, polyether polyol, polyester polyol, polypropylene glycol and the like. Representative low molecular weight polyols include: ethanol, ethylene glycol, propylene glycol, glycerol, trimethylolpropane, butanediol and the like.

In one embodiment, the pharmaceutical composition of the disclosure (e.g., an aqueous composition or a frozen composition) comprises propylene glycol. For example, the concentration of propylene glycol in the pharmaceutical composition ranges from about 0.05% w/v to about 50% w/v, from about 0.05% w/v to about 25% w/v, from about 0.5% w/v to 10% w/v, or from about 1% w/v to about 5% w/v. For example, the concentration of glycerol in the formulation ranges from about 1.5% w/v to about 3% w/v, or from about 2% w/v to about 2.5% w/v.

Representative amino acids include: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

Representative peptides include: diglycine, and triglycine. Proteins comprise: albumin, collagen, casein, and gelatin.

Nanoparticle compositions and/or pharmaceutical compositions including one or more nanoparticle compositions can be administered to any patient or subject, including those patients or subjects that can benefit from a therapeutic effect provided by the delivery of a therapeutic and/or prophylactic agent to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more nanoparticle compositions can be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

In some embodiments, provided herein is a pharmaceutical composition comprising a nucleic acid described herein (e.g., a nucleic acid described in Section 5.4 or 6). In some embodiments, provided herein is a pharmaceutical composition comprising a nucleic acid encoding a protein described herein (e.g., a protein described in Section 5.3). In some embodiments, provided herein is a pharmaceutical composition comprising a nucleic acid encoding a fragment as described herein, a nucleic acid encoding a mutant described herein, a nucleic acid encoding a protein described herein, or a nucleic acid encoding a fusion protein described herein). In some embodiments, provided herein is a pharmaceutical composition comprising a vector (e.g., a vector described in Section 5.4 or 6) comprising a nucleic acid described herein (e.g., a nucleic acid described in Section 5.4 or 6). The vector may be a viral vector or a non-viral vector (e.g., a plasmid). In some embodiments, provided herein is a pharmaceutical composition comprising a protein described herein (e.g., a protein described in Section 5.3). In some embodiments, provided herein is a pharmaceutical composition comprising a fragment as described herein, a mutant described herein, a protein described herein, or a fusion protein described herein. The pharmaceutical composition may include buffer (e.g., a buffer disclosed herein) and/or one or more cryoprotectants (e.g., one or more cryoprotectants disclosed herein).

In some embodiments, provided herein is a pharmaceutical composition comprising a nucleic acid described herein (e.g., a nucleic acid described in Section 5.4 or 6), and a pharmaceutically acceptable carrier, diluent or excipient. In some embodiments, provided herein is a pharmaceutical composition comprising a nucleic acid encoding a protein described herein (e.g., a protein described in Section 5.3), and a pharmaceutically acceptable carrier, diluent or excipient. In some embodiments, provided herein is a pharmaceutical composition comprising a nucleic acid encoding a fragment as described herein, a nucleic acid encoding a mutant described herein, a nucleic acid encoding a protein described herein, or a nucleic acid encoding a fusion protein described herein, and a pharmaceutically acceptable carrier, diluent or excipient. In some embodiments, provided herein is a pharmaceutical composition comprising a vector comprising a nucleic acid described herein.

In some embodiments, provided herein is a pharmaceutical composition comprising a protein described herein (e.g., a protein described in Section 5.3), and a pharmaceutically acceptable carrier, diluent or excipient. In some embodiments, provided herein is a pharmaceutical composition comprising a fragment as described herein, a mutant described herein, a protein described herein, or a fusion protein described herein, and a pharmaceutically acceptable carrier, diluent or excipient.

In specific embodiments, a pharmaceutical composition described herein is an immunogenic composition. For example, in some embodiments, a pharmaceutical composition induces an immune response in a subject (e.g., a human subject) such as described in Section 6 (e.g., a gE specific antibody). In specific embodiments, a pharmaceutical composition described herein is a vaccine composition.

A pharmaceutical composition in accordance with the present disclosure can be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions can be prepared in a variety of forms suitable for a variety of routes and methods of administration. For example, pharmaceutical compositions can be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms can comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutic and/or prophylactic agents, additional agents such as wetting agents, emulsifying and suspending agents, sweetening, flavoring, and/or perfuming agents. In certain embodiments for parenteral administration, compositions are mixed with solubilizing agents such as Cremophor™, alcohols, oils, modified oils, glycols, polysorbates, cyclodextrins, polymers, and/or combinations thereof.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, the pharmaceutical composition comprises a nucleic acid described herein (e.g., a nucleic acid described in Section 5.4 or 6). In some embodiments, the pharmaceutical composition comprises a non-naturally occurring nucleic acid described herein (e.g., a non-naturally occurring nucleic acid described in Section 5.4 or 6). In some embodiments, the pharmaceutical composition comprises a nucleic acid encoding a protein described herein (e.g., a protein described in Section 5.3). In some embodiments, the pharmaceutical composition comprises a nucleic acid encoding a fragment as described herein, a nucleic acid encoding a mutant described herein, a nucleic acid encoding a protein described herein, or a nucleic acid encoding a fusion protein described herein. In some embodiments, the pharmaceutical composition comprises a protein described herein (e.g., a protein described in Section 5.3). In some embodiments, the pharmaceutical composition comprises a fragment as described herein, a mutant described herein, a protein described herein, or a fusion protein described herein. In some embodiments, the pharmaceutical composition comprises the fragment as described herein, or the nucleic acid as described herein. In some embodiments, the pharmaceutical composition comprises the non-naturally occurring nucleic acid as described herein.

In specific embodiments, provided herein is a pharmaceutical composition comprising a non-naturally occurring nucleic acid described herein, and at least a first lipid, optionally, wherein the first lipid is a compound according to Formula 01-I or Formula 01-I; or a compound listed in Table 01-1; or a compound according to Formula 02-I; or a compound listed in Table 02-1; or a compound according to Formula 03-I; or a compound listed in Table 03-1; or a compound according to Formula 04-I; or a compound listed in Table 04-1. In specific embodiments, the pharmaceutical composition further comprises a second lipid, optionally, wherein the second lipid is a compound according to Formula 05-I. In specific embodiments, the pharmaceutical composition is formulated as lipid nanoparticles encapsulating the nucleic acid in a lipid shell.

The disclosure features methods of delivering a therapeutic and/or prophylactic agent (i.e., a polypeptide of interest) to a mammalian cell or organ, and treating a disease or disorder in a mammal in need thereof comprising administering to a mammal and/or contacting a mammalian cell with a therapeutic and/or prophylactic agent (i.e., a polypeptide of interest).

The disclosure features methods of delivering a therapeutic and/or prophylactic agent to a mammalian cell or organ, producing a polypeptide of interest in a mammalian cell, and treating a disease or disorder in a mammal in need thereof comprising administering to a mammal and/or contacting a mammalian cell with a nanoparticle composition including a therapeutic and/or prophylactic agent.

### 7.6 Methods

In one aspect, provided herein are also methods for managing, preventing and treating a disease or disorder caused by VZV or by infection with VZV in a subject (e.g., a human subject). In some embodiments, provided herein is a method for managing, preventing and/or treating a disease or disorder caused by VZV orby infection with VZV in a subject (e.g., a human subject), comprising administering to the subject a protein described herein (e.g., in Section 5.3 or 6), a fragment described herein (e.g., in Section 5.3 or 6), a fusion protein described herein (e.g., in Section 5.3 or 6), a nucleic acid (including a non-naturally occurring nucleic acid described herein) (e.g., in Section 5.4 or 6), a vector described herein (e.g., in Section 5.4 or 6), or a pharmaceutical composition described herein (e.g., in Section 5.5 or 6). In some embodiments, the disease or disorder being managed, prevented or treated with the methods described herein is caused by VZV or by infection with VZV. In specific embodiments, the methods provided herein are for the prevention of a disease or disorder caused by VZV. In some embodiments, the methods inhibit VZV infection. In some embodiments, the methods inhibit reactivation of a latent VZV infection.

In specific embodiments, the disease or disorder being managed, prevented or treated with the methods described herein is one or more of varicella, zoster, postherpetic neuralgia (PHN), meningoencephalitis, myelitis, cranial nerve palsy, angiopathy, keratitis, retinopathy, ulcers, hepatitis and pancreatitis.

In some embodiments, the present method for managing, preventing and treating a disease or disorder caused by VZV or by infection with VZV in a subject comprises administering to the subject a therapeutic effective amount of a fragment, a nucleic acid or a therapeutic nucleic acid as described herein. In specific embodiments, the therapeutic nucleic acid is an mRNA molecule as described herein.

In some embodiments, the present method for managing, preventing and treating a disease or disorder caused by VZV or by infection with VZV in a subject comprises administering to the subject a therapeutic effective amount of a therapeutic composition comprising a fragment, a nucleic acid or a therapeutic nucleic acid as described herein. In specific embodiments, the therapeutic nucleic acid is an mRNA molecule as described herein.

In some embodiments, the present method for managing, preventing and treating a disease or disorder caused by VZV or by infection with VZV in a subject comprises administering to the subject a therapeutic effective amount of a vaccine composition comprising a fragment, a nucleic acid or a therapeutic nucleic acid as described herein. In specific embodiments, the therapeutic nucleic acid is an mRNA molecule as described herein.

In some embodiments, the present method for managing, preventing and treating a disease or disorder caused by VZV or by infection with VZV in a subject comprises administering to the subject a therapeutic effective amount of a lipid-containing composition comprising a therapeutic nucleic acid as described herein. In specific embodiments, the therapeutic nucleic acid is an mRNA molecule as described herein.

In some embodiments, the present method for managing, preventing and treating a disease or disorder caused by VZV or by infection with VZV in a subject comprises administering to the subject a therapeutic effective amount of a lipid-containing composition comprising a therapeutic nucleic acid as described herein, wherein the lipid-containing composition is formulated as a lipid nanoparticle encapsulating the therapeutic nucleic acid in a lipid shell. In specific embodiments, the therapeutic nucleic acid is an mRNA molecule as described herein. In specific embodiments, the cells in the subject effectively intake the lipid-containing composition (e.g., lipid nanoparticles) described herein upon administration. In specific embodiments, lipid-containing composition (e.g., lipid nanoparticles) described herein are endocytosed by cells of the subject.

In some embodiments, provided herein is a method for preventing a disease or disorder caused by VZV in a subject (e.g., a human subject), comprising administering to the subject a prophylactically effective amount of a protein described herein (e.g., a protein described in Section 5.3 or 6), or a nucleic acid described herein (e.g., a nucleic acid described in Section 5.4 or 6). In some embodiments, provided herein is a method for preventing a disease or disorder caused by VZV in a subject (e.g., a human subject), comprising administering to the subject a prophylactically effective amount of a fragment (e.g., a fragment described in Section 5.4 or 6), a mutant described herein (e.g., a mutant described in Section 5.3 or 6), or a fusion protein described herein (e.g., a fusion protein described in Section 5.3 or 6). In some embodiments, provided herein is a method for preventing a disease or disorder caused by VZV in a subject (e.g., a human subject), comprising administering to the subject a prophylactically effective amount of a vector described herein (e.g., a vector described in Section 5.4.10). In some embodiments, provided herein is a method for preventing a disease or disorder caused by VZV in a subject (e.g., a human subject), comprising administering to the subject a prophylactically effective amount of a nucleic acid described herein (e.g., a nucleic acid described in Section 5.4 or 6). In some embodiments, the nucleic acid is non-naturally occurring. In some embodiments, the nucleic acid is mRNA. In specific embodiments, the nucleic acid is a non-naturally occurring mRNA. In specific embodiments, the disease or disorder includes one or more of varicella, herpes zoster (or shingles), postherpetic neuralgia (PHN), meningoencephalitis, myelitis, cranial nerve palsy, angiopathy, keratitis, retinopathy, ulcers, hepatitis and pancreatitis. In specific embodiments, the disease or disorder is herpes zoster (or shingles).

In some embodiments, provided herein is a method for preventing a disease or disorder caused by VZV in a subject (e.g., a human subject), comprising administering to the subject a prophylactically effective amount of a lipid-containing composition comprising a nucleic acid described herein. In some embodiments, the nucleic acid is non-naturally occurring. In some embodiments, the nucleic acid is mRNA. In specific embodiments, the nucleic acid is a non-naturally occurring mRNA. In specific embodiments, the disease or disorder includes one or more of varicella, herpes zoster (or shingles), postherpetic neuralgia (PHN), meningoencephalitis, myelitis, cranial nerve palsy, angiopathy, keratitis, retinopathy, ulcers, hepatitis and pancreatitis. In specific embodiments, the disease or disorder is herpes zoster (or shingles).

In some embodiments, provided herein is a method for preventing a disease or disorder caused by VZV in a subject (e.g., a human subject), comprising administering to the subject a prophylactically effective amount of a lipid-containing composition comprising a nucleic acid described herein, wherein the lipid-containing composition is formulated as a lipid nanoparticle encapsulating the nucleic acid in a lipid shell. In some embodiments, the nucleic acid is non-naturally occurring. In some embodiments, the nucleic acid is mRNA. In specific embodiments, the nucleic acid is a non-naturally occurring mRNA. In specific embodiments, the cells in the subject effectively intake the lipid-containing composition (e.g., lipid nanoparticles) described herein upon administration. In specific embodiments, lipid-containing composition (e.g., lipid nanoparticles) described herein are endocytosed by cells of the subject. In specific embodiments, the disease or disorder includes one or more of varicella, herpes zoster (or shingles), postherpetic neuralgia (PHN), meningoencephalitis, myelitis, cranial nerve palsy, angiopathy, keratitis, retinopathy, ulcers, hepatitis and pancreatitis. In specific embodiments, the disease or disorder is herpes zoster (or shingles).

In some embodiments, provided herein is a method for preventing a disease or disorder caused by VZV in a subject (e.g., a human subject), comprising administering to the subject a prophylactically effective amount of a nanoparticle composition described herein (e.g, a nanoparticle composition described in Section 5.5 or 6), wherein the nanoparticle composition encapsidates a nucleic acid described herein (e.g., a nucleic acid described in Section 5.4 or 6). In some embodiments, the nucleic acid is non-naturally occurring. In some embodiments, the nucleic acid is mRNA. In specific embodiments, the nucleic acid is a non-naturally occurring mRNA. In specific embodiments, the cells in the subject effectively intake the nanoparticle composition upon administration. In specific embodiments, the nanoparticle composition is endocytosed by cells of the subject. In specific embodiments, the disease or disorder includes one or more of varicella, herpes zoster (or shingles), postherpetic neuralgia (PHN), meningoencephalitis, myelitis, cranial nerve palsy, angiopathy, keratitis, retinopathy, ulcers, hepatitis and pancreatitis. In specific embodiments, the disease or disorder is herpes zoster (or shingles).

In some embodiments, provided herein is a method for preventing a disease or disorder caused by VZV in a subject (e.g., a human subject), comprising administering to the subject a prophylactically effective amount of a pharmaceutical composition described herein (e.g., a pharmaceutical composition described in Section 5.5.7). In specific embodiments, the disease or disorder includes one or more of varicella, herpes zoster, postherpetic neuralgia (PHN), meningoencephalitis, myelitis, cranial nerve palsy, angiopathy, keratitis, retinopathy, ulcers, hepatitis and pancreatitis. In specific embodiments, the disease or disorder is herpes zoster (or shingles).

Without being bound by any theory, the nucleic acid described herein which is administered to the subject (e.g., human subject) is taken up by cells in the subject, the protein encoded by the nucleic acid is expressed, and the protein induces an immune response in the subject. In some embodiments, the protein induces antibody specific to VZV gE (e.g., IgG). In some embodiments, the antibody specific to VZV gE is a neutralizing antibody. In some embodiments, the immune response induced by the protein includes one or more responses described in Section 5.6.1 or 6. Without being bound by any theory, the immune response induced by the protein provides protection against a disease or disorder caused by VZV. The protection can be full or partial protection against a disease or disorder caused by VZV (e.g., herpes zoster).

In some embodiments, upon administration to a subject in need thereof of the therapeutic nucleic acid as described herein, a vaccine composition comprising the therapeutic nucleic acids described herein, a lipid-containing composition (e.g., lipid nanoparticles) comprising the therapeutic nucleic acids described herein, the cells in the subject uptake and express the administered therapeutic nucleic acids to produce a peptide or polypeptide encoded by the nucleic acid. In some embodiments, the encoded peptide or polypeptide is derived from VZV causing the disease or disorder being managed, prevented, or treated by the method.

### 7.6.1 Immune Responses

In some embodiments, upon administration of a nucleic acid described herein (e.g., a nucleic acid described in Section 5.4 or 6) or a vector described herein (e.g., a vector described in Section 5.4.10) to a subject in need thereof, one or more immune responses against VZV is elicited in the subject. In some embodiments, upon administration of a lipid-containing composition comprising a nucleic acid described herein, wherein the lipid-containing composition is formulated as a lipid nanoparticle encapsulating the nucleic acid in a lipid shell, to a subject in need thereof, one or more immune responses against VZV is elicited in the subject. In some embodiments, upon administration of a nanoparticle composition described herein (e.g., a nanoparticle composition described in Section 5.5 or 6), wherein the nanoparticle composition encapsidates a nucleic acid described herein (e.g., a nucleic acid described in Section 5.4 or 6), to a subject in need thereof, one or more immune responses against VZV is elicited in the subject. In some embodiments, upon administration of a protein described herein (e.g., a protein described in Section 5.3 or 6) to a subject in need thereof, one or more immune responses against VZV is elicited in the subject. In some embodiments, upon administration of a fragment described herein (e.g., a fragment described in Section 5.3 or 6), a mutant (e.g., a mutant described in Section 5.3 or 6), or a fusion protein described herein (e.g., a fusion protein described in Section 5.3 or 6) to a subject in need thereof, one or more immune responses against VZV is elicited in the subject. In some embodiments, upon administration of a pharmaceutical composition described herein (e.g., a pharmaceutical composition described in Section 5.5.7) to a subject in need thereof, one or more immune responses against VZV is elicited in the subject. The one or more immune responses can be in the form of, e.g., an antibody response (humoral response) or a cellular immune response, e.g., cytokine secretion (e.g., interferon-gamma), helper activity or cellular cytotoxicity. In some embodiments, expression of an activation marker on immune cells, expression of a co-stimulatory receptor on immune cells, expression of a ligand for a co-stimulatory receptor, cytokine secretion, infiltration of immune cells (e.g., T-lymphocytes, B lymphocytes and/or NK cells) to a infected cell, production of antibody specifically recognizing one or more viral proteins (e.g., the viral peptide or protein encoded by the therapeutic nucleic acid), effector function, T cell activation, T cell differentiation, T cell proliferation, B cell differentiation, B cell proliferation, and/or NK cell proliferation is induced, activated and/or enhanced. In some embodiments, activation and proliferation of myeloid-derived suppressor cell (MDSC) and Treg cells are inhibited. In some embodiments, the one or more immune responses include an antibody response. In some embodiments, the antibody response includes the production of an antibody that specifically binds to VZV gE (e.g., a VZV gE neutralizing antibody). In some embodiments, the one or more immune responses include production of cytokine in lymphocytes (e.g., increased production of cytokine in lymphocytes) and/or increased proportion of cytokine-expressing lymphocytes). In some embodiments, the immune response includes an increase in the production of one or more cytokines (e.g., IFN-γ, IL-2, and/or TNF-α) by one or more populations of lymphocytes. In some embodiments, the lymphocytes are CD4⁺ T cells and/or CD8⁺ T cells. In some embodiments, the one or more immune responses is one or more of the immune responses described in Section 6.

In some embodiments, upon administration to a subject in need thereof of the fragment, the nucleic acid or the therapeutic nucleic acid as described herein, a vaccine composition comprising the fragment, the nucleic acid or the therapeutic nucleic acids described herein, a lipid-containing composition (e.g., lipid nanoparticles) comprising the therapeutic nucleic acids described herein, one or more immune responses against VZV is elicited in the subject. In some embodiments, the elicited immune response comprises one or more adaptive immune responses against VZV. In some embodiments, the elicited immune response comprises one or more innate immune responses against VZV. The one or more immune responses can be in the form of, e.g., an antibody response (humoral response) or a cellular immune response, e.g., cytokine secretion (e.g., interferon-gamma), helper activity or cellular cytotoxicity. In some embodiments, expression of an activation marker on immune cells, expression of a co-stimulatory receptor on immune cells, expression of a ligand for a co-stimulatory receptor, cytokine secretion, infiltration of immune cells (e.g., T-lymphocytes, B lymphocytes and/or NK cells) to a infected cell, production of antibody specifically recognizing one or more viral proteins (e.g., the viral peptide or protein encoded by the therapeutic nucleic acid), effector function, T cell activation, T cell differentiation, T cell proliferation, B cell differentiation, B cell proliferation, and/or NK cell proliferation is induced, activated and/or enhanced. In some embodiments, activation and proliferation of myeloid-derived suppressor cell (MDSC) and Treg cells are inhibited.

In some embodiments, upon administration to a subject in need thereof of the fragment, the nucleic acid or the therapeutic nucleic acid as described herein, a vaccine composition comprising the fragment, the nucleic acid or the therapeutic nucleic acids described herein, a lipid-containing composition (e.g., lipid nanoparticles) comprising the therapeutic nucleic acids described herein, one or more populations of lymphocytes producing cytokines is increased in the subject. In some embodiments, the lymphocytes are CD4⁺ T cells and/or CD8⁺ T cells. In some embodiments, the cytokine is one or more of IFN-γ, IL-2, and TNF-α. In some embodiments, the proportion of IFN-γ expressing CD4+ cells is increased. In some embodiments, the proportion of IL-2 expressing CD4+ cells is increased. In some embodiments, the proportion of TNF-α expressing CD4+ cells is increased. In some embodiments, the proportion of IFN-γ expressing CD8+ cells is increased. In some embodiments, the proportion of IL-2 expressing CD8+ cells is increased. In some embodiments, the proportion of TNF-α expressing CD8+ cells is increased.

In specific embodiments, the neutralizing antibody specifically binds to one or more epitopes of the VZV gE protein and inhibits or reduces one or more gE protein function or activity.

In specific embodiments, the neutralizing antibody binds to one or more viral proteins present on a viral particle or the surface of infected cells, and mark the viral particles or infected cells for destruction by the subject's immune system. In some embodiments, endocytosis of viral particles by white blood cells (e.g., macrophage) is induced or enhanced. In some embodiments, antibody-dependent cell-mediated cytotoxicity (ADCC) against infected cells in the subject is induced or enhanced. In some embodiments, antibody-dependent cellular phagocytosis (ADCP) against infected cells in the subject is induced or enhanced. In some embodiments, complement dependent cytotoxicity (CDC) against infected cells in the subject is induced or enhanced.

### 7.6.2 Combination Therapy

In some embodiments, the composition of the present disclosure can further comprise one or more additional therapeutic agents. In some embodiments, the additional therapeutic agent is an adjuvant capable of bolstering immunogenicity of the composition (e.g., a genetic vaccine). In some embodiments, the additional therapeutic agent is an immune modulator that enhances immune responses in a subject. In some embodiments, the adjuvant and the therapeutic nucleic acid in the composition can have a synergistic action in eliciting an immune response in a subject.

In some embodiments, the additional therapeutic agent and the therapeutic nucleic acid of the present disclosure can be co-formulated in one composition. For example, the additional therapeutic agent can be formulated as part of the composition comprising the therapeutic nucleic acid of the present disclosure. Alternatively, in some embodiments, the additional therapeutic agent and therapeutic nucleic acid of the present disclosure can be formulated as separate compositions or dose units for co-administration either sequentially or simultaneously to a subject.

In particular embodiments, the therapeutic nucleic acid of the present disclosure is formulated as part of a lipid-containing composition as described in Section 5.5, and the additional therapeutic agent is formulated as a separate composition. In particular embodiments, the therapeutic nucleic acid of the present disclosure is formulated as part of a lipid-containing composition as described in Section 5.5, wherein the additional therapeutic agent is also formulated as part of the lipid-containing composition.

In particular embodiments, the therapeutic nucleic acid of the present disclosure is formulated so that the therapeutic nucleic acid is encapsulated in a lipid shell of a lipid nanoparticle as described in Section 5.5, and the additional therapeutic agent is formulated as a separate composition. In particular embodiments, the therapeutic nucleic acid of the present disclosure is formulated so that the therapeutic nucleic acid is encapsulated in a lipid shell of a lipid nanoparticle as described in Section 5.5, wherein the lipid nanoparticles also enclose the additional therapeutic agent molecule or a nucleic acid encoding the additional therapeutic agent molecule. In particular embodiments, the therapeutic nucleic acid of the present disclosure is formulated so that the therapeutic nucleic acid is encapsulated in a lipid shell of a lipid nanoparticle as described in Section 5.5, wherein the lipid nanoparticles and the additional therapeutic agent are formulated into a single composition.

In specific embodiments, the additional therapeutic agent is an adjuvant. In some embodiments, the adjuvant comprises an agent that promotes maturation of dendritic cells (DCs) in a vaccinated subject, such as but not limited to lipopolysaccharides, TNF-alpha or CD40 ligand. In some embodiments, the adjuvant is an agent that recognized by the immune system of the vaccinated subject as a “danger signal,” such as LPS, GP96, etc.

In some embodiments, the adjuvant comprises an immunostimulating cytokine such as but not limited to IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, INF-gamma, GM-CSF, G-CSF, M-CSF, LT-beta or TNF-alpha, growth factors, such as hGH.

In some embodiments, the adjuvant comprises a compound known as capable of eliciting an innate immune response. One exemplary class of such compound are Toll-like receptor ligands, such as ligands of human Toll-like receptors TLR1, TLR2, TLR3, TLR4, TLR5, TLR6, TLR7, TLR8, TLR9, TLR10, and ligands of murine Toll-like receptors TLR1, TLR2, TLR3, TLR4, TLR5, TLR6, TLR7, TLR8, TLR9, TLR10, TLR11, TLR12 or TLR13. Another exemplar class of such compounds are immuno-stimulating nucleic acids, such as oligonucleotides containing the CpG motif. CpG containing nucleic acids can be DNA (CpG-DNA) or RNA (CpG-RNA) molecules. A CpG-RNA or CpG-DNA can be a single-stranded CpG-DNA (ss CpG-DNA), a double-stranded CpG-DNA (dsDNA), a single-stranded CpG-RNA (ss CpG-RNA) or a double-stranded CpG-RNA (ds CpG-RNA). In some embodiments, the CpG nucleic acid is in the form of CpG-RNA. In particular embodiments, the CpG nucleic acid is in the form of single-stranded CpG-RNA (ss CpG-RNA). In some embodiments, the CpG nucleic acid contains at least one or more (mitogenic) cytosine/guanine dinucleotide sequence(s) (CpG motif(s)). In some embodiments, at least one CpG motif contained in these sequences (i.e., the C (cytosine) and/or the G (guanine) forming the CpG motif) is unmethylated.

In some embodiments, the additional therapeutic agent is an immune modulator that activate, boost or restore normal immune functions. In specific embodiments, the immune modulator is an agonist of a co-stimulatory signal of an immune cell, such as a T-lymphocyte, NK cell or antigen-presenting cell (e.g., a dendritic cell or macrophage). In specific embodiments, the immune modulator is an antagonist of an inhibitory signal of an immune cell, such as a T-lymphocyte, NK cell or antigen-presenting cell (e.g., a dendritic cell or macrophage).

Various immune cell stimulatory agents are known to one of skill in the art and can be used in connection with the present disclosure. In certain embodiments, the agonist of a co-stimulatory signal is an agonist of a co-stimulatory molecule (e.g., co-stimulatory receptor) found on immune cells, such as, T-lymphocytes (e.g., CD4+ or CD8+T-lymphocytes), NK cells and/or antigen-presenting cells (e.g., dendritic cells or macrophages). Specific examples of co-stimulatory molecules include glucocorticoid-induced tumor necrosis factor receptor (GITR), Inducible T-cell costimulator (ICOS or CD278), OX40 (CD134), CD27, CD28, 4-IBB (CD137), CD40, lymphotoxin alpha (LT alpha), LIGHT (lymphotoxin-like, exhibits inducible expression, and competes with herpes simplex virus glycoprotein D for HVEM, a receptor expressed by T lymphocytes), CD226, cytotoxic and regulatory T cell molecule (CRT AM), death receptor 3 (DR3), lymphotoxin-beta receptor (LTBR), transmembrane activator and CAML interactor (TACI), B cell-activating factor receptor (BAFFR), and B cell maturation protein (BCMA).

In specific embodiments, the agonist of a co-stimulatory receptor is an antibody or antigen-binding fragment thereof that specifically binds to the co-stimulatory receptor. Specific examples of co-stimulatory receptors include GITR, ICOS, OX40, CD27, CD28, 4-1BB, CD40, LT alpha, LIGHT, CD226, CRT AM, DR3, LTBR, TACI, BAFFR, and BCMA. In certain specific embodiments, the antibody is a monoclonal antibody. In other specific embodiments, the antibody is an sc-Fv. In a specific embodiment, the antibody is a bispecific antibody that binds to two receptors on an immune cell. In other embodiments, the bispecific antibody binds to a receptor on an immune cell and to another receptor on a virus infected diseased cell. In specific embodiments, the antibody is a human or humanized antibody.

In another embodiment, the agonist of a co-stimulatory receptor is a ligand of the co-stimulatory receptor or a functional derivative thereof. In certain embodiments, the ligand is fragment of a native ligand. Specific examples of native ligands include ICOSL, B7RP1, CD137L, OX40L, CD70, herpes virus entry mediator (HVEM), CD80, and CD86. The nucleotide sequences encoding native ligands as well as the amino acid sequences of native ligands are known in the art.

In specific embodiments, the antagonist is an antagonist of an inhibitory molecule (e.g., inhibitory receptor) found on immune cells, such as, e.g., T-lymphocytes (e.g., CD4+ or CD8+T-lymphocytes), NK cells and/or antigen-presenting cells (e.g., dendritic cells or macrophages). Specific examples of inhibitory molecules include cytotoxic T-lymphocyte-associated antigen 4 (CTLA-4 or CD52), programmed cell death protein 1 (PD1 or CD279), B and T-lymphocyte attenuator (BTLA), killer cell immunoglobulin-like receptor (KIR), lymphocyte activation gene 3 (LAG3), T-cell membrane protein 3 (TIM3), CD 160, adenosine A2a receptor (A2aR), T cell immunoreceptor with immunoglobulin and ITIM domains (TIGIT), leukocyte-associated immunoglobulin-like receptor 1 (LAIR1), and CD 160.

In another embodiment, the antagonist of an inhibitory receptor is an antibody (or an antigen-binding fragment) that specifically binds to the native ligand for the inhibitory receptor and blocks the native ligand from binding to the inhibitory receptor and transducing an inhibitory signal(s). In certain specific embodiments, the antibody is a monoclonal antibody. In other specific embodiments, the antibody is an sc-Fv. In a specific embodiment, the antibody is a bispecific antibody that binds to two receptors on an immune cell. In other embodiments, the bispecific antibody binds to a receptor on an immune cell and to another receptor on a vims infected diseased cell. In specific embodiments, the antibody is a human or humanized antibody.

In another embodiment, the antagonist of an inhibitory receptor is a soluble receptor or a functional derivative thereof that specifically binds to the native ligand for the inhibitory receptor and blocks the native ligand from binding to the inhibitory receptor and transducing an inhibitory signal(s). Specific examples of native ligands for inhibitory receptors include PDL-1, PDL-2, B7-H3, B7-H4, HVEM, Gal9 and adenosine. Specific examples of inhibitory receptors that bind to a native ligand include CTLA-4, PD-1, BTLA, KIR, LAG3, TIM3, and A2aR.

In another embodiment, the antagonist of an inhibitory receptor is an antibody (or an antigen-binding fragment) or ligand that binds to the inhibitory receptor, but does not transduce an inhibitory signal(s). Specific examples of inhibitory receptors include CTLA-4, PD1, BTLA, KIR, LAG3, TIM3, and A2aR. In certain specific embodiments, the antibody is a monoclonal antibody. In other specific embodiments, the antibody is an scFv. In particular embodiments, the antibody is a human or humanized antibody. A specific example of an antibody to inhibitory receptor is anti-CTLA-4 antibody (Leach D R, et al. Science 1996; 271: 1734-1736). Another example of an antibody to inhibitory receptor is anti-PD-1 antibody (Topalian SL, NEJM 2012; 28:3167-75).

### 7.6.3 Patient Population

In some embodiments, a nucleic acid described herein (e.g., a nucleic acid described in, e.g., Section 5.4 or 6) is administered to a subject in need thereof. In some embodiments, the nucleic acid is non-naturally occurring. In some embodiments, the nucleic acid is mRNA. In some embodiments, the nucleic acid is a non-naturally occurring mRNA. In some embodiments, a vector described herein (e.g., a vector described in Section 0.5.3.10) is administered to a subject in need thereof. In some embodiments, a lipid-containing composition comprising a nucleic acid described herein is administered to a subject in need thereof. In some embodiments, a nanoparticle composition described herein (e.g., a nanoparticle composition described in Section 5.5 or 6) is administered to a subject in need thereof. In some embodiments, a protein described herein (e.g., a protein described in e.g., Section 5.3 or 6) is administered to a subject in need thereof. In some embodiments, a fragment described herein (e.g., a fragment described in e.g., Section 5.3 or 6) is administered to a subject in need thereof. In some embodiments, a mutant described herein (e.g., a mutant described in e.g., Section 5.3 or 6) is administered to a subject in need thereof. In some embodiments, a fusion protein described herein (e.g., a fusion protein described in e.g., Section 5.3 or 6) is administered to a subject in need thereof. In some embodiments, a pharmaceutical composition described herein (e.g., a pharmaceutical composition described in Section 5.5.7) is administered to a subject in need thereof.

In some embodiments, the subject is human. In some embodiments, the subject is a human adult. In some embodiments, the subject is a human at least 40 years old, at least 45 years, at least 50 years old, at least 55 years old, or at least 60 years old. In some embodiments, the subject is a human adult. In some embodiments, the subject is a human about 40 years old, about 41 years old, about 42 years old, about 43 years old, about 44 years old, or about 45 years. In some embodiments, the subject is a human about 45 years old, about 46 years old, about 47 years old, about 48 years old, about 49 years old, or about 50 years old. In some embodiments, the subject is a human about 51 years old, about 52 years old, about 53 years old, about 54 years old, or about 55 years old. In some embodiments, the subject is a human about 56 years old, about 57 years old, about 58 years old, about 59 years old, or about 60 years old. In some embodiments, the subject is an elderly human. In some embodiments, the subject is a human infant. In some embodiments, the subject is a human toddler. In some embodiments, non-human mammal. In some embodiments, the subject (e.g., human, such as, e.g., a human adult or elderly human) has a condition impacting the subject's immune system. In some embodiments, the subject (e.g., human, such as, e.g., a human adult or elderly human) is immunodeficient or immunosuppressed. In some embodiments, the subject (e.g., human, such as, e.g., a human adult or elderly human) is at risk for herpes zoster (or shingles). In some embodiments, the subject (e.g., human, such as, e.g., a human adult or elderly human) has a latent VZV infection. In some embodiments, the subject (e.g., human, such as, e.g., a human adult or elderly human) is susceptible for reactivation of a latent VZV infection.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein is administered to a subject in need thereof.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein is administered to a human subject. In some embodiments, a subject administered with a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising a therapeutic nucleic acids described herein, or a combination therapy described herein is an elderly human. In some embodiments, a subject administered with a fragment, a nucleic acid or a therapeutic nucleic acid described herein, a vaccine composition comprising the fragment the nucleic acid or the therapeutic nucleic acids described herein, a lipid-containing composition (e.g., lipid nanoparticles) comprising the therapeutic nucleic acids described herein or a combination therapy described herein is a human adult. In some embodiments, a subject administered with a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein or a combination therapy described herein is human child. In some embodiments, a subject administered with a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein or a combination therapy described herein is human toddler. In some embodiments, a subject administered with a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein or a combination therapy described herein is human infant.

In some embodiments, a subject administered with a fragment, a nucleic acid or a therapeutic nucleic acid described herein, a vaccine composition comprising the fragment the nucleic acid or the therapeutic nucleic acids described herein, a lipid-containing composition (e.g., lipid nanoparticles) comprising the therapeutic nucleic acids described herein or a combination therapy described herein is administered to a non-human mammal.

In some embodiments, a subject administered with a fragment, a nucleic acid or a therapeutic nucleic acid described herein, a vaccine composition comprising the fragment the nucleic acid or the therapeutic nucleic acids described herein, a lipid-containing composition (e.g., lipid nanoparticles) comprising the therapeutic nucleic acids described herein, or the combination therapy described herein is administered to a subject exhibiting at least one symptom associated with VZV infection. In some embodiments, the subject receiving administration of a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein exhibits one or more symptoms of varicella, zoster, postherpetic neuralgia (PHN), meningoencephalitis, myelitis, cranial nerve palsy, angiopathy, keratitis, retinopathy, ulcers, hepatitis and pancreatitis.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy as described herein is administered to a subject that is asymptomatic for VZV infection.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein is administered to a subject who is at risk of, or susceptible to, VZV infection. In some embodiments, a subject at risk of, or susceptible to, VZV infection is an elderly human. In some embodiments, a subject at risk of, or susceptible to, VZV infection is a human adult. In some embodiments, a subject at risk of, or susceptible to, VZV infection is a human child. In some embodiments, a subject at risk of, or susceptible to, VZV infection is a human adult toddler. In some embodiments, a subject at risk of, or susceptible to, VZV infection is a human adult infant. In some embodiments, a subject at risk of, or susceptible to, VZV infection is a human subject having existing health condition that affects the subject's immune system. In some embodiments, a subject at risk of, or susceptible to, VZV infection is a human subject having existing health condition that affects the subject's major organs. In some embodiments, a subject at risk of, or susceptible to, VZV infection is a human subject having existing health condition that affects the subject's lung function. In some embodiments, a subject at risk of, or susceptible to, VZV infection is an elderly human subject having an existing health condition that affects the subject's immune system, or a major organ, such as lung function. In various embodiments described in this paragraph, a subject at risk of, or susceptible to, VZV infection can be either exhibiting symptoms of VZV infection or asymptomatic for VZV infection.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein is administered to a subject who has been diagnosed positive for VZV infection. In some embodiments, the subject diagnosed positive for VZV infection is asymptomatic for VZV infection, and the diagnosis is based on detecting the presence of a viral nucleic acid or protein from a sample taken from the subject. In some embodiments, the diagnosis is based on clinical symptoms exhibited by the patient. Exemplary symptoms that may serve as the basis of diagnosis include but are not limited to upper respiratory tract infection, lower respiratory tract infection, lung infection, renal infection, liver infection, enteric infection, hepatic infection, neurologic infections, respiratory syndrome, pneumonia, gastroenteritis, encephalomyelitis, encephalitis, sarcoidosis, diarrhea, hepatitis, and demyelinating disease. In some embodiments, the diagnosis is based on a subject's exhibited clinical symptom combined with the subject's history of being in contact with a geographical location, population, and/or individual considered of having a high risk of carrying VZV, such as another individual diagnosed positive for VZV infection.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein is administered to a subject who has not previously received administration of the fragment, the nucleic acid or the therapeutic nucleic acid, the vaccine composition, the lipid-containing composition (e.g., lipid nanoparticles), or the combination therapy.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein is administered to a subject who has previously received administration of the fragment, the nucleic acid or the therapeutic nucleic acid, the vaccine composition, the lipid-containing composition (e.g., lipid nanoparticles), or the combination therapy. In specific embodiments, the subject has been previously administered a fragment a nucleic acid or a therapeutic nucleic acid described herein, the vaccine composition comprising the fragment, the nucleic acid or the therapeutic nucleic acids described herein, the lipid-containing composition (e.g., lipid nanoparticles) comprising the therapeutic nucleic acids described herein, or the combination therapy as described herein once, twice, three times or more.

In some embodiments, a fragment, a nucleic acid or a therapeutic nucleic acid described herein, a vaccine composition comprising the fragment, the nucleic acid or the therapeutic nucleic acids described herein, a lipid-containing composition (e.g., lipid nanoparticles) comprising the therapeutic nucleic acids described herein, or a combination therapy described herein is administered to a subject who has received a therapy prior to administration of the fragment, the nucleic acid or the therapeutic nucleic acid, the vaccine composition, the lipid-containing composition (e.g., lipid nanoparticles), or the combination therapy. In some embodiments, the subject administered with a fragment, a nucleic acid or a therapeutic nucleic acid described herein, a vaccine composition comprising the fragment; the nucleic acid or the therapeutic nucleic acids described herein, a lipid-containing composition (e.g., lipid nanoparticles) comprising the therapeutic nucleic acids described herein, or a combination therapy described herein experienced adverse side effects to a prior therapy or a prior therapy was discontinued due to unacceptable levels of toxicity to the subject.

### 7.6.4 Administration Dosage and Frequency

The amount of fragment, nucleic acid or therapeutic nucleic acid or a composition thereof which will be effective in the management, prevention and/or treatment of infectious disease will depend on the nature of the disease being treated, the route of administration, the general health of the subject, etc. and should be decided according to the judgment of a medical practitioner. Standard clinical techniques, such as in vitro assays, may optionally be employed to help identify optimal dosage ranges. Nevertheless, suitable dosage ranges of a therapeutic protein or nucleic acid as described herein for administration are generally about 0.001 mg, 0.005 mg, 0.01 mg, 0.05 mg. 0.1 mg. 0.5 mg, 1.0 mg, 2.0 mg. 3.0 mg, 4.0 mg, 5.0 mg, 10.0 mg, 0.001 mg to 10.0 mg, 0.01 mg to 1.0 mg, 0.1 mg to 1 mg, and 0.1 mg to 5.0 mg. The therapeutic protein or nucleic acid or a composition thereof can be administered to a subject once, twice, three, four or more times with intervals as often as needed. In some embodiments, a nucleic acid described herein (e.g., a nucleic acid described in Section 5.4 or 6), a protein, fusion protein, fragment or mutant described herein (e.g., a protein, fusion protein, fragment or mutant described in Section 5.3 or 6), a vector described herein (e.g., a vector described in Section 5.4.10), a nanoparticle composition described herein (e.g., a nanoparticle composition described in Section 5.5 or 6), or a pharmaceutical composition described herein (e.g., in Section 5.5.7) is administered at least once to a subject. In some embodiments, a nucleic acid described herein (e.g., a nucleic acid described in Section 5.4 or 6), a protein, fusion protein, fragment or mutant described herein (e.g., a protein, fusion protein, fragment or mutant described in Section 5.3 or 6), a vector described herein (e.g., a vector described in Section 5.4.10), a nanoparticle composition described herein (e.g., a nanoparticle composition described in Section 5.5 or 6), or a pharmaceutical composition described herein (e.g., in Section 5.5.7) is administered twice to a subject. In some embodiments, the second administration is administered 1 to 2 months, 2 to 3 months, 2 to 6 months, 3 to 6 months, 6 to 12 months after the first administration. Effective doses may be extrapolated from dose response curves derived from in vitro or animal model test systems.

In certain embodiments, a therapeutic protein or nucleic acid or a composition thereof is administered to a subject as a single dose followed by a second dose 1 to 6 weeks, 1 to 5 weeks, 1 to 4 weeks, 1 to 3 weeks, 1 to 2 weeks later. In accordance with these embodiments, booster inoculations may be administered to the subject at 6 to 12 month intervals following the second inoculation.

In certain embodiments, administration of a therapeutic protein or nucleic acid or a composition thereof may be repeated and the administrations may be separated by at least 1 day, 2 days, 3 days, 5 days, 6 says, 7 days, 10 days, 14 days, 15 days, 21 days, 28 days, 30 days, 45 days, 2 months, 75 days, 3 months, or at least 6 months. In other embodiments, administration of therapeutic protein or nucleic acid or a composition thereof may be repeated and the administrations may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months. In some embodiments, a first therapeutic protein or nucleic acid or a composition thereof is administered to a subject followed by the administration of a second therapeutic protein or nucleic acid or a composition thereof. In certain embodiments, the first and second therapeutic proteins or nucleic acids or compositions thereof may be separated by at least 1 day, 2 days, 3 days, 5 days, 6 days, 7 days, 10 days, 14 days, 15 days, 21 days, 28 days, 30 days, 45 days, 2 months, 75 days, 3 months, or at least 6 months. In other embodiments, the first and second therapeutic proteins or nucleic acids or compositions thereof may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months.

In certain embodiments, a therapeutic protein or nucleic acid or composition thereof is administered to a subject in combination with one or more additional therapies, such as a therapy described in Section 5.6.2. The dosage of the other one or more additional therapies will depend upon various factors including, e.g., the therapy, the nature of the infectious disease, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. In other embodiments, the dose of the other therapy is a lower dose and/or less frequent administration of the therapy than recommended for the therapy for use as a single agent is used in accordance with the methods disclosed herein. Recommended doses for approved therapies can be found in the Physician's Desk Reference.

In certain embodiments, a therapeutic protein or nucleic acid or composition thereof is administered to a subject concurrently with the administration of one or more additional therapies. In other embodiments, a therapeutic protein or nucleic acid or composition thereof is administered to a subject every 3 to 7 days, 1 to 6 weeks, 1 to 5 weeks, 1 to 4 weeks, 2 to 4 weeks, 1 to 3 weeks, or 1 to 2 weeks and one or more additional therapies (such as described in Section 5.6.2) is administered every 3 to 7 days, 1 to 6 weeks, 1 to 5 weeks, 1 to 4 weeks, 1 to 3 weeks, or 1 to 2 weeks. In certain embodiments, a therapeutic protein or nucleic acid or composition thereof is administered to a subject every 1 to 2 weeks and one or more additional therapies (such as described in Section 5.6.2) is administered every 2 to 4 weeks. In some embodiments, a therapeutic protein or nucleic acid or composition thereof is administered to a subject every week and one or more additional therapies (such as described in Section 5.6.2) is administered every 2 weeks.

### 7.7 Illustrative Embodiments

### 5.7.1 Set 1

1. A fragment of the gE protein of VZV, wherein the fragment comprises a truncation of at least one and at most 49, 48, 47, 46, 45, 44, 43, or 42 amino acid residues from the C-terminal as compared with the mature gE protein.
2. The fragment of embodiment 1, wherein the fragment comprises the substitution Y569A, position numbered according to the full length gE protein.
3. The fragment of embodiment 1 or 2, wherein the fragment comprises a truncation of at most 41, 40, 39, 38, 37, 36, 35, 34, 33, 32, or 31 amino acid residues from the C-terminal as compared with the mature gE protein.
4. The fragment of embodiment 3, wherein the fragment comprises the substitution Y582G, position numbered according to the full length gE protein.
5. The fragment of any one of embodiments 1 to 4, wherein the fragment comprises a truncation of at most 30 or 29 amino acid residues from the C-terminal as compared with the mature gE protein.
6. The fragment of embodiment 5, wherein the fragment comprises the substitution S593A, position numbered according to the full length gE protein.
7. The fragment of any one of embodiments 1 to 6, wherein the fragment comprises a truncation of at most 28 amino acid residues from the C-terminal as compared with the mature gE protein.
8. The fragment of embodiment 7, wherein the fragment comprises the substitution S595A, position numbered according to the full length gE protein.
9. The fragment of any one of embodiments 1 to 8, wherein the fragment comprises a truncation of at most 27 or 26 amino acid residues from the C-terminal as compared with the mature gE protein.
10. The fragment of embodiment 9, wherein the fragment comprises the substitution T596A, position numbered according to the full length gE protein.
11. The fragment of any one of embodiments 1 to 10, wherein the fragment comprises a truncation of at most 25, 24, 23, 22, 21, 20, 19, 18, 17, 16, 15, 14, 13, 12, 11, 10, 9, 8, 7, 6, 5, 4, 3, or 2 amino acid residues or at most one amino acid residue from the C-terminal as compared with the mature gE protein.
12. The fragment of embodiment 11, wherein the fragment comprises the substitution T598A, position numbered according to the full length gE protein.
13. The fragment of any one of embodiments 1 to 12, wherein the fragment comprises a truncation of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues from the C-terminal as compared with the mature gE protein.
14. The fragment of any one of embodiments 1 to 12, wherein the fragment comprises a truncation of 11, 12, 13, 14, 15, 16, 17, 18, 34, 35, 36, 37, 38, 39, 40, 41, 42, 43, or 44 amino acid residues from the C-terminal as compared with the mature gE protein.
15. The fragment of any one of embodiments 1 to 12, wherein the fragment comprises a truncation of 14 or 37 amino acid residues from the C-terminal as compared with the mature gE protein.
16. The fragment of any one of embodiments 1 to 15, wherein the mature gE protein comprises the amino acid sequence set forth in SEQ ID NO: 1 and/or the full length gE protein comprises the amino acid sequence set forth in SEQ ID NO: 55.
17. The fragment of any one of embodiments 1-16, wherein the fragment comprises the amino acid sequence set forth in SEQ ID NO: 3, 6, 8, 10, or 12, or an amino acid sequence having at least 80%, 85%, 90%, 91%, 92%, 93%, 94%, 95%, 96%, 97%, 98%, or 99% identity with the amino acid sequence set forth in SEQ ID NO: 3, 6, 8, 10, or 12.
18. The fragment of any one of embodiments 1 to 17, wherein the N-terminal of the fragment is fused to the C-terminal of a signal peptide.
19. The fragment of any one of embodiments 1 to 18, wherein the N-terminal of the fragment is fused to the native signal peptide of the gE protein of VZV.
20. The fragment of embodiment 19, wherein the native signal peptide comprise the amino acid sequence set forth in SEQ ID NO: 18.
21. The fragment of any one of embodiments 1 to 18, wherein the N-terminal of the fragment is fused to the C-terminal of a human tPA signal peptide.
22. The fragment of embodiment 21, wherein the human tPA signal peptide comprise the amino acid sequence set forth in SEQ ID NO: 27.
23. The fragment of any one of embodiments 1 to 18, wherein the N-terminal of the fragment is fused to the C-terminal of a human IgE signal peptide.
24. The fragment of embodiment 23, wherein the human IgE signal peptide comprise the amino acid sequence set forth in SEQ ID NO: 23.
25. A nucleic acid encoding the fragment of any one of embodiments 1 to 24.
26. The nucleic acid of embodiment 25, wherein the fragment is encoded by the nucleotide sequence set forth in SEQ ID NO: 4, 5, 7, 9, 11, or 13, or a nucleotide sequence having at least 80%, 85%, 90%, 91%, 92%, 93%, 94%, 95%, 96%, 97%, 98%, or 99% identity with the nucleotide sequence set forth in SEQ ID NO: 4, 5, 7, 9, 11, or 13.
27. The nucleic acid of claim 25 or 26, wherein the native signal peptide of the gE protein of VZV is encoded by the nucleotide sequence set forth in SEQ ID NO: 19, 20, 21, or 22, or a nucleotide sequence having at least 80%, 85%, 90%, 91%, 92%, 93%, 94%, 95%, 96%, 97%, 98%, or 99% identity with the nucleotide sequence set forth in SEQ ID NO: 19, 20, 21, or 22.
28. The nucleic acid of embodiment 25 or 26, wherein the human tPA signal peptide is encoded by the nucleotide sequence set forth in SEQ ID NO: 28, or a nucleotide sequence having at least 80%, 85%, 90%, 91%, 92%, 93%, 94%, 95%, 96%, 97%, 98%, or 99% identity with the nucleotide sequence set forth in SEQ ID NO: 28.
29. The nucleic acid of embodiment 25 or 26, wherein the human IgE signal peptide is encoded by the nucleotide sequence set forth in SEQ ID NO: 24, 25, or 26, or a nucleotide sequence having at least 80%, 85%, 90%, 91%, 92%, 93%, 94%, 95%, 96%, 97%, 98%, or 99% identity with the nucleotide sequence set forth in SEQ ID NO: 24, 25, or 26.
30. A non-naturally occurring nucleic acid comprising a coding nucleotide sequence encoding the fragment of any one of embodiments 1 to 24.
31. The non-naturally occurring nucleic acid of embodiment 30, wherein the coding nucleotide sequence has been codon optimized for expression in cells of a subject, optionally, wherein the subject is a non-human mammal or a human.
32. The non-naturally occurring nucleic acid of embodiment 30 or 31, wherein the fragment is encoded by the nucleotide sequence set forth in SEQ ID NO: 4, 5, 7, 9, 11, or 13, or a nucleotide sequence having at least 80%, 85%, 90%, 91%, 92%, 93%, 94%, 95%, 96%, 97%, 98%, or 99% identity with the nucleotide sequence set forth in SEQ ID NO: 4, 5, 7, 9, 11, or 13.
33. The non-naturally occurring nucleic acid of any one of embodiments 30 to 32, wherein the native signal peptide of the gE protein of VZV is encoded by the nucleotide sequence set forth in SEQ ID NO: 19, 20, 21, or 22, or a nucleotide sequence having at least 80%, 85%, 90%, 91%, 92%, 93%, 94%, 95%, 96%, 97%, 98%, or 99% identity with the nucleotide sequence set forth in SEQ ID NO: 19, 20, 21, or 22.
34. The non-naturally occurring nucleic acid of any one of embodiments 30 to 32, wherein the human tPA signal peptide is encoded by the nucleotide sequence set forth in SEQ ID NO: 28, or a nucleotide sequence having at least 80%, 85%, 90%, 91%, 92%, 93%, 94%, 95%, 96%, 97%, 98%, or 99% identity with the nucleotide sequence set forth in SEQ ID NO: 28.
35. The non-naturally occurring nucleic acid of any one of claims 30 to 32, wherein the human IgE signal peptide is encoded by the nucleotide sequence set forth in SEQ ID NO: 24, 25, or 26, or a nucleotide sequence having at least 80%, 85%, 90%, 91%, 92%, 93%, 94%, 95%, 96%, 97%, 98%, or 99% identity with the nucleotide sequence set forth in SEQ ID NO: 24, 25, or 26.
36. The non-naturally occurring nucleic acid of any one of embodiments 30 to 35,
further comprising a 5′ untranslated region (5′-UTR), wherein the 5′-UTR comprises the sequence set forth in any one of SEQ ID NOS: 29-38,
and/or,
further comprising a 3′ untranslated region (3′-UTR), wherein the 3′-UTR comprises the sequence set forth in any one of SEQ ID NOS: 39-46,
   optionally, wherein the 3′-UTR further comprises a poly-A tail or a polyadenylation signal.
37. The non-naturally occurring nucleic acid of any one of embodiments 30 to 36, wherein the nucleic acid consists of, essentially consists of or comprises the nucleotide sequence set forth in SEQ ID NO: 49, 50, 51, 52, 53, 54, 60, 61, 62, 63 or 64 or comprises a nucleotide sequence having at least 80%, 85%, 90%, 91%, 92%, 93%, 94%, 95%, 96%, 97%, 98%, or 99% identity with the nucleotide sequence set forth in SEQ ID NO: 49, 50, 51, 52, 53, 54, 60, 61, 62, 63 or 64.
38. The non-naturally occurring nucleic acid of any one of embodiments 30 to 37, wherein the nucleic acid is DNA or mRNA.
39. The non-naturally occurring nucleic acid of any one of embodiments 30 to 38, wherein the nucleic acid comprises one or more functional nucleotide analogs.
40. The non-naturally occurring nucleic acid of embodiment 39, wherein the nucleic acid comprises one or more functional nucleotide analogs that are selected from pseudouridine, 1-methyl-pseudouridine and 5-methylcytosine.
41. The non-naturally occurring nucleic acid of any one of embodiments 30 to 40, wherein the nucleic acid consists of, essentially consists of or comprises the nucleotide sequence set forth in SEQ ID NO: 63 or comprises a nucleotide sequence having at least 80%, 85%, 90%, 91%, 92%, 93%, 94%, 95%, 96%, 97%, 98%, or 99% identity with the nucleotide sequence set forth in SEQ ID NO: 63, wherein all thymines (T) are substituted for uracil (U) or N1-methylpseudouridine, and/or the first nucleotide G is substituted for m⁷ GpppAmpU.
42. A vector comprising the nucleic acid of any one of embodiments 25 to 29 or the non-naturally occurring nucleic acid of any one of claims 30 to 41; preferably, an IVT (In Vitro Transcription) plasmid.
43. A cell comprising the nucleic acid of any one of embodiments 25 to 29, the non-naturally occurring nucleic acid of any one of claims 30 to 41, or the vector of claim 42.
44. A pharmaceutical composition comprising the fragment of any one of embodiments 1 to 24.
45. A pharmaceutical composition comprising the nucleic acid of any one of embodiments 25 to 29.
46. A pharmaceutical composition comprising the non-naturally occurring nucleic acid of any one of embodiments 30 to 41 and at least a first lipid,
optionally, wherein the first lipid is a compound according to Formula 01-I or Formula 01-II; or a compound listed in Table 01-1; or a compound according to Formula 02-I; or a compound listed in Table 02-1; or a compound according to Formula 03-I; or a compound listed in Table 03-1; or a compound according to Formula 04-I; or a compound listed in Table 04-1.
47. The pharmaceutical composition of embodiment 46 further comprising a second lipid, optionally, wherein the second lipid is a compound according to Formula 05-I.
48. The pharmaceutical composition of embodiment 46 or 47 formulated as lipid nanoparticles encapsulating the nucleic acid in a lipid shell.
49. The pharmaceutical composition of any one of embodiments 46 to 48, wherein the composition is a vaccine.
50. A method for managing, preventing or treating a disease or disorder caused by VZV or by infection with VZV in a subject, comprising administering to the subject a therapeutically effective amount of the fragment of any one of embodiments 1 to 24, the nucleic acid of any one of claims 25 to 29, the non-naturally occurring nucleic acid of any one of claims 30 to 41, or the pharmaceutical composition of any one of embodiments 44 to 49.
51. The method of embodiment 50, wherein an immune response against the VZV is elicited in the subject.
52. The method of embodiment 51, wherein the immune response comprises production of cytokine in lymphocytes.
53. The method of embodiment 52, wherein the immune response comprises increased proportion of cytokine-expressing lymphocytes.
54. The method of embodiment 53, wherein the lymphocytes are CD4⁺ T cells and/or CD8⁺ T cells, and/or, wherein the cytokine is one or more of IFN-γ, IL-2, and TNF-α.
55. The method of embodiment 54, wherein the production of cytokine in lymphocytes is increased.
56. The method of embodiment 55, wherein the immune response comprises production of an antibody specifically binds to the viral gE protein.
57. The method of any one of embodiments 50 to 56, wherein the disease or disorder caused by VZV is
(a) varicella and/or zoster;
(b) postherpetic neuralgia (PHN); and/or
(c) one or more of meningoencephalitis, myelitis, cranial nerve palsy, angiopathy, keratitis, retinopathy, ulcers, hepatitis and pancreatitis.

### 5.7.2 Set 2

1. A protein comprising a mutant of a mature glycoprotein E (gE) of varicella-zoster virus (VZV), wherein the mutant comprises:
   (a) (i) a truncation of 37 amino acid residues from the C-terminus of the mature gE, and (ii) amino acid residue substitutions Y569A and Y582G, wherein amino acid residue positions 569 and 582 are the amino acid residue position numbers of full length VSV gE;
   (b) amino acid residue substitutions Y569A and Y582G, wherein amino acid residue positions 569 and 582 are the amino acid residue position numbers of full length VSV gE;
   (c) amino acid residue substitutions Y569A, Y582G, S593A, S595A, T596A, and T598A, wherein amino acid residue positions 569, 582, 593, 595, 596, and 598 are the amino acid residue position numbers of full length VSV gE;
   (d) amino acid residue substitutions Y582G, S593A, S595A, T596A, and T598A, wherein amino acid residue positions 582, 593, 595, 596, and 598 are the amino acid residue position numbers of full length VSV gE; or
   (e) (i) a truncation of 50 amino acid residues from the C-terminus of the mature gE protein, and (ii) amino acid residue substitution Y569A, wherein amino acid residue position 569 is the amino acid residue position number of full length VSV gE.
2. The protein of embodiment 1, wherein the mutant comprises the amino acid sequence of SEQ ID NO: 6, 8, 10, 12, or 3.
3. The protein of embodiment 1, wherein the amino acid sequence of the mutant consists of the amino acid sequence of SEQ ID NO: 6, 8, 10, 12, or 3.
4. The protein of embodiment 1, wherein the mutant comprises the amino acid sequence of SEQ ID NO: 6.
5. The protein of embodiment 1, wherein the amino acid sequence of the mutant consists of the amino acid sequence of SEQ ID NO: 6.
6. The protein of embodiment 1, wherein the mutant comprises an amino acid sequence that is at least 90%, at least 91%, at least 92%, at least 93%, at least 94%, or at least 95% identical to SEQ ID NO: 6, 8, 10, or 12.
7. The protein of embodiment 1, wherein the mutant comprises an amino acid sequence that is at least 96%, at least 97%, at least 98%, or at least 99% identical to SEQ ID NO: 6, 8, 10, or 12.
8. The protein of embodiment 1, wherein the mutant comprises an amino acid sequence that is at least 90%, at least 91%, at least 92%, at least 93%, at least 94%, or at least 95% identical to SEQ ID NO: 6.
9. The protein of embodiment 1, wherein the mutant comprises an amino acid sequence that is at least 96%, at least 97%, at least 98%, or at least 99% identical to SEQ ID NO: 6.
10. The protein of any one of embodiments 1 to 9, which further comprises a VZV gE signal peptide.
11. The protein of embodiment 10, wherein the signal peptide of the VZV gE comprises the amino acid sequence set forth in SEQ ID NO: 18.
12. The protein of embodiment 11, wherein the signal peptide of the VZV gE comprises an amino acid sequence that is at least 95%, at least 96%, at least 97%, at least 98%, or at least 99% identical to the amino acid sequence set forth in SEQ ID NO: 18.
13. The protein of any one of embodiments 1 to 9, which further comprises a heterologous signal peptide, wherein the N-terminal of the mutant is fused to the C-terminal of the heterologous signal peptide.
14. The protein of embodiment 13, wherein the heterologous signal peptide is a human IgE signal peptide.
15. The protein of embodiment 14, wherein the human IgE signal peptide comprises: (i) the amino acid sequence set forth in SEQ ID NO: 23, or (ii) an amino acid sequence that is at least 95%, at least 96%, at least 97%, at least 98%, or at least 99% identical to the amino acid sequence set forth in SEQ ID NO: 23.
16. The protein of embodiment 14, wherein the amino acid sequence of the human IgE signal peptide consists of the amino acid sequence set forth in SEQ ID NO: 23.
17. The protein of embodiment 14, wherein the amino acid sequence of the mutant consists of the amino acid sequence set forth in SEQ ID NO: 6, and the amino acid sequence of the human IgE signal peptide consists of the amino acid sequence set forth in SEQ ID NO: 23.
18. The protein of embodiment 14, which comprises the amino acid sequence set forth in SEQ ID NO: 59.
19. The protein of embodiment 14, wherein the amino acid sequence of the protein consists of the amino acid sequence set forth in SEQ ID NO: 59.
20. The protein of embodiment 13, wherein the heterologous signal peptide is a human tPA signal peptide.
21. The protein of embodiment 20, wherein the human tPA signal peptide comprises: (i) the amino acid sequence set forth in SEQ ID NO: 27, or (ii) an amino acid sequence that is at least 95%, at least 96%, at least 97%, at least 98%, or at least 99% identical to the amino acid sequence set forth in SEQ ID NO: 27.
22. The protein of any one of embodiments 1 to 21, wherein the mature gE comprises the amino acid sequence set forth in SEQ ID NO: 1 and/or the full length VZV gE comprises the amino acid sequence set forth in SEQ ID NO: 55.
23. A fragment of a mature glycoprotein E (gE) of varicella-zoster virus (VZV), wherein the fragment comprises a truncation of at least one and at most 50, 49, 48, 47, 46, 45, 44, 43, or 42 amino acid residues from the C-terminal of the mature gE, optionally the truncation is a truncation of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues from the C-terminal of the mature gE, or optionally the truncation is a truncation of 11, 12, 13, 14, 15, 16, 17, 18, 34, 35, 36, 37, 38, 39, 40, 41, 42, 43, or 44 amino acid residues from the C-terminal of the mature gE, or optionally the truncation is a truncation of 14 or 37 amino acid residues from the C-terminal of the mature gE.
24. The fragment of embodiment 23, wherein the fragment further comprises the amino acid residue substitution Y569A, and wherein amino acid residue position 569 is the amino acid residue position number according to the full length VZV gE.
25. The fragment of embodiment 23 or 24, wherein the fragment comprises a truncation of at most 41, 40, 39, 38, 37, 36, 35, 34, 33, 32, or 31 amino acid residues from the C-terminal of the mature gE.
26. The fragment of embodiment 25, wherein the fragment further comprises the amino acid residue substitution Y582G, and wherein amino acid residue position 582 is the amino acid residue position number according to the full length VZV gE protein.
27. The fragment of any one of embodiments 23 to 26, wherein the fragment comprises a truncation of at most 30 or 29 amino acid residues from the C-terminal of the mature gE.
28. The fragment of embodiment 27, wherein the fragment further comprises the amino acid residue substitution S593A, and wherein amino acid residue position 593 is the amino acid residue position number according to the full length VZV gE.
29. The fragment of any one of embodiments 23 to 28, wherein the fragment comprises a truncation of at most 28 amino acid residues from the C-terminal of the mature gE.
30. The fragment of embodiment 29, wherein the fragment comprises the amino acid residue substitution S595A, and wherein amino acid residue position 595 is the amino acid residue position number according to the full length VSV gE.
31. The fragment of any one of embodiments 23 to 30, wherein the fragment comprises a truncation of at most 27 or 26 amino acid residues from the C-terminal of the mature gE.
32. The fragment of embodiment 31, wherein the fragment further comprises the amino acid residue substitution T596A, and wherein amino acid residue position 596 is the amino acid residue position number according to the full length VZV gE.
33. The fragment of any one of embodiments 23 to 32, wherein the fragment comprises a truncation of at most 25, 24, 23, 22, 21, 20, 19, 18, 17, 16, 15, 14, 13, 12, 11, 10, 9, 8, 7, 6, 5, 4, 3, or 2 amino acid residues or at most one amino acid residue from the C-terminal of the mature gE.
34. The fragment of embodiment 33, wherein the fragment further comprises the amino acid residue substitution T598A, and wherein amino acid residue position 598 is the amino acid residue position number according to the full length VZV gE.
35. The fragment of embodiment 23, wherein the fragment comprises a truncation of 37 amino acid residues from the C-terminal of the mature gE.
36. The fragment of embodiment 23, wherein the fragment comprises: (1) a truncation of 37 amino acid residues from the C-terminal of the mature gE, and (2) the amino acid residue substitutions Y569A and Y582G, and wherein amino acid residue positions 569 and 582 are amino acid residue position numbers according to the full length VZV gE.
37. The fragment of any one of embodiments 23 to 36, wherein the mature gE comprises the amino acid sequence set forth in SEQ ID NO: 1 and/or the full length VZV gE comprises the amino acid sequence set forth in SEQ ID NO: 55.
38. The fragment of any one of embodiments 23 to 37, wherein the fragment comprises an amino acid sequence having at least 80%, 85%, 90%, 91%, 92%, 93%, 94%, 95%, 96%, 97%, 98%, or 99% identity with the amino acid sequence set forth in SEQ ID NO: 6, 3, 8, 10, or 12.
39. A fusion protein comprising the fragment of any one of embodiments 23 to 38, and a heterologous signal peptide, wherein the N-terminal of the fragment is fused to the C-terminal of the heterologous signal peptide.
40. The fusion protein of embodiment 39, wherein the heterologous signal peptide is a human tPA signal peptide.
41. The fusion protein of embodiment 40, wherein the human tPA signal peptide comprises the amino acid sequence set forth in SEQ ID NO: 27.
42. The fusion protein of embodiment 39, wherein the heterologous signal peptide is a human IgE signal peptide.
43. The fusion protein of embodiment 42, wherein the human IgE signal peptide comprises the amino acid sequence set forth in SEQ ID NO: 23.
44. A fusion protein comprising a fragment of a mature glycoprotein (gE) of varicella-zoster virus (VZV) and a human IgE signal peptide, wherein the fragment comprises a truncation of 37 amino acid residues from the C-terminal of the mature gE, and the N-terminal of the fragment is fused to the C-terminal of the human IgE signal peptide.
45. A nucleic acid encoding the protein of any of embodiments 1 to 22, fragment of any one of embodiments 23 to 38, or the fusion protein of any one of embodiments 39 to 44.
46. The nucleic acid of embodiment 45, which comprises a nucleotide sequence set forth in SEQ ID NO: 7, 9, 11, 13, 4, or 5, or a nucleotide sequence having at least 80%, 85%, 90%, 91%, 92%, 93%, 94%, 95%, 96%, 97%, 98%, or 99% identity with the nucleotide sequence set forth in SEQ ID NO: 7, 9, 11, 13, 4 or 5.
47. A nucleic acid encoding the protein of any one of embodiments 14 to 19, or the fusion protein of any one of embodiments 42 to 44, wherein the nucleotide sequence encoding the IgE signal peptide comprises: (a) the nucleotide sequence set forth in SEQ ID NO: 24, 25, or 26, or (b) a nucleotide sequence that is at least 80%, 85%, 90%, 910%, 92%, 93%, 94%, 95%, 96%, 97%, 98%, or 99% identical to the nucleotide sequence set forth in SEQ ID NO: 24, 25, or 26.
48. A nucleic acid encoding the protein of any one of embodiments 10 to 12, wherein the nucleotide sequence of the signal peptide comprises: (a) the nucleotide sequence set forth in SEQ ID NO: 19, 20, 21, or 22, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 19, 20, 21, or 22.
49. A nucleic acid encoding the protein of embodiment 20 or 21, or the fusion protein of embodiment 40 or 41, wherein the nucleotide sequence encoding a human tPA signal peptide comprises: (a) the nucleotide sequence set forth in SEQ ID NO: 28, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 28.
50. A non-naturally occurring nucleic acid comprising a coding nucleotide sequence encoding the protein of any one of embodiments 1 to 22, the fragment of any one of embodiments 23 to 38, or the fusion protein of any one of embodiments 39 to 44.
51. The non-naturally occurring nucleic acid of embodiment 50, wherein the coding nucleotide sequence has been codon optimized for expression in cells of a subject, optionally, wherein the subject is a non-human mammal or a human.
52. The non-naturally occurring nucleic acid of embodiment 50 or 51, which comprises: (a) the nucleotide sequence set forth in SEQ ID NO: 7, 9, 11, 13, 4 or 5, or (b) a nucleotide sequence that is at least 80%, 85%, 90%, 910%, 92%, 93%, 94%, 95%, 96%, 97%, 98%, or 99% identical to the nucleotide sequence set forth in SEQ ID NO: 7, 9, 11, 13, 4 or 5.
53. A non-naturally occurring nucleic acid encoding the protein of any one of embodiments 14 to 19, or the fusion protein of any one of embodiments 42 to 44, wherein the nucleotide sequence encoding the IgE signal peptide comprises: (a) the nucleotide sequence set forth in SEQ ID NO: 24, 25, or 26, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 24, 25, or 26.
54. A non-naturally occurring nucleic acid encoding the protein of any one of embodiments 10 to 12, wherein the nucleotide sequence of the signal peptide of the full length VSV gE protein comprises: (a) the nucleotide sequence set forth in SEQ ID NO: 19, 20, 21, or 22, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 19, 20, 21, or 22.
55. A non-naturally occurring nucleic acid encoding the protein of embodiment 20 or 21, or the fusion protein of embodiment 40 or 41, wherein the nucleotide sequence encoding a human tPA signal peptide comprises: (a) the nucleotide sequence set forth in SEQ ID NO: 28, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 28.
56. A non-naturally occurring nucleic acid comprising the nucleotide sequence of SEQ ID NO: 63, 7, 51, 60, 62, 62, 64, 9, 11, 13, 52, 53, 54, 58, 49, 50, 4, or 5.
57. The non-naturally occurring nucleic acid of embodiment 56, which consists of the nucleotide sequence of SEQ ID NO: 63, 7, 51, 60, 62, 62, 64, 9, 11, 13, 52, 53, 54, 58, 49, 50, 4, or 5.
58. The non-naturally occurring nucleic acid of any one of embodiments 50 to 55, wherein the nucleic acid consists of, consists essentially of, or comprises: (1) the nucleotide sequence set forth in SEQ ID NO: 63, 7, 51, 60, 62, 62, 64, 9, 11, 13, 52, 53, 54, 58, 49, 50, 4, or 5; or (2) a nucleotide sequence having at least 80%, 85%, 90%, 91%, 92%, 93%, 94%, 95%, 96%, 97%, 98%, or 99% identity with the nucleotide sequence set forth in SEQ ID NO: 63, 7, 51, 60, 62, 62, 64, 9, 11, 13, 52, 53, 54, 58, 49, 50, 4, or 5.
59. The non-naturally occurring nucleic acid of any one of embodiments 50 to 58, further comprising a 5′ untranslated region (5′-UTR), wherein the 5′-UTR comprises the nucleotide sequence set forth in any one of SEQ ID NOS: 29-38, and/or, further comprising a 3′ untranslated region (3′-UTR), wherein the 3′-UTR comprises the nucleotide sequence set forth in any one of SEQ ID NOS: 39-46, optionally, wherein the 3′-UTR further comprises a poly-A tail or a polyadenylation signal.
60. The non-naturally occurring nucleic acid of any one of embodiments 50 to 59, wherein the nucleic acid is DNA.
61. The non-naturally occurring nucleic acid of any one of embodiments 50 to 60, wherein the nucleic acid comprises one or more functional nucleotide analogs.
62. The non-naturally occurring nucleic acid of any one 50 to 59, wherein the nucleic acid is mRNA, and wherein thymines are substituted for uracils or a functional analog thereof in the nucleic acid.
63. The non-naturally occurring nucleic acid of embodiment 62, wherein the nucleic acid comprises one or more functional nucleotide analogs that are selected from pseudouridine, 1-methyl-pseudouridine and 5-methylcytosine.
64. The non-naturally occurring nucleic acid of embodiment 62, wherein the nucleic acid consists of, consists essentially of, or comprises: (1) the nucleotide sequence set forth in SEQ ID NO: 63, or (2) a nucleotide sequence that is at least 80%, 85%, 90%, 91%, 92%, 93%, 94%, 95%, 96%, 97%, 98%, or 99% identical to the nucleotide sequence set forth in SEQ ID NO: 63; except that all thymines (T) are substituted for uracil (U) or N1-methylpseudouridine, and/or the first nucleotide G is substituted for m⁷ GpppAmpU.
65. The non-naturally occurring nucleic acid of embodiment 62, wherein the nucleic acid consists of, essentially consists of, or comprises the nucleotide sequence set forth in SEQ ID NO: 63, except that all thymines (T) are substituted for uracil (U) or N1-methylpseudouridine, and/or the first nucleotide G is substituted for m⁷ GpppAmpU.
66. The non-naturally occurring nucleic acid of embodiment 62, wherein the nucleic acid consists of, essentially consists of or comprises the nucleotide sequence set forth in SEQ ID NO: 63, except that all thymines (T) are substituted for N1-methylpseudouridine, and the first nucleotide G is substituted for m⁷ GpppAmpU.
67. A vector comprising the nucleic acid of any one of embodiments 45 to 49, or the non-naturally occurring nucleic acid of any one of embodiments 50 to 66; preferably, an IVT (In Vitro Transcription) plasmid.
68. A host cell comprising the nucleic acid of any one of embodiments 45 to 49, the non-naturally occurring nucleic acid of any one of embodiments 50 to 66, or the vector of embodiment 67.
69. The host cell of embodiment 68, which is in vitro, ex vivo, or isolated.
70. A pharmaceutical composition comprising the protein of any one of embodiments 1 to 22, the fragment of any one of embodiments 23 to 38, or the fusion protein of any one of embodiments 39 to 44.
71. A pharmaceutical composition comprising the nucleic acid of any one of embodiments 45 to 49, the non-naturally occurring nucleic acid of any one of embodiments 50 to 66, or the vector of embodiment 67.
72. A pharmaceutical composition comprising the non-naturally occurring nucleic acid of any one of embodiments 50 to 66, and at least a first lipid, optionally, wherein the first lipid is a compound according to Formula 01-I or Formula 01-II; or a compound listed in Table 01-1; or a compound according to Formula 02-I; or a compound listed in Table 02-1; or a compound according to Formula 03-I; or a compound listed in Table 03-1; or a compound according to Formula 04-I; or a compound listed in Table 04-1.
73. The pharmaceutical composition of embodiment 72, further comprising a second lipid, optionally, wherein the second lipid is a compound according to Formula 05-I.
74. The pharmaceutical composition of embodiment 72 or 73, formulated as lipid nanoparticles encapsulating the nucleic acid in a lipid shell.
75. The pharmaceutical composition of any one of embodiments 70 to 74, wherein the composition is a vaccine.
76. A method for managing, preventing or treating a disease or disorder caused by VZV or by infection with VZV in a subject, comprising administering to the subject a therapeutically effective amount of the protein of any one of embodiments 1 to 22, the fragment of any one of embodiments 23 to 38, the fusion protein of any one of embodiments 39 to 44, the nucleic acid of any one of embodiments 45 to 49, the non-naturally occurring nucleic acid of any one of embodiments 50 to 66, the vector of embodiment 67, or the pharmaceutical composition of any one of embodiments 70 to 75.
77. The method of embodiment 76, wherein the method is for preventing a disease or disorder caused by VZV or by infection with VZV in the subject.
78. The method of embodiment 76 or 77, wherein an immune response against the VZV is elicited in the subject.
79. The method of embodiment 78, wherein the immune response comprises production of cytokine in lymphocytes.
80. The method of embodiment 78 or 79, wherein the immune response comprises increased proportion of cytokine-expressing lymphocytes.
81. The method of embodiment 79 or 80, wherein the lymphocytes are CD4⁺ T cells and/or CD8⁺ T cells, and/or, wherein the cytokine is one or more of IFN-γ, IL-2, and TNF-α.
82. The method of any one of embodiments 79 to 81, wherein the production of cytokine in lymphocytes is increased.
83. The method of any one of embodiments 78 to 82, wherein the immune response comprises production of an antibody that specifically binds to VZV gE.
84. The method of any one of embodiments 76 to 83, wherein the disease or disorder caused by VZV is
(a) varicella and/or zoster;
(b) postherpetic neuralgia (PHN); and/or
(c) one or more of meningoencephalitis, myelitis, cranial nerve palsy, angiopathy, keratitis, retinopathy, ulcers, hepatitis and pancreatitis.
85. The method of any one of embodiments 76 to 84, wherein the subject is a human.
86. The method of embodiment 85, wherein the human is a human adult.
87. The method of embodiment 86, wherein the adult is at least 40 years old.
88. The method of embodiment 85, wherein the human is an elderly human.
89. A protein as defined in any one of embodiments 1 to 22, a fragment as defined in any one of embodiments 23 to 38, a fusion protein as defined in any one of embodiments 39 to 44, a nucleic acid as defined in any one of embodiments 45 to 49, a non-naturally occurring nucleic acid as defined in any one of embodiments 50 to 66, a vector as defined in embodiment 67, or a pharmaceutical composition as defined in any one of embodiments 70 to 75, for use in a method for managing, preventing or treating a disease or disorder caused by VZV or by infection with VZV in a subject.
90. The protein, fragment, fusion protein, nucleic acid, non-naturally occurring nucleic acid, vector, or pharmaceutical composition for use according to embodiment 89, wherein the subject is human, optionally a human adult or elderly human.
91. Use of the protein as defined in any one of embodiments 1 to 22, the fragment as defined in any one of embodiments 23 to 38, the fusion protein as defined in any one of embodiments 39 to 44, the nucleic acid as defined in any one of embodiments 45 to 49, the non-naturally occurring nucleic acid as defined in any one of embodiments 50 to 66, the vector as defined in embodiment 67, or the pharmaceutical composition as defined in any one of embodiments 70 to 75, for the manufacture of a medicament for managing, preventing or treating a disease or disorder caused by VZV or by infection with VZV in a subject.
92. The use of embodiment 91, wherein the subject is human, optionally a human adult or elderly human.

### 6. EXAMPLES

The examples in this section are offered by way of illustration, and not by way of limitation.

The following examples are provided to exemplify the preparation of series 01 to series 04 of cationic lipids.

General preparative HPLC method: HPLC purification is carried out on Waters 2767 equipped with a diode array detector (DAD) on an Inertsil Pre-C8 OBD column, generally with water containing 0.1% TFA as solvent A and acetonitrile as solvent B. General LCMS method: LCMS analysis is conducted on a Shimadzu (LC-MS2020) System. Chromatography is performed on a SunFire C18, generally with water containing 0.1% formic acid as solvent A and acetonitrile containing 0.1% formic acid as solvent B.

### Preparation Example 01: Preparation of Compounds of Series 01

Please refer to International Patent Application Publication No. WO 2021/204175, which is incorporated by reference in its entirety.

### Preparation Example 02-1: Preparation of Compound 02-1 (i.e. Compound 1 in the Following Scheme)

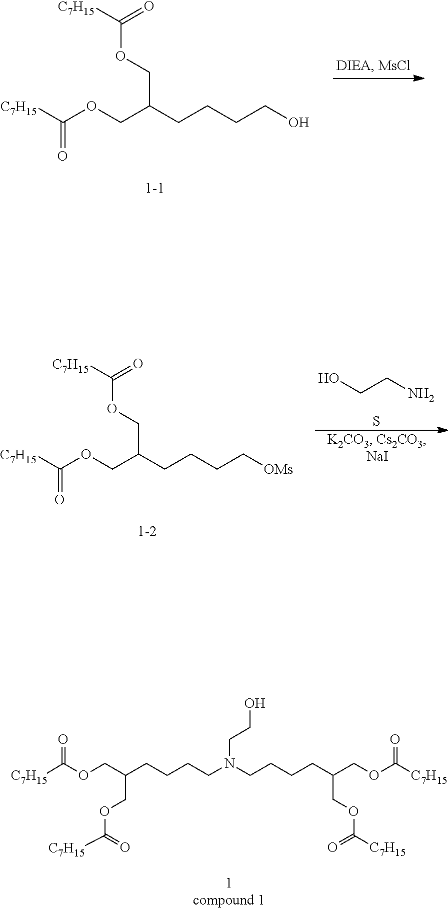

¹H NMR (400 MHz, CDCl₃) δ: 0.86-0.90 (m, 12H), 1.27-1.63 (m, 53H), 1.97-2.01 (m, 2H), 2.28-2.64 (m, 14H), 3.52-3.58 (m, 2H), 4.00-4.10 (m, 8H). LCMS: Rt: 1.080 min; MS m/z (ESI): 826.0 [M+H]⁺.

The following compounds were prepared in analogous fashion as Compound 02-1, using corresponding starting material.

| Compound | Characterization |
|---|---|
| 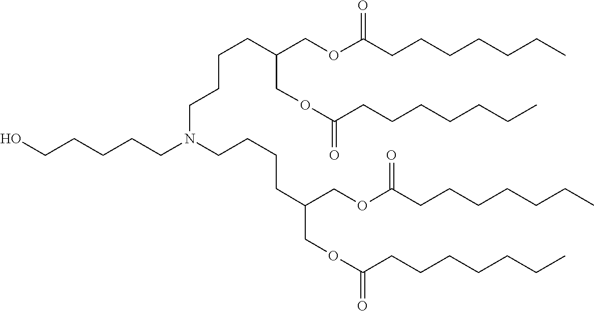 [SMILES: CCCCCCCC(=O)OCC(CCCCN(CCCCCO)CCCCC(COC(=O)CCCCCCC)COC(=O)CCCCCCC)COC(=O)CCCCCCC] Compound 02-3 | ¹H NMR (400 MHz, CDCl₃) δ: 0.86-0.90 (m, 12H), 1.28-1.32 (m, 30H), 1.35-1.44 (m, 12H), 1.57-1.66 (m, 17H), 1.95-2.01 (m, 2H), 2.28-2.40 (m, 14H), 3.63-3.66 (m, 2H), 4.00-4.11 (m, 8H). LCMS: Rt: 1.140 min; MS m/z (ESI): 868.1 [M + H]⁺. |

### Preparation Example 02-2: Preparation of Compound 02-2 (i.e. Compound 2 in the Following Scheme)

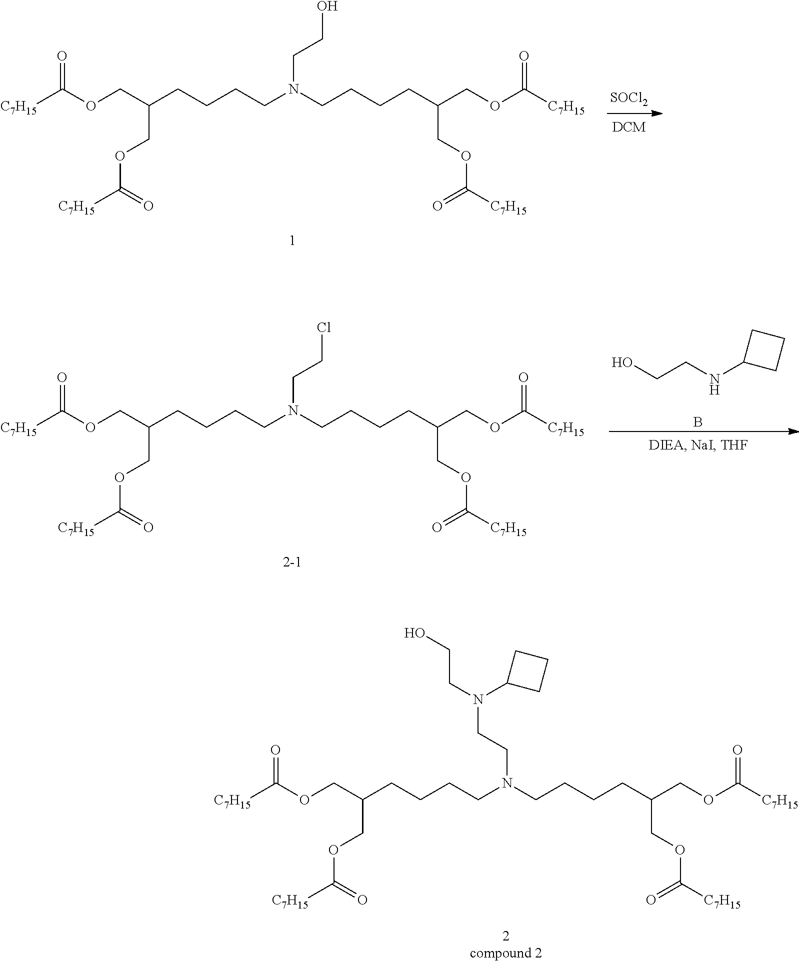

¹H NMR (400 MHz, CDCl₃) δ: 0.86-0.90 (m, 12H), 1.28-1.67 (m, 54H), 1.88-2.01 (m, 7H), 2.28-2.56 (m, 18H), 3.16-3.20 (m, 1H), 3.52-3.54 (m, 2H), 4.00-4.10 (m, 8H). LCMS: Rt: 1.060 min; MS m/z (ESI): 923.0 [M+H]⁺.

### Preparation Example 02-3: Preparation of Compound 02-4 (i.e. Compound 4 in the Following Scheme)

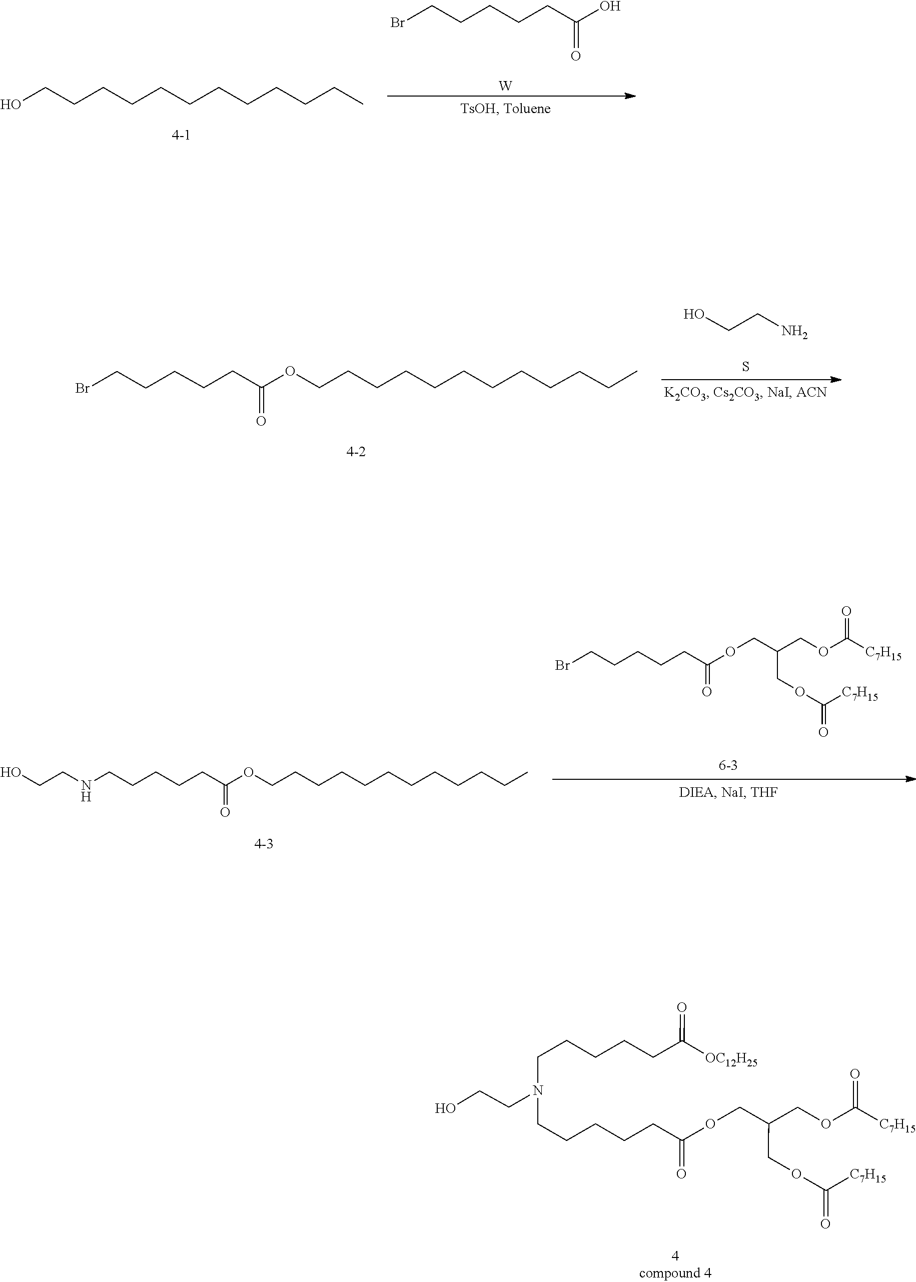

¹H NMR (400 MHz, CDCl₃) δ: 0.86-0.90 (m, 9H), 1.26-1.32 (m, 34H), 1.41-1.49 (m, 4H), 1.61-1.66 (m, 15H), 2.00-2.03 (m, 1H), 2.21-2.38 (m, 8H), 2.43-2.47 (m, 4H), 2.56-2.60 (m, 2H), 3.50-3.54 (m, 2H), 4.03-4.14 (m, 8H). LCMS: Rt: 1.030 min; MS m/z (ESI): 798.0 [M+H]⁺.

### Preparation Example 02-4: Preparation of Compound 02-9 (i.e. Compound 9 in the Following Scheme)

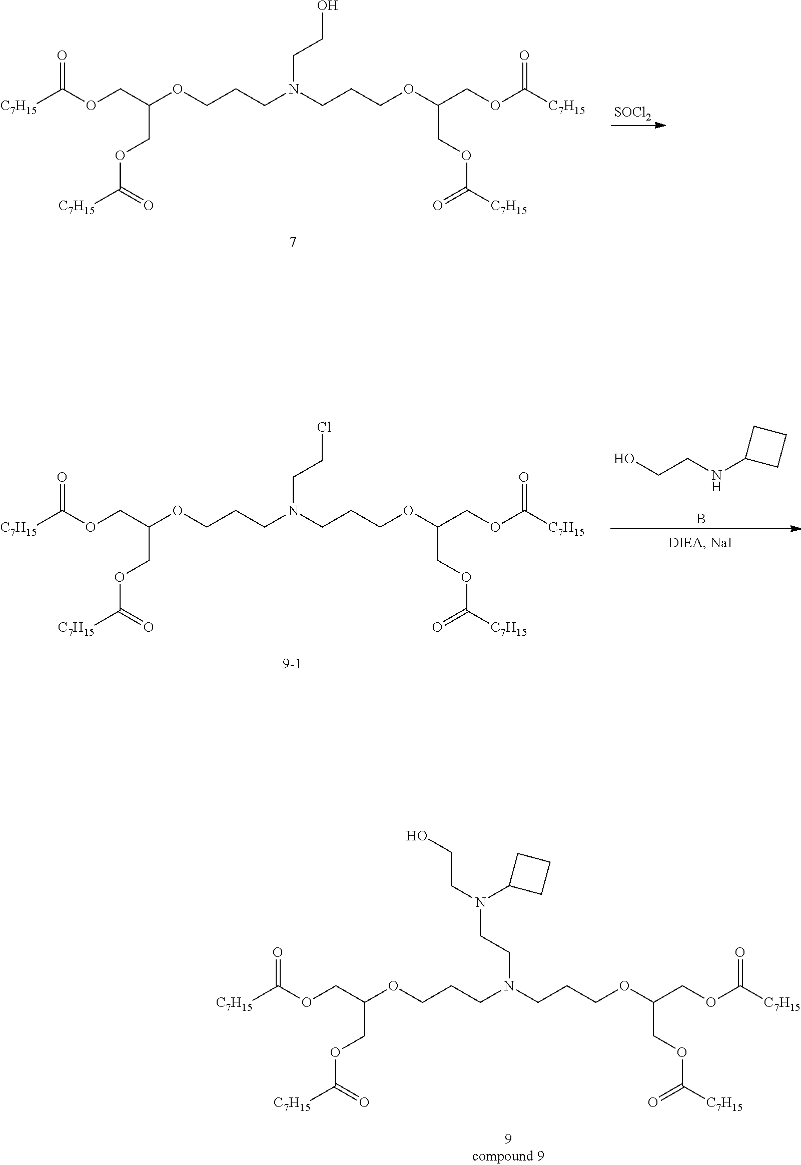

¹H NMR (400 MHz, CDCl₃) δ: 0.86-0.90 (m, 12H), 1.28-1.30 (m, 33H), 1.58-2.01 (m, 18H), 2.30-2.54 (m, 18H), 3.10-3.19 (m, 1H), 3.52-3.68 (m, 8H), 4.09-4.20 (m, 8H). LCMS: Rt: 1.677 min; MS m/z (ESI): 927.7 [M+H]⁺.

The following compounds were prepared in analogous fashion as Compound 02-9, using corresponding starting material.

| Compound | Characterization |
|---|---|
| 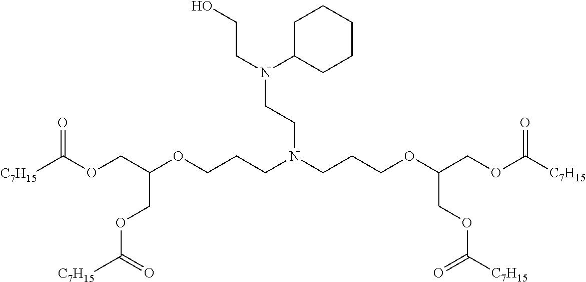 Compound 02-14 | ¹H NMR (400 MHz, CDCl₃) δ: 0.86-0.90 (m, 12H), 1.28-1.30 (m, 40H), 1.58-1.80 (m, 16H), 2.30-2.60 (m, 18H), 3.58-3.68 (m, 8H), 4.08-4.20 (m, 8H). LCMS: Rt: 1.000 min; MS m/z (ESI): 955.7 [M + H]⁺. |

### Preparation Example 02-5: Preparation of Compound 02-10 (i.e. Compound 10 in the Following Scheme)

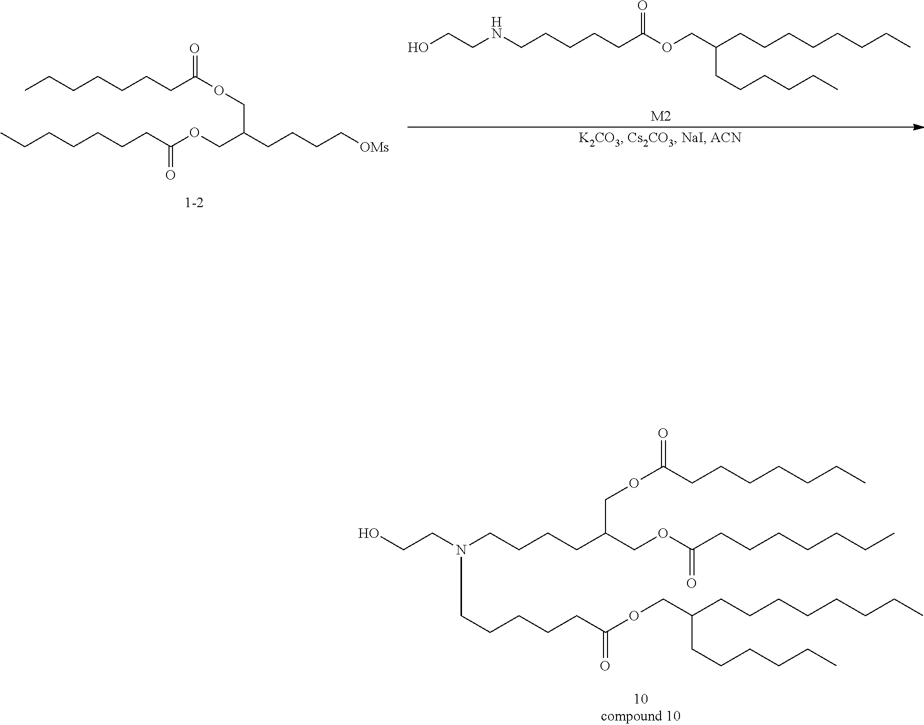 [SMILES: CCCCCCCC(=O)OCC(CCCCOS(C)(=O)=O)COC(=O)CCCCCCC ; CCCCCCCCC(CCCCCC)COC(=O)CCCCCNCCO ; "M2" ; CCCCCCCCC(CCCCCC)COC(=O)CCCCCN(CCO)CCCCC(COC(=O)CCCCCCC)COC(=O)CCCCCCC]

¹H NMR (400 MHz, CDCl₃) δ: 0.86-0.90 (m, 12H), 1.26-1.41 (m, 48H), 1.51-1.72 (m, 11H), 1.94-2.03 (m, 1H), 2.29-2.32 (m, 6H), 2.41-2.91 (m, 5H), 3.51-3.76 (m, 2H), 3.96-4.10 (m, 6H). LCMS: Rt: 1.327 min; MS m/z (ESI): 782.6 [M+H]+.

The following compounds were prepared in analogous fashion as Compound 02-10, using corresponding starting material.

| Compound | Characterization |
|---|---|
| 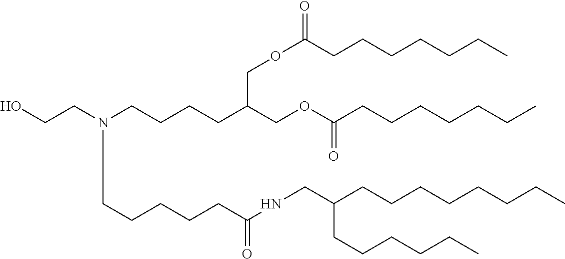 [SMILES: CCCCCCCCC(CCCCCC)CNC(=O)CCCCCN(CCO)CCCCC(COC(=O)CCCCCCC)COC(=O)CCCCCCC] Compound 02-11 | ¹H NMR (400 MHz, CDCl₃) δ: 0.86-0.90 (m, 12H), 1.26-1.30 (m, 46H), 1.43-1.50 (m, 8H), 1.69-1.73 (m, 3H), 1.83-1.90 (m, 3H), 1.98-2.02 (m, 1H), 2.20-2.33 (m, 6H), 3.05-3.19 (m, 7H), 3.98-4.10 (m, 6H). LCMS: Rt: 1.205 min; MS m/z (ESI): 781.7 [M + H]⁺. |

### Preparation Example 02-6: Preparation of Compound 02-12 (i.e. Compound 12 in the Following Scheme)

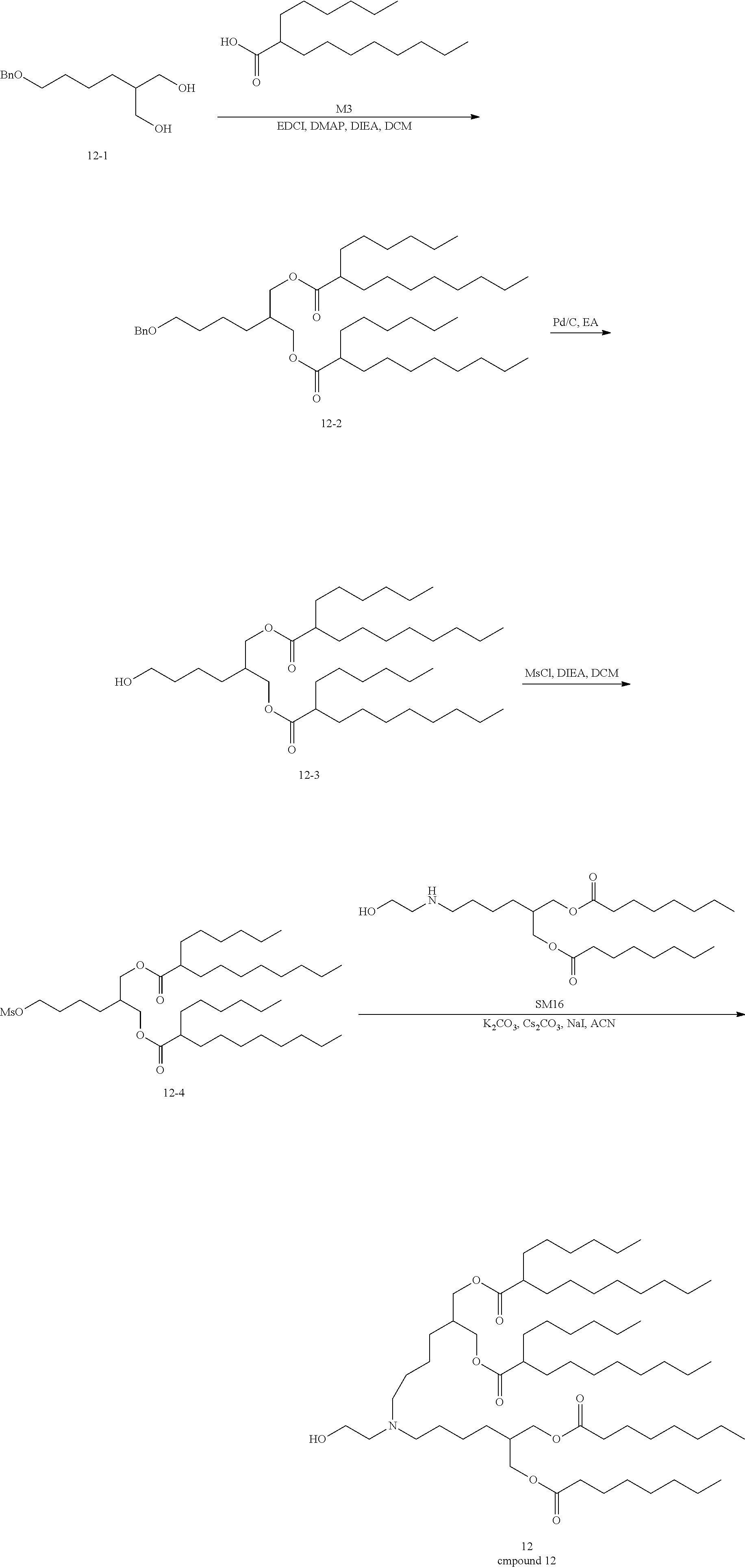

¹H NMR (400 MHz, CDCl₃) δ: 0.86-0.89 (m, 18H), 1.25-1.35 (m, 53H), 1.41-1.48 (m, 8H), 1.56-1.61 (m, 20H), 1.95-2.01 (m, 2H), 2.28-2.35 (m, 6H), 2.43-2.46 (m, 4H), 2.56-2.58 (m, 2H), 3.51-3.54 (m, 2H), 4.00-4.10 (m, 8H). LCMS: Rt: 0.080 min; MS m/z (ESI): 1050.8 [M+H]⁺.

### Preparation Example 02-7: Preparation of Compound 02-20 (i.e. Compound 20 in the Following Scheme)

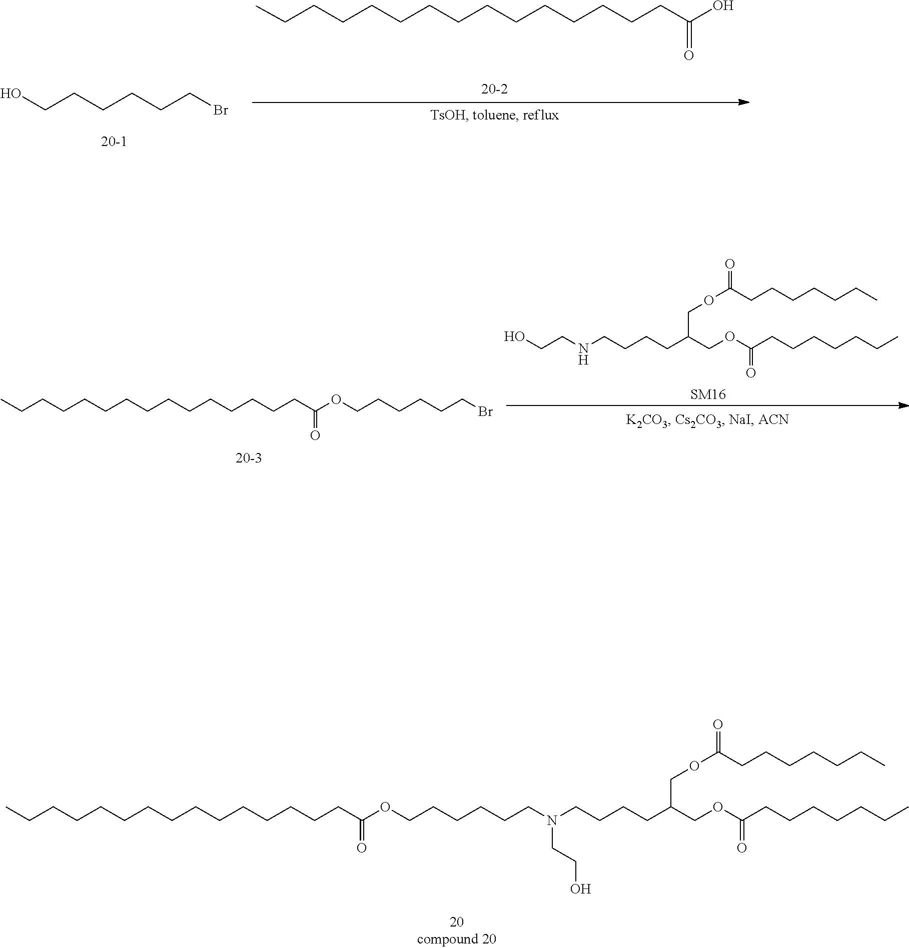 [SMILES: CCCCCCCCCCCCCCCC(=O)O ; OCCCCCCBr ; CCCCCCCC(=O)OCC(CCCCNCCO)COC(=O)CCCCCCC ; CCCCCCCCCCCCCCCC(=O)OCCCCCCBr ; CCCCCCCCCCCCCCCC(=O)OCCCCCCN(CCO)CCCCC(COC(=O)CCCCCCC)COC(=O)CCCCCCC]

¹H NMR (400 MHz, CDCl3) δ: 0.86-0.90 (m, 9H), 1.25-1.36 (m, 48H), 1.41-1.48 (m, 5H), 1.60-1.62 (m, 8H), 1.97-2.00 (m, 1H), 2.27-2.32 (m, 6H), 2.43-2.46 (m, 4H), 2.56-2.59 (m, 2H), 3.52-3.54 (m, 2H), 4.01-4.10 (m, 6H). LCMS: Rt: 0.093 min; MS m/z (ESI): 782.6 [M+H]⁺.

### Preparation Example 03: Preparation of Compounds of Series 03

Please refer to International Patent Application Publication No. WO 2022/152109, which is incorporated by reference in its entirety.

### Preparation Example 04: Preparation of Compounds of Series 04

Please refer to International Patent Application Publication No. WO 2022/247755, which is incorporated by reference in its entirety.

### Example 05: Preparation and Characterization of Lipid Nanoparticles

Briefly, cationic lipid (e.g. compound 01-1), DSPC, cholesterol, and PEG-lipid were solubilized in ethanol at a molar ratio as described herein, and mRNA were diluted in 10 to 50 mM citrate buffer, pH=4. The LNPs were prepared at a total lipid to mRNA weight ratio of approximately 10:1 to 30:1 by mixing the ethanolic lipid solution with the aqueous mRNA solution at a volume ratio of 1:3 using a microfluidic apparatus, total flow rate ranging from 9-30 mL/min. Ethanol was thereby removed and replaced by DPBS using dialysis. Finally, the lipid nanoparticles were filtered through a 0.2 m sterile filter.

Lipid nanoparticle size were determined by dynamic light scattering using a Malvern Zetasizer Nano ZS (Malvern UK) using a 173° backscatter detection mode. The encapsulation efficiency of lipid nanoparticles was determined using a Quant-it Ribogreen RNA quantification assay kit (Thermo Fisher Scientific, UK) according to the manufacturer's instructions.

As reported in literature, the apparent pKa of LNP formulations correlates with the delivery efficiency of LNPs for nucleic acids in vivo. The apparent pKa of each formulation was determined using an assay based on fluorescence of 2-(p-toluidino)-6-napthalene sulfonic acid (TNS). LNP formulations comprising of cationic lipid/DSPC/cholesterol/DMG-PEG (50/10/38.5/1.5 mol %) in PBS were prepared as described above. TNS was prepared as a 300 uM stock solution in distilled water. LNP formulations were diluted to 0.1 mg/ml total lipid in 3 mL of buffered solutions containing 50 mM sodium citrate, 50 mM sodium phosphate, 50 mM sodium borate, and 30 mM sodium chloride where the pH ranged from 3 to 9. An aliquot of the TNS solution was added to give a final concentration of 0.1 mg/ml and following vortex mixing fluorescence intensity was measured at room temperature in a Molecular Devices Spectramax iD3 spectrometer using excitation and mission wavelengths of 325 nm and 435 nm. A sigmoidal best fit analysis was applied to the fluorescence data and the pKa value was measured as the pH giving rise to half—maximal fluorescent intensity.

### Example B1: MRNA Synthesis and Purification

DNA Linearization. IVT plasmid pJ241 (constructed in house, contains a kanamycin resistance gene, a T7 promoter sequence, a poly(A) track, and a unique type-IIS restriction site downstream of poly(A) sequence) containing the target sequence (e.g., SEQ ID NO:4, 5, 7, 9, 11, or 13) encoding the mutant of the VZV gE protein (e.g., SEQ ID NO:3, 6, 8, 10, or 12), 5′-UTR (e.g., any one of SEQ ID NO:29-38), 3′-UTR (e.g., any one of SEQ ID NO:39-46) and polyA signal was linearized with type-IIS restriction enzyme digestion (full sequence of the constructs are shown in table 5). Every 10 μg of plasmid was mixed with 10 U of Esp3I/BsmBI, incubated at 37° C. for 4 hours to ensure complete linearization. The reaction was terminated by adding 1/10th volume of 3 M Na acetate (pH 5.5) and 2.5 volumes of ethanol, mix well and chill at −20° C. for 1 h. Linearized DNA was precipitated by centrifugation at 13800 g for 15 minutes at 4° C., washed twice with 70% ethanol, resuspended in nuclease-free H₂O.

In vitro Transcription of mRNA. Contents of a typical 20 μL reaction mixture are shown in the table below:

| Nuclease-free H₂O | Up to 20 μL | |
|---|---|---|
| RNase Inhibitor (40 U/μL) | 0.5 | μL |
| rNTP mixture (100 mM each) * | 8 μL (10 mM each final) | |
| 10X IVT Reaction Buffer | 2 | μL |
| 1M MgCl₂ | 0.8 | μL |
| 0.1M DTT | 2 | μL |
| 100 U/mL Pyrophosphatase Inorganic | 0.8 | μL |
| 100 mM NaCl | 1 | μL |
| Linearized DNA | 1 | μg |
| T7 RNA Polymerase (50 U/μL) | 2 | μL |

* UTP is replaced with pseudo-UTP and N1-Methyl-Pseudo-UTP for synthesis of modified mRNA.

The reaction mixture was incubated at 37° C. for 6 hours followed by addition of 1 μL of DNase I (RNase-free, 1 U/μL) to remove the DNA template, incubate for 30 minutes at 37° C. The synthesized RNA was purified by adding 0.5 volume of 7.5 M LiCl, 50 mM EDTA and incubating at −20° C. for 45 minutes, followed by centrifugation at 4° C. for 15 minutes at 13800 g to pellet the mRNA. Then the supernatant was removed and the pellet was rinsed twice with 500 μL of ice cold 70% ethanol, mRNA was resuspended in nuclease-free H₂O, adjusted concentration to 1 mg/mL, and stored at −20° C.

mRNA Capping. Each 10 μg uncapped mRNA was heated at 65° C. for 10 minutes, placed on ice for 5 minutes, and mixed with 10 U Vaccinia Capping Enzyme, 50 U mRNA Cap 2′-O-Methyltransferase, 0.2 mM SAM, 0.5 mM GTP and 1 U RNase inhibitor, and incubated at 37° C. for 60 minutes to generate cap 1 modification structure. The modified mRNA was precipitated by LiCl as previously described and the RNA was resuspended in nuclease-free H₂O, and stored at −20° C.

HPLC Purification. RNA was purified by high performance liquid chromatography (HPLC) using a C4 column (5 μm) (10 mm×250 mm column). Buffer A contained 0.1 M triethylammonium acetate (TEAA), pH=7.0 and Buffer B contained 0.1 M TEAA, pH=7.0 and 25% acetonitrile.

As expected, mRNA molecules were successfully produced by the in vitro transcription and maturation processes described above and were purified from the reaction system using HPLC.

### Example B2: In Vitro Transfection and Antigen Expression Analysis

Different mRNA molecules encoding the mutant of the VZV gE protein produced in Example B1 were transfected into expression cell lines such as HEK293 T cultured cells to evaluate the efficiency of in vitro expression of the mRNA molecules.

### Expression Analysis by FACS.

To assemble the mRNA-lipid complex, two separate Axygen® 1.5 mL microcentrifuge tubes (Corning, #MCT-150-C—S) were setup with 9 μL Lipofectamine®2000 (Invitrogen, #11668019) and 141 μL Opti-MEM™ I Reduced Serum Medium (Gibco, #11058021), and with 9 μg mRNA (or an equivalent volume of Opti-MEM™ I Reduced Serum Medium for the “Lipo only” blank control) and Opti-MEM™ I Reduced Serum Medium added to 150 μL in total, respectively. The contents of the two tubes were mixed together and incubated at room temperature for 5 minutes. One hundred microliter of the resultant complex was added to each well of a Costar®6-well clear TC-treated plate (Corning, #3516) containing HEK293T cells grown to ≥80% confluence and 3 mL fresh Opti-MEM™ I Reduced Serum Medium. The plate was incubated in humidified 5% CO₂ incubator at 37° C. for 24 hours.

The culture was transferred into a 15 mL centrifuge tube (Thermo, #339650), and centrifuged at 200×g for 5 minutes at room temperature. The cells were resuspended in PBS/1% BSA at a density of 1×10⁶ cells/mL. One hundred microliter of the cell suspension was added to each well of a Corning®96-well clear round bottom microplate (Corning, #3788). The microplate was centrifuged at 300×g for 5 minutes at 4° C., and the supernatant was discarded. One hundred microliter of the primary antibody mouse monoclonal antibody to VZV gE protein (abcam, #Ab272686) diluted in PBS/1% BSA to 10 g/mL or 1 g/mL was added. The microplate was incubated at 4° C. for 1 hour in dark. Then, the microplate was centrifuged at 300×g for 5 minutes at 4° C., and the supernatant was discarded. The cells were washed twice with 180 μL PBS/1% BSA each. One hundred microliter of the secondary antibody goat anti-mouse IgG antibody (FITC) (Jackson, #115-095-003) 1:100 diluted in PBS/1% BSA was added. The microplate was incubated at 4° C. for 30 minutes in dark. Then, the microplate was centrifuged at 300×g for 5 minutes at 4° C., and the supernatant was discarded. The cells were washed twice with 180 μL PBS/1% BSA each. Then, the cells were resuspended with 100 μL PBS/1% BSA, ready for FACS (BD, Fortessa).

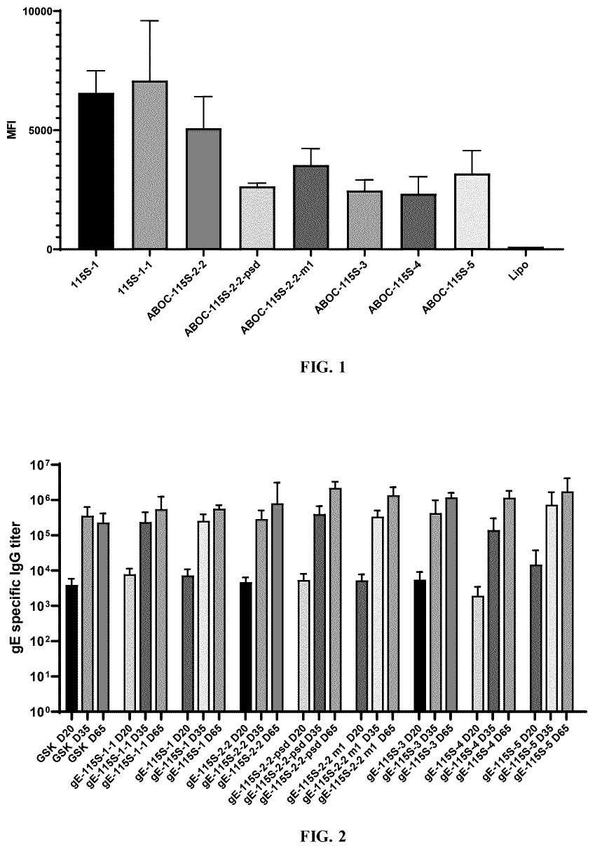

As shown in FIG. 1, in vitro transcribed mRNA molecules encoding the mutant of the VZV gE protein effectively transfected HEK293T cells. The transfected HEK293T cells expressed the encoded viral antigen on the cell membrane at a higher level than wild gE protein.

### Example B3. Preparation of mRNA-LNP Vaccine

mRNA-LNP vaccine was prepared according to the procedure provided in Example 05 above with a lipid prepared according to the procedure provided in Examples 01 to 04 above and mRNA prepared according to the procedure provided in Example B1 above.

### Example B4: Antigen Immunogenicity Analysis

The purpose of the following experiment was to evaluate the immunogenicity of the mutant of the VZV gE protein expressed from the gE-mRNA-LNP of the present invention.

On Day 0, female C57BL/6 mice aged 6-8 week and weighted 17.5-21.0 g (Lingchang Systems, Shanghai, China) were randomly assigned to each group with a minimal variation in average weight among groups (10 mice for each group). The mice were vaccinated on Days 0, 21 and 42 by intramuscular injection of 50 μL of a LNP formulation containing 0.03 μg/μL mRNA encoding the mutant of the VZV gE protein. Group 1 (Shingrix (GSK) positive control) was vaccinated on Days 0 and 21, and PBS was used as placebo. Blood samples were collected on Days 20, 35, 65 (before vaccination, where applicable), and spleens were collected on Day 35 from half of each group for Example B5.

Serum samples were collected on Days 20, 35, and 65 from the mice for determining the gE-specific IgG titers by ELISA. In this experiment, 100 μL of the coating solution (0.1 μg/ml VZV gE protein in PBS) (Bionintron, #B483001) was added to each well of a 96-well microplate (Costar, #42592), sealed, and incubated at 4° C. for 16 hrs. After three washings with 0.5% PBST, 200 μL of the blocking solution (5% BSA) was added to each well, sealed, and incubated at 37° C. for 1 hr. After washing three times, 100 μL of the diluted serum samples (serial dilution in 1% BSA with a factor of 4) or controls was added to each well, shaken gently to mix well, sealed, and incubated at 37° C. for 1 hr. After washing three times, 100 μL of the secondary antibody solution (Peroxidase-conjugated affinipure donkey-anti-mouse IgG, Jackson, #715-035-151) was added to each well, shaken gently to mix well, sealed, and incubated at 37° C. for 1 hr. Following three washings, 100 μL of the color developer solution (TMB substrate, Solarbio, #PR1200) was added to each well, shaken gently to mix well, and developed at room temperature for 3 min under protection from the light. After washing three times, 100 μL of the stop solution (Solarbio, #C1058) was added to each well, and shaken gently to mix well. The result was measured within 10 min. The wavelength of the microplate reader (SpectraMax ID5, Molecular Devices) was set at 450 nm. The maximum dilution factor for which the detection result was positive was selected, and titer result was the OD value of the positive maximum dilution factor/0.1*corresponding dilution factor.

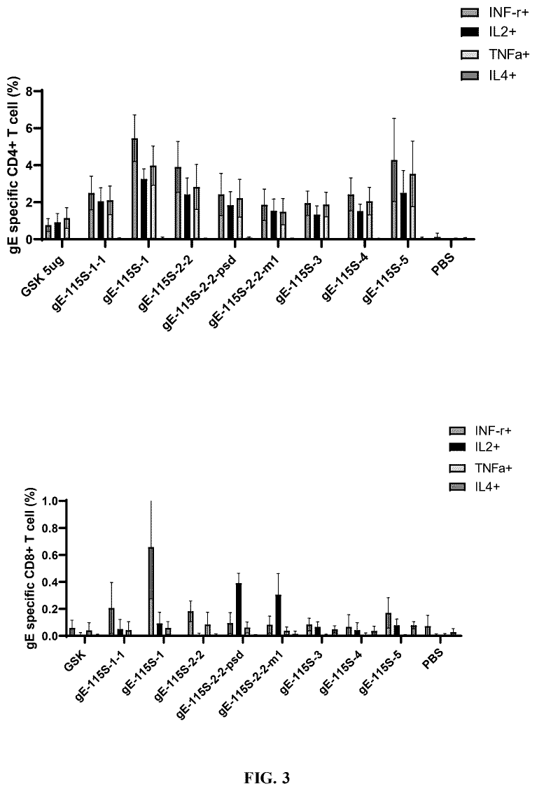

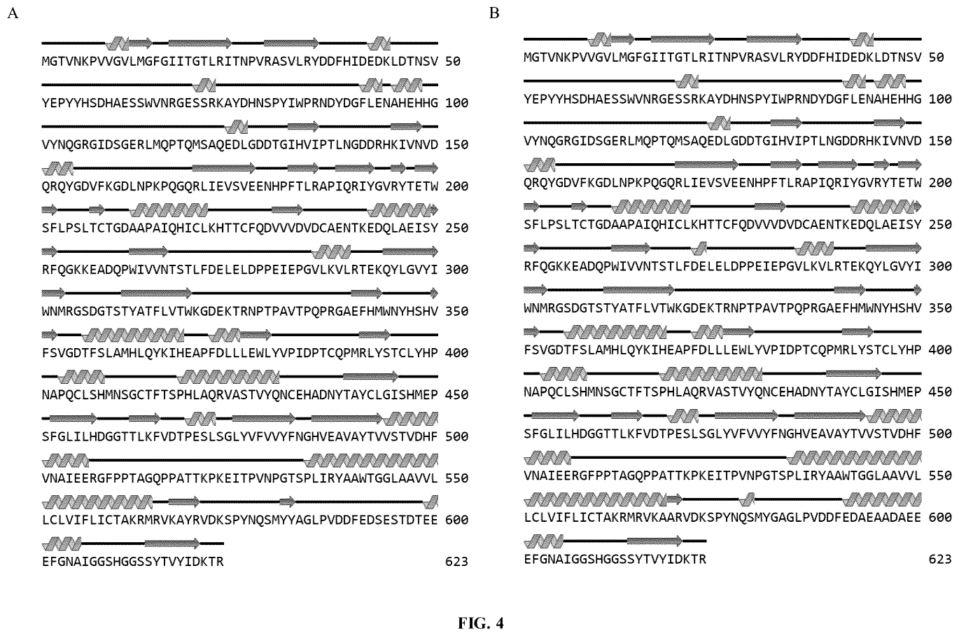

As shown in FIG. 2, all candidates stimulated robust humoral response and 115S-5 induced the highest antibody titer among the candidates and Shingrix (GSK) after each immunization.

### Example B5. Production of Cytokines in Mice Vaccinated with mRNA-LNP Vaccine

Single splenocyte was prepared from spleens and resuspended in complete medium (RMPI 1640 containing 2% FBS, Gibco, #22400-089, #10099141C) at 3×10⁷ cells/mL. 100 μL spleen cell suspension was added to each well of a 96-well culture plate (Corning, #3788). Then, 100 μL complete medium containing a final concentration of 2.5 g/mL of each peptide in a library of 113 15mer gE peptides, 2 g/mL anti-mCD28 antibody (Biolegend, cat #102102) and 2 g/mL anti-mCD49d antibody (BD, cat #553153) was added for stimulation. The plates were incubated for 2 hours in a 5% CO₂ incubator at 37° C. followed by addition of Brefeldin A (Biolegend, cat #420601) to a final concentration of 10 g/mL. The plates were incubated for additional 4 hours. For the positive control, 4 μL Pharmingen™ Leukocyte Activation Cocktail (BD Biosciences, #550583) was added for stimulation, and the plate was incubated for 6 hours in a 5% CO₂ incubator at 37° C.

The stimulated cells were washed with FACS buffer (PBS containing 2% FBS, Gibco, #10099141C) and resuspended in 100 μL antibody mixture per well.

For cell surface staining, 100 μL of an antibody mixture (Zombie green fixable viability kit, Biolegend, cat #423112; BV510-labelled anti-mCD45, Biolegend, cat #103138; Alexa Fluor 700-labelled anti-mCD3, Biolegend, cat #100216; PerCP/Cyanine5.5-labelled anti-mCD4, Biolegend, cat #100434; APC/Cyanine7-labelled anti-mCD8, Biolegend, cat #100714; all 1:50 diluted) was added into each well, and the cells were incubated at 4° C. for 30 minutes in dark. Then, the cells were washed twice with 200 μL FACS buffer per well.

For intracellular staining, cells were resuspended in 200 μL 1× Fixation/Permeabilization buffer (Thermo Fisher, #88-8824-00) and incubated at room temperature for 30 minutes in dark. The cells were washed twice with 2 mL 1× Permeabilization Buffer (Thermo Fisher, #88-8824-00) each. 100 μL antibody mixture (PE-labelled anti-mIFN-γ, Biolegend, cat #505808; APC-labelled anti-mIL-2, Biolegend, cat #503810; PE/Cyanine7-labelled anti-mIL-4, Biolegend, cat #504118; BV421-labelled anti-m TNF-α, Biolegend, cat #506328; all 1:50 diluted) was added, and the cells were incubated at room temperature for 30 minutes in dark. Then, the cells were washed twice with 200 μL FACS buffer each. Cells were resuspended in 200 μL FACS buffer for data acquisition using BD Fortessa flowcytometer.

The data of the stained samples was analyzed using Flowjo software for the proportion of CD4+ and CD8+ cells that express IL-2, IL-4, TNF-α or IFN-γ. The analysis showed that mRNA vaccine induced higher IL-2, TNF-α and IFN-γ expression than Shingrix with gE-115S-1 and gE-115S-5 showing the highest CD4+ T cell responses. The frequency of gE-specific CD8⁺ T cells was overall lower than that of CD4⁺ T cells. However, some gE-mRNA-LNP vaccines such as gE-115S-1-1, gE-115S-1, gE-115S-5 induced higher CD8+ T cell response than other candidate and Shingrix. There were almost no IL-4 secreting T cells detected, supporting that mRNA vaccine induced a strong Th1-based T cell response.

### Example B6. Screening of Codon-Optimized mRNA Sequences by Expression In Vitro

Different codon optimization mRNA molecules encoding the VZV gE variant protein were transfected into expression cell lines such as HEK293T cultured cells to evaluate expression efficiency in vitro.

### Expression Analysis by FACS

To assemble the mRNA-lipid complex, two separate Axygen® 1.5 mL microcentrifuge tubes (Corning, #MCT-150-C—S) were setup with 9 μL Lipofectamine®2000 (Invitrogen, #11668019) and 141 μL Opti-MEM™ I Reduced Serum Medium (Gibco, #11058021), and with 9 μg mRNA (or an equivalent volume of Opti-MEM™ I Reduced Serum Medium for the “Lipo only” blank control) and Opti-MEM™ I Reduced Serum Medium added to 150 μL in total, respectively. The contents of the two tubes were mixed together and incubated at room temperature for 5 minutes. 100 μL of the resultant complex was added to each well of a Costar® 6-well clear TC-treated plate (Corning, #3516) containing HEK293T cells grown to ≥80% confluence and 3 mL fresh Opti-MEM™ I Reduced Serum Medium. The plate was incubated in humidified 5% CO₂ incubator at 37° C. for 24 hours.

The culture was transferred into a 1.5 mL microcentrifuge tubes (Corning, #MCT-150-C—S), and centrifuged at 200×g for 5 minutes at room temperature. The cells were resuspended in PBS/1% BSA at a density of 1×10⁶ cells/mL. 100 μL of the cell suspension was added to each well of a Corning® 96-well clear round bottom microplate (Corning, #3788). The microplate was centrifuged at 300×g for 5 minutes at 4° C., and the supernatant was discarded. 100 μL of the primary antibody mouse monoclonal antibody to VZV gE protein (abcam, #Ab272686) diluted in PBS/1% BSA to 10 g/mL or 1 g/mL was added. The microplate was incubated at 4° C. for 1 hour in dark. Then, the microplate was centrifuged at 300×g for 5 minutes at 4° C., and the supernatant was discarded. The cells were washed twice with 180 μL PBS/1% BSA each. 100 μL of the secondary antibody goat anti-mouse IgG antibody (FITC) (Jackson, #115-095-003) 1:100 diluted in PBS/1% BSA was added. The microplate was incubated at 4° C. for 30 minutes in dark. Then, the microplate was centrifuged at 300×g for 5 minutes at 4° C., and the supernatant was discarded. The cells were washed twice with 180 μL PBS/1% BSA each. Then, the cells were resuspended with 100 μL PBS/1% BSA, ready for FACS (BD, Aria).

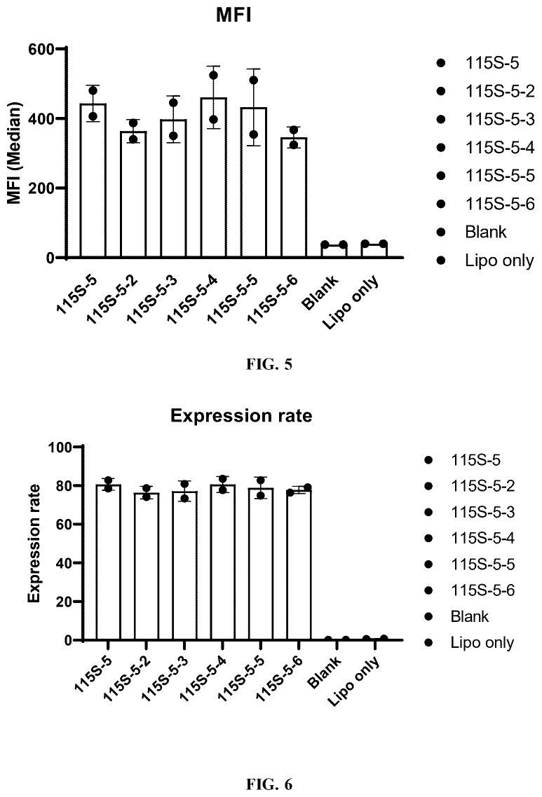

As shown in FIGS. 5 and 6, the mRNA molecules encoding the VZV gE protein effectively transfected HEK293T cells. All optimized mRNA sequences translated equivalent target protein.

### Example B7. Screening of Codon-Optimized Nucleic Acid Sequences by Immunogenicity

The purpose of the following experiment was to evaluate the immunogenicity of the different gE-mRNA-sequence capsulated by LNP.

On Day 0, female C57BL/6 mice aged 6-8 week were randomly assigned to each group with a minimal variation in average weight among groups (5 mice for each of Group). Mice were vaccinated on Day 0 and 21 by intramuscular injection with 50 μL of a LNP formulation containing 0.01 μg/μL mRNA encoding the VZV gE protein. Shingrix (GSK) was as positive control at the dose of 1.7 μg and PBS was vaccinated as placebo. 115S molecular was wild type gE protein, and 115S-5 shared a similar nuclear acids sequence with 115S except individual base mutations. Blood samples were collected on Days 20 and 29, and spleens were collected on Day 35 to analysis T cells response.

### 1. gE Specific IgG Titer Induced by Candidates

Serum samples were collected on Day 20 and 29 to analysis the gE-specific IgG titers by ELISA. 100 μL of the coating solution (0.1 g/ml VZV gE protein) (Bionintron #B483001) was added to each well of a microplate (Costar, #42592), sealed, and incubated at 4° C. for 16 hrs. After wash three times, 200 μL of the blocking solution (5% BSA) was added to each well, sealed, and incubated at 37° C. for 1 hr. After wash three times, 100 μL of the diluted samples (serial dilution in 1% BSA with a factor of 4) or controls was added to each well, shaken gently to mix well, sealed, and incubated at 37° C. for 1 hr. After wash three times, 100 μL of the secondary antibody solution (Peroxidase-conjugated affinipure donkey-anti-mouse IgG, Jackson, #715-035-151) was added to each well, shaken gently to mix well, sealed, and incubated at 37° C. for 1 hr. After wash three times, 100 μL of the TMB substrate (Solarbio, #PR1200) was added to each well, shaken gently to mix well, and incubated at room temperature for 3 min in dark. After wash three times, 100 μL of the stop solution (Solarbio, #C1058) was added to each well, and shaken gently to mix well. The result was measured within 10 min. The wavelength of the microplate reader (SpectraMax ID5, Molecular Devices) was set at 450 nm. The maximum dilution factor for which the detection result was positive was selected, and titer result was the OD value of the positive maximum dilution factor/0.1*corresponding dilution factor.

As shown in FIG. 7, all candidates could stimulate robust humoral response and the titer induced by 115S-5-5 was highest among the candidates.

### 2. Specific T Cells Response Induced by Candidates

Splenocytes were prepared from spleens and resuspended in complete medium (RMPI 1640, Gibco, #22400-089) at 2×10⁷ cells/mL. 100 μL spleen cell suspension was added to each well of a 96-well culture plate (Corning, #3788). Then, 100 μL complete medium containing 2.5 g/mL of a library of 113 15mer gE peptides, 2 g/mL anti-mCD28 antibody (Biolegend, cat #102102) and 2 g/mL anti-mCD49d antibody (BD, cat #553153) was added for stimulation, and the plate was incubated for 2 hours in a 5% CO₂ incubator at 37° C. Brefeldin A (Biolegend, cat #420601) was added to 10 μg/mL, and the plate was incubated for additional 4 hours. The positive control is Pharmingen™ Leukocyte Activation Cocktail (BD Biosciences, #550583). The stimulated cells were pelleted and resuspended in 3 mL PBS. One to two million live cells were transferred to a flow tube.

For cell surface staining, 100 μL of an antibody mixture (ef780-labelled live/dead marker, eBiosciences, cat #65-0865-18; PerCP/CY5.5-labelled anti-mCD45 Rat IgG2b, κ, clone 17A2, Biolegend, cat #103132; BUV395-labelled anti-mCD3 Rat IgG2b, κ, clone GK1.5, BD, cat #740268; BV421-labelled anti-mCD4 Rat IgG2b, κ, clone 53-6.7, Biolegend, cat #100438; PE-eFluor610-labelled anti-mCD8 Rat IgG2a, κ, clone 30-F11, eBiosciences, cat #61-0081-82; all 1:50 diluted) was added, and the cells were incubated at 4° C. for 30 minutes in dark. Then, the cells were washed twice with 2 mL PBS each. Cells were resuspended in 250 μL PBS, ready for FACS.

For intracellular staining, cells were resuspended in 200 μL 1× Fixation/Permeabilization buffer (Thermo Fisher, #88-8824-00) and incubated at room temperature for 30 minutes in dark. The cells were washed twice with 2 mL 1=× Permeabilization Buffer (Thermo Fisher, #88-8824-00) each. μL of an antibody mixture (PE-labelled anti-mIFN-γ, Rat IgG1, κ, clone XMG1.2, eBiosciences, cat #12-7311-82; APC-labelled anti-mIL-2, Rat IgG2b, κ, clone JES6-5H4, eBiosciences, cat #17-7021-82; FITC-labelled anti-mIL-4, Rat IgG1, κ, clone BVD6-24G2, eBiosciences, cat #11-7042-82; BV421-labelled anti-mTNF-α, Rat IgG1, κ, clone MP6-XT22, eBiosciences, cat #11-7042-82 all 1:50 diluted) was added, and the cells were incubated at room temperature for 30 minutes in dark. Then, the cells were washed twice with 2 mL PBA each. Cells were resuspended in 250 μL PBS, ready for FACS.

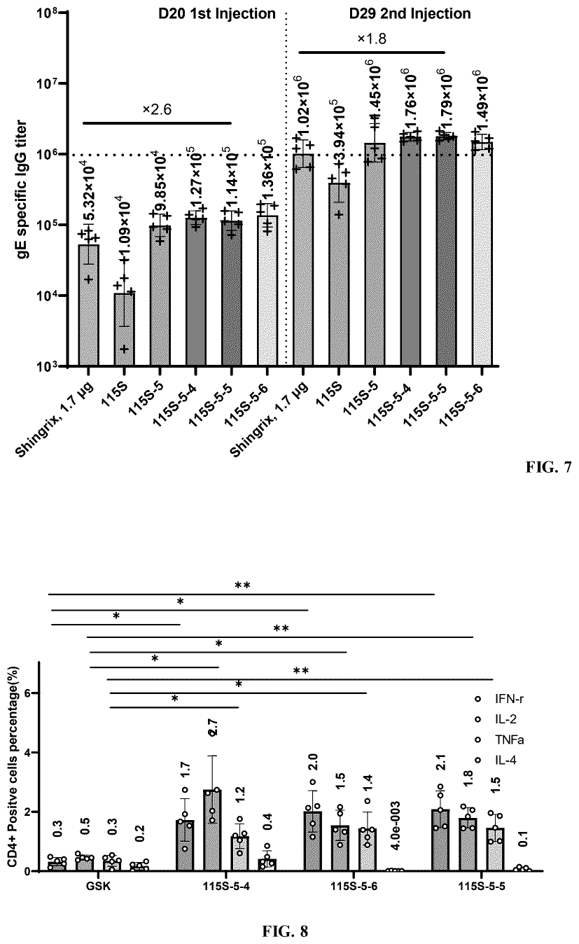

After staining, stimulated splenocytes were subjected to flow cytometry. The data was analyzed with Flowjo for the proportion of CD4+ cells expressing IL-2, IL-4, TNF-α or IFN-γ as FIG. 8. The highest IFN-γ+ response was observed in 115S-5-5 and other Th-1 indicated cytokines positive percentages in 115S-5-5 was acceptable. All cytokines positive percentages except IL-4 stimulated by mRNA vaccines showed higher than that by GSK.

### Example B8. Variant gE Antigen Distribution in 293T Cell

The gE protein's cytoplasmic region containing three specific motifs (motifs) provides signals for the protein's internalization through endocytosis and its targeting to the trans-Golgi Network (TGN), thereby promoting the assembly of VZV viral particles. These three motifs are YAGL (amino acids 582 to 585, capable of mediating the internalization of gE into the cell), AYRV (amino acids 568 to 571, which direct gE towards the TGN region), and SSTT (amino acids 588 to 601, which include a phosphorylation site; upon phosphorylation, this motif initiates related signaling pathways that also contribute to the endocytosis of gE protein). Employing confocal microscopy evaluated whether to increase the expression level of gE protein on the cell membrane by the 115S-5-5 mutation sites involving the three motifs above and further enhance its capacity to activate immunogenicity.

293T cells were transfected with the construct as description above. The transfected cells were stained with antibodies for gE (Abcam, Cat #Ab272686) and golgi markers GM130 (Ab cam, Cat #ab195303), and cell nucleus were stained with DAPI (Beyotime, Cat #P0131). Staining methods followed manufacture's protocol. Confocal microscopy was used to visualize the stained cells. FIG. 9 showed the results of higher level gE in the 115S-5-5 group was expressed on cell membrane than that in wild type construct.

### Example B9. Bridging Assay In Vitro Between Uridine Modification and Unmodification Molecular

### 1. Inflammatory Response Inhibition Experiment In Vitro

mRNA vaccine requires uridine chemistry modification and/or tuning of the production process to prevent activation of cellular innate immune sensors and a concomitant such as dsRNA reduction. The cytosolic RNA sensors retinoic acid-inducible gene I (RIG-I) bind to dsRNA and induce expression of proinflammatory cytokines such as IFN-β through activation of the adaptor protein mitochondrial antiviral signaling protein (MAVS). It is reported that the lowest stimulation of immune endpoints was with m1ψ made by the modified process, while mRNA containing canonical uridine was immunostimulatory regardless of process. Here using RIG-I signal reporting cell line and BJ cell lines to test RIG-I pathway activation and IFN-α release evaluate the inflammatory response induced by modified and unmodified mRNA vaccine.

115S-5-5 molecular uridines were replaced with N1-methyl-pseudouridine using abogen's innovation techniques to reduce inflammatory response. Particularly, as for the in vitro transcription of 115S-5-5 (m1ψ, cap analog:m7 GpppAmpU), UTP was replaced with m1ψ in the rNTP mixture, and the capping was performed by addition of m7 GpppAmpU according to the HiScribe T7 High Yield RNA Synthesis Kit (NEB, #E2040S) instructions to synthesize mRNA with a Cap1 “cap” structure.

BJ fibroblast cells were purchased from ATCC and cultured in EMEM medium supplemented with L-glutamine and 10% heat-inactivated fetal bovine serum (Gib co-FBS, Life Technologies, Thermofisher). The cells were seeded at a density of 20,000 cells per well into 96-well flat-bottomed cell culture plates (Corning) for 24 hours prior to transfection. mRNA was transfected into the cells using Lipofectamine 2000 (ThermoFisher Scientific). After a 48-hour transfection period, the cell culture supernatants were collected and the expression level of IFN—P was analyzed using an ELISA kit (R&D Systems).

The HEK-Lucia™ RIG-I cells were purchased from Invivogen and cultured in DMEM medium supplemented with L-glutamine and 10% heat-inactivated fetal bovine serum (Gibco-FBS) (Life Technologies, Thermo Fisher). The cells were seeded at a density of 50,000 cells per well into 96-well flat-bottom cell culture plates (Corning) and incubated for 24 hours prior to transfection. mRNA was transfected into the cells using Lipofectamine 2000 (Thermo Fisher Scientific). Approximately 24 hours post-transfection, 20 microliters of supernatant was aspirated and 50 microliters of QUANTILuc™4 Lucia/Gaussia Luciferase Detection Reagent (Invivogen) was added. The chemiluminescent signal was then detected using a microplate reader (MD).

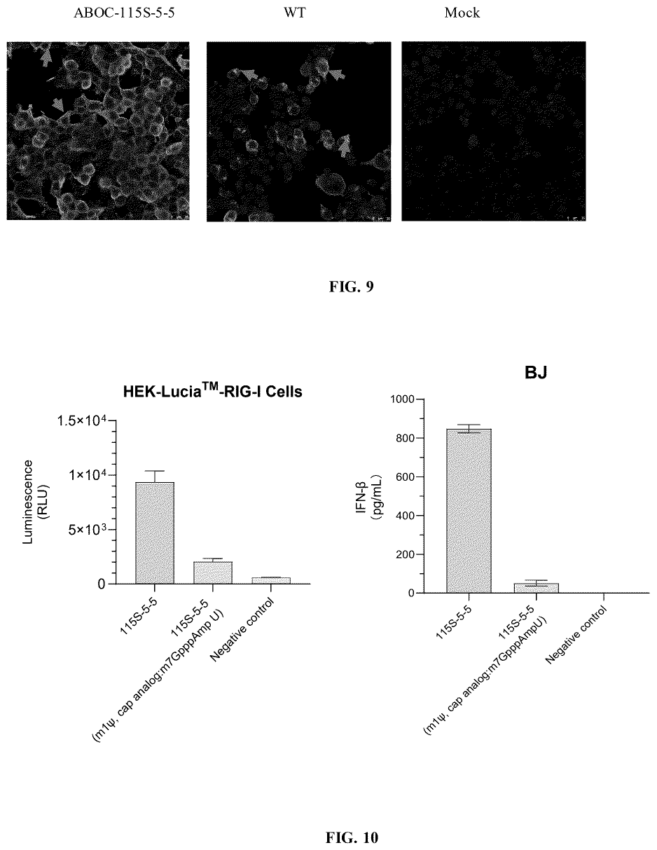

The results as FIG. 10 showed that the innovation modification could significantly reduce the level of RIG-I signaling pathway activation and decrease the release of the pro-inflammatory factor IFN-β after transfection with respective mRNAs.

### 2. Expression Level Bridging Between Modified and Unmodified Molecular In Vitro

To compare modified molecular expression level with unmodified molecular, both mRNA molecules were transfected respectively into HEK293T cell lines and analysis expression using FACS as described above.

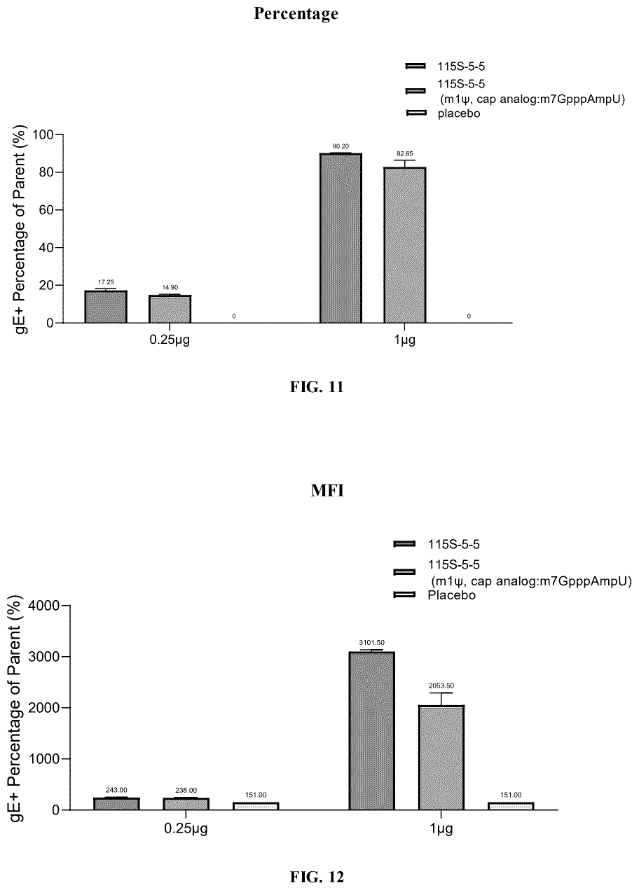

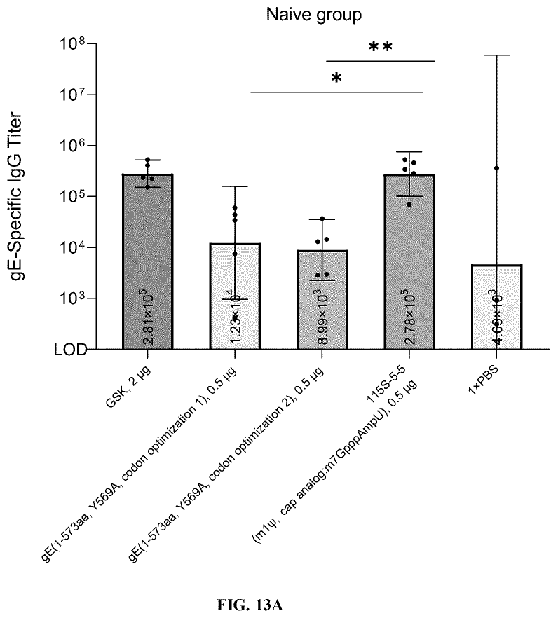

The results as FIGS. 11 and 12 showed that modified and unmodified molecular expressed equivalently antigen indicating no influence of modified bases on expression in vitro.

3. Immunology Response of Modified mRNA Vaccines In Vivo

The purpose of the following experiment was to compare the immunogenicity of the other mRNA vaccines with 115S-5-5 (m1ψ, cap analog:m7GpppAmpU). The assay was performed in LAV-experienced and naïve mice models respectively.

### Naïve Animal Model

On Day 0, female C57BL/6 mice aged 6-8 week were randomly assigned to each group with a minimal variation in average weight among groups (5 mice for each of Group). Mice were vaccinated on Days 0 and 14 by intramuscular injection of 50 μL at the dose of 0.5 μg. Shingrix (GSK) was as positive control at a dose of 1.7 μg and PBS was vaccinated as placebo. Moderna-128 and Moderna-178 means other company mRNA constructs. Blood samples were collected on Days 13 and 21, and spleens were collected on Day 28 to analysis T cells response.

### LAV-Experienced Animal Model

On Day 0, female C57BL/6 mice aged 6-8 week were randomly assigned to each group with a minimal variation in average weight among groups (5 mice for each of Group). Mice were immunized on Days 0 via s.c. with a live-attenuated varicella (LAV) vaccine (Ganwei, BCHT) using 1/10 human dose per mouse (2000 pfu), and mRNA vaccines at dose of 0.5 μg per mouse on Day 28 and 42. Shingrix (GSK) was as positive control at the dose of 1.7 μg and PBS was vaccinated as placebo. Blood samples were collected on Days 27, 41 and 49, and spleens were collected on Day 56 to analysis T cells response.

### 3.1 Serum Titers Analysis

Serum titers analysis protocol was as described above.

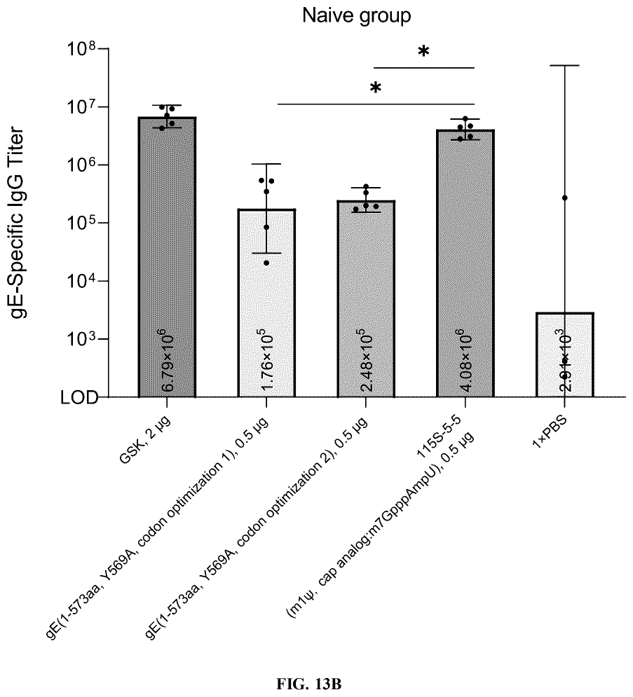

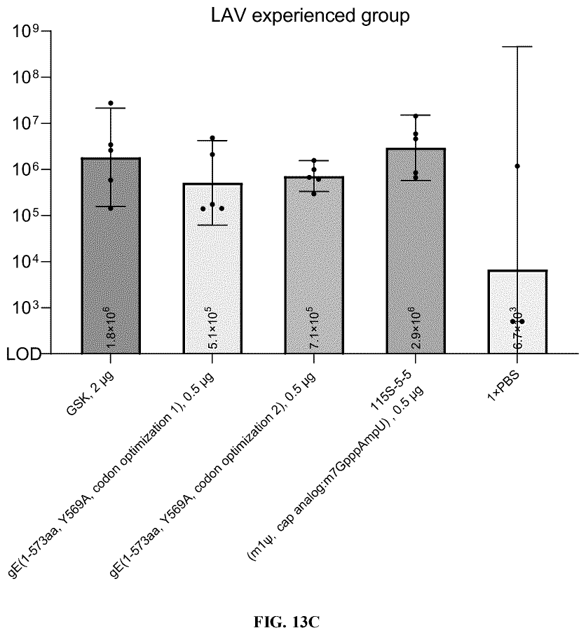

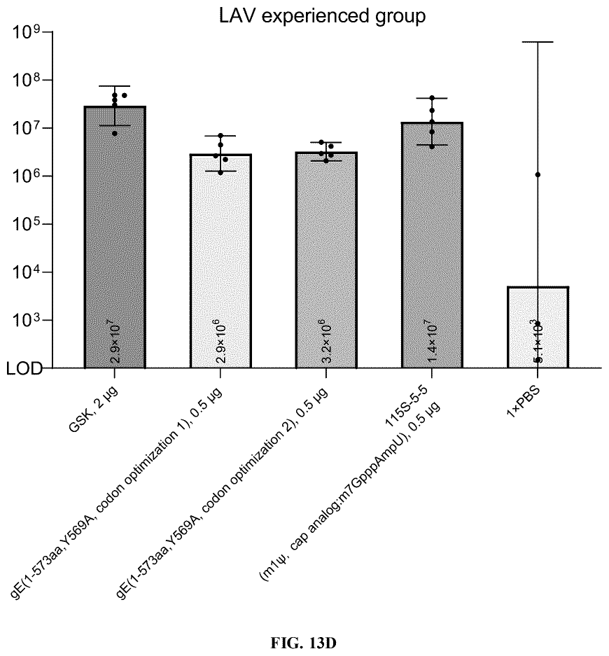

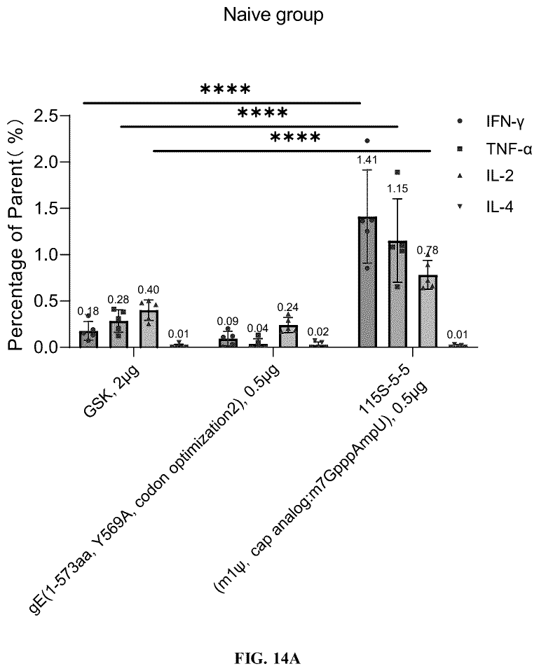

As shown in FIGS. 13A and 13B, IgG titers on Day 13 (Prime immunization) and Day 21 (Boost immunization) similarly reveal GMT in mice receiving 115S-5-5 was significantly higher than gE (1-573 aa, Y569A, codon optimization 1) and gE (1-573 aa, Y569A, codon optimization 2) in naïve mice.

As shown in FIGS. 13C and 13D, IgG titers on Day 34 (Prime immunization) and Day 49 (Boost immunization) showed a similar trend with titers in Naïve mice. GMT in 115S-5-5 group was higher than that in gE (1-573aa, Y569A, codon optimization 1) and gE (1-573aa, Y569A, codon optimization 2), but there was no significant difference.

### 3.2 T Cells Response Analysis

T cells response analysis protocol was as described above.

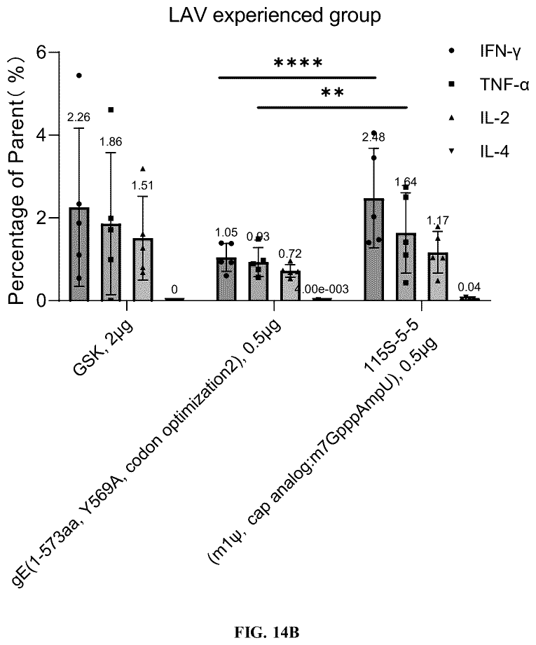

The data was analyzed with Flowjo for the proportion of CD4+ cells expressing IL-2, IL-4, TNF-α or IFN-γ as FIGS. 14A and 14B. The results revealed a strong Th-1 biased response indicated by IFN-γ, IL-2, TNF-α positive and IL-4 negative in groups receiving RNA vaccines in both animal models, the response induced by Shingrix was minimal in Naïve mice but robust in LAV-experienced mice. 115S-5-5 stimulated significant higher T cell response than gE (1-573aa, Y569A, codon optimization 2) in both animal models.

What is claimed:

1. A protein comprising a mutant of a mature glycoprotein E (gE) of varicella-zoster virus (VZV), wherein the mutant comprises:
(a) (i) a truncation of 37 amino acid residues from the C-terminus of the mature gE, and (ii) amino acid residue substitutions Y569A and Y582G, wherein amino acid residue positions 569 and 582 are the amino acid residue position numbers of full length VSV gE;
(b) amino acid residue substitutions Y569A and Y582G, wherein amino acid residue positions 569 and 582 are the amino acid residue position numbers of full length VSV gE;
(c) amino acid residue substitutions Y569A, Y582G, S593A, S595A, T596A, and T598A, wherein amino acid residue positions 569, 582, 593, 595, 596, and 598 are the amino acid residue position numbers of full length VSV gE;
(d) amino acid residue substitutions Y582G, S593A, S595A, T596A, and T598A, wherein amino acid residue positions 582, 593, 595, 596, and 598 are the amino acid residue position numbers of full length VSV gE; or
(e) (i) a truncation of 50 amino acid residues from the C-terminus of the mature gE protein, and (ii) amino acid residue substitution Y569A, wherein amino acid residue position 569 is the amino acid residue position number of full length VSV gE.

2. The protein of claim 1, wherein the mutant comprises the amino acid sequence of SEQ ID NO: 6, 8, 10, 12, or 3.

3. The protein of claim 1, wherein the amino acid sequence of the mutant consists of the amino acid sequence of SEQ ID NO: 6, 8, 10, 12, or 3.

4. The protein of claim 1, wherein the mutant comprises the amino acid sequence of SEQ ID NO: 6.

5. The protein of claim 1, wherein the amino acid sequence of the mutant consists of the amino acid sequence of SEQ ID NO: 6.

6. The protein of claim 1, wherein the mutant comprises an amino acid sequence that is at least 90%, at least 91%, at least 92%, at least 93%, at least 94%, or at least 95% identical to SEQ ID NO: 6, 8, 10, or 12.

7. The protein of claim 1, wherein the mutant comprises an amino acid sequence that is at least 96%, at least 97%, at least 98%, or at least 99% identical to SEQ ID NO: 6, 8, 10, or 12.

8. The protein of claim 1, wherein the mutant comprises an amino acid sequence that is at least 90%, at least 91%, at least 92%, at least 93%, at least 94%, or at least 95% identical to SEQ ID NO: 6.

9. The protein of claim 1, wherein the mutant comprises an amino acid sequence that is at least 96%, at least 97%, at least 98%, or at least 99% identical to SEQ ID NO: 6.

10. The protein of any one of claims 1 to 9, which further comprises a VZV gE signal peptide.

11. The protein of claim 10, wherein the signal peptide of the VZV gE comprises the amino acid sequence set forth in SEQ ID NO: 18.

12. The protein of claim 11, wherein the signal peptide of the VZV gE comprises an amino acid sequence that is at least 95%, at least 96%, at least 97%, at least 98%, or at least 99% identical to the amino acid sequence set forth in SEQ ID NO: 18.

13. The protein of any one of claims 1 to 9, which further comprises a heterologous signal peptide, wherein the N-terminal of the mutant is fused to the C-terminal of the heterologous signal peptide.

14. The protein of claim 13, wherein the heterologous signal peptide is a human IgE signal peptide.

15. The protein of claim 14, wherein the human IgE signal peptide comprises: (i) the amino acid sequence set forth in SEQ ID NO: 23, or (ii) an amino acid sequence that is at least 95%, at least 96%, at least 97%, at least 98%, or at least 99% identical to the amino acid sequence set forth in SEQ ID NO: 23.

16. The protein of claim 14, wherein the amino acid sequence of the human IgE signal peptide consists of the amino acid sequence set forth in SEQ ID NO: 23.

17. The protein of claim 14, wherein the amino acid sequence of the mutant consists of the amino acid sequence set forth in SEQ ID NO: 6, and the amino acid sequence of the human IgE signal peptide consists of the amino acid sequence set forth in SEQ ID NO: 23.

18. The protein of claim 14, which comprises the amino acid sequence set forth in SEQ ID NO: 59.

19. The protein of claim 14, wherein the amino acid sequence of the protein consists of the amino acid sequence set forth in SEQ ID NO: 59.

20. The protein of claim 13, wherein the heterologous signal peptide is a human tPA signal peptide.

21. The protein of claim 20, wherein the human tPA signal peptide comprises: (i) the amino acid sequence set forth in SEQ ID NO: 27, or (ii) an amino acid sequence that is at least 95%, at least 96%, at least 97%, at least 98%, or at least 99% identical to the amino acid sequence set forth in SEQ ID NO: 27.

22. The protein of any one of claims 1 to 21, wherein the mature gE comprises the amino acid sequence set forth in SEQ ID NO: 1 and/or the full length VZV gE comprises the amino acid sequence set forth in SEQ ID NO: 55.

23. A fragment of a mature glycoprotein E (gE) of varicella-zoster virus (VZV), wherein the fragment comprises a truncation of at least one and at most 50, 49, 48, 47, 46, 45, 44, 43, or 42 amino acid residues from the C-terminal of the mature gE, optionally the truncation is a truncation of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues from the C-terminal of the mature gE, or optionally the truncation is a truncation of 11, 12, 13, 14, 15, 16, 17, 18, 34, 35, 36, 37, 38, 39, 40, 41, 42, 43, or 44 amino acid residues from the C-terminal of the mature gE, or optionally the truncation is a truncation of 14 or 37 amino acid residues from the C-terminal of the mature gE.

24. The fragment of claim 23, wherein the fragment further comprises the amino acid residue substitution Y569A, and wherein amino acid residue position 569 is the amino acid residue position number according to the full length VZV gE.

25. The fragment of claim 23 or 24, wherein the fragment comprises a truncation of at most 41, 40, 39, 38, 37, 36, 35, 34, 33, 32, or 31 amino acid residues from the C-terminal of the mature gE.

26. The fragment of claim 25, wherein the fragment further comprises the amino acid residue substitution Y582G, and wherein amino acid residue position 582 is the amino acid residue position number according to the full length VZV gE protein.

27. The fragment of any one of claims 23 to 26, wherein the fragment comprises a truncation of at most 30 or 29 amino acid residues from the C-terminal of the mature gE.

28. The fragment of claim 27, wherein the fragment further comprises the amino acid residue substitution S593A, and wherein amino acid residue position 593 is the amino acid residue position number according to the full length VZV gE.

29. The fragment of any one of claims 23 to 28, wherein the fragment comprises a truncation of at most 28 amino acid residues from the C-terminal of the mature gE.

30. The fragment of claim 29, wherein the fragment comprises the amino acid residue substitution S595A, and wherein amino acid residue position 595 is the amino acid residue position number according to the full length VSV gE.

31. The fragment of any one of claims 23 to 30, wherein the fragment comprises a truncation of at most 27 or 26 amino acid residues from the C-terminal of the mature gE.

32. The fragment of claim 31, wherein the fragment further comprises the amino acid residue substitution T596A, and wherein amino acid residue position 596 is the amino acid residue position number according to the full length VZV gE.

33. The fragment of any one of claims 23 to 32, wherein the fragment comprises a truncation of at most 25, 24, 23, 22, 21, 20, 19, 18, 17, 16, 15, 14, 13, 12, 11, 10, 9, 8, 7, 6, 5, 4, 3, or 2 amino acid residues or at most one amino acid residue from the C-terminal of the mature gE.

34. The fragment of claim 33, wherein the fragment further comprises the amino acid residue substitution T598A, and wherein amino acid residue position 598 is the amino acid residue position number according to the full length VZV gE.

35. The fragment of claim 23, wherein the fragment comprises a truncation of 37 amino acid residues from the C-terminal of the mature gE.

36. The fragment of claim 23, wherein the fragment comprises: (1) a truncation of 37 amino acid residues from the C-terminal of the mature gE, and (2) the amino acid residue substitutions Y569A and Y582G, and wherein amino acid residue positions 569 and 582 are amino acid residue position numbers according to the full length VZV gE.

37. The fragment of any one of claims 23 to 36, wherein the mature gE comprises the amino acid sequence set forth in SEQ ID NO: 1 and/or the full length VZV gE comprises the amino acid sequence set forth in SEQ ID NO: 55.

38. The fragment of any one of claims 23 to 37, wherein the fragment comprises an amino acid sequence having at least 80%, 85%, 90%, 91%, 92%, 93%, 94%, 95%, 96%, 97%, 98%, or 99% identity with the amino acid sequence set forth in SEQ ID NO: 6, 3, 8, 10, or 12.

39. A fusion protein comprising the fragment of any one of claims 23 to 38, and a heterologous signal peptide, wherein the N-terminal of the fragment is fused to the C-terminal of the heterologous signal peptide.

40. The fusion protein of claim 39, wherein the heterologous signal peptide is a human tPA signal peptide.

41. The fusion protein of claim 40, wherein the human tPA signal peptide comprises the amino acid sequence set forth in SEQ ID NO: 27.

42. The fusion protein of claim 39, wherein the heterologous signal peptide is a human IgE signal peptide.

43. The fusion protein of claim 42, wherein the human IgE signal peptide comprises the amino acid sequence set forth in SEQ ID NO: 23.

44. A fusion protein comprising a fragment of a mature glycoprotein (gE) of varicella-zoster virus (VZV) and a human IgE signal peptide, wherein the fragment comprises a truncation of 37 amino acid residues from the C-terminal of the mature gE, and the N-terminal of the fragment is fused to the C-terminal of the human IgE signal peptide.

45. A nucleic acid encoding the protein of any of claims 1 to 22, fragment of any one of claims 23 to 38, or the fusion protein of any one of claims 39 to 44.

46. The nucleic acid of claim 45, which comprises a nucleotide sequence set forth in SEQ ID NO: 7, 9, 11, 13, 4, or 5, or a nucleotide sequence having at least 80%, 85%, 90%, 91%, 92%, 93%, 94%, 95%, 96%, 97%, 98%, or 99% identity with the nucleotide sequence set forth in SEQ ID NO: 7, 9, 11, 13, 4 or 5.

47. A nucleic acid encoding the protein of any one of claims 14 to 19, or the fusion protein of any one of claims 42 to 44, wherein the nucleotide sequence encoding the IgE signal peptide comprises: (a) the nucleotide sequence set forth in SEQ ID NO: 24, 25, or 26, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 24, 25, or 26.

48. A nucleic acid encoding the protein of any one of claims 10 to 12, wherein the nucleotide sequence of the signal peptide comprises: (a) the nucleotide sequence set forth in SEQ ID NO: 19, 20, 21, or 22, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 19, 20, 21, or 22.

49. A nucleic acid encoding the protein of claim 20 or 21, or the fusion protein of claim 40 or 41, wherein the nucleotide sequence encoding a human tPA signal peptide comprises: (a) the nucleotide sequence set forth in SEQ ID NO: 28, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 28.

50. A non-naturally occurring nucleic acid comprising a coding nucleotide sequence encoding the protein of any one of claims 1 to 22, the fragment of any one of claims 23 to 38, or the fusion protein of any one of claims 39 to 44.

51. The non-naturally occurring nucleic acid of claim 50, wherein the coding nucleotide sequence has been codon optimized for expression in cells of a subject, optionally, wherein the subject is a non-human mammal or a human.

52. The non-naturally occurring nucleic acid of claim 50 or 51, which comprises: (a) the nucleotide sequence set forth in SEQ ID NO: 7, 9, 11, 13, 4 or 5, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 7, 9, 11, 13, 4 or 5.

53. A non-naturally occurring nucleic acid encoding the protein of any one of claims 14 to 19, or the fusion protein of any one of claims 42 to 44, wherein the nucleotide sequence encoding the IgE signal peptide comprises: (a) the nucleotide sequence set forth in SEQ ID NO: 24, 25, or 26, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 24, 25, or 26.

54. A non-naturally occurring nucleic acid encoding the protein of any one of claims 10 to 12, wherein the nucleotide sequence of the signal peptide of the full length VSV gE protein comprises: (a) the nucleotide sequence set forth in SEQ ID NO: 19, 20, 21, or 22, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 19, 20, 21, or 22.

55. A non-naturally occurring nucleic acid encoding the protein of claim 20 or 21, or the fusion protein of claim 40 or 41, wherein the nucleotide sequence encoding a human tPA signal peptide comprises: (a) the nucleotide sequence set forth in SEQ ID NO: 28, or (b) a nucleotide sequence that is at least 80%, 85%, 90%, 91%, 92%, 93%, 94%, 95%, 96%, 97%, 98%, or 99% identical to the nucleotide sequence set forth in SEQ ID NO: 28.

56. A non-naturally occurring nucleic acid comprising the nucleotide sequence of SEQ ID NO: 63, 7, 51, 60, 62, 62, 64, 9, 11, 13, 52, 53, 54, 58, 49, 50, 4, or 5.

57. The non-naturally occurring nucleic acid of claim 56, which consists of the nucleotide sequence of SEQ ID NO: 63, 7, 51, 60, 62, 62, 64, 9, 11, 13, 52, 53, 54, 58, 49, 50, 4, or 5.

58. The non-naturally occurring nucleic acid of any one of claims 50 to 55, wherein the nucleic acid consists of, consists essentially of, or comprises: (1) the nucleotide sequence set forth in SEQ ID NO: 63, 7, 51, 60, 62, 62, 64, 9, 11, 13, 52, 53, 54, 58, 49, 50, 4, or 5; or (2) a nucleotide sequence having at least 80%, 85%, 90%, 91%, 92%, 93%, 94%, 95%, 96%, 97%, 98%, or 99% identity with the nucleotide sequence set forth in SEQ ID NO: 63, 7, 51, 60, 62, 62, 64, 9, 11, 13, 52, 53, 54, 58, 49, 50, 4, or 5.

59. The non-naturally occurring nucleic acid of any one of claims 50 to 58,
further comprising a 5′ untranslated region (5′-UTR), wherein the 5′-UTR comprises the nucleotide sequence set forth in any one of SEQ ID NOS: 29-38,
and/or,
further comprising a 3′ untranslated region (3′-UTR), wherein the 3′-UTR comprises the nucleotide sequence set forth in any one of SEQ ID NOS: 39-46,
optionally, wherein the 3′-UTR further comprises a poly-A tail or a polyadenylation signal.

60. The non-naturally occurring nucleic acid of any one of claims 50 to 59, wherein the nucleic acid is DNA.

61. The non-naturally occurring nucleic acid of any one of claims 50 to 60, wherein the nucleic acid comprises one or more functional nucleotide analogs.

62. The non-naturally occurring nucleic acid of any one 50 to 59, wherein the nucleic acid is mRNA, and wherein thymines are substituted for uracils or a functional analog thereof in the nucleic acid.

63. The non-naturally occurring nucleic acid of claim 62, wherein the nucleic acid comprises one or more functional nucleotide analogs that are selected from pseudouridine, 1-methyl-pseudouridine and 5-methylcytosine.

64. The non-naturally occurring nucleic acid of claim 62, wherein the nucleic acid consists of, consists essentially of, or comprises: (1) the nucleotide sequence set forth in SEQ ID NO: 63, or (2) a nucleotide sequence that is at least 80%, 85%, 90%, 91%, 92%, 93%, 94%, 95%, 96%, 97%, 98%, or 99% identical to the nucleotide sequence set forth in SEQ ID NO: 63; except that all thymines (T) are substituted for uracil (U) or N1-methylpseudouridine, and/or the first nucleotide G is substituted for m⁷ GpppAmpU.

65. The non-naturally occurring nucleic acid of claim 62, wherein the nucleic acid consists of, essentially consists of, or comprises the nucleotide sequence set forth in SEQ ID NO: 63, except that all thymines (T) are substituted for uracil (U) or N1-methylpseudouridine, and/or the first nucleotide G is substituted for m⁷ GpppAmpU.

66. The non-naturally occurring nucleic acid of claim 62, wherein the nucleic acid consists of, essentially consists of or comprises the nucleotide sequence set forth in SEQ ID NO: 63, except that all thymines (T) are substituted for N1-methylpseudouridine, and the first nucleotide G is substituted for m⁷ GpppAmpU.

67. A vector comprising the nucleic acid of any one of claims 45 to 49, or the non-naturally occurring nucleic acid of any one of claims 50 to 66; preferably, an IVT (In Vitro Transcription) plasmid.

68. A host cell comprising the nucleic acid of any one of claims 45 to 49, the non-naturally occurring nucleic acid of any one of claims 50 to 66, or the vector of claim 67.

69. The host cell of claim 68, which is in vitro, ex vivo, or isolated.

70. A pharmaceutical composition comprising the protein of any one of claims 1 to 22, the fragment of any one of claims 23 to 38, or the fusion protein of any one of claims 39 to 44.

71. A pharmaceutical composition comprising the nucleic acid of any one of claims 45 to 49, the non-naturally occurring nucleic acid of any one of claims 50 to 66, or the vector of claim 67.

72. A pharmaceutical composition comprising the non-naturally occurring nucleic acid of any one of claims 50 to 66, and at least a first lipid,
optionally, wherein the first lipid is a compound according to Formula 01-I or Formula 01-II; or a compound listed in Table 01-1; or a compound according to Formula 02-I; or a compound listed in Table 02-1; or a compound according to Formula 03-I; or a compound listed in Table 03-1; or a compound according to Formula 04-I; or a compound listed in Table 04-1.

73. The pharmaceutical composition of claim 72, further comprising a second lipid, optionally, wherein the second lipid is a compound according to Formula 05-I.

74. The pharmaceutical composition of claim 72 or 73, formulated as lipid nanoparticles encapsulating the nucleic acid in a lipid shell.

75. The pharmaceutical composition of any one of claims 70 to 74, wherein the composition is a vaccine.

76. A method for managing, preventing or treating a disease or disorder caused by VZV or by infection with VZV in a subject, comprising administering to the subject a therapeutically effective amount of the protein of any one of claims 1 to 22, the fragment of any one of claims 23 to 38, the fusion protein of any one of claims 39 to 44, the nucleic acid of any one of claims 45 to 49, the non-naturally occurring nucleic acid of any one of claims 50 to 66, the vector of claim 67, or the pharmaceutical composition of any one of claims 70 to 75.

77. The method of claim 76, wherein the method is for preventing a disease or disorder caused by VZV or by infection with VZV in the subject.

78. The method of claim 76 or 77, wherein an immune response against the VZV is elicited in the subject.

79. The method of claim 78, wherein the immune response comprises production of cytokine in lymphocytes.

80. The method of claim 78 or 79, wherein the immune response comprises increased proportion of cytokine-expressing lymphocytes.

81. The method of claim 79 or 80, wherein the lymphocytes are CD4⁺ T cells and/or CD8⁺ T cells, and/or, wherein the cytokine is one or more of IFN-γ, IL-2, and TNF-α.

82. The method of any one of claims 79 to 81, wherein the production of cytokine in lymphocytes is increased.

83. The method of any one of claims 78 to 82, wherein the immune response comprises production of an antibody that specifically binds to VZV gE.

84. The method of any one of claims 76 to 83, wherein the disease or disorder caused by VZV is
(a) varicella and/or zoster;
(b) postherpetic neuralgia (PHN); and/or
(c) one or more of meningoencephalitis, myelitis, cranial nerve palsy, angiopathy, keratitis, retinopathy, ulcers, hepatitis and pancreatitis.

85. The method of any one of claims 76 to 84, wherein the subject is a human.

86. The method of claim 85, wherein the human is a human adult.

87. The method of claim 86, wherein the adult is at least 40 years old.

88. The method of claim 85, wherein the human is an elderly human.

89. A protein as defined in any one of claims 1 to 22, a fragment as defined in any one of claims 23 to 38, a fusion protein as defined in any one of claims 39 to 44, a nucleic acid as defined in any one of claims 45 to 49, a non-naturally occurring nucleic acid as defined in any one of claims 50 to 66, a vector as defined in claim 67, or a pharmaceutical composition as defined in any one of claims 70 to 75, for use in a method for managing, preventing or treating a disease or disorder caused by VZV or by infection with VZV in a subject.

90. The protein, fragment, fusion protein, nucleic acid, non-naturally occurring nucleic acid, vector, or pharmaceutical composition for use according to claim 89, wherein the subject is human, optionally a human adult or elderly human.

91. Use of the protein as defined in any one of claims 1 to 22, the fragment as defined in any one of claims 23 to 38, the fusion protein as defined in any one of claims 39 to 44, the nucleic acid as defined in any one of claims 45 to 49, the non-naturally occurring nucleic acid as defined in any one of claims 50 to 66, the vector as defined in claim 67, or the pharmaceutical composition as defined in any one of claims 70 to 75, for the manufacture of a medicament for managing, preventing or treating a disease or disorder caused by VZV or by infection with VZV in a subject.

92. The use of claim 91, wherein the subject is human, optionally a human adult or elderly human.
